(19) United States
(12) Patent Application Publication
(10) Pub. No.: US 2021/0300900 A1
(43) Pub. Date: Sep. 30, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andrés Avelino Trabanco-Suárez, Olias del Rey (ES); Francisca Delgado-Jiménez, Madrid (ES); Ana Isabel De Lucas Olivares, Bargas (ES); Juan Antonio Vega Ramiro, Olias del Rey (ES)

(21) Appl. No.: 17/253,446

(22) PCT Filed: Jun. 20, 2019
(86) PCT No.: PCT/EP2019/066390

§ 371 Date: Dec. 17, 2020

(30) Foreign Application Priority Data
Jun. 20, 2018 (EP) 18382450.7

(51) Int. Cl. C07D 401/14; C07D 409/14; C07D 413/14; C07D 403/14

(52) U.S. Cl. CPC C07D 401/14; C07D 403/14; C07D 413/14; C07D 409/14

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

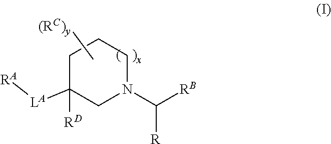

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA)O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in the N-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying O-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (AD) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors; WO2016/0300443 (Asceneuron S.A., published 3 Mar. 2016), WO2017/144633 and WO2017/0114639 (Asceneuron S.A., published 31 Aug. 2017) disclose 1,4-disubstituted piperidines or piperazines as OGA inhibitors; WO2017/144637 (Asceneuron S. A, published 31 Aug. 2017) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors; WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

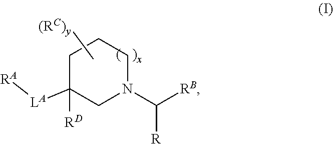

and the tautomers and the stereoisomeric forms thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —O—, —CH₂—, —OCH₂—, —CH₂O—, —NH—, —N(CH₃)—, —NH—CH₂—, and —CH₂—NH—;
x represents 0 or 1;

### R is H or CH₃; and

R^{B} is a radical selected from the group consisting of (b-1) to (b-4)

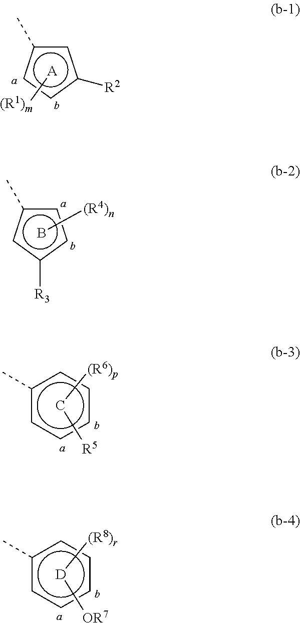

wherein
m, n, p and r each represent 0 or 1;
ring A represents a 5-membered heteroaromatic selected from the group consisting of 1H-pyrazolyl, imidazolyl, isoxazolyl and thienyl;
R¹ when present, is C₁₋₄alkyl, bound at position a or b of the A ring;
R² is selected from the group consisting of C₁₋₄alkyl, C₃₋₆cycloalkyl, —NR^{a}R^{aa}, —NR^{a}COC₁₋₄alkyl, and —CONR^{a}R^{aa}; wherein R^{a} represents hydrogen or C₁₋₄alkyl; and
R^{aa} is C₁₋₄alkyl;
ring B represents a 5-membered heteroaromatic selected from the group consisting of oxazolyl, thiazolyl, imidazolyl, 1H-pyrazolyl, isoxazolyl and thienyl; wherein
R³ is —OC₁₋₄alkyl or —C₁₋₄alkyloxyC₁₋₄alkyl;
R⁴ when present, is a halo substituent bound to a carbon atom at position a or b of the B ring, or is a C₁₋₄alkyl substituent bound to a Nitrogen atom at position a or b of the B ring;
rings C and D each represent a 6-membered heteroaromatic selected from the group consisting of pyridinyl, pyridazinyl, pyrazinyl, and pyrimidinyl; wherein
R⁵ is bound at position a or b and is selected from the group consisting of C₁₋₄alkyl optionally substituted with 1, 2, or 3 independently selected halo substituents;
C₃₋₆cycloalkyl; —NR^{b}COC₁₋₄alkyl; and —CONR^{b}R^{bb}; wherein R^{b} represents hydrogen or C₁₋₄alkyl; and R^{bb} is C₁₋₄alkyl;
R⁶, when present, is C₁₋₄alkyl;
OR⁷ is bound at position a or b, wherein R⁷ is C₁₋₄alkyl or C₃₋₆cycloalkyl;
R⁸ when present, is halo or C₁₋₄alkyl, bound to a carbon atom;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D}; and
c) R^{D} is not hydroxy or methoxy when L^{A} is —O—, —OCH₂—, —CH₂O—, —NH—, —N(CH₃)—, —NHCH₂— or —CH₂NH—;
   and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or may be useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as defined hereinbefore, and the tautomers and the stereoisomeric forms thereof, wherein R^{A} is a heteroaryl radical selected from the group consisting of pyridin-2-yl, pyridin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents.

In a further particular embodiment, the invention is directed to compounds of Formula (I) as defined hereinbefore, and the tautomers and the stereoisomeric forms thereof, wherein R^{A} is a heteroaryl radical selected from the group consisting of pyridin-4-yl, and pyrazin-2-yl, each of which may be optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents.

In a further particular embodiment, the invention is directed to compounds of Formula (I) as defined hereinbefore, and the tautomers and the stereoisomeric forms thereof, wherein R^{A} is pyridin-4-yl optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents.

In a further particular embodiment, the invention is directed to compounds of Formula (I) as defined hereinbefore, and the tautomers and the stereoisomeric forms thereof, wherein R^{A} is pyridin-4-yl optionally substituted with 1, 2 or 3 substituents each independently selected from the group consisting of C₁₋₄alkyl optionally substituted with 1, 2, or 3 independently selected halo substituents; and C₁₋₄alkyloxy optionally substituted with 1, 2, or 3 independently selected halo substituents.

In a further particular embodiment, the invention is directed to compounds of Formula (I) as defined hereinbefore, and the tautomers and the stereoisomeric forms thereof, wherein L^{A} is selected from the group consisting of a —O—, —CH₂—, —OCH₂—, —CH₂O—, —NH—, —N(CH₃)—, —NH—CH₂—, and —CH₂—NH—.

In a particular embodiment, the invention is directed to compounds of Formula (I) as defined hereinbefore, and the tautomers and the stereoisomeric forms thereof, wherein L^{A} is selected from the group consisting of a covalent bond, —O—, —CH₂—, —NH—CH₂—.

In an additional embodiment, the invention is directed to compounds of Formula (I) as defined hereinbefore, and the tautomers and the stereoisomeric forms thereof, wherein L^{A} is selected from the group consisting of —O—, —CH₂—, —NH—CH₂—.

In a further embodiment, the invention is directed to compounds of Formula (I) as defined hereinbefore, and the tautomers and the stereoisomeric forms thereof, wherein L^{A} is selected from the group consisting of —CH₂—, —O—CH₂—, and —NH—CH₂—.

In a further embodiment, the invention is directed to compounds of Formula (I) as defined hereinbefore, and the tautomers and the stereoisomeric forms thereof, wherein L^{A} is selected from the group consisting of —CH₂—, —NH—CH₂—.

In a further embodiment, the invention is directed to compounds of Formula (I) as defined hereinbefore, and the tautomers and the stereoisomeric forms thereof, wherein L^{A} is —CH₂—.

In a further embodiment, the invention is directed to compounds of Formula (I) as defined hereinbefore, and the tautomers and the stereoisomeric forms thereof, wherein R^{B} is (b-1), (b-2), (b-3) or (b-4), wherein

m, n, and r each represent 0 or 1;
ring A represents a 5-membered heteroaromatic selected from the group consisting of 1H-pyrazolyl, imidazolyl, and thienyl; wherein
R¹ when present, is C₁₋₄alkyl bound to a Nitrogen atom at position a or b;
R² is selected from the group consisting of C₁₋₄alkyl, C₃₋₆cycloalkyl, —NR^{a}R^{aa}, and —NR^{a}COC₁₋₄alkyl; wherein
R^{a} represents hydrogen or C₁₋₄alkyl; and R^{aa} is C₁₋₄alkyl;
ring B represents a 5-membered heteroaromatic selected from the group consisting of oxazolyl, thiazolyl, imidazolyl, 1H-pyrazolyl, and isoxazolyl; wherein
R³ is —OC₁₋₄alkyl or —C₁₋₄alkyloxyC₁₋₄alkyl;
R⁴ when present, is a halo substituent bound to a carbon atom at position a or b or is a C₁₋₄alkyl substituent bound to a Nitrogen atom at position a or b;
rings C and D each represent a 6-membered heteroaromatic selected from the group consisting of pyridinyl, pyridazinyl, pyrazinyl, and pyrimidinyl;
R⁵ is bound at position a or b and is selected from the group consisting of —NR^{b}COC₁₋₄alkyl and —CONR^{b}R^{bb}; wherein R^{b} represents hydrogen or C₁₋₄alkyl; and R^{bb} is C₁₋₄alkyl;
OR⁷ is bound at position a or b, wherein R⁷ is C₁₋₄alkyl; and
R⁸ when present, is a halo or C₁₋₄alkyl substituent, bound to a carbon atom.

In a further embodiment, the invention is directed to compounds of Formula (I) as defined hereinbefore, and the tautomers and the stereoisomeric forms thereof, wherein R^{B} is (b-1), (b-2), (b-3a) or (b-4a)

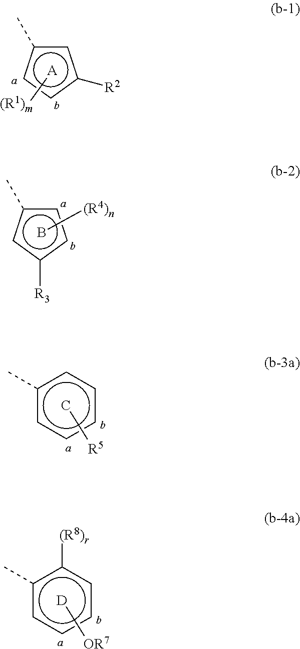

wherein
m, n, and r each represent 0 or 1;
ring A represents a 5-membered heteroaromatic selected from the group consisting of 1H-pyrazolyl, imidazolyl, and thienyl;
R¹ when present, is C₁₋₄alkyl, bound to a Nitrogen atom at position a or b;
R² is selected from the group consisting of C₃₋₆cycloalkyl, and —NR^{a}COC₁₋₄alkyl; wherein R^{a} represents hydrogen or C₁₋₄alkyl;
ring B represents a 5-membered heteroaromatic selected from the group consisting of oxazolyl, thiazolyl, and imidazolyl; wherein
R³ is —OC₁₋₄alkyl;
R⁴ when present, is a halo substituent bound to a carbon atom at position a or b, or is a C₁₋₄alkyl substituent bound to a Nitrogen atom at position a or b;
rings C and D each represent pyridinyl; wherein
R⁵ is bound at position a or b and is selected from the group consisting of —NR^{b}COC₁₋₄alkyl and —CONR^{b}R^{bb}; wherein R^{b} represents hydrogen or C₁₋₄alkyl; and R^{bb} is C₁₋₄alkyl;
OR⁷ is bound at position a or b, wherein R⁷ is C₁₋₄alkyl; and
R⁸ when present, is halo, bound to a carbon atom.

In a further embodiment, the invention is directed to compounds of Formula (I), as referred to herein, and the tautomers and the stereoisomeric forms thereof, wherein R^{B} is (b-2), wherein ring B represents a 5-membered heteroaromatic selected from the group consisting of imidazolyl, 1H-pyrazolyl, isoxazolyl and thienyl; wherein

R³ is —OC₁₋₄alkyl or —C₁₋₄alkyloxyC₁₋₄alkyl; and R⁴ when present, is a halo substituent bound to a carbon atom at position a or b of the B ring, or is a C₁₋₄alkyl substituent bound to a Nitrogen atom at position a or b of the B ring.

In a further embodiment, the invention is directed to compounds of Formula (I), as referred to herein, and the tautomers and the stereoisomeric forms thereof, wherein R^{B} is (b-1), (b-2), (b-3a) or (b-4a), wherein m, and r each represent 0 or 1; and n is 0.

In a further embodiment, the invention is directed to compounds of Formula (I), as referred to herein, and the tautomers and the stereoisomeric forms thereof, wherein R^{B} is (b-1), (b-2), (b-3a) or (b-4a), wherein m, and r each represent 0 or 1; and n and p are each 0; wherein ring A represents 1H-pyrazol-3-yl, or thiophen-3-yl; ring B represents 1H-imidazol-2-yl or oxazol-2-yl; and rings C and D represent pyridin-2-yl or pyridin-3-yl; R¹ is —C₁₋₄alkyl, in particular methyl; R² is C₃₋₆cycloalkyl or —NR^{a}COC₁₋₄alkyl, wherein R^{a} is hydrogen or methyl, in particular R² is cyclopropyl or —NHC(═O)CH₃; R³ is —OC₁₋₄alkyl, in particular —OCH₃; R⁵ is bound at position a and is —CONR^{b}R^{bb}; wherein R^{b} is hydrogen or methyl, in particular R³ is —C(═O)NHCH₃; —OR⁷ is bound at position a and is C₁₋₄alkyl, in particular CH₃; and R⁸ is halo, in particular fluoro.

In a further embodiment, the invention is directed to compounds of Formula (I), as referred to herein, and the tautomers and the stereoisomeric forms thereof, wherein R^{B} is (b-1), (b-2), (b-3a) or (b-4a), wherein m, and r each represent 0 or 1; and n is 0; wherein ring A represents 1H-pyrazol-3-yl, or thiophen-3-yl; ring B represents 1H-imidazol-2-yl or oxazol-2-yl; and rings C and D represent pyridin-2-yl or pyridin-3-yl.

In a further embodiment, the invention is directed to compounds of Formula (I), as referred to herein, and the tautomers and the stereoisomeric forms thereof, wherein R^{D} is hydrogen, and the pharmaceutically acceptable salts and the solvates thereof.

In a further embodiment, the invention is directed to compounds of Formula (I), as referred to herein, and the tautomers and the stereoisomeric forms thereof, wherein y is 0, and the pharmaceutically acceptable salts and the solvates thereof.

In a further embodiment, the invention is directed to compounds of Formula (I), as referred to herein, and the tautomers and the stereoisomeric forms thereof, having the following stereoconfirguration

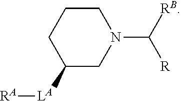

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C₁₋₄alkyl is as defined before, “C₃₋₆cycloalkyl” shall denote a saturated, cyclic hydrocarbon radical having from 3 to 6 carbon atoms, such as cyclopropyl, cyclobutyl, cyclopentyl, and cyclohexyl. A particular C₃₋₆cycloalkyl group is cyclopropyl. When reference is made to L^{A}, the definition is to be read from left to right, with the left part of the linker bound to R^{A} and the right part of the linker bound to the pyrrolidinediyl or piperidinediyl ring. Thus, when L^{A} is, for example, —O—CH₂—, then R^{A}-L^{A}- is R^{A}—O—CH₂—. When R^{C} is present more than once, where possible, it may be bound at the same carbon atom of the pyrrolidinediyl or piperidinediyl ring, and each instance may be different.

In general, whenever the term “substituted” is used in the present invention, it is meant, unless otherwise indicated or is clear from the context, to indicate that one or more hydrogens, in particular 1 to 3 hydrogens, preferably 1 or 2 hydrogens, more preferably 1 hydrogen, on the atom or radical indicated in the expression using “substituted” are replaced with a selection of substituents from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (+)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds of Formula (I) can be prepared by reacting an intermediate compound of Formula (II) with a compound of Formula (III) according to reaction scheme (1). The reaction is performed in a suitable reaction-inert solvent, such as, for example, dichloromethane or 1,2-dichloroethane, a metal hydride, such as, for example sodium triacetoxyborohydride, sodium cyanoborohydride or sodium borohydride and may require the presence of a suitable base, such as, for example, triethylamine or diisopropylethylamine, and/or a Lewis acid, such as, for example titanium tetraisopropoxide, under thermal conditions, such as, 0° C. or room temperature, or 80° C., for example for 1 hour or 24 hours. In reaction scheme (1) all variables are defined as in Formula (I).

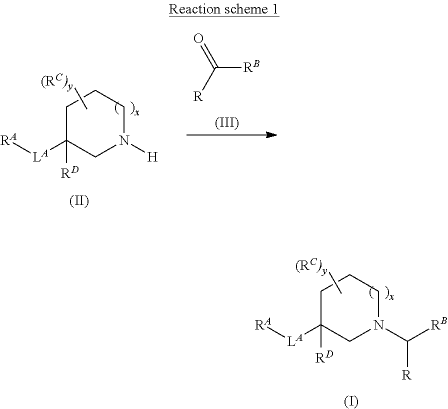

### Experimental Procedure 2

In particular, final compounds of Formula (I) wherein R^{B} is (b-1) wherein ring A is 1H-pyrazolyl, m is 0, and R² is NHCH₂CH₃, herein referred to as (I-b), can be prepared by reacting final compounds of Formula (I), wherein R^{B} is (b-1) wherein ring A is 1H-pyrazolyl, m is 0, and R² is NH(CO)CH₃, herein referred to as (I-a), with a suitable reducing agent such as lithium aluminium hydride, in a suitable reaction-inert solvent, such as, for example, anhydrous tetrahydrofuran, under thermal conditions, such as, 0° C. to room temperature, for example at 0° C. or at room temperature, for a sufficient period of time to bring the reaction to completion, for example for 1 hour to 24 hours according to reaction scheme (2). In reaction scheme (2) all variables are defined as in Formula (I).

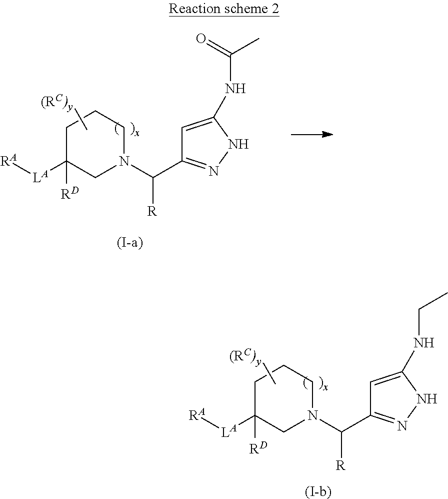

### Experimental Procedure 3

The final compounds of Formula (I-a) can be prepared by cleaving a protecting group in intermediate compounds of Formula (IV) according to reaction scheme (3). In reaction scheme (3) all variables are defined as in Formula (I), and PG is a suitable protecting group of the nitrogen function such as, for example, dimethylaminesulphonamide, 2-(trimethylsilyl)ethoxymethyl (SEM), tert-butoxycarbonyl (Boc), ethoxycarbonyl, benzyl, benzyloxycarbonyl (Cbz). Suitable methods for removing such protecting groups are widely known to the person skilled in the art and comprise but are not limited to: SEM deprotection: treatment with a protic acid, such as, for example, trifluoroacetic acid, in a reaction inert solvent, such as, for example, dichloromethane; Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol. In reaction scheme (3) all variables are defined as in Formula (I).

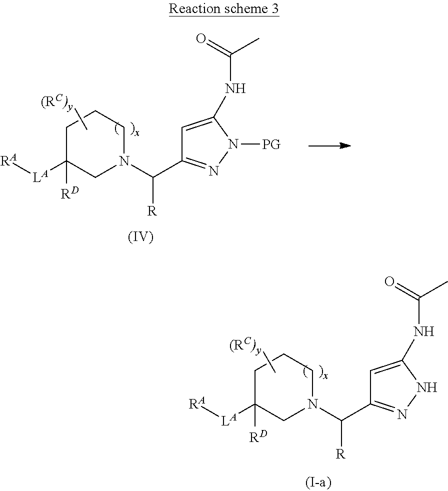

### Experimental Procedure 4

Additionally, final compounds of Formula (I) can be prepared by reacting an intermediate compound of Formula (II) with a compound of Formula (V) followed by reaction of the formed imine derivative with and intermediate compound of Formula (VI) according to reaction scheme (4). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. to room temperature, for example at 0° C. or at room temperature, for a sufficient time to bring the reaction to completion, for example for 1 hour to 24 hours. In reaction scheme (4) all variables are defined as in Formula (I), and wherein halo is chloro, bromo or iodo.

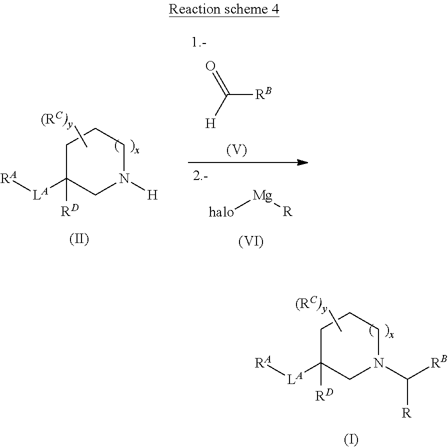

### Experimental Procedure 5

Additionally, final compounds of Formula (I) wherein R^{B} is (b-3) wherein R⁵ is a —NH(CO)C₁₋₄alkyl substituent at position a, herein referred to as (I-c), can be prepared by reacting an intermediate compound of Formula (VII) according to reaction scheme 5. The reaction is performed in the presence of an acylating reagent of Formula (VIII) such as alkyl anhydride in the presence of a suitable reaction-inert solvent, such as for example, 1,4-dioxane, under thermal conditions, such as, 0° C. to room temperature, for example at 0° C. or room temperature, for a sufficient period of time, for example for 1 hour to 24 hours. In reaction scheme (5) all variables are defined as in Formula (I) and wherein R⁵ is defined as NR^{b}COC₁₋₄alkyl.

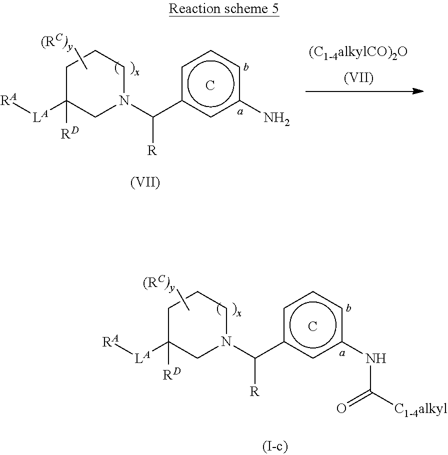

### Experimental Procedure 6

Additionally, final compounds of Formula (I-c) can be prepared in two steps by reacting an intermediate compound of Formula (IX) according to reaction scheme 6. The reaction is performed first by reducing a compound of Formula (IX) in the presence of a reducing agent such as iron in the presence of a salt such as aqueous solution of ammonium chloride in the presence of a suitable reaction-inert solvent, such as for example, mixture of ethanol and tetrahydrofuran, under thermal conditions, such as, 0° C. to room temperature, for example at 0° C. or at room temperature, for a sufficient period of time, for example for 1 hour to 24 hours. In a second step final compounds of Formula (I-c) can be prepared by reacting a compound of Formula (VII) with an acylating reagent of Formula (VIII) such as an alkyl anhydride, in the presence of a base, such as triethylamine, in the presence of a suitable reaction-inert solvent, such as for example, dichloromethane, under thermal conditions, such as, 0° C. to room temperature, for example at 0° C. or room temperature, for a sufficient period of time, for example for 1 hour to 24 hours. In reaction scheme (6) all variables are defined as in Formula (I). and wherein R⁵ is defined as NR^{b}COC₁₋₄alkyl.

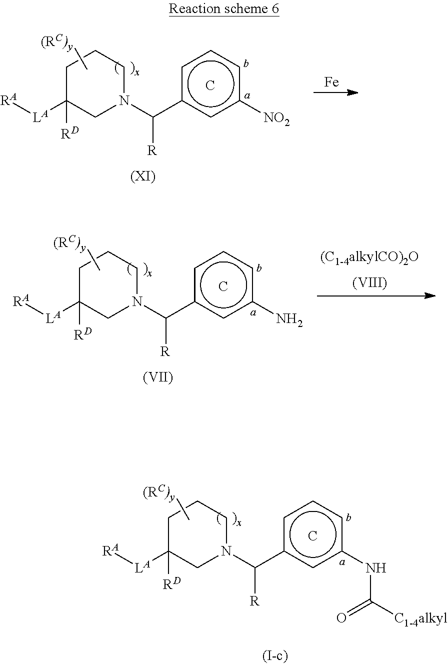

### Experimental Procedure 7

Additionally, final compounds of Formula (I-c) can be prepared by reacting an intermediate compound of Formula (X) with a compound of Formula (XI) according to reaction scheme (7). The reaction is performed in the presence of a palladium catalyst, such as, for example, palladium(II)acetate, a ligand, such as, for example bis[(2-diphenylphosphino)phenyl] ether, DPEPhos, in the presence of an amine, such as for example, methylamine, a base, such as, for example cesium hydroxy hydrate, in a suitable reaction-inert solvent, such as, for example, anhydrous toluene, under thermal conditions, such as, 110° C., for example for 4 hour to 24 hours. In reaction scheme (7) all variables are defined as in Formula (I), and wherein halo is chloro, bromo or iodo

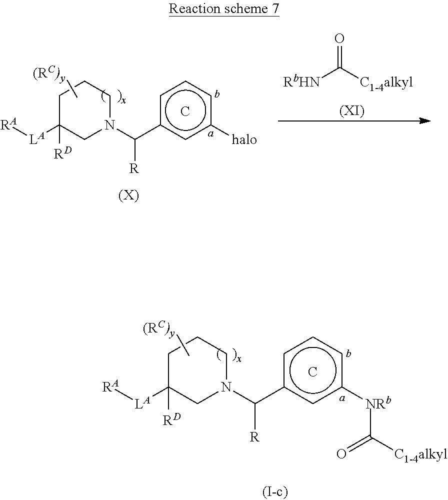

### Experimental Procedure 8

Final compounds of Formula (I), wherein R^{B} is (b-3), wherein R⁵ is a C(O)NR^{b}R^{bb} substituent at the a position, herein referred to as (I-d), can be prepared by reacting an intermediate compound of Formula (X) according to reaction scheme (8). The reaction is performed by a carbonylation reaction in the presence of a palladium catalyst, such as, for example palladium(II)acetate, a ligand, such as, for example, bis[(2-diphenylphosphino)phenyl] ether, DPEPhos, in the presence of cesium hydroxide hydrate and an amine such as methylamine in a suitable reaction-inert solvent, such as, for example, anhydrous toluene, under thermal conditions, such as, 95° C., for a sufficient time, for example for 4 hour to 24 hours. In reaction scheme (8) all variables are defined as in Formula (I), and wherein halo is chloro, bromo or iodo.

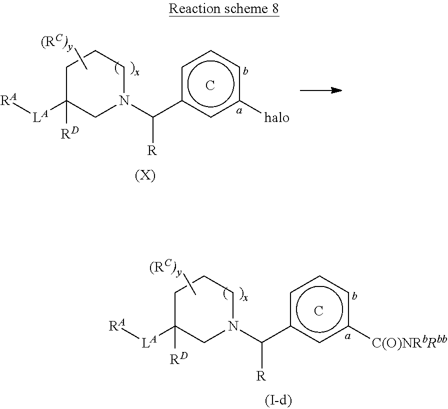

### Experimental Procedure 9

Additionally, final compounds of Formula (I), wherein L^{A} is —NHCH₂—, herein referred to as (I-e) can be prepared by reacting an intermediate compound of Formula (XI) with a compound of Formula (XII) according to reaction scheme (9). The reaction is performed in the presence of a palladium catalyst, such as, for example tris(dibenzylideneacetone)dipalladium(0), a ligand, such as, for example 2-dicyclohexylphosphino-2′-(N,N-dimethylamino)biphenyl, a base, such as, for example sodium tert-butoxide, a suitable reaction-inert solvent, such as, for example, anhydrous 1,4-dioxane, under thermal conditions, such as, 100° C., for example for 4 hour or 24 hours. In reaction scheme (9) all variables are defined as in Formula (I), and wherein halo is chloro, bromo or iodo.

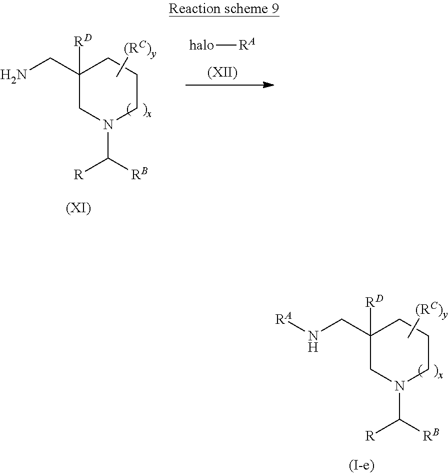

### Experimental Procedure 10

In particular, final compounds of Formula (I), wherein R^{A} is a 4-pyridinyl substituted with two independently selected C₁₋₄alkyl substituents, optionally fluorinated, such as CF₃, and L^{A} is —NHCH₂—, herein referred to as (I-f), can be prepared by reacting an intermediate compound of Formula (XIII) with a boronic acid derivative of Formula (XIV), C₁₋₄alkyl-B(OR^{x})₂, wherein each R^{X} is H, OH, or C₁₋₄alkyl, or wherein the two instances of R^{x} are taken together to form for example a bivalent radical of formula —CH₂CH₂—, —CH₂CH₂CH₂— or —C(CH₃)₂C(CH₃)₂—, or alternatively a cyclic derivative (R^{y}OB)₃, wherein R^{y} is hydrogen, hydroxy or methyl, such as trimethylboraxine, according to reaction scheme (10). The reaction is performed in the presence of a palladium catalyst, such as, for example palladium acetate, a ligand, such as, for example tricyclohexylphosphine tetrafluoroborate, a base, such as, for example potassium carbonate, a suitable reaction-inert solvent, such as, for example, anhydrous 1,4-dioxane, under thermal conditions, such as, 100° C., for example for 4 hour to 24 hours. In reaction scheme (10) all variables are defined as in Formula (I), and wherein halo is chloro, bromo or iodo.

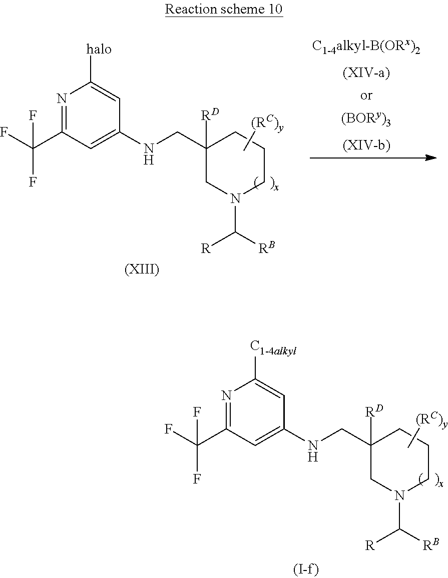

### Experimental Procedure 11

Intermediate compounds of Formula (II) can be prepared cleaving a protecting group in an intermediate compound of Formula (XV) according to reaction scheme (11). In reaction scheme (11) all variables are defined as in Formula (I), and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known to the person skilled in the art and comprise but are not limited to: Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

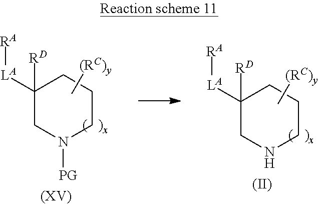

### Experimental Procedure 12

Intermediate compounds of Formula (XV), wherein L^{A} is CH₂, herein referred to as (XV-a) can be prepared by “Negishi coupling” reaction of a halo compound of Formula (XVI) with an organozinc compound of Formula (XVII) according to reaction scheme (12). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl, under thermal conditions, such as, for example, room temperature, for example for 1 hour. In reaction scheme (12) all variables are defined as in Formula (I), and halo is preferably bromo or iodo. PG is defined as in Formula (IV).

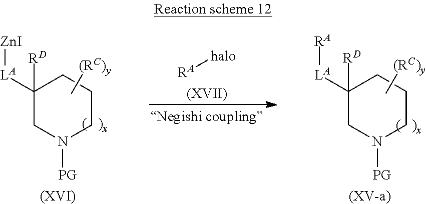

### Experimental Procedure 13

Intermediate compounds of Formula (XVI) can be prepared by reaction of a halo compound of Formula (XVIII) with zinc according to reaction scheme (13). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (13) all variables are defined as in Formula (I), L^{A} is a bond or CH₂ and halo is preferably iodo. PG is defined as in Formula (IV).

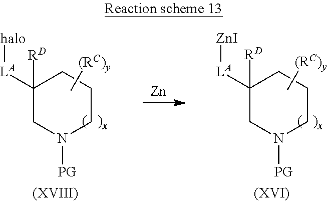

### Experimental Procedure 14

Intermediate compounds of Formula (XV), wherein L^{A} is a bond and R^{D} is hydrogen, herein referred to as (XV-b) can be prepared by hydrogenation reaction of an alkene compound of Formula (XIX) according to reaction scheme (14). The reaction is performed in a suitable reaction-inert solvent, such as, for example, methanol, and a suitable catalyst, such as, for example, palladium on carbon, and hydrogen, under thermal conditions, such as, for example, room temperature, for example for 3 hours. In reaction scheme (14), R^{A} and x are defined as in Formula (I), halo is preferably bromo or iodo, and PG is defined as in Formula (IV).

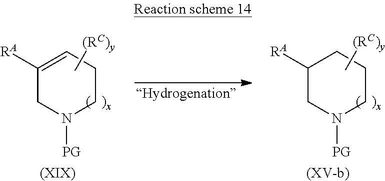

### Experimental Procedure 15

Intermediate compounds of Formula (XIX) can be prepared by “Suzuki coupling” reaction of an alkene compound of Formula (XX) and a halo derivative of Formula (XVII) according to reaction scheme (15). The reaction is performed in a suitable reaction-inert solvent, such as, for example, 1,4-dioxane, and a suitable catalyst, such as, for example, tetrakis(triphenylphosphine)palladium(0), a suitable base, such as, for example, NaHCO₃ (aq. sat. soltn.), under thermal conditions, such as, for example, 130° C., for example for 30 min under microwave irradiation. In reaction scheme (15), R^{A} and x are defined as in Formula (I), R^{D} is hydrogen, halo is preferably bromo or iodo, and PG is defined as in Formula (IV).

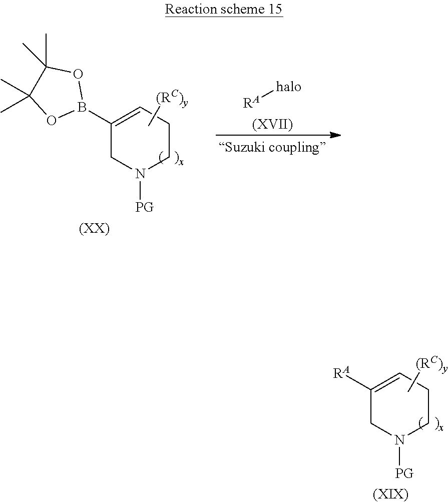

### Experimental Procedure 16

Intermediate compounds of Formula (XV), wherein L^{A} is —O— or —OCH₂—, herein referred to as (XV-c) can be prepared by reaction of a hydroxy compound of Formula (XXI) and a halo derivative of Formula (XVII) according to reaction scheme (16). The reaction is performed in a suitable reaction-inert solvent, such as, for example, dimethylformamide or dimethylsulfoxide, and a suitable base, such as, sodium hydride or potassium tert-butoxide, under thermal conditions, such as, for example, 50° C., for example for 48 h. In reaction scheme (16), R^{A} and x are defined as in Formula (I), q represents 0 or 1 and halo is preferably chloro, bromo or fluoro. PG is defined as in Formula (IV).

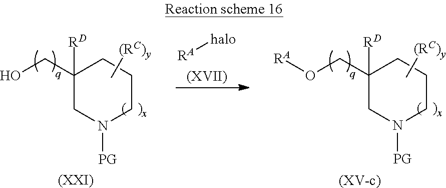

### Experimental Procedure 17

Alternatively, intermediate compounds of Formula (XV) wherein L^{A} is —O— or —OCH₂—, herein referred to as (XV-c) can be prepared by “Mitsunobu reaction” of a hydroxy compound of Formula (XXI) and a hydroxy derivative of Formula (XXII) according to reaction scheme (17). The reaction is performed in a suitable reaction-inert solvent, such as, for example, toluene, a phosphine, such as, triphenylphosphine, a suitable coupling agent, such as, for example DIAD, under thermal conditions, such as, for example, 70° C., for example for 17 h. In reaction scheme (17), R^{A} and x are defined as in Formula (I) and q represents 0 or 1. PG is defined as in Formula (IV).

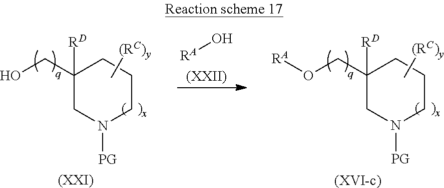

### Experimental Procedure 18

Intermediate compounds of Formula (IV) can be prepared by reacting an intermediate compound of Formula (II) with a compound of Formula (XIII) according to reaction scheme (18). The reaction is performed in a suitable reaction-inert solvent, such as, for example, dichloromethane or 1,2-dichloroethane, a metal hydride, such as, for example sodium triacetoxyborohydride, sodium cyanoborohydride or sodium borohydride and may require the presence of a suitable base, such as, for example, triethylamine or diisopropylethylamine, and/or a Lewis acid, such as, for example titanium tetraisopropoxide, under thermal conditions, such as 0° C. to 80° C., for example at 0° C. or room temperature, or 80° C., for example for 1 hour or 24 hours. In reaction scheme (18) all variables are defined as in Formula (I) and PG is defined in Formula IV.

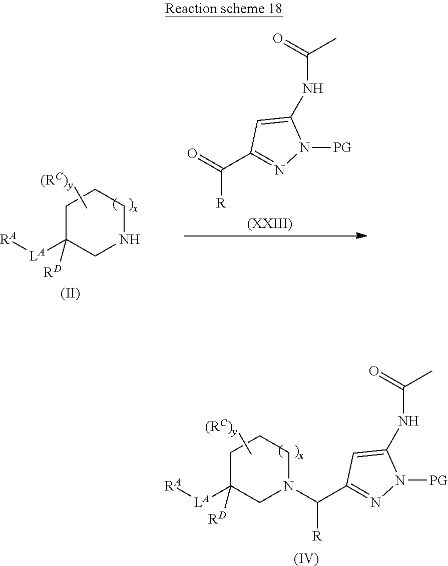

### Experimental Procedure 19

Intermediate compounds of Formula (VIII), (IX) or (X) can be prepared by reacting an intermediate compound of Formula (II) with a compound of Formula (XXIV) according to reaction scheme (19). The reaction is performed in a suitable reaction-inert solvent, such as, for example, dichloromethane or 1,2-dichloroethane, a metal hydride, such as, for example sodium triacetoxyborohydride, sodium cyanoborohydride or sodium borohydride and may require the presence of a suitable base, such as, for example, triethylamine or diisopropylethylamine, and/or a Lewis acid, such as, for example titanium tetraisopropoxide, under thermal conditions, such as, 0° C. or room temperature, or 80° C., for example for 1 hour or 24 hours. In reaction scheme (19) all variables are defined as in Formula (I) and Q represents halo, nitro or NHBoc. Halo can be represents chloro, bromo or iodo.

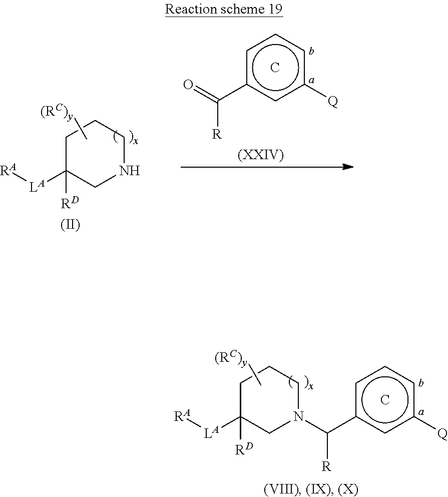

### Experimental Procedure 20

Intermediate compounds of Formula (XIII) can be prepared by reacting an intermediate compound of Formula (XXV) according to reaction scheme (20). The reaction is performed in the presence of a palladium catalyst, such as, for example tris(dibenzylideneacetone)dipalladium(0), a ligand, such as, for example 2-dicyclohexylphosphino-2′-(N,N-dimethylamino)biphenyl, a base, such as, for example sodium tert-butoxide, a suitable reaction-inert solvent, such as, for example, anhydrous 1,4-dioxane, under thermal conditions, such as, 100° C., for example for 4 hour or 24 hours. In reaction scheme (20) all variables are defined as in Formula (I), and halo is chloro, bromo or iodo.

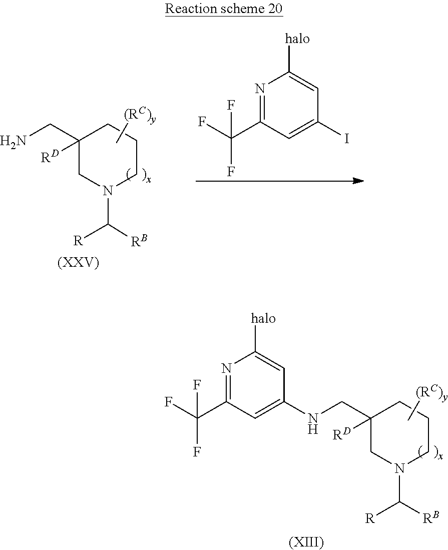

### Experimental Procedure 21

Intermediate compounds of Formula (XXV) can be prepared cleaving the protecting group in an intermediate compound of Formula (XXVI) according to reaction scheme (21). The reaction is performed in the presence of hydrazine hydrate in a suitable reaction-inert solvent, such as, for example, ethanol, under thermal conditions, such as, for example, 80° C., for example for 2 h. In reaction scheme (21), all variables are defined as in Formula (I).

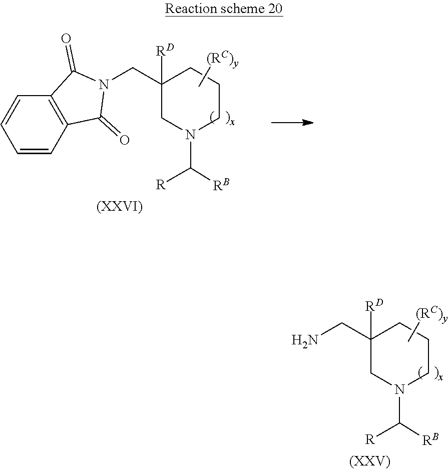

### Experimental Procedure 21

Intermediate compounds of Formula (XXVI) can be prepared by reacting an intermediate compound of Formula (XXVII) with phthalimide according to reaction scheme (21). The reaction is performed in the presence of a phosphine, such as, for example triphenylphosphine, a suitable coupling agent, such as, for example diisopropyl azodicarboxylate in a suitable reaction-inert solvent, such as, for example, dry tetrahydrofuran, under thermal conditions, such as, for example, room temperature, for example for 24 h. In reaction scheme (21) all variables are defined as in Formula (I).

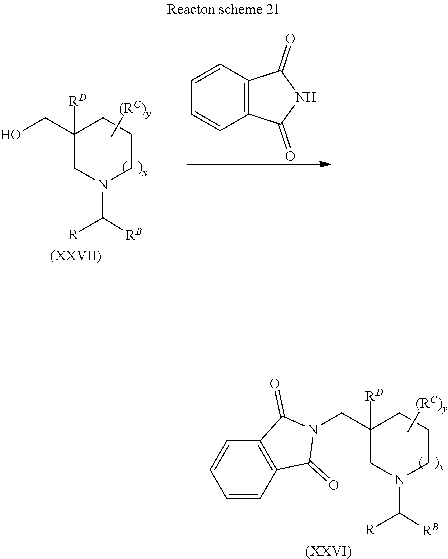

### Experimental Procedure 22

Intermediate compounds of Formula (XXVII) can be prepared by deprotecting the alcohol group in an intermediate compound of Formula (XXVIII) according to reaction scheme (22). The reaction is performed in the presence of a fluoride source, such as, for example tetrabutylammonium fluoride, in a suitable reaction-inert solvent, such as, for example, dry tetrahydrofuran, under thermal conditions, such as, for example, room temperature, for example for 16 h. In reaction scheme (22) all variables are defined as in Formula (I) and PG¹ is selected from the group consisting of trimethylsilyl, tert-butyldimethylsilyl, triisopropylsilyl or tert-butyldiphenylsilyl.

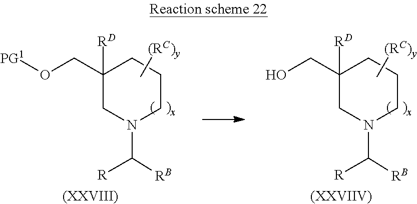

### Experimental Procedure 23

Intermediate compounds of Formula (XXVIII) can be prepared by reacting an intermediate compound of Formula (XXIX) with a compound of Formula (III) according to reaction scheme (23). The reaction is performed in a suitable reaction-inert solvent, such as, for example, dichloromethane or 1,2-dichloroethane, a metal hydride, such as, for example sodium triacetoxyborohydride, sodium cyanoborohydride or sodium borohydride and may require the presence of a suitable base, such as, for example, triethylamine or diisopropylethylamine, and/or a Lewis acid, such as, for example titanium tetraisopropoxide, under thermal conditions, such as, 0° C. or room temperature, or 80° C., for example for 1 hour or 24 hours. In reaction scheme (23) all variables are defined as in Formula (I) and PG is defined in Formula IV.

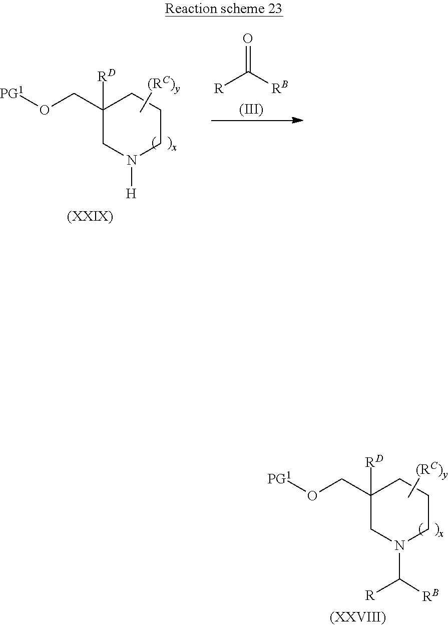

Intermediates of Formulae (III), (V), (VI), (VIII), (XII), (XIV), (XVII), (XX), (XXI), (XXII), (XXIII), (XXIV) and (XXIX) are commercially available or can be prepared by known procedures to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Sträussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

### Preclinical States in Alzheimer's and Tauopathy Diseases:

In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that A accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases A production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “m.p.” means melting point, “min” means min, “ACN” means acetonitrile, “aq.” means aqueous, “DABCO” means 1,4-diazabicyclo[2.2.2]octane, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “iPrOH” means isopropyl alcohol, “RP” means reversed phase, “Rt” means retention time (in min), “[M+H]+” means the protonated mass of the free base of the compound, “wt” means weight, “THF” means tetrahydrofuran, “DIPE” means diisopropyl ether, “EtOAc” means EtOAc, “DCM” means dichloromethane, “MeOH” means EtOH, “sat” means saturated, “soltn” means solution, “sol.” means solution, “EtOH” means EtOH, “THF” means tetrahydrofuran, and “NMP” means N-methylpyrrolidone, and “Pd2(dba)3” means tris(dibenzylideneacetone)dipalladium(0).

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “R*” or “S*” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Flow chemistry reactions were performed in a Vapourtec R2+R4 unit using standard reactors provided by the vendor.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques.

Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera 1SV systems from Biotage.

### A. Preparation of the Intermediates

### Preparation of Intermediate 1

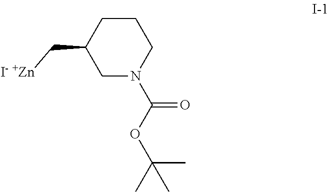

A solution of (S)-3-iodomethylpiperidine-1-carboxylic acid tert-butyl ester (CAS 384829-99-6, 50.2 g, 154.37 mmol) was pumped through a column containing activated Zn (10.1 g, 154.37 mmol) at 40° C. with flow of 0.5 mL/min. The outcome solution was collected under N₂ atmosphere to yield intermediate 1 (0.326 M) as a clear solution that was used without any further manipulation.

For the above reaction Zn was activated as follows: A solution of TMSCl (2.2 mL) and 1-bromo-2-chloroethane (0.5 mL) in THF (10 mL) was passed through the column containing Zn at a flow of 1 mL/min.

### Preparation of Intermediate 2

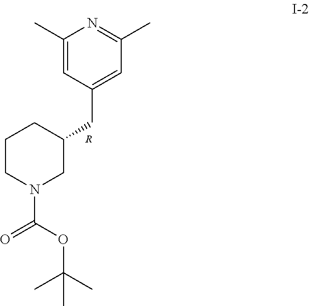

To a 400 mL EasyMax reactor equipped with overhead stirrer and temperature probe, 4-bromo-2,6-dimethylpyridine (CAS 5093-70-9, 20.6 g, 111.13 mmol) was charged under N₂ at rt. A THF solution of intermediate 1 (0.326M, 375 mL)) was then added followed by N,N,N′,N′-tetramethylethylenediamine (CAS 110-18-9, 18.3 mL, 122.25 mmol) (—previously dried over molecular sieves—exotherm observed, internal temperature rose to 24° C.) and contents degassed by N₂ sparging (5 min). Bis(triphenylphosphine)palladium(II)dichloride (CAS 13965-03-2, 1.56 g, 2.22 mmol) was then added (solution turned red) and contents degassed again for 5 min. After this, batch was warmed to 50° C. During this process an exotherm was observed starting at 45° C. approx. Internal temperature increased rapidly to 58° C., palladium black was formed immediately after. Reaction mixture was aged overnight at 20° C. and quenched with a 1:1 mixture of 32% aq. NH₃ and sat. NH₄Cl (200 mL). Reaction exothermic (internal temp. rose to 25° C.). H₂O (100 mL) was then added followed by EtOAc (200 mL) to ease phase separation. The resulting biphasic solution was filtered through a pad of diatomaceous earth to remove the palladium black residue. Phases were then separated and aqueous back-extracted with EtOAc (200 mL). Combined organics were dried over MgSO₄, solids filtered and solvents distilled under reduced pressure to dryness. Crude material was purified by normal phase column chromatography (silica; EtOAc in heptane 0/100 to 50/50). The desired fractions were collected and concentrated under reduced pressure to yield intermediate 2 (29.48 g, 87%) as an orange oil.

### Preparation of Intermediate 3

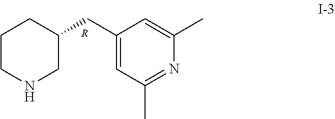

Amberlyst® 15 hydrogen form, strongly acidic, cation exchanger resin (CAS 39389-20-3, 4 meq/g, 27.4 g) was added to a stirred solution of intermediate 2 (8.0 g, 28.8 mmol) in MeOH (500 mL). The mixture was shaken in a solid phase reactor at rt for 24 h. The resin was washed with MeOH (filtrate discarded) and then with a 7N solution of NH₃ in MeOH. The filtrate was concentrated in vacuo to yield intermediate 3 (5.7 g, 96%) as a brown oil.

### Preparation of Intermediate 4

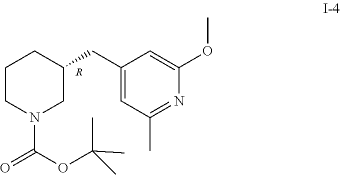

Intermediate 1 (42 mL, 15.12 mmol), followed by N,N,N′,N′-Tetramethylethylenediamine (CAS 110-18-9, 2.44 mL, 16.3 mmol) and bis(triphenylphosphine)palladium(II)dichloride (CAS 13965-03-2, 0.22 g, 0.31 mmol) were added to 4-bromo-2-methoxy-6-methylpyridine (CAS 1083169-00-9, 2.98 g, 14.75 mmol) in a round-bottom flask under a condenser and under N₂. The mixture was stirred at reflux temperature for 16 h. The mixture was quenched with a 1:1 solution of sat NH₄Cl/26% aq NH₃ and extracted with EtOAc. The organic layer was separated, dried (MgSO₄), filtered and the solvent evaporated in vacuo. The crude product was purified by flash column chromatography (silica; EtOAc in heptane 0/100 to 50/50). The desired fractions were collected and concentrated in vacuo to yield intermediate 4 (4.34 g, 92%) as a colorless oil.

### Preparation of Intermediate 5

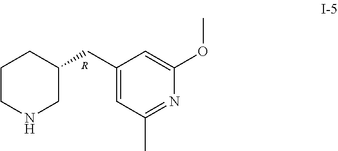

Amberlyst® 15 hydrogen form, strongly acidic, cation exchanger resin (CAS 39389-20-3, 4 meq/g, 14.4 g) was added to a stirred solution of intermediate 4 (4.3 g, 13.5 mmol) in MeOH (104 mL). The mixture was shaken in a solid phase reactor at rt for 16 h. The resin was washed with MeOH (filtrate discarded) and then with a 7N solution of NH₃ in MeOH. The filtrate was concentrated in vacuo to yield intermediate 5 (2.8 g, 95%) as a brown oil.

### Preparation of Intermediate 6

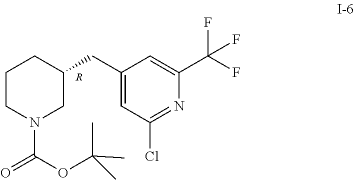

Intermediate 1 (47 mL, 15.98 mmol), was added to 2-chloro-4-iodo-6-trifluoromethyl-pyridine (CAS 205444-22-0, 4.67 g, 15.22 mmol) and bis(tri-tert-butylphosphine)palladium(0) (0.388 g, 0.76 mmol) at rt under N₂ atmosphere. The mixture was stirred at rt for 1 h. Then, the mixture was treated with a mixture of sat. NH₄Cl and NH₄OH (1:1) and extracted with EtOAc. The organic layer was separated, dried (Na₂SO₄), filtered and the solvents evaporated in vacuo. The crude product was purified by flash column chromatography (silica; EtOAc in heptane: 0/100 to 20/80). The desired fractions were collected and concentrated in vacuo to yield intermediate 6 (4 g, 69%) as pale brown oil.

### Preparation of Intermediate 7

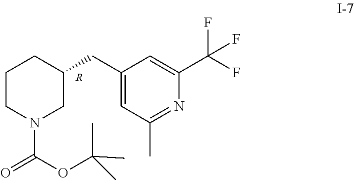

Pd(OAc)₂ (CAS 3375-31-3, 0.089 g, 0.39 mmol) and tricyclohexylphosphonium tetrafluoroborate (CAS 58656-04-5, 0.29 g, 0.79 mmol) were added to a stirred solution of intermediate 6 (2.0 g, 5.27 mmol), trimethylboroxine (CAS 823-96-1, 1.99 mL, 14.25 mmol) and K₂CO₃ (1.46 g, 10.56 mmol) in deoxygenated 1,4-dioxane (15 mL). The mixture was stirred at 100° C. for 4 h under N₂. Then more trimethylboroxine (CAS 823-96-1, 1.8 eq, 1.32 mL, 9.48 mmol), Pd(OAc)₂ (CAS 3375-31-3; 0.025 eq, 0.029 g, 0.13 mmol) and tricyclohexylphosphonium tetrafluoroborate (CAS 58656-04-5; 0.05 eq, 0.097 g, 0.26 mmol). The mixture was stirred at 100° C. for 1.5 h. After cooling to rt, the mixture was washed with H₂O and extracted with DCM. The organic layer was separated, dried (MgSO₄), filtered and the solvents evaporated in vacuo. The crude product was purified by flash column chromatography (silica; EtOAc in heptane: 0/100 to 30/70). The desired fractions were collected and concentrated in vacuo to yield intermediate 7 (1.48 g, 78%) as a dark brown oil.

### Preparation of Intermediate 8

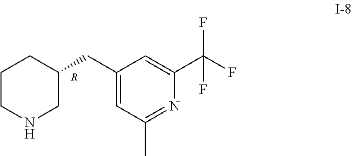

Amberlyst® 15 hydrogen form, strongly acidic, cation exchanger resin (CAS 39389-20-3, 4 meq/g, 4.39 g) was added to a stirred solution of intermediate 7 (1.5 g, 4.13 mmol) in MeOH (32 mL). The mixture was shaken in a solid phase reactor at rt for 16 h. The resin was washed with MeOH (filtrate discarded) and then with a 7N solution of NH₃ in MeOH. The filtrate was concentrated in vacuo to yield intermediate 8 (1.0 g, 94%) as a brown oil.

### Preparation of Intermediate 9

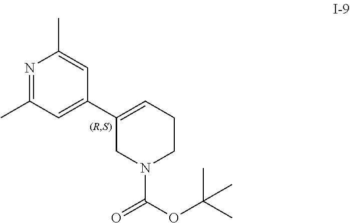 [SMILES: Cc1cc(C2=CCCN(C(=O)OC(C)(C)C)C2)cc(C)n1]

A mixture of 4-chloro-2,6-dimethylpyridine (CAS 3512-75-2, 2 g, 14.1 mmol), tert-butyl-3-(4,4,5,5-tetramethyl-1,3,2-dioxaborolan-2-yl)-5,6-dihydropyridine-1(2H)-carboxylate (CAS 1251537-34-4; 4.8 g, 15.5 mmol) and Pd(PPh₃)₄ (CAS 14221-01-3, 0.98 g, 0.85 mmol) in a deoxygenated mixture of a saturated solution of NaHCO₃ (3 mL) and 1,4-dioxane (24 mL) was stirred in a sealed tube at 130° C. for 30 min under N₂. Then, the mixture was treated with H₂O and extracted with DCM. The organic layer was separated, dried (Na₂SO₄), filtered and the solvents evaporated in vacuo. The crude product was purified by flash column chromatography (silica; EtOAc in heptane 0/100 to 100/0). The desired fractions were collected and concentrated in vacuo to intermediate 9 (3.8 g, 93%) as a colorless oil.

### Preparation of Intermediate 10

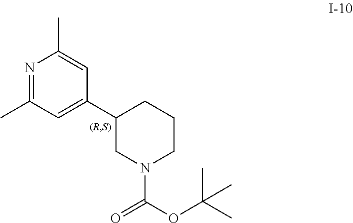 [SMILES: Cc1cc(C2CCCN(C(=O)OC(C)(C)C)C2)cc(C)n1]

A solution of intermediate 9 (3.8 g, 13.18 mmol) in EtOH (250 mL) was hydrogenated in a H-cube® (Pd/C 10%, 2 cycles, rt, full H₂, 1 mL/min). The solvent was evaporated to yield intermediate 10 (2.70 g, 70%) as a colorless oil that was used in the next step without further purification.

### Preparation of Intermediate 11

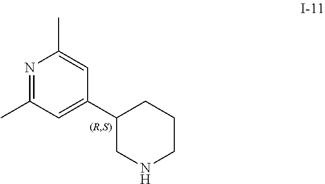

Amberlyst® 15 hydrogen form, strongly acidic, cation exchanger resin (CAS 39389-20-3; 4 meq/g, 9.3 g) was added to a stirred solution of intermediate 10 (2.7 g, 9.30 mmol) in MeOH (47 mL). The mixture was shaken in a solid phase reactor at rt for 16 h. The resin was washed with MeOH (filtrate discarded) and then with a 7N solution of NH₃ in MeOH. The filtrate was concentrated in vacuo to intermediate 11 as an orange oil (1.2 g, 68%).

### Preparation of Intermediate 12

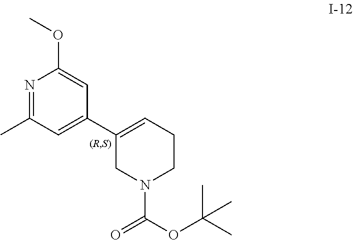 [SMILES: COc1cc(C2=CCCN(C(=O)OC(C)(C)C)C2)cc(C)n1]

Intermediate 12 was prepared following an analogous procedure to the one described for the synthesis of intermediate 9 using 4-bromo-2-methoxy-6-methylpyridine (CAS 1083169-00-9) as starting material.

### Preparation of Intermediate 13

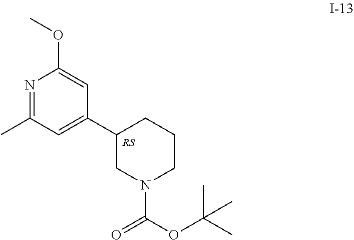 [SMILES: COc1cc(C2CCCN(C(=O)OC(C)(C)C)C2)cc(C)n1]

Intermediate 13 was prepared following an analogous procedure to the one described for the synthesis of intermediate 10.

### Preparation of Intermediate 14

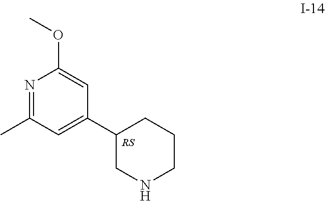

Intermediate 14 was prepared following an analogous procedure to the one described for the synthesis of intermediate 11.

### Preparation of Intermediate 15

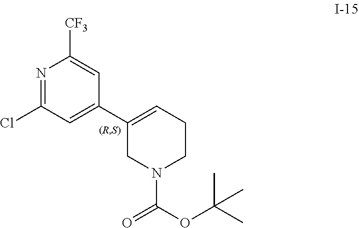 [SMILES: CC(C)(C)OC(=O)N1CCC=C(c2cc(Cl)nc(C(F)(F)F)c2)C1]

To a mixture of 2-chloro-4-iodo-6-trifluoromethylpyridine (CAS 205444-22-0, 3 g, 9.76 mmol), tert-butyl-3-(4,4,5,5-tetramethyl-1,3,2-dioxaborolan-2-yl)-5,6-dihydropyridine-1(2H)-carboxylate (CAS; 1251537-34-4, 3.62 g, 11.71 mmol) and K₃PO₄ (6.21 g, 29.27 mmol) in EtOH (24 mL), trans-bis(dicyclohexylamine)palladium(II) acetate (DAPcy, CAS 628339-96-8, 0.114 g, 0.20 mmol) was added. The mixture was stirred at rt for 18 h under N₂ and then filtered through Celite®. The Celite® pad was washed with EtOAc and the filtrate evaporated in vacuo. The crude product was purified by flash column chromatography (silica; EtOAc in heptane 0/100 to 20/80). The desired fractions were collected and concentrated in vacuo to intermediate 15 (3.8 g, 93%) as a colorless oil.

### Preparation of Intermediate 16

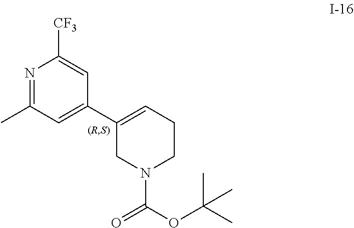 [SMILES: Cc1cc(C2=CCCN(C(=O)OC(C)(C)C)C2)cc(C(F)(F)F)n1]

Pd(OAc)₂ (CAS 3375-31-3; 0.105 g, 0.47 mmol) and tricyclohexylphosphonium tetrafluoroborate (CAS 58656-04-5, 0.345 g, 0.94 mmol) were added to a stirred solution of intermediate 15 (3.4 g, 9.37 mmol), trimethylboroxine (CAS 823-96-1, 2.36 mL, 16.87 mmol) and K₂C03 (2.59 g, 18.74 mmol) in deoxygenated 1,4-dioxane (35 mL). The mixture was stirred at 100° C. for 2 h under N₂. After cooling to rt, the mixture was washed with H₂O and extracted with DCM. The organic layer was separated, dried (MgSO₄), filtered and the solvents evaporated in vacuo. The crude product was purified by flash column chromatography (silica; EtOAc in heptane 0/100 to 15/85). The desired fractions were collected and concentrated in vacuo to yield intermediate 16 (2.8 g, 87%) as a pale-yellow oil that crystallized upon standing.

### Preparation of Intermediate 17

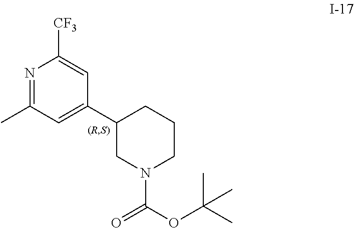 [SMILES: Cc1cc(C2CCCN(C(=O)OC(C)(C)C)C2)cc(C(F)(F)F)n1]

A solution of intermediate 16 (2.8 g, 8.18 mmol) in EtOH (160 mL) was hydrogenated in a H-cube® (Pd/C 10%, rt, full H₂, 1 ml/min). The solvent was evaporated to yield intermediate 17 (2.2 g, 68%) as a colorless oil that crystallized upon standing and was used in the next step without further purification.

### Preparation of Intermediate 18

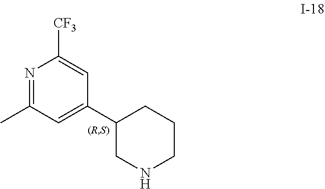 [SMILES: Cc1cc(C2CCCNC2)cc(C(F)(F)F)n1]

Amberlyst® 15 hydrogen form, strongly acidic, cation exchanger resin (CAS 39389-20-3; 4 meq/g, 6.4 g) was added to a stirred solution of intermediate 17 (2.2 g, 6.39 mmol) in MeOH (32 mL). The mixture was shaken in a solid phase reactor at rt for 16 h. The resin was washed with MeOH (filtrate discarded) and then with a 7N solution of NH₃ in MeOH. The filtrate was concentrated in vacuo and the residue was purified by reverse phase HPLC (stationary phase: C18 XBridge 30×100 mm 5 μm, mobile phase: gradient from 80% NH₄HCO₃ 0.25% solution in H₂O, 20% CH₃CN to 60% NH₄HCO₃ 0.25% solution in H₂O, 40% CH₃CN), to yield intermediate 18 (1.28 g, 82%) as a colorless oil.

### Preparation of Intermediate 19

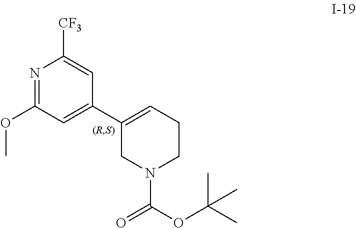 [SMILES: COc1cc(C2=CCCN(C(=O)OC(C)(C)C)C2)cc(C(F)(F)F)n1]

Intermediate 18 (3.4 g, 9.37 mmol) was dissolved in MeOH (50 mL) and a 25% solution of sodium methoxide in MeOH (2.14 mL, 9.37 mmol) was added. The mixture was stirred at rt for 16 h. Then H₂O was added and the desired product extracted with DCM. The organic layer was separated, dried (Na₂SO₄), filtered and the solvents evaporated in vacuo. The crude product was purified by flash column chromatography (silica; DCM in heptane: 20/80 to 100/0). The desired fractions were collected and concentrated in vacuo to intermediate 19 (3.1 g, 92%) as a colorless oil.

### Preparation of Intermediate 20

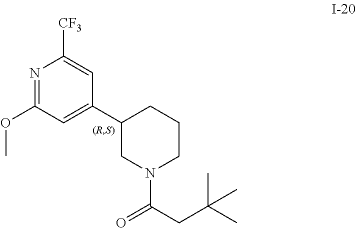 [SMILES: COc1cc(C2CCCN(C(=O)CC(C)(C)C)C2)cc(C(F)(F)F)n1]

A solution of intermediate 19 (3.1 g, 8.65 mmol) in EtOH (170 mL) was hydrogenated in a H-cube® (Pd/C 10%, rt, full H₂, 1 ml/min). The solvent was evaporated to yield intermediate 20 (3.0 g, 96%) as a colorless oil that crystallized upon standing used in the next step without further purification.

### Preparation of Intermediate 21

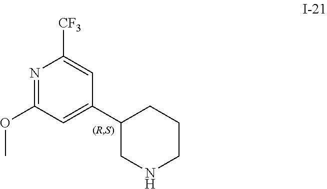 [SMILES: COc1cc(C2CCCNC2)cc(C(F)(F)F)n1]

Amberlyst® 15 hydrogen form, strongly acidic, cation exchanger resin (CAS 39389-20-3, 4 meq/g, 7 g) was added to a stirred solution of intermediate 20 (3.0 g, 8.33 mmol) in MeOH (42 mL). The mixture was shaken in a solid phase reactor at rt for 16 h. The resin was washed with MeOH (filtrate discarded) and then with a 7N solution of NH₃ in MeOH. The filtrate was concentrated in vacuo and the residue was purified by reverse phase HPLC (stationary phase: C18 XBridge 30×100 mm 5 μm, mobile phase: gradient from 80% NH₄HCO₃ 0.25% solution in H₂O, 20% CH₃CN to 60% NH₄HCO₃ 0.25% solution in H₂O, 40% CH₃CN), to yield intermediate 21 (1.70 g, 78%) as a colorless oil.

### Preparation of Intermediate 22

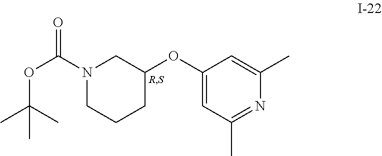 [SMILES: Cc1cc(OC2CCCN(C(=O)OC(C)(C)C)C2)cc(C)n1]

1-Boc-3-hydroxypiperidine (CAS 85175-45-2, 0.41 g, 2.05 mmol) was stirred in DMF (1.65 mL), at rt, and then a 60% NaH dispersion in mineral oil (0.082 g, 2.05 mmol) was added. Then 4-chloro-2,6-dimethylpyridine (CAS 3512-75-2, 0.26 mL, 2.05 mmol) in DMF (0.64 mL) was added dropwise at rt. The mixture was stirred overnight at 60° C. The mixture was evaporated diluted with H₂O and was extracted with EtOAc. The organic layer was separated, dried (Na₂SO₄), filtered and evaporated, in vacuo. The residue was purified by flash column chromatography (silica; EtOAc in heptane 0/100 to 30/70). The desired fractions were collected and concentrated in vacuo to yield intermediate 22 (0.32 g, 51%) as a colorless oil.

### Preparation of Intermediate 23

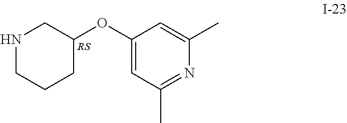

TFA (0.4 mL, 2.19 mmol) was added to a stirred solution of intermediate 34 (0.32 g, 1.04 mmol) in DCM (0.86 mL) at 0° C. The mixture was stirred at rt for 16 h. The reaction was concentrated to dryness and the residue was purified first by ion exchange chromatography using an ISOLUTE SCX2 cartridge eluting first with EtOH and then with 7M solution of ammonia in EtOH, the desired fractions were collected and evaporated to give intermediate 23 (0.11 g, 53%) a colorless oil.

### Preparation of Intermediate 24

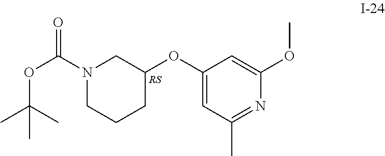 [SMILES: COc1cc(OC2CCCN(C(=O)OC(C)(C)C)C2)cc(C)n1]

Intermediate 24 was prepared following an analogous procedure to the one described for the synthesis of intermediate 22 using 4-bromo-2-methoxy-6-methylpyridine (CAS 1083169-00-9) as starting material.

### Preparation of Intermediate 25

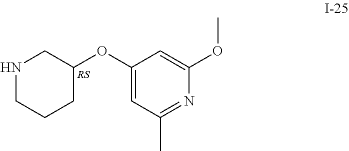 [SMILES: COc1cc(OC2CCCNC2)cc(C)n1]

Intermediate 25 was prepared following an analogous procedure to the one described for the intermediate 21 but with intermediate 24 as starting material.

### Preparation of Intermediate 26

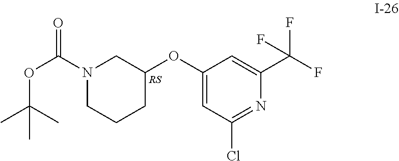 [SMILES: CC(C)(C)OC(=O)N1CCCC(Oc2cc(Cl)nc(C(F)(F)F)c2)C1]

To a stirred solution of tert-butyl 3-hydroxy-1-piperidinecarboxylate (2500 mg, 12.42 mmol) in DMF (10 mL) at −40° C., was added 2-chloro-4-iodo-6-trifluoromethyl-pyridine (CAS 205444-22-0, 3.62 g, 12.42 mmol) in DMF (4 mL) dropwise. The mixture was gradually warmed to rt and stirred for 16 h. The mixture was diluted with EtOAc and washed with H₂O and brine. The organic layer was separated, dried (Na₂SO₄), filtered and concentrated in vacuo. The residue was purified by flash column chromatography (silica; EtOAc in heptane 0/100 to 50/50). The desired fractions were collected and concentrated in vacuo to yield intermediate 26 (2.8 g, 59%) as a light-yellow oil.

### Preparation of Intermediate 27

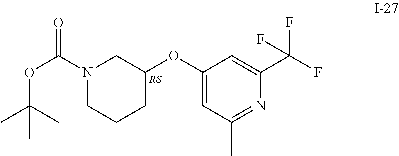 [SMILES: Cc1cc(OC2CCCN(C(=O)OC(C)(C)C)C2)cc(C(F)(F)F)n1]

K₂CO₃ (2.03 g, 14.70 mmol) was added to a stirred solution of intermediate 26 (2.8 g, 7.35 mmol) in 1,4-dioxane (21.43 mL) and it was deoxygenated with a N₂ flow for 5 min. Then, trimethylboroxine (CAS 823-96-1, 2.77 mL, 19.85 mmol), Pd(OAc)₂ (CAS 3375-31-3, 0.123 g, 0.55 mmol) and tricyclohexylphosphonium tetrafluoroborate (CAS 58656-04-5, 0.406 g, 1.10 mmol were added. The mixture was stirred at 100° C. for 4 h under N₂. After cooling, the mixture was washed with H₂O and extracted with DCM. The organic layer was separated, dried (MgSO₄), filtered and the solvents evaporated in vacuo. The crude product was purified by flash column chromatography (silica; EtOAc in heptane: 0/100 to 30/70). The desired fractions were collected and concentrated in vacuo to yield intermediate 27 (2.63 g, 99%) as a dark brown oil.

### Preparation of Intermediate 28

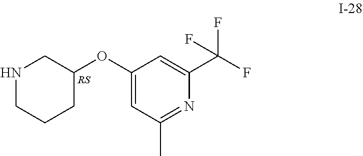 [SMILES: Cc1cc(OC2CCCNC2)cc(C(F)(F)F)n1]

Amberlyst® 15 hydrogen form, strongly acidic, cation exchanger resin (CAS 39389-20-3, 4 meq/g, 7.76 g) was added to a stirred solution of intermediate 27 (2.63 g, 7.29 mmol) in MeOH (56 mL). The mixture was shaken in a solid phase reactor at rt for 16 h. The resin was washed with MeOH (filtrate discarded) and then with a 7N solution of NH₃ in MeOH. The filtrate was concentrated in vacuo to yield intermediate 28 (1.66 g, 87%) as a dark oil.

### Preparation of Intermediate 29

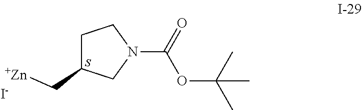

A solution of (3S)-iodomethylpyrrolidine-1-carboxylic acid tert-butyl ester (CAS 224168-68-7, 6.33 g, 20.34 mmol) in THF (40 mL) was pumped through a column containing activated Zn (30 g, 188.1 mmol) at 40° C. with flow of 0.5 mL/min. The outcome solution was collected under N₂ atmosphere to yield intermediate 29 as a clear solution that was used without any further manipulation.

For the above reaction Zn was activated as follows: A solution of TMSCl (2.2 mL) and 1-bromo-2-chloroethane (0.5 mL) in THF (10 mL) was passed through the column containing Zn at a flow of 1 mL/min.

### Preparation of Intermediate 30

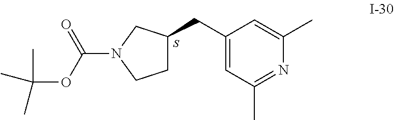

N,N,N′,N′-Tetramethylethylenediamine (4.4 mL, 29.34 mmol) followed by 4-bromo-2,6-dimethylpyridine (CAS 5093-70-9, 1.92 g, 26.4 mmol) and bis(triphenylphosphine)palladium(II) dichloride (CAS 13965-03-2, 0.45 g, 0.64 mmol) were added to intermediate 29 (83 mL, 29.38 mmol, 0.35 M in THF) in a round-bottom flask under a condenser and under N₂. The mixture was stirred at reflux temperature for 16 h. The mixture was quenched with a 1:1 solution of sat NH₄Cl/26% aq NH₃ and extracted with EtOAc. The organic layer was separated, dried (MgSO₄), filtered and the solvent evaporated in vacuo. The crude product was purified by flash column chromatography (silica; EtOAc in DCM 0/100 to 100/0). The desired fractions were collected and concentrated in vacuo to yield intermediate 30 (7.0 g, 92%) as an orange oil.

### Preparation of Intermediate 31

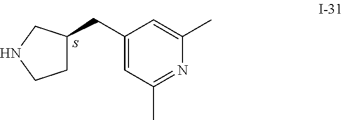

Amberlyst® 15 hydrogen form, strongly acidic, cation exchanger resin (CAS 39389-20-3, 4 meq/g, 25.7 g) was added to a stirred solution of intermediate 30 (7.46 g, 25.68 mmol) in MeOH (129 mL). The mixture was shaken in a solid phase reactor at rt for 16 h. The resin was washed with MeOH (filtrate discarded) and then with a 7N solution of NH₃ in MeOH. The filtrate was concentrated in vacuo to yield intermediate 31 (4.25 g, 87%) as a dark oil.

### Preparation of Intermediate 32

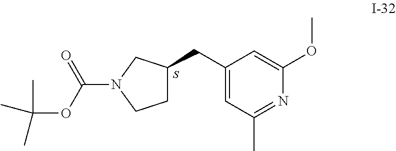

Intermediate 32 was prepared following an analogous procedure to the one described for the synthesis of intermediate 30 using 4-bromo-2-methoxy-6-methylpyridine (CAS 1083169-00-9) as starting material.

### Preparation of Intermediate 33

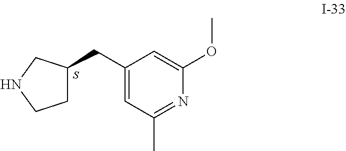

Intermediate 33 was prepared following an analogous procedure to the one described for the synthesis of intermediate 31 using intermediate 32 as starting material.

### Preparation of Intermediate 34

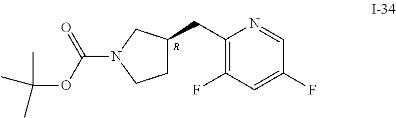

Intermediate 34 was prepared following an analogous procedure to the one described for the synthesis of intermediate 30 using 2-bromo-3,5-difluoropyridine (CAS 660425-16-1) as starting material.

### Preparation of Intermediate 35

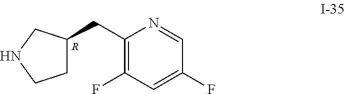

Intermediate 35 was prepared following an analogous procedure to the one described for the synthesis of intermediate 31 using intermediate 34 as starting material

### Preparation of Intermediate 36

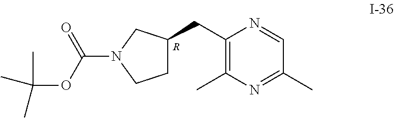

Intermediate 36 was prepared following an analogous procedure to the one described for the synthesis of intermediate 30 using 2-chloro-3,5-dimethylpyrazine (CAS 38557-72-1) as starting material.

### Preparation of Intermediate 37

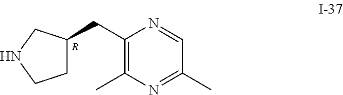

Intermediate 37 was prepared following an analogous procedure to the one described for the synthesis of intermediate 31 using intermediate 36 as starting material

### Preparation of Intermediate 38

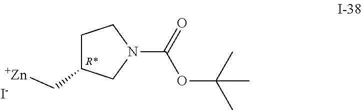

Intermediate 38 was prepared following an analogous procedure to the one described for the synthesis of intermediates 29 and 30 using (3R)-iodomethylpyrrolidine-1-carboxylic acid tert-butyl ester (CAS 1187932-69-9) as starting material.

### Preparation of Intermediate 39

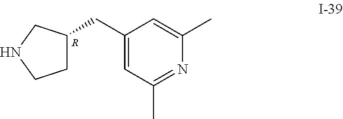

Intermediate 39 was prepared following an analogous procedure to the one described for the synthesis of intermediate 31 using intermediate 38 as starting material.

### Preparation of Intermediate 40

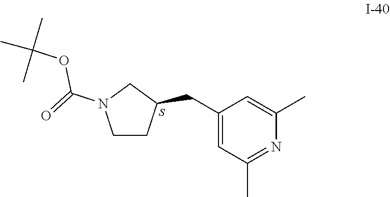

1-Boc-(3S)-hydroxypyrrolidine (CAS, 1.60 g, 8.54 mmol) stirred in DMF (4.12 mL) at rt. A 60% NaH dispersion in mineral oil (0.34 g, 8.54 mmol) was added followed by chloro-2,6-dimethylpyridine (CAS 3512-75-2, 1.09 mL, 8.54 mmol) in DMF (2.78 mL) was added dropwise at rt. The mixture was stirred overnight at 60° C. The mixture was evaporated diluted with H₂O and was extracted with EtOAc. The organic layer was separated, dried (Na₂SO₄), filtered and evaporated, in vacuo. The residue was purified by flash column chromatography (silica; EtOAc in heptane 0/100 to 30/70). The desired fractions were collected and concentrated in vacuo to yield intermediate 40 (1.67 g, 67%) as a colorless oil.

### Preparation of Intermediate 41

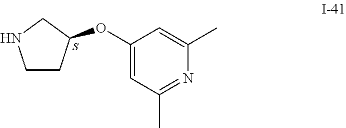

Amberlyst® 15 hydrogen form, strongly acidic, cation exchanger resin (CAS 39389-20-3; 4 meq/g, 6.08 g) was added to a stirred solution of intermediate 40 (1.67 g, 5.71 mmol) in MeOH (44 mL). The mixture was shaken in a solid phase reactor at rt for 16 h. The resin was washed with MeOH (filtrate discarded) and then with a 7N solution of NH₃ in MeOH. The filtrate was concentrated in vacuo to yield intermediate 41 (0.96 g, 87%) as a dark oil.

### Preparation of Intermediate 42

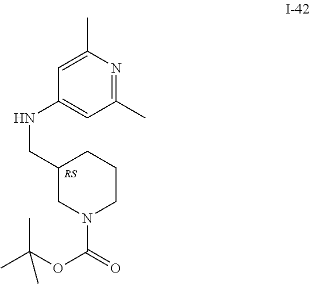

Pd₂(dba)₃ (0.44 g, 0.48 mmol), Dave-Phos (CAS 213697-53-1, 0.39 g, 0.97 mmol) and sodium tert-butoxide (1.86 g, 19.35 mmol) was added under N₂ at solution of 4-bromo-2,6-dimethylpyridine (CAS 5093-70-9, 1.8 g, 9.67 mmol) in anhydrous THF (40 mL). Then, 1-boc-2-(aminomethyl)piperidine (CAS 162167-97-7, 2.49 g, 11.61 mmol) was added at rt in a seale tube and the mixture was stirred at 100° C. for 16 h. The mixture was diluted with EtOAc and of NH₄Cl sat (0.5 mL), filtered over a pad of diatomaceous earth and the solvents evaporated in vacuo. The crude product was purified by flash column chromatography (silica; MeOH in DCM 0/100 to 50/50). The desired fractions were collected and concentrated in vacuo. The crude product was purified by RP HPLC 72% [25 mM NH₄HCO₃]—28% [ACN:MeOH 1:1] to 36% [25 mM NH₄HCO₃]—64% [ACN: MeOH 1:1]. The desired fractions were collected and concentrated in vacuo at 60° C. ACN (3×10 mL) was added and concentrated at 60° C. in vacuo to yield intermediate 42 (1.0 g, 32%) as a yellow oil that precipitates upon standing.

### Preparation of Intermediate 43

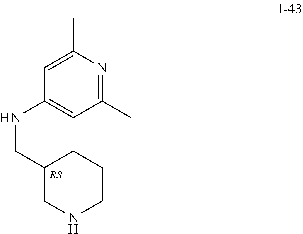 [SMILES: Cc1cc(NCC2CCCNC2)cc(C)n1]

A 4M solution of HCl in 1,4-dioxane (14.9 mL, 59.6 mmol) was added dropwise to a stirred solution of intermediate 41 (1.29 g, 3.97 mmol) in MeOH (11.3 mL) at 0° C. The mixture was stirred at rt for 16 h. The solvent was evaporated in vacuo. The crude was purified by flash chromatography (silica; DCM/MeOH (9:1) in DCM 0/100 to 100/0). The desired fractions were collected and concentrated in vacuo and dried to yield a compound that was repurified by RP-HPLC 95% [25 mM NH₄HCO₃]-5% [MeCN:MeOH (1:1)] to 63% [25 mM NH₄HCO₃]-37% [MeCN: MeOH (1:1)]. The desired fractions were collected and concentrated in vacuo at 60° C. ACN (10 mL×3 times) was added and the solvents was concentrated in vacuo to yield a compound that was repurified by flash chromatography (silica; DCM/MeOH/NH₃ (20/7/1) in DCM 0/100 to 100/0). The desired fractions were collected and concentrated in vacuo to yield and dried to yield intermediate 43 (0.37 g, 43%) as an oil that precipitates upon standing.

### Preparation of Intermediate 44

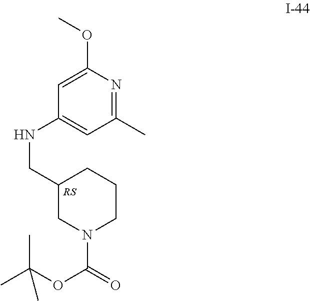 [SMILES: COc1cc(NCC2CCCN(C(=O)OC(C)(C)C)C2)cc(C)n1]

Intermediate 44 was prepared following an analogous procedure to the one described for the synthesis of intermediate 41 using 2-bromo-3,5-difluoropyridine (CAS 660425-16-1) as starting material.

### Preparation of Intermediate 45

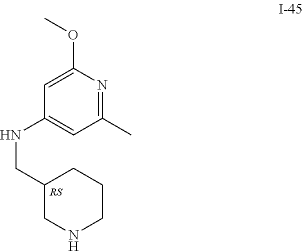 [SMILES: COc1cc(NCC2CCCNC2)cc(C)n1]

Intermediate 45 was prepared following an analogous procedure to the one described for the synthesis of intermediate 43 as starting material.

### Preparation of Intermediate 46

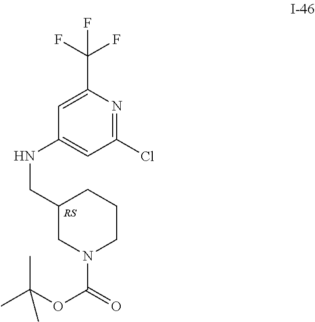 [SMILES: CC(C)(C)OC(=O)N1CCCC(CNc2cc(Cl)nc(C(F)(F)F)c2)C1]

N₂ was bubbled through a solution of 4-bromo-2,6-dimethylpyridine (CAS 5093-70-9, 1.47 g, 4.79 mmol) in 1,4-dioxane. Then sodium tert-butoxide (CAS 865-48-5, 0.92 g, 9.58 mmol), Dave-Phos (CAS 213697-53-1, 94 mg, 0.24 mmol) and Pd₂dba₃ (CAS 52364-51-3, 0.10 g, 0.12 mmol) were added at rt while N₂ was bubbled. 1-Boc-2-(aminomethyl)piperidine (CAS 162167-97-7, 7.10 g, 5.0 mmol) was added and the mixture was stirred at 100° C. overnight in a closed tube. The mixture was diluted with NH₄Cl sat. and extracted with EtOAc. The organic layer was separated, dried (MgSO₄), filtered and the solvents evaporated in vacuo. The crude product was purified by flash column chromatography (silica; EtOAc in heptane 0/100 to 30/70). The desired fractions were collected and concentrated in vacuo to yield intermediate 46 (1.16 g, 60%) as an orange sticky solid.

### Preparation of Intermediate 47

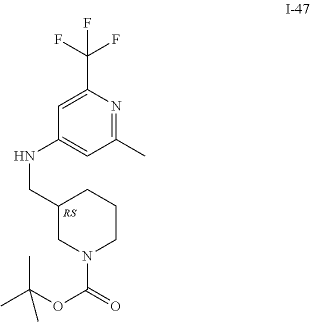 [SMILES: Cc1cc(NCC2CCCN(C(=O)OC(C)(C)C)C2)cc(C(F)(F)F)n1]

Trimethylboroxine (CAS 823-96-1, 0.49 mL, 3.53 mmol) were added to a stirred suspension of intermediate 45 (1.16 g, 2.94 mmol), K₃PO₄ (1.25 g, 5.89 mmol), X-Phos (0.14 g, 0.29 mmol) and Pd₂(dba)₃ (0.13 g, 0.14 mmol) in 1,4-dioxane (25 mL) under N₂. The mixture was stirred at 95° C. overnight. H₂O and EtOAc were added. The organic layer was separated, dried (MgSO₄) and filtered and the solvents evaporated in vacuo. The crude was purified by flash column chromatography (silica; EtOAc in heptane 0/100 to 50/50). The desired fractions were collected and concentrated in vacuo to yield intermediate 47 (1.10 g, 95%) a pale-yellow sticky solid.

### Preparation of Intermediate 48

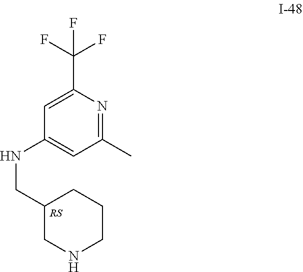 [SMILES: Cc1cc(NCC2CCCNC2)cc(C(F)(F)F)n1]

Intermediate 48 was prepared following an analogous procedure to the one described for the synthesis of intermediate 43.

### Preparation of Intermediate 49

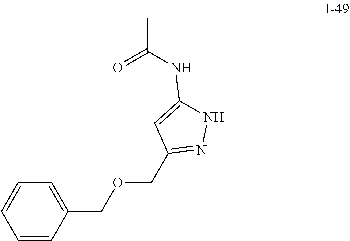 [SMILES: CC(=O)Nc1cc(COCc2ccccc2)n[nH]1]

To a solution of 5-[(phenylmethoxy)methyl]-1H-pyrazol-3-amine (CAS 393590-62-0, 1.2 g, 5.90 mmol) in 1,4-dioxane (8.3 mL) was added acetic anhydride (CAS 108-24-7, 0.67 mL, 7.08 mmol) dropwise and the reaction mixture was stirred at rt for 3 h. The reaction was concentrated to dryness. The residue was dissolved in EtOH (4 mL). Then a sat. solution of K₂CO₃ (2 mL) was added and the mixture was stirred at rt for 18 h. The reaction was partially concentrated in vacuo to remove the EtOH and then the residue was diluted with H₂O and the product extracted with ethyl acetate. The organic layer was separated, dried (MgSO₄), filtered and the solvents removed in vacuo to yield intermediate 49 (1.2 g, 83%) as an oil.

### Preparation of Intermediate 50

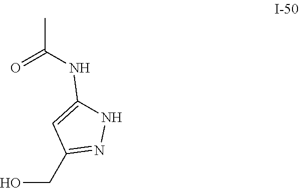

A solution of intermediate 49 (1.2 g, 4.89 mmol) in EtOH (21 mL) was hydrogenated using a 10% Pd/(C) cartridge at 80° C. for 5 h (recirculation of the solution). The solvent was removed in vacuo to give intermediate 50 (0.71 g, 93%) as a white solid that was used in the next step without further purification.

### Preparation of Intermediate 51

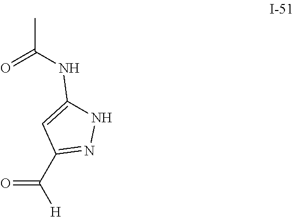

To a suspension of intermediate 50 (0.15 g, 0.97 mmol) in DCE (3 mL) and 1,4-dioxane (1 mL) was added manganese(IV)oxide (CAS 1313-13-9, 0.42 g, 4.83 mmol) and the reaction mixture was stirred at 80° C. for 18 h. The solid was filtered off and washed with DCE and THF, and the filtrate was concentrated under reduced pressure to give a solid that was further washed with MeOH and the filtrate concentrated under reduced pressure to give intermediate 51 (60 mg, 40%) as an off-white solid.

### Preparation of Intermediate 52

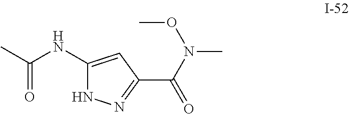

To a solution of 5-amino-N-methoxy-N-methyl-1H-pyrazole-3-carboxamide (CAS 1290181-42-1, 3.0 g, 17.63 mmol) in 1,4-dioxane (30 mL) was added acetic anhydride (3.66 mL, 37.78 mmol) dropwise and the reaction mixture was stirred at rt for 3 h. The precipitate was filtered, washed with DIPE, and dried in vacuo to give intermediate 51 (1.9 g, 51%) as a white solid. Then the filtrate was concentrated in vacuo and the residue was triturated with DIPE, and dried in vacuo to give intermediate 52 (1.6 g, 42%) as a white solid. The product was used in the next step without further purification.

### Preparation of Intermediate 53

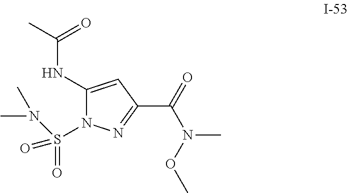

DABCO (CAS 280-57-9, 0.58 g, 5.18 mmol) was added followed by N,N-dimethylsulfamoyl chloride (CAS 13360-57-1, 0.51 mL, 4.76 mmol) to a solution of intermediate 52 (1.0 g, 4.71 mmol) in ACN (20 mL) at 0° C. The mixture was allowed to warm to rt and stirred for 18 h. The mixture was concentrated in vacuo and the residue was diluted with sat. NH₄Cl and the product extracted with ethyl acetate. The organic layer was separated, dried (MgSO₄), filtered and the solvents removed in vacuo. The residue was purified by flash column chromatography (silica; EtOAc in DCM: 0/100 to 20/80). The desired fractions were collected and concentrated in vacuo to yield intermediate 53 (0.14 g, 9%) as a colorless oil which solidified upon standing.

### Preparation of Intermediate 54

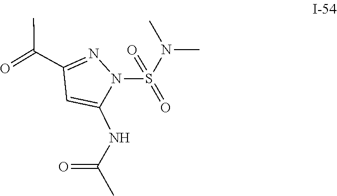 [SMILES: CC(=O)Nc1cc(C(C)=O)nn1S(=O)(=O)N(C)C]

To a stirred solution of intermediate 53 (0.13 g, 0.43 mmol) in dry THF (2.7 mL), under a N₂ atmosphere, a 3M solution of methylmagnesium bromide in diethylether (0.42 mL, 1.27 mmol) was added dropwise at 0° C., and the reaction mixture was stirred for 2 h. The reaction mixture was quenched with sat. NH₄Cl solution and the product extracted with EtOAc. The organic layer was separated, dried (MgSO₄), filtered and the solvents removed in vacuo. The residue was purified by flash column chromatography (silica; EtOAc in DCM: 0/100 to 20/80). The desired fractions were collected and concentrated in vacuo to yield intermediate 54 (0.10 g, 86%) as a colorless oil which solidified upon standing.

### Preparation of Intermediate 55

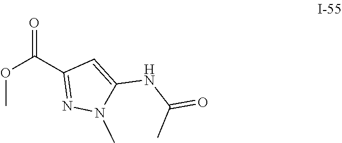

Acetic anhydride (1.1 mL, 11.6 mmol) was added dropwise to a solution of the methyl 5-amino-1-methyl-1H-pyrazole-3-carboxylate (CAS 1064783-29-4, 1 g, 6.4 mmol) in 1,4-dioxane (9 mL) and the reaction mixture was stirred at rt for 3 h. The precipitate was filtered, washed with diethyl ether and dried in vacuo to give a white solid. Then the filtrate was concentrated in vacuo and the residue was triturated with diethyl ether. The solid was filtered, washed with diethyl ether and dried in vacuo to give intermediate 55 (1.17 g, 92%) as a white solid. The product was used in the next step without further purification.

### Preparation of Intermediate 56

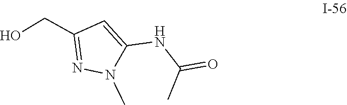

Lithium borohydride (0.11 g, 5.32 mmol) and MeOH (0.21 mL, 5.32 mmol) were added to a stirred solution of intermediate 55 (0.52 g, 2.66 mmol) in THF (5 mL) at 0° C. The mixture was allowed to warm to rt and stirred overnight. Then more lithium borohydride (0.11 g, 5.32 mmol) and MeOH (0.21 mL, 5.32 mmol) were added at 0° C. and the mixture was allowed to warm to rt and stirred overnight. The mixture was concentrated in vacuo and then dissolved in MeOH (5 mL) and lithium borohydride (0.11 g, 5.32 mmol) was added at 0° C. and the mixture was allowed to warm to rt and stirred overnight. The mixture was diluted with MeOH and concentrated in vacuo. The crude product was purified by RP-Flash (Stationary phase: YMC 100 g, 25 μm, mobile phase: Gradient from 80% NH₄HCO₃ 0.25% solution in H₂O, 20% CH₃OH to 20% NH₄HCO₃ 0.25% solution in H₂O, 80% MeOH). The desired fractions were collected and concentrated in vacuo to yield intermediate 56 (0.52 g, quantitative yield) as a white solid.

### Preparation of Intermediate 57

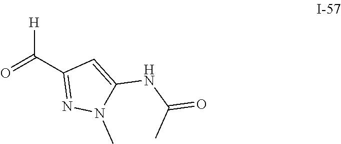

Manganese(IV)oxide (1.33 g, 15.37 mmol) was added to a stirred solution of intermediate 56 (0.52 g, 3.0 mmol) in 1,4-dioxane (9 mL). The reaction mixture was stirred at 80° C. for 2 h. The solid was filtered off through a pad of diatomaceous earth, washed with MeOH, and the filtrate was concentrated under reduced pressure. The crude product was purified by flash column chromatography (silica; MeOH in DCM 0/100 to 10/90). The desired fractions were collected and the solvents evaporated in vacuo to yield intermediate 57 (0.38 g, 75%) as a white solid.

### Preparation of Intermediate 58

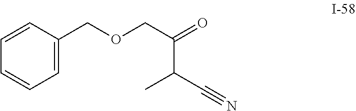

Propionitrile (3.0 mL, 0.04 mmol) was added dropwise to a stirred solution of 2.5 M of n-butyllithium in hexane (15.8 mL, 0.04 mmol) in THF (50 ml) at −78° C. The mixture was stirred at −78° C. for 2 h. Then, a solution of ethyl benzyloxyacetate (CAS 32122-09-1, 6.16 g, 0.03 mmol) in THF (10 mL) was added dropwise and the mixture was stirred at −78° C. for 1 h. The mixture was quenched with NH₄Cl sat. Then, the mixture was poured into ice-H₂O and acidified with a 4N HCl solution and extracted with diethyl ether. The organic layer was separated, dried (MgSO₄), filtered and the solvents evaporated in vacuo. The crude product was purified by flash column chromatography (silica; EtOAc in heptane: 0/100 to 30/70). The desired fractions were collected and concentrated in vacuo to yield intermediate 58 (4.27 g, 36%, 55% pure) as a pale-yellow oil.

### Preparation of Intermediate 59

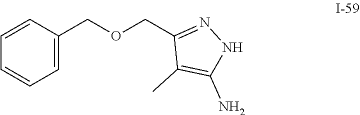 [SMILES: Cc1c(COCc2ccccc2)n[nH]c1N]

Hydrazine hydrate (7.9 mL, 106 mmol) was added to a stirred solution of intermediate 58 (4.1 g, 20.1 mmol) in EtOH (82 mL) at rt. The mixture was stirred at 70° C. for 2.5 h. The mixture was evaporated to dryness. The crude product was purified by flash column chromatography (silica; MeOH in EtOAc: 0/100 to 5/95). The desired fractions were collected and concentrated in vacuo to yield intermediate 59 (2.13 g, 41%, 84% pure) as a yellow oil.

### Preparation of Intermediate 60

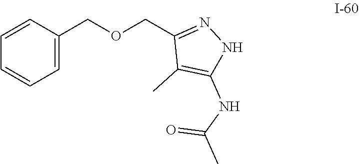 [SMILES: CC(=O)Nc1[nH]nc(COCc2ccccc2)c1C]

To a solution of intermediate 59 (2.1 g, 9.66 mmol) in 1,4-dioxane (10.5 mL) was added acetic anhydride (2.0 mL, 21.2 mmol) dropwise and the reaction mixture was stirred at rt for 4 h. The reaction was concentrated to dryness. The residue was dissolved in EtOH (15 mL). Then a sat. solution of K₂CO₃ (20 mL) was added and the mixture was stirred at rt for 30 h. The reaction was partially concentrated in vacuo to remove the EtOH and then the residue was diluted with H₂O and the product was extracted with EtOAc. The organic layer was separated, dried (MgSO₄), filtered and the solvents removed in vacuo to yield intermediate 60 (2.33 g, 93%) as a white solid.

### Preparation of Intermediate 61

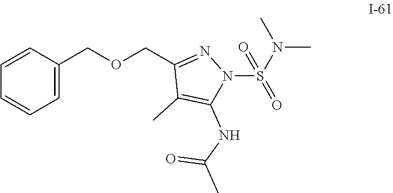 [SMILES: CC(=O)Nc1c(C)c(COCc2ccccc2)nn1S(=O)(=O)N(C)C]

DABCO (1.1 eq, 0.47 g, 4.2 mmol)) was added followed by dimethylsulfamoyl chloride (1.0 eq, 0.55 g, 3.85 mmol) to a solution of intermediate 60 (1 g, 3.85 mmol) in ACN (10 mL) at 0° C. The mixture was allowed to warm to rt and stirred for 3 days. Then, more DABCO (0.6 eq, 0.26 g, 2.31 mmol) and dimethylsulfamoyl chloride (0.5 eq, 0.20 mL, 1.92 mmol) were added and the mixture was stirred at rt for 5 h and then at 70° C. for 16 h. Then, more DABCO (0.6 eq, 0.26 g, 2.31 mmol)) and dimethylsulfamoyl chloride (0.5 eq, 0.20 mL, 1.92 mmol) were added and the mixture was stirred at 70° C. for 2 days. The mixture was concentrated in vacuo and the residue was diluted with H₂O and extracted with EtOAc. The organic layer was separated, dried (MgSO₄), filtered and the solvents removed in vacuo. The crude product was purified by flash column chromatography (silica; EtOAc in heptane 0/100 to 100/0). The desired fractions were collected and the solvents evaporated in vacuo to yield intermediate 61 (1.15 g, 81%) as a yellow oil.

### Preparation of Intermediate 62

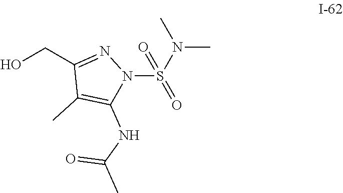 [SMILES: CC(=O)Nc1c(C)c(CO)nn1S(=O)(=O)N(C)C]

To a solution of intermediate 61 (1.15 g, 3.14 mmol) in EtOH (25 mL) was added Pd/C (10%) (0.57 g, 0.63 mmol) and the reaction mixture was hydrogenated (atmospheric pressure) for 3 days. The solvent was removed in vacuo to give intermediate 62 (0.86 g, 99%) as a colorless oil. The product was used in the next step without further purification.

### Preparation of Intermediate 63

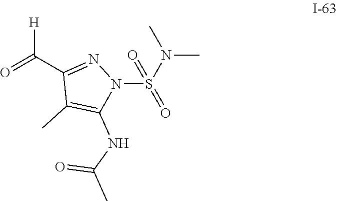 [SMILES: CC(=O)Nc1c(C)c(C=O)nn1S(=O)(=O)N(C)C]

Manganese(IV)oxide (7.5 eq, 1.9 g, 22.7 mmol) was added to a solution of intermediate 62 (0.84 g, 3.0 mmol) in 1,4-dioxane (8 mL). The mixture was stirred at 80° C. for 6 h. Then, more manganese(IV)oxide (2.5 eq, 0.65 g, 7.5 mmol) was added and the mixture was stirred at 80° C. for 24 h. Then more manganese(IV)oxide (2.5 eq, 0.65 g, 7.5 mmol) was added and the mixture was stirred at 80° C. for 40 h. The solid was filtered off and washed with EtOAc and MeOH, and the filtrate was concentrated under reduced pressure. The residue was dissolved in 1,4-dioxane (8 mL) and manganese(IV)oxide (2.5 eq, 0.65 g, 7.5 mmol) was added to the mixture. The mixture was stirred at 80° C. for 3 days. The solid was filtered-off and washed with EtOAc and MeOH, and the filtrate was concentrated under reduced pressure. The crude product was purified by flash column chromatography (silica; EtOAc in heptane 0/100 to 100/0). The desired fractions were collected and the solvents evaporated in vacuo to yield intermediate 63 (0.23 g, 27%) as a colorless oil.

### Preparation of Intermediate 64

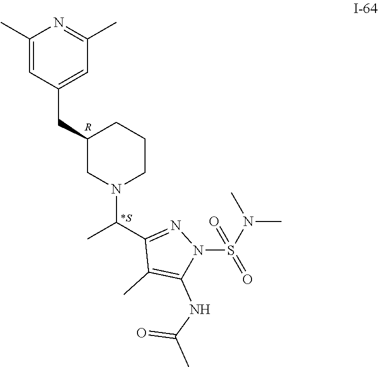

To a solution of intermediate 3 (0.04 g, 0.20 mmol) in DCM (1 mL), intermediate 63 (0.06 g, 0.22 mmol) and titanium(IV)isopropoxide (0.09 mL, 0.30 mmol) were added and the reaction mixture was stirred at rt for 3 h. Then the reaction was cooled to 0° C. and a 1.4M solution of methylmagnesium bromide in THF/toluene (0.71 mL, 0.99 mmol) was added dropwise and the reaction mixture was stirred at 0° C. for 5 min and at rt for 21 h. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 6/94). The desired fractions were collected and the solvents evaporated in vacuo to yield intermediate 64 (0.06 g, 63%) as a colorless oil.

### Preparation of Intermediate 65

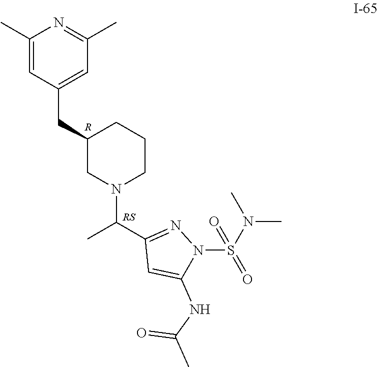

To a solution of intermediate 3 (0.10 g, 0.52 mmol) in DCE (2 mL), titanium(IV)isopropoxide (0.23 mL, 0.78 mmol), intermediate 54 (0.15 g, 0.54 g) and sodium cyanoborohydride (0.39 g, 0.62 mmol) were added and the reaction mixture was stirred at 80° C. for 2 h. The mixture was concentrated in vacuo and the residue purified by flash column chromatography (silica; 7M ammonia solution in EtOH in DCM 0/100 to 05/95) The desired fractions were collected and concentrated in vacuo to yield intermediate 65 (0.20 g, 69%, 70% pure) as a white solid.

### Preparation of Intermediate 66

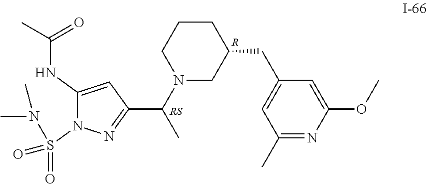

Intermediate 66 was prepared following an analogous procedure to the one described for the synthesis of intermediate 65 using intermediate 5 as starting material.

### Preparation of Intermediate 67

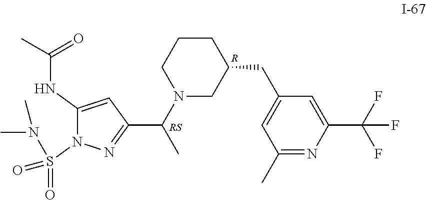

Intermediate 67 was prepared following an analogous procedure to the one described for the synthesis of intermediate 65 using intermediate 8 as starting material.

### Preparation of Intermediate 68

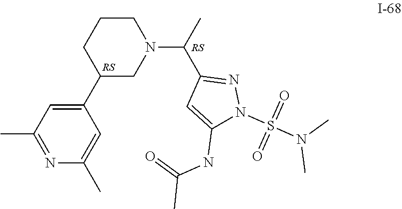

Intermediate 68 was prepared following an analogous procedure to the one described for the synthesis of intermediate 65 using intermediate 11 as starting material.

### Preparation of Intermediate 69

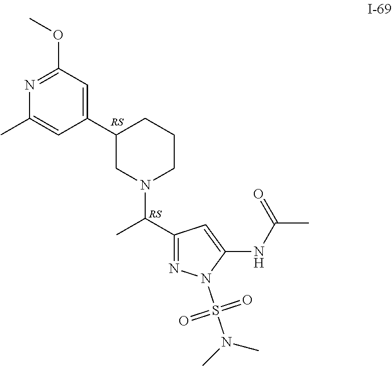

Intermediate 69 was prepared following an analogous procedure to the one described for the synthesis of intermediate 65 using intermediate 14 as starting material.

### Preparation of Intermediate 70

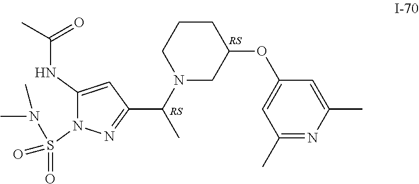

Intermediate 70 was prepared following an analogous procedure to the one described for the synthesis of intermediate 65 using intermediate 23 as starting material.

### Preparation of Intermediate 71

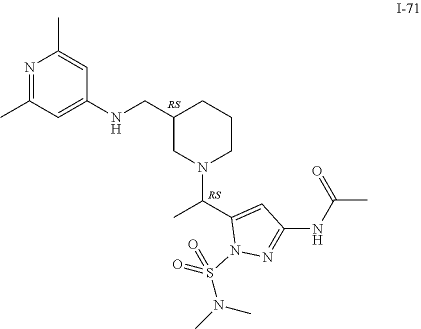 [SMILES: CC(=O)Nc1cc(C(C)N2CCCC(CNc3cc(C)nc(C)c3)C2)n(S(=O)(=O)N(C)C)n1]

Intermediate 71 was prepared following an analogous procedure to the one described for the synthesis of intermediate 65 using intermediate 43 as starting material.

### Preparation of Intermediate 72

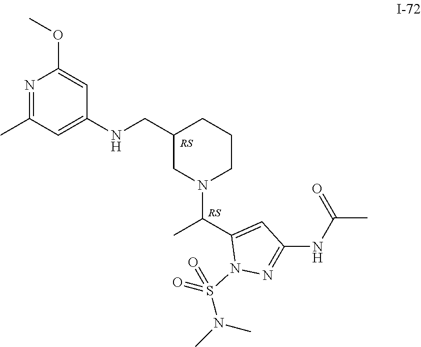 [SMILES: COc1cc(NCC2CCCN(C(C)c3cc(NC(C)=O)nn3S(=O)(=O)N(C)C)C2)cc(C)n1]

Intermediate 72 was prepared following an analogous procedure to the one described for the synthesis of intermediate 65 using intermediate 45 as starting material.

### Preparation of Intermediate 73

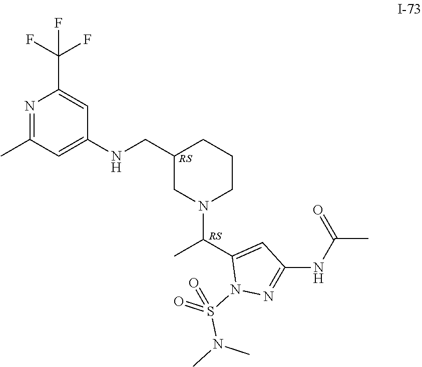 [SMILES: CC(=O)Nc1cc(C(C)N2CCCC(CNc3cc(C)nc(C(F)(F)F)c3)C2)n(S(=O)(=O)N(C)C)n1]

Intermediate 73 was prepared following an analogous procedure to the one described for the synthesis of intermediate 65 using intermediate 48 as starting material.

### Preparation of Intermediate 74

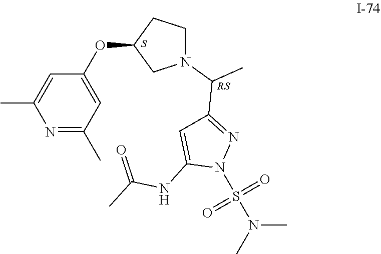

Intermediate 74 was prepared following an analogous procedure to the one described for the synthesis of intermediate 65 using intermediate 41 as starting material.

### Preparation of Intermediate 75

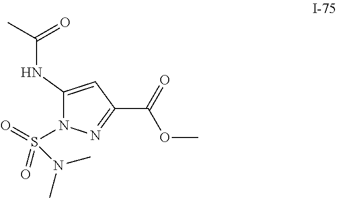 [SMILES: COC(=O)c1cc(NC(C)=O)n(S(=O)(=O)N(C)C)n1]

DABCO (0.13 g, 1.2 mmol) was added followed by N,N-dimethylsulfamoyl chloride (0.12 mL, 1.1 mmol) to a solution of methyl 3-(acetylamino)-1H-pyrazole-5-carboxylate (CAS 1202657-29-1, 0.2 g, 1.09 mmol) in ACN (4 mL) at 0° C. The mixture was allowed to warm to rt and stirred for 18 h. The mixture was concentrated in vacuo and the residue was diluted with sat. NH₄Cl and the product extracted with EtOAc. The organic layer was separated, dried (MgSO₄), filtered and the solvents removed in vacuo to yield intermediate 75 (0.31 g, 99) as a colorless oil which solidified upon standing. The product was used in the following reaction without further purification.

### Preparation of Intermediate 76

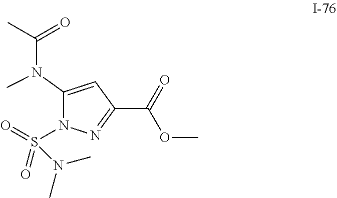 [SMILES: COC(=O)c1cc(N(C)C(C)=O)n(S(=O)(=O)N(C)C)n1]

To a solution of intermediate 75 (0.30 g, 1.03 mmol) in anhydrous DMF (6 mL), a 60% sodium hydride dispersion in mineral oil (0.06 g, 1.55 mmol) was added under a N₂ atmosphere and the reaction mixture was allowed to warm to rt and stirred at rt for 30 min. Then the reaction was cooled to 0° C. and iodomethane (0.13 mL, 2.07 mmol) was added. The reaction mixture was allowed to warm to rt and stirred at rt for 2 h. The reaction mixture was then diluted with H₂O and the product extracted with EtOAc. The combined organic layers were dried (MgSO₄), filtered and the solvents evaporated in vacuo. The product was purified by flash chromatography (silica; EtOAc in heptane, 1:90 to 50:50). The desired fractions were collected and the solvents evaporated in vacuo to yield intermediate 76 (0.25 g, 81%) as a colorless oil which solidified upon standing.

### Preparation of Intermediate 77

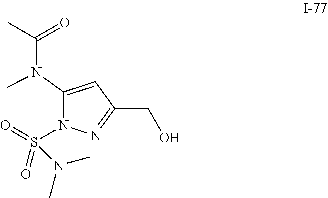 [SMILES: CC(=O)N(C)c1cc(CO)nn1S(=O)(=O)N(C)C]

Lithium borohydride (0.03 g, 1.64 mmol) was added portionwise to a stirred solution of intermediate 76 (0.25 g, 0.82 mmol) in dry THF (1 mL) at 0° C. and under a N₂ atmosphere. After the addition was completed, MeOH (10 uL) was added and the reaction mixture was warm to rt and stirred for 2 h. The reaction was cooled to 0° C. and EtOAc (5 mL) was added followed by slow addition of H₂O (25 mL). The organic layer was separated and the aqueous layer was further extracted with EtOAc (3×25 mL). The combined extracts were dried (MgSO₄), filtered and the solvents removed in vacuo to yield intermediate 77 (0.22 g, 97%) as a white solid (mixture of isomers ˜8:2). The product was used in the following reaction without further purification.

### Preparation of Intermediate 78

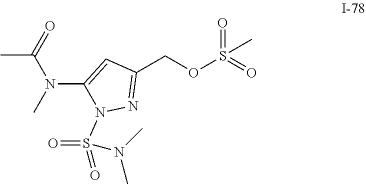 [SMILES: CC(=O)N(C)c1cc(COS(C)(=O)=O)nn1S(=O)(=O)N(C)C]

A mixture of intermediate 77 (0.22 g, 0.79 mmol) and TEA (0.22 mL, 1.59 mmol) in dry DCM (4.8 mL) was cooled under N₂ to 0-5° C. Then methanesulfonic anhydride (0.23 g, 1.35 mmol) was added and the mixture was allowed to warm to rt and stirred for 18 h. The reaction was diluted with DCM, and washed with aqueous 1N NaHSO₄. After separating the layers, the aqueous phase was back-extracted with DCM. The combined organic layers were washed with aqueous K₂CO₃ (5% w/v), dried over anhydrous MgSO₄, filtered and evaporated in vacuo. The crude material was purified by flash chromatography (silica; EtOAc in heptane 0/100 to 20/80). Fractions containing the product were combined, evaporated in vacuo and dried under high vacuo to yield intermediate 78 (50 mg, 18%).

### Preparation of Intermediate 79

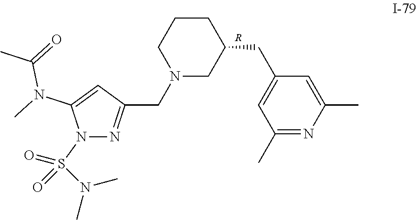

To a solution of intermediate 3 (0.03 g, 0.15 mmol) in ACN (1 mL); intermediate 78 (0.05 g, 0.15 mmol) and K₂C03 (0.06 g, 0.44 mmol) were added and the reaction mixture was stirred at 75° C. for 18 h. Then the reaction was diluted with DCM and washed with H₂O. The organic layer was separated, dried (MgSO₄), filtered and the solvents evaporated in vacuo. The product was purified by RP-HPLC (Stationary phase: C18 XBridge 30×100 mm 5 um, mobile phase: Gradient from 80% 10 mM NH₄CO₃H pH 9 solution in H₂O, 20% ACN to 63% 10 mM NH₄CO₃H pH 9 solution in H₂O, 37% ACN) to yield intermediate 79 (0.04 g, 59%) as a white solid.

### Preparation of Intermediate 80

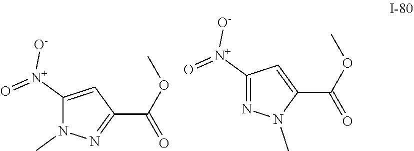

To a solution of the 5-nitro-1H-pyrazole-3-carboxylic acid, methyl ester (CAS 181585-93-3, 2.0 g, 11.69 mmol) in anhydrous DMF (15.4 mL), K₂CO₃ (3.23 g, 23.38 mmol) and iodomethane (0.95 mL, 15.19 mml) were added under a N₂ atmosphere and the reaction mixture was stirred at rt for 18 h. The reaction mixture was then diluted with H₂O and the product extracted with DCM. The combined organic layers were, dried (MgSO₄), filtered and the solvents evaporated in vacuo. The product was purified by flash chromatography (silica; EtOAc in heptane, 1:90 to 50:50). The desired fractions were collected and the solvents evaporated in vacuo to yield intermediate 80 (1.10 g, 51%) as a mixture of isomers (˜8:2).

### Preparation of Intermediate 81

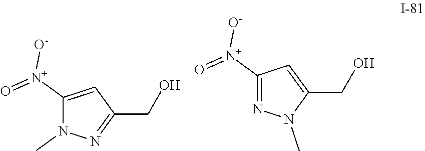

Lithium borohydride (0.23 g, 10.8 mmol) was added portionwise to a stirred solution of intermediate 80 (1.0 g, 5.4 mmol) in dry THF (11 mL) at 0° C. and under a N₂ atmosphere. After the addition was completed, MeOH (0.08 mL, 1.97 mmol) was added and then the reaction mixture was warmed to rt and stirred for 2 h. The reaction was cooled to 0° C. and EtOAc (5 mL) was added followed by slow addition of H₂O (25 mL). The organic layer was separated and the aqueous layer was further extracted with EtOAc (3×25 mL). The combined extracts were dried (MgSO₄), filtered and the solvents removed in vacuo to yield intermediate 81 (0.82 g, 97%) as a white solid (mixture of isomers ˜8:2). The product was used in the following reaction without further purification.

### Preparation of Intermediate 82

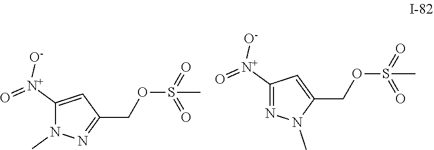

A mixture of intermediate 81 (0.10 g, 0.64 mmol) and TEA (0.18 mL, 1.27 mmol) in dry DCM (2 mL) was cooled under N₂ to 0-5° C. Then methanesulfonic anhydride (CAS 7143-01-3, 0.19 g, 1.08 mmol) was added and the mixture was stirred for approximately 10 minutes at 0° C. and at rt for 3 h. The reaction was diluted with DCM, and washed with sat NH₄Cl solution. After separating the layers, the aqueous phase was back-extracted with DCM. The combined organic layers were, dried over anhydrous MgSO₄, filtered and evaporated in vacuo to yield intermediate 82 (0.12 g, 80%, mixture 83/13) as an oil. The crude material was used in the next step without further purification.

### Preparation of Intermediates 83 and 84

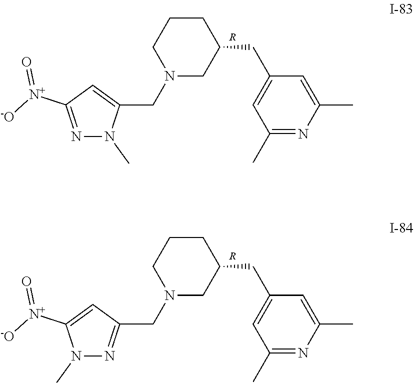

To a solution of intermediate 3 (0.12 g, 0.59 mmol) in ACN (0.12 mL); intermediate 82 (0.14 g, 0.63 mmol) and K₂CO₃ (0.24 g, 1.76 mmol) were added and the reaction mixture was stirred at 75° C. for 18 h. Then the reaction was diluted with DCM and washed with H₂O. The organic layer was separated, dried (MgSO₄), filtered and the solvents evaporated in vacuo. The product was purified by RP HPLC (Stationary phase: C18 XBridge 30×100 mm 5 um, mobile phase: Gradient from 80% 10 mM NH₄CO₃H pH 9 solution in H₂O, 20% CH₃CN to 63% 10 mM NH₄CO₃H pH 9 solution in H₂O, 37% CH₃CN) to intermediate 83 (0.13 g, 64%) and intermediate 84 (20 mg, 10%) as white solids.

### Preparation of Intermediate 85

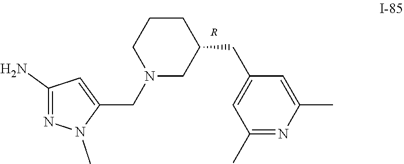

A suspension of intermediate 83 (0.13 g, 0.38 mmol) in MeOH (7.6 mL), Pd/C (10%) (0.08 g, 0.07 mmol) was added and the mixture was hydrogenated (atmospheric pressure) at rt for 18 h. The reaction was filtered and the filtrate concentrated in vacuo to give intermediate 85 (115 mg, 97%) as a white solid. The product was used in the next step reaction without further purification.

### Preparation of Product 86

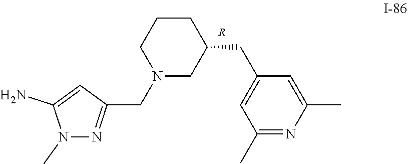

A suspension of intermediate 84 (20 mg, 0.06 mmol) in MeOH (1.2 mL), Pd/C (10%) (12 mg, 0.012 mmol) was added and the mixture was hydrogenated (atmospheric pressure) at rt for 18 h. The reaction was filtered and the filtrate concentrated in vacuo to give intermediate 86 (17 mg, 93%) as a white solid. The product was used in the next reaction without further purification.

### Preparation of Intermediate 87

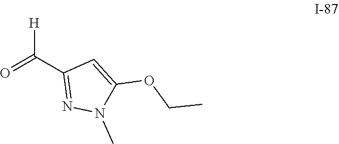

Manganese(IV)oxide (2.13 g, 24.55 mmol) was added to a stirred solution of (5-ethoxy-1-methyl-1H-pyrazol-3-yl)ethanol (CAS 1365940-38-0, 0.77 g, 4.9 mmol) in 1,4-dioxane (15 mL). The reaction mixture was stirred at 80° C. overnight. The solid was filtered off through a pad of diatomaceous earth, washed with MeOH, and the filtrate was concentrated under reduced pressure. The crude product was purified by flash column chromatography (silica; EtOAc in heptane 0/100 to 30/70). The desired fractions were collected and the solvents evaporated in vacuo to yield intermediate 87 (0.59 g, 77%) as a white solid.

### Preparation of Intermediate 88

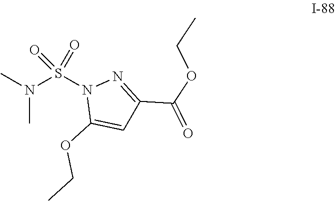 [SMILES: CCOC(=O)c1cc(OCC)n(S(=O)(=O)N(C)C)n1]

1,4-Diazabicyclo[2.2.2]octane (1.1 eq, 0.20 g, 1.8 mmol) was added followed by dimethylsulfamoyl chloride (1.0 eq, 0.72 mL, 1.8 mmol) to a solution of 5-ethoxy-1H-pyrazole-3-carboxylic acid, ethyl ester (CAS 1116656-05-3, 0.42 g, 1.61 mmol) in ACN (2.9 mL) at 0° C. The mixture was allowed to warm to rt and stirred for 4 h. Then, more 1,4-diazabicyclo[2.2.2]octane (0.5 eq, 0.15 g, 0.8 mmol) and dimethylsulfamoyl chloride (0.4 eq, 0.17 mL, 0.9 mmol) were added to the mixture. The mixture was stirred at rt for 16 h. The mixture was concentrated in vacuo and the residue was diluted with H₂O and extracted with EtOAc. The organic layer was separated, dried (MgSO₄), filtered and the solvents removed in vacuo. The crude product was purified by flash column chromatography (silica; EtOAc in heptane 0/100 to 50/50). The desired fractions were collected and the solvents evaporated in vacuo to yield intermediate 88 (0.43 g, 93%) as a pale-yellow oil.

### Preparation of Intermediate 89

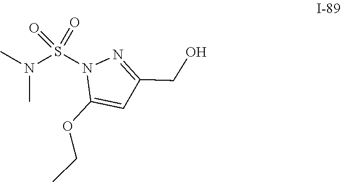 [SMILES: CCOc1cc(CO)nn1S(=O)(=O)N(C)C]

LAH 1M solution in THF (1.8 mL, 1.8 mmol) was added to a stirred solution of intermediate 88 (0.43 g, 1.5 mmol) in THF (1.6 mL) at 0° C. and under N₂. The mixture was left warming slowly to rt and stirred for 2 h. The mixture was carefully treated with 1N HCl until pH 7 at 0° C. and the product was extracted with EtOAc. The organic layer was separated, dried (MgSO₄), filtered and the solvents removed in vacuo to yield intermediate 89 (0.34 g, 92%) as a colorless oil.

### Preparation of Intermediate 90

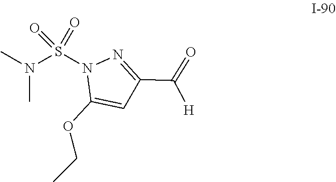 [SMILES: CCOc1cc(C=O)nn1S(=O)(=O)N(C)C]

To a solution of intermediate 89 (0.3 g, 1.2 mmol) in 1,4-dioxane (5 mL), was added manganese(IV)oxide (5.0 eq, 0.52 g, 5.95 mmol) and the reaction mixture was stirred at 70° C. for 4 h. More manganese(IV)oxide (2.5 eq, 0.26 g, 3.0 mmol) was added to the mixture at rt and the mixture was stirred at 70° C. for 25 h. The solid was filtered off and washed with EtOAc, and the filtrate was concentrated under reduced pressure. The crude product was purified by flash column chromatography (silica; EtOAc in heptane 0/100 to 35/65). The desired fractions were collected and the solvents evaporated in vacuo to yield intermediate 90 (0.17 g, 58%) as a pale orange oil.

### Preparation of Intermediate 91

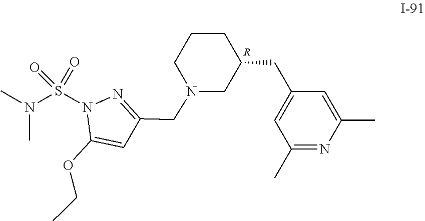

To a solution of intermediate 3 (0.13 g, 0.62 mmol) in DCM (2 mL), intermediate 90 (0.16 g, 0.65 g) was added and the reaction mixture was stirred at rt for 1 h. Then sodium triacetoxyborohydride (2.0 eq, 0.26 g, 1.25 mmol) was added and the reaction mixture was stirred at rt for 3 h. Then more sodium triacetoxyborohydride (2.0 eq, 0.26 g, 1.25 mmol) was added and the reaction mixture was stirred at rt for 16 h. Then saturated solution of NaHCO₃ was added and the product extracted with DCM. The organic layer was separated, dried (MgSO₄), filtered and the solvents evaporated in vacuo. The crude product was purified by flash column chromatography (silica; MeOH in DCM 0/100 to 5/95). The desired fractions were collected and the solvents evaporated in vacuo to intermediate 91 (0.23 g, 84%) yield as a colorless oil.

### Preparation of Intermediate 92

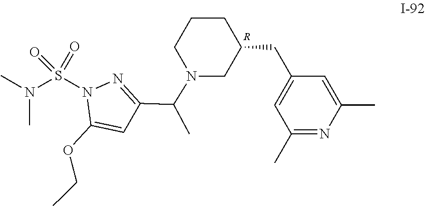

To a solution of Intermediate 3 (0.10 g, 0.45 mmol) in DCM (1.5 mL), intermediate 90 (0.13 g, 0.54 mmol) and titanium(IV)isopropoxide (0.21 mL, 0.73 mmol) were added and the reaction mixture was stirred at rt for 3 h. Then the reaction was cooled to 0° C. and 1.4M solution of methylmagnesium bromide in THF:toluene (1.7 mL, 2.44 mmol) was added dropwise and the reaction mixture was stirred at 0° C. for 5 min. and at rt for 1.3 h. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 1/99). The desired fractions were collected and the solvents evaporated in vacuo to yield intermediate 92 (0.08 g, 61%) as a colorless oil.

### Preparation of Intermediate 93

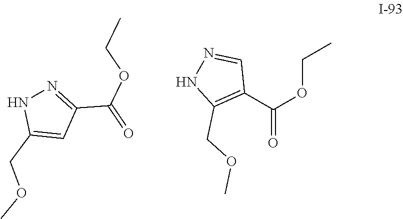

Ethyl diazoacetate (7.5 mL, 72.0 mmol) was added dropwise to a stirred solution of methyl propargyl ether (5 g, 71.33 mmol) in anhydrous toluene (70 mL) at 0° C. The mixture was stirred at rt for 10 min and then at 115° C. for 5 h. The solvent was evaporated in vacuo and the crude product was purified by flash column chromatography (silica; EtOAc in heptane 0/100 to 50/50). The desired fractions were collected and the solvents evaporated in vacuo to yield intermediate 93 (a mixture of two products) (5.2 g, 98%) as yellow oils.

### Preparation of Intermediates 94 and 95

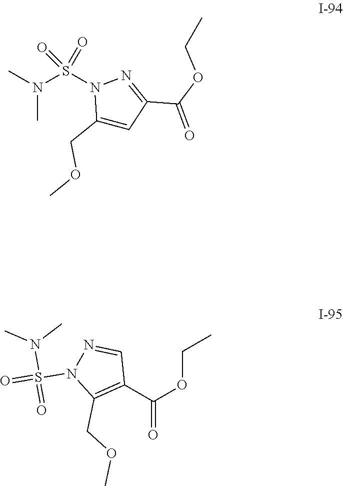

DABCO (0.91 g, 8.14 mmol) was added followed by dimethylsulfamoyl chloride (0.76 mL, 7.06 mmol) to a solution of intermediate 93 (1.0 g, 5.42 mmol) in ACN (25 mL) at 0° C. The mixture was allowed to warm to rt and stirred for 23 h. The mixture was concentrated in vacuo and the residue was diluted with H₂O and was extracted with EtOAc. The organic layer was separated, dried (MgSO₄), filtered and the solvents removed in vacuo. The crude product was purified by flash column chromatography (silica; EtOAc in heptane 0/100 to 50/50). The desired fractions were collected and the solvents evaporated in vacuo to yield intermediate 94 (0.76 g, 48%) and intermediate 95 (0.65 g, 41%) as a pale-yellow oils.

### Preparation of Intermediate 96

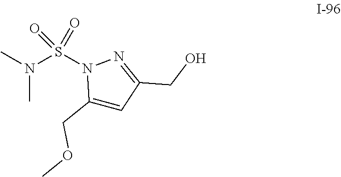 [SMILES: COCc1cc(CO)nn1S(=O)(=O)N(C)C]

LAH (2.9 mL, 2.9 mmol) was added to a stirred solution of intermediate 94 (0.70 g, 2.43 mmol) in THF (2.7 mL) at 0° C. and under N₂. The mixture was left warming slowly to rt and stirred for 2 h. The mixture was diluted with EtOAc and Na₂SO₄.10H₂O was added at 0° C. The mixture was stirred for 15 min at 0° C., filtered through a pad of diatomaceous earth and washed with additional EtOAc. The solvents were evaporated in vacuo to yield intermediate 96 (0.35 g, 57%) as a colorless oil.

### Preparation of Intermediate 97

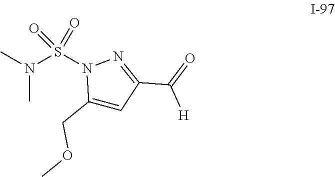 [SMILES: COCc1cc(C=O)nn1S(=O)(=O)N(C)C]

Manganese(IV)oxide (0.68 g, 7.82 mmol) was added to a solution of intermediate 96 (0.35 g, 1.39 mmol) in DCE (5 mL) and the reaction mixture was stirred at 80° C. for 2 h. Then, more manganese(IV)oxide (0.6 equiv, 73 mg, 0.83 mmol) was added and the mixture was stirred at rt for 48 h. The solid was filtered off and washed with DCE and MeOH, and the filtrate was evaporated in vacuo. The crude product was purified by flash column chromatography (silica; EtOAc in heptane 0/100 to 50/50). The desired fractions were collected and the solvents evaporated in vacuo to yield intermediate 97 (0.20 g, 60%) as a colorless oil.

### Preparation of Intermediate 98

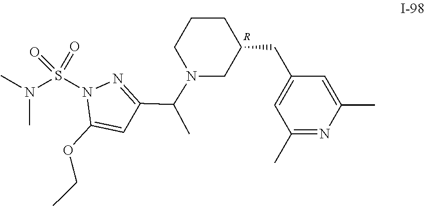

To a solution of intermediate 3 (0.06 g, 0.28 mmol) in DCM (1 mL), intermediate 97 (0.09 mg, 0.36 mmol) and titanium(IV)isopropoxide (0.12 mL, 0.42 mmol) were added and the reaction mixture was stirred at rt for 24 h. Then the reaction was cooled to 0° C. and a 1.4M solution of methylmagnesium bromide in THF:toluene (1 mL, 1.4 mmol) was added dropwise and the reaction mixture was stirred at 0° C. for 5 min and at rt for 1.3 h. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 10/90). The desired fractions were collected and the solvents evaporated in vacuo to yield intermediate 98 (0.21 g, 97%) as a colorless oil.

### Preparation of Intermediate 99

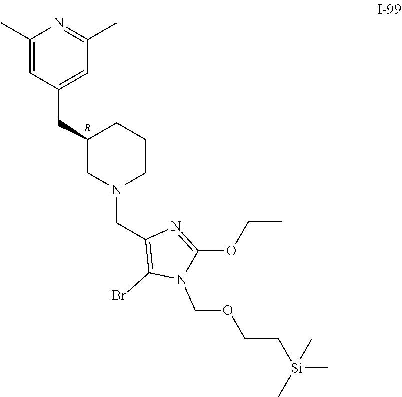

To a solution of intermediate 3 (0.2 g, 0.72 mmol) and TEA (0.4 mL, 2.88 mmol) in DCM (11 mL), 4-bromo-2-ethoxy-1-[[2-(trimethysilyl)ethoxy]methyl]-1H-imidazole-5-carboxaldehyde (CAS 1073543-59-5, 0.30 g, 0.87 mmol) and sodium triacetoxyborohydride (0.35 g, 1.65 mmol) were added and the reaction mixture was stirred at rt for 18 h. Then saturated solution of NaHCO₃ was added and the product extracted with EtOAc. The organic layer was separated and evaporated in vacuo. The residue was purified by flash column chromatography (silica; 7M solution of ammonia in MeOH in DCM 0/100 to 6/94). The desired fractions were collected and concentrated in vacuo to yield intermediate 99 (14.3 g, 94%) as transparent oil.

### Preparation of Intermediate 100

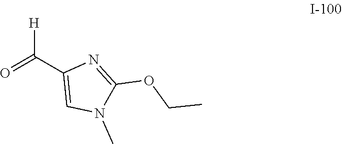

A 2.5 M solution of n-butyllithium in hexanes (0.15 mL, 0.37 mmol) was added dropwise to a stirred solution of 4-bromo-2-ethoxy-1-methyl-11H-imidazole (CAS 1895273-39-8, 0.72 g, 0.35 mmol) in THF (3.5 mL), under N₂ and at −78° C. The mixture was stirred at −78° C. for 20 min and then DMF (0.08 mL, 1.05 mmol) was added dropwise. The resulting mixture was stirred at −78° C. for 15 min and then warmed to rt and stirred for 1 h. The reaction was quenched with H₂O and sat. NH₄Cl and extracted with EtOAc. The organic layer was separated, dried (Na₂SO₄), filtered and the solvents evaporated in vacuo to yield intermediate 100 (46 mg, 76%) as a yellowish oil, that was used in next step without further purification.

### Preparation of Intermediate 101

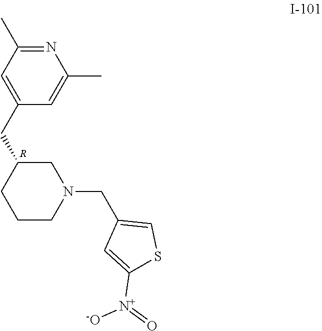

DIPEA (0.15 mL, 0.87 mmol) followed by 4-(chloromethyl)-2-nitrothiophene (CAS 1092561-29-9, 0.04 mL, 0.33 mmol) were added to a stirred solution of intermediate 3 (0.067 g, 0.24 mmol) in ACN (1.2 mL) in a sealed tube and under N₂. The mixture was stirred at rt for 16 h. The mixture was treated with sat NaHCO₃ and extracted with DCM. The organic layer was separated, dried (MgSO₄), filtered and the solvents evaporated in vacuo. The crude product was purified by flash column chromatography (1st SiO₂ NH₂ functionalized, EtOAc in heptane 0/100 to 100/0 and then silica; 7N solution of NH₃ in MeOH in DCM 0/100 to 10/90). The desired fractions were collected and concentrated in vacuo to yield intermediate 101 (54 mg, 65%) as a dark oil.

### Preparation of Intermediate 102

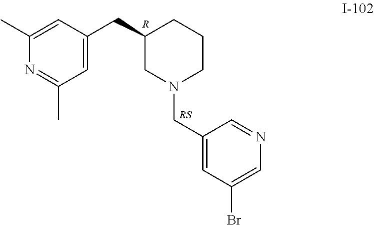

Sodium cyanoborohydride (0.025 g, 0.39 mmol) was added to a stirred solution of intermediate 3 (0.1 mg, 0.36 mmol), 5-bromonicotinaldehyde (CAS 113118-81-3, 0.09 g, 0.50 mmol) and sodium acetate (0.09 g, 1.08 mmol) in EtOH (1 mL) at rt. The reaction mixture was stirred at rt for 16 h. Bromonicotinaldehyde (CAS 113118-81-3, 0.09 g, 0.50 mmol) and sodium cyanoborohydride (0.025 g, 0.39 mmol) were added. The reaction mixture was stirred at rt for 16 h. NaHCO₃ (5 mL) was added and the mixture was extracted with EtOAc (10 mL×3 times). The organic layer was separated, dried (MgSO₄), filtered and the solvents evaporated in vacuo. The crude was purified by flash column chromatography (silica; EtOAc in heptane 0/100 to 100/0). The desired fractions were collected and concentrated in vacuo to yield intermediate 102 (0.11 g, 85%) as a colorless sticky solid.

### Preparation of Intermediate 103

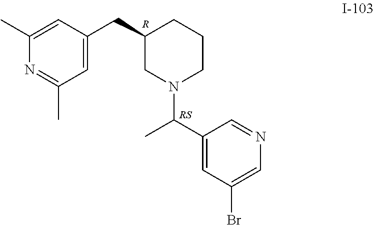

5-Bromonicotinaldehyde (CAS 113118-81-3, 0.21 g, 1.1 mmol) and titanium(IV)isopropoxide (0.65 mL, 2.20 mmol) were added to a solution of intermediate 3 (0.15 g, 0.73 mmol) in anhydrous THF (1 mL) at rt and the reaction mixture was stirred at rt for 18 h. The mixture was distillated and dried in vacuo. Then, anhydrous THF (1 mL) was added and the reaction was cooled to 0° C. and a 1.4M solution of methylmagnesium bromide in THF:toluene (2.6 mL) was added dropwise. The reaction mixture was stirred at 0° C. for 15 min and at rt for 15 h. A saturated solution of NH₄Cl was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo to yield intermediate 103 (64 mg, 21%) as a colorless sticky solid.

### Preparation of Intermediate 104

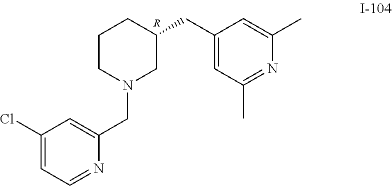

Acetic acid (0.086 mL, 1.51 mmol) and sodium cyanoborohydride (47 mg, 0.76 mmol) was added to a stirred solution of intermediate 3 (0.21 g, 0.76 mmol), 4-chloropyridine-2-carbaldehyde (CAS 63071-13-6, 0.12 g, 0.83 mmol) and sodium acetate anhydrous (0.24 g, 2.95 mmol) in EtOH (5 mL) at rt. The reaction mixture was stirred at rt for 6 h. Then, 4-chloropyridine-2-carbaldehyde (CAS 63071-13-6, 0.12 g, 0.83 mmol) and sodium cyanoborohydride (0.04 g, 0.76 mmol) were added and the mixture was stirred at rt for 48 h. The mixture was diluted with a saturated NaHCO₃ solution and extracted with EtOAc. The organic layer was separated, dried (MgSO₄), filtered and the solvents evaporated in vacuo and the crude was purified by flash column chromatography (silica; MeOH/DCM (9:1) in DCM 0/100 to 50/50). The desired fractions were collected and concentrated in vacuo to yield expected product as a yellow sticky solid. A fraction of this compound (50 mg) was purified by RP-HPLC (from 90% (H₂O 25 mM NH₄HCO₃)—10% MeCN-MeOH to 54% H₂O (25 mM NH₄HCO₃)—46% MeCN-MeOH). The desired fractions were collected concentrated in vacuo to yield expected compound as a pale-yellow sticky solid. The material was taken into DCM and treated with 4N solution of HCl in 1,4-dioxane (0.05 mL). The solvents evaporated in vacuo and the product was triturated with diethyl ether to yield intermediate 104 (27 mg, 9%) as a beige solid.

### Preparation of Intermediate 105

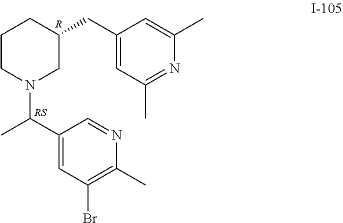

5-Bromo-6-methyl-3-pyridinecarboxaldehyde (CAS 1174028-20-6, 0.19 g, 0.95 mmol) and titanium(IV)isopropoxide (0.56 mL, 1.90 mmol) were added to a solution of intermediate 3 (0.13 g, 0.63 mmol) in anhydrous THF (2 mL) at rt and the reaction mixture was stirred at rt for 18 h. Then, the solvent was concentrated in vacuo and the mixture was added anhydrous THF (2 mL) under N₂. The mixture was cooled to 0° C. and a 1.4 M solution of methylmagnesium bromide in THF:toluene (2.27 mL, 3.18 mmol) was added. The reaction mixture was stirred at 0° C. for 15 min and at rt for 3 h. The mixture was stirred at rt for 16 h more. NH₄Cl sat was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo intermediate 105 (78 mg, 30%) to yield as a yellow oil.

### Preparation of Intermediate 106

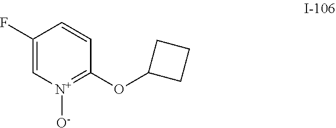 [SMILES: [O-][n+]1cc(F)ccc1OC1CCC1]

To a mixture of 2-(cyclobutyloxy)-5-fluoro-pyri dine (CAS 1824652-46-1, 1.7 g, 10.1 mmol) in DCM (20 mL), m-CPBA (2 g, 12.1 mmol) was added at rt. The mixture was stirred 36 h at 25° C. The solvent was removed in vacuo, and the residue was purified by silica gel column chromatography (silica; EtOAc in heptane 0/100 to 30/70 then MeOH in DCM 0/100 to 4/96). The desired fractions were collected and concentrated in vacuo to afford intermediate 106 (0.65 g, 35%) as a white solid.

### Preparation of Intermediate 107

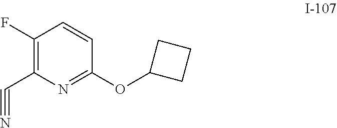 [SMILES: N#Cc1nc(OC2CCC2)ccc1F]

To a mixture of intermediate 106 (0.6 g, 3.3 mmol) in ACN (10 mL), trimethylsilyl cyanide (0.9 mL, 77.5 mmol) and TEA (0.7 mL, 4.9 mmol) were added. The mixture was stirred at 90° C. 24 h. The mixture was cooled and treated with H₂O and extracted with EtOAc (2×10 ml). The organic layers were dried over MgSO₄ and the solvent was removed in vacuo to yield an oil which was purified by flash column chromatography (silica; EtOAc in Heptane 0/100 to 30/60). The desired fractions were collected and concentrated in vacuo to yield intermediate 107 (0.3 g, 48%) as an oil.

### Preparation of Intermediate 108

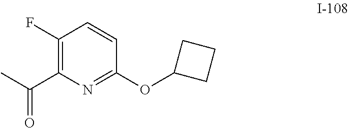

To a solution of intermediate 107 (0.16 g, 0.83 mmol) in dry THF (2 mL), a 1.4M solution of methylmagnesium bromide in THF:toluene (1.25 mL, 1.75 mmol) was added at 0° C. After completion of the addition, the reaction was stirred for 16 h at rt. The mixture was quenched with 1M aq HCl and stirred for 30 min, then the crude was basified with NH₄OH until pH 8. The solution was extracted with EtOAc (2×5 mL) The combined organics were dried (Na₂SO₄), filtered and evaporated to dryness to give an oil. The crude was purified by flash column chromatography (silica; EtOAc in Heptane 0/100 to 10/90). The desired fractions were collected and concentrated to yield intermediate 108 (0.16 g, 92%) as a transparent oil.

### Preparation of Intermediate 109

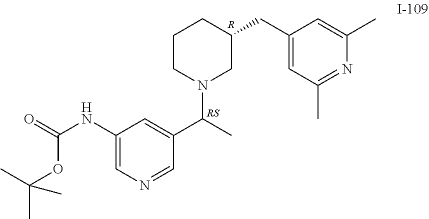

Tert-butyl(5-formylpyridin-3-yl)carbamate (CAS 337904-94-6, 0.135 g, 0.59 mmol) and titanium(IV)isopropoxide (0.35 mL, 1.18 mmol) were added to a solution of intermediate 3 (0.080 g, 0.39 mmol) in anhydrous THF (1 mL) at rt and the reaction mixture was stirred at rt for 18 h. The mixture was distillated and dried in vacuo. Then, anhydrous THF (1 mL) was added and the reaction was cooled to 0° C. and a 1.4M solution of methylmagnesium bromide in THF:toluene (1.40 mL, 1.97 mmol) was added dropwise and the reaction mixture was stirred at 0° C. for 15 min and at rt for 15 h. NH₄Cl sat was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo to yield intermediate 109 (126 mg, 75%) as a colorless sticky solid.

### Preparation of Intermediate 110

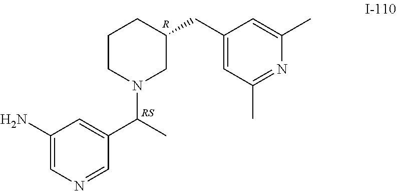

A 4N solution of HCl in 1,4-dioxane (3.7 mL, 14.84 mmol) was added to a stirred solution of intermediate 109 (0.126 g, 0.29 mmol) in MeOH (1 mL) and 1,4-dioxane (1 mL) at 0° C. under N₂. The reaction mixture was stirred at rt for overnight. The solvent was evaporated in vacuo. The crude product was dissolved with DCM and washed with sat Na₂CO₃. The organic layer was separated, dried (MgSO₄), filtered and the solvents evaporated in vacuo to yield intermediate 110 (97 mg, quantitative yield) as a colorless sticky solid. The crude product was used without further purification for the next reaction step.

### Preparation of Intermediates 111 and 112

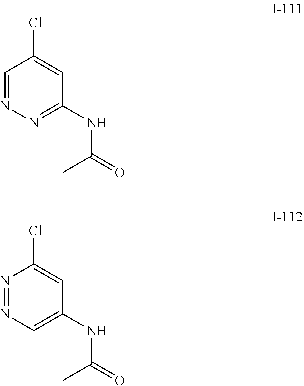

[1,1′-Bis(diphenylphosphino)ferrocene]dichloropalladium(II)dichloromethane (0.27 g, 0.33 mmol), 9,9-dimethyl-4,5-bis(diphenylphosphino)xanthene (0.39 g, 0.67 mmol) and Cs₂CO₃ (13 g, 40.3 mmol) in dry toluene (65 mL) were heated at 40° C. for 15 min while N₂ was bubbling. Then, tert-butyl carbamate (3.1 g, 26.8 mmol) and 3,5-dichloropyridazine (CAS 1837-55-4, 2.5 g, 13.4 mmol) were added while N₂ was bubbling. The mixture was stirred at 80° C. for 16 h. The mixture was diluted with H₂O and extracted with EtOAc. The organic layer was separated, dried (MgSO₄), filtered and the solvents evaporated in vacuo. The crude product was purified by flash column chromatography (silica; EtOAc in heptane, from 0/100 to 50/50). The desired fractions were collected and the solvents evaporated in vacuo to yield intermediate 111 (1.4 g, 45%) and intermediate 112 (0.46 g, 15%) as white solids.

### Preparation of Intermediate 113

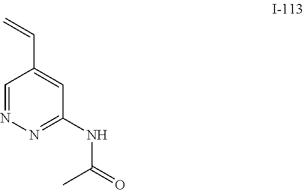

[1,1′-Bis(diphenylphosphino)ferrocene]dichloropalladium(II)dichloromethane (0.1 g, 0.12 mmol) was added to mixture of intermediate 110 (0.46 g, 2.0 mmol), potassium vinyltrifluoroborate (0.42 g, 3.2 mmol), Cs₂CO₃ (2.8 g, 6.0 mmol) in H₂O (2 mL) and 1,4-dioxane (16 mL) at rt for 15 min while N₂ was bubbling. The mixture was stirred at 95° C. for 15 h. The mixture was diluted with H₂O and extracted with EtOAc. The organic layer was separated, washed with H₂O, dried (MgSO₄), filtered and the solvents evaporated in vacuo. The crude product was purified by flash column chromatography (silica; EtOAc in heptane 0/100 to 60/40). The desired fractions were collected and concentrated in vacuo to yield intermediate 113 (0.3 g, 66%) yield as a beige solid.

### Preparation of Intermediate 114

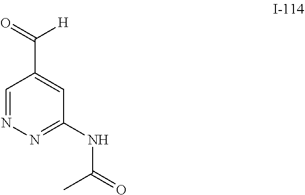

Osmium tetroxide (0.48 mL, 0.04 mmol) was added to a stirred solution of intermediate X (0.3 g, 0.97 mmol) and sodium periodate (0.52 g, 2.4 mmol) in a (1:1) mixture of THF/H₂O (12 mL) at rt and the mixture was stirred for 16 h. The mixture was diluted with H₂O and extracted with DCM. The organic layer was separated, dried (MgSO₄), filtered and the solvents evaporated in vacuo. The crude product was purified by flash column chromatography (silica; EtOAc in heptane 0/100 to 50/50). The desired fractions were collected and concentrated in vacuo to yield intermediate 114 (0.16 g, 76%) as a pale yellow solid.

### Preparation of Intermediate 115

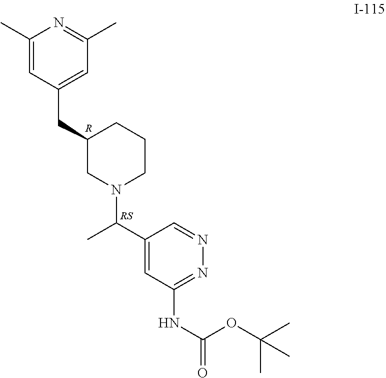

Acetic acid (0.06 mL, 0.98 mmol) and sodium cyanoborohydride (0.05 g, 0.74 mmol) were added to a stirred solution of intermediate 3 (0.14 g, 0.49 mmol), intermediate 113 (0.12 g, 0.54 mmol) and sodium acetate anhydrous (0.16 g, 1.92 mmol) in EtOH (15 mL) at rt. The reaction mixture was stirred at rt for 16 h. Intermediate 112 (0.04 g, 0.19 mmol) and sodium cyanoborohydride (0.03 g, 0.49 mmol) were added and the mixture was stirred at rt for 16 h. The mixture was diluted with sat. NaHCO₃ and extracted with EtOAc. The organic layer was separated, dried (MgSO₄), filtered and the solvents evaporated in vacuo. The crude was purified by flash column chromatography (silica; MeOH/DCM (9:1) in DCM 0/100 to 50/50). The desired fractions were collected and concentrated in vacuo to yield intermediate 115 (0.06 g, 30%) as a colorless oil.

### Preparation of Intermediate 116

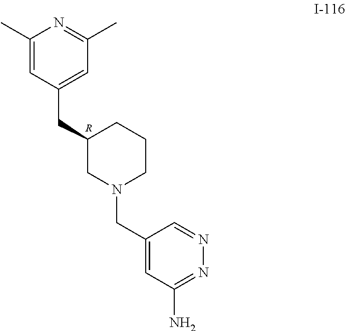

A 4N solution of HCl in 1,4-dioxane (1.8 mL) was added to a stirred solution of intermediate 115 (0.06 mg, 0.14 mmol) in MeOH (1 ml) at 0° C. under N₂. The reaction mixture was stirred at rt for overnight. The solvent was evaporated in vacuo. The crude product was dissolved with DCM and washed with sat Na₂CO₃. The organic layer was separated, dried (MgSO₄), filtered and the solvents evaporated in vacuo to yield intermediate 116 (0.031 g, 68%) as a colorless sticky solid. The crude product was used without further purification in the next reaction step.

### Preparation of Intermediate 117

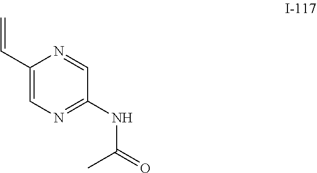

[1,1′-Bis(diphenylphosphino)ferrocene]dichloropalladium(II)dichloromethane (0.14 g, 0.17 mmol) was added to the mixture of N-(5-bromopyrazin-2-yl)acetamide (CAS 174680-67-2, 0.4 g, 1.85 mmol), potassium vinyltrifluoroborate (0.37 g, 2.8 mmol), Cs₂CO₃ (2.65 g, 5.55 mmol) in H₂O (0.5 mL) and 1,4-dioxane (8 mL) at rt. The mixture was stirred at 90° C. for 60 min. The mixture was cooled to ambient temperature and then was filtered through a pad of diatomaceous earth and washed with DCM. MgSO₄ was added, the mixture was filtered and the solvents were concentrated in vacuo. The crude was purified by flash column chromatography (silica; EtOAc in heptane 0/100 to 30/70). The desired fractions were collected and concentrated in vacuo to yield intermediate 117 (0.23 g, 66%) as a white solid.

### Preparation of Intermediate 118

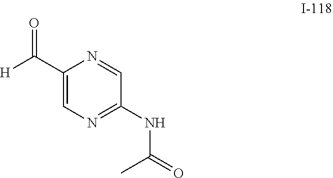

Osmium tetroxide (0.62 mL, 0.05 mmol) was added to a stirred solution of intermediate 117 (0.2 g, 1.24 mmol) and sodium periodate (0.66 g, 3.11 mmol) in a (1:1) mixture of THF/H₂O (10 mL) at rt and the mixture was stirred for 16 h. The mixture was diluted with H₂O and extracted with DCM. The organic layer was separated, dried (MgSO₄), filtered and the solvents evaporated in vacuo. The crude product was purified by flash column chromatography (silica; MeOH in DCM 0/100 to 0/100 to 10/90). The desired fractions were collected and concentrated in vacuo to yield intermediate 118 (0.13 g, 63%) as a brown solid.

### Preparation of Intermediate 119

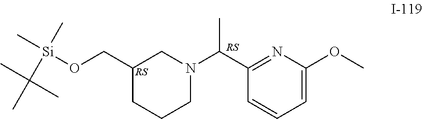 [SMILES: COc1cccc(C(C)N2CCCC(CO[Si](C)(C)C(C)(C)C)C2)n1]

6-Methoxy-2-pyridinecarboxaldehyde (CAS 54221-96-4, 0.65 mL, 5.23 mmol) and titanium(IV)isopropoxide (3.87 mL, 13.07 mmol) were added to a solution of 3-[[[(1,1-dimethylethyl)dimethylsilyl]oxy]methyl]piperidine (CAS 876147-50-1, 1.0 g, 4.36 mmol) in anhydrous THF (11.17 mL) at rt and the reaction mixture was stirred at rt for 5 hours. The mixture was distillated and dried in vacuo. Then, anhydrous THF (11.17 mL) was added and the reaction was cooled to 0° C. and 1.4M solution of methylmagnesium bromide in THF:toluene (15.56 mL, 21.79 mmol) was added dropwise and the reaction mixture was stirred at 0° C. for 15 min and at rt for 15 h. NH₄Cl sat was added and the mixture was extracted with EtOAc (3 times). The organics layers were combined, dried over MgSO₄, filtered and concentrated in vacuo. The crude product was purified by flash column chromatography (silica; EtOAc in Heptane 0/100 to 25/75). The desired fractions were collected and concentrated in vacuo to yield intermediate 119 (0.77 g, 49%) as a colorless oil.

### Preparation of Intermediate 120

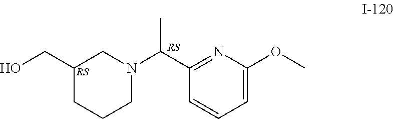 [SMILES: COc1cccc(C(C)N2CCCC(CO)C2)n1]

TBAF (CAS 2206-57-1, 1.19 g, 4.26 mmol) was added to a stirred solution of intermediate 119 (0.78 g, 2.13 mmol) in THF (10 mL) at rt. The mixture was stirred at rt for 8 h. The mixture was diluted with sat NaHCO₃ and extracted with EtOAc. The organic layer was separated, dried (MgSO₄), filtered and the solvents evaporated in vacuo. The crude product was purified by flash column chromatography (silica; MeOH/DCM (1:10) in DCM 0/100 to 10/90). The desired fractions were collected and concentrated in vacuo to yield intermediate 120 (0.42 g, 79%) as a yellow solid.

### Preparation of Intermediate 121

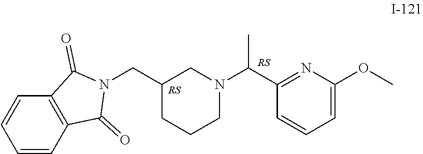 [SMILES: COc1cccc(C(C)N2CCCC(CN3C(=O)c4ccccc4C3=O)C2)n1]

A solution of intermediate 120 (0.42 g, 1.68 mmol), phthalimide (CAS 85-41-6, 0.72 g, 1.85 mmol) and triphenylphosphine (CAS 603-35-0, 0.66 g, 2.52 mmol) in dry THF (20 mL) was stirred under N₂ gas. DIAD (CAS 2446-83-5, 0.49 mL, 2.52 mmol) was added and it was stirred at rt overnight. The solvents evaporated in vacuo. The crude product was purified by flash column chromatography (silica; MeOH in DCM 0/100 to 5/95). The desired fractions were collected and concentrated in vacuo to yield intermediate 121 (0.64 g, quant. yield) as a brown sticky solid.

### Preparation of Intermediate 122

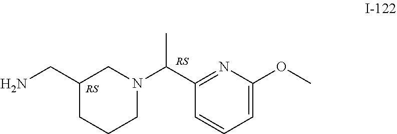 [SMILES: COc1cccc(C(C)N2CCCC(CN)C2)n1]

Hydrazine hydrate (0.47 mL, 8.41 mmol) was added at solution of intermediate 120 (0.64 g, 1.68 mmol) in EtOH (10 mL) at rt and the mixture was stirred at 80° C. for 2 h. The solvents evaporated in vacuo and the crude triturated with DIPE. The filtrate was concentrated in vacuo purified by flash column chromatography (silica; NH₃ 7N in MeOH in DCM 0/100 to 10/90). The desired fractions were collected and concentrated in vacuo to yield intermediate 122 (0.33 g, 77%) as a pale-yellow sticky solid.

### Preparation of Intermediate 123

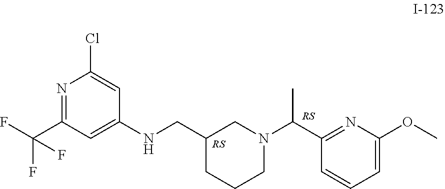 [SMILES: COc1cccc(C(C)N2CCCC(CNc3cc(Cl)nc(C(F)(F)F)c3)C2)n1]

Sodium tert-butoxide (0.097 g, 1.01 mmol), Dave-phos (CAS 213697-53-1, 0.020 g, 0.051 mmol) and Pd₂(dba)₃ (0.023 g, 0.025 mmol) were added to a solution of 6-chloro-4-iodo-6-trifluoromethylpyridine (CAS 20544-22-0, 0.155 g, 0.50 mmol) in 1,4-dioxane (9 mL) under N₂ at rt in a closed tube. Intermediate 122 (0.135 g, 0.53 mmol) was added and the mixture stirred at 100° C. overnight. The mixture was diluted with EtOAc and NH₄Cl sat. filtered through a pad of diatomaceous earth and the solvents evaporated in vacuo. The crude product was purified by flash column chromatography (silica; MeOH in DCM 0/100 to 10/90). The desired fractions were collected and the solvent evaporated in vacuo to yield intermediate 123 (141 mg, 65%) as a brown solid.

### Preparation of Intermediate 124

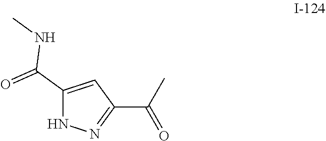

HATU (CAS 148893-10-1, 0.64 g, 1.68 mmol) was added to a stirred solution of 3-acetyl-1H-pyrazole-5-carboxylic acid (CAS 949034-45-1, 0.20 g, 1.29 mmol) in DMF (1 mL). The mixture was stirred at rt for 30 min. Then a suspension of methylamine hydrochloride (96 mg, 1.43 mmol) and TEA (0.54 mL, 3.89 mmol) in DMF (1.66 mL) was added and the mixture was stirred at rt for 16 h. Then H₂O and EtOAc were added. The organic layer was separated, dried (Na₂SO₄), filtered and concentrated in vacuo. The residue was purified by flash column chromatography (silica; MeOH in DCM 0/100 to 6/94). The desired fractions were collected and concentrated in vacuo to afford intermediate 124 (50 mg, 23%) as a white solid.

### Preparation of Intermediate 125

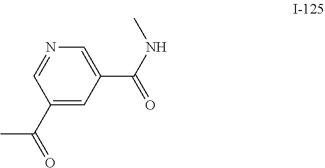

Tributyl(1-ethoxyvinyl)tin (CAS 13965-03-02, 0.14 g, 0.19 mmol) followed by bis(triphenylphosphine)palladium(II) dichloride (CAS 13965-03-2, 0.1 eq, 0.138 g, 0.12 mmol) were added to a stirred solution of 5-bromo-N-methylnicotinamide (CAS 153435-68-8, 0.42 g, 1.98 mmol) in toluene (10 mL) in a sealed tube and under N₂. The mixture was stirred at 80° C. for 16 h. Then, more tributyl(1-ethoxyvinyl)tin (CAS 13965-03-02, 0.14 g, 0.19 mmol) and bis(triphenylphosphine)palladium(ii) dichloride (CAS 13965-03-2, 0.1 eq, 0.138 g, 0.12 mmol) were added and stirred at 80° C. for 6 h. Then a 1M HCl solution in diethyl ether (3.9 mL) was added and the mixture was stirred at rt for 1 h. The mixture was added to a stirred solution of sat NaHCO₃ and ice and extracted with DCM. The organic layer was separated, dried (MgSO₄), filtered and the solvents evaporated in vacuo. The crude product was purified by flash column chromatography (silica; MeOH in DCM 0/100 to 10/90). The desired fractions were collected and concentrated in vacuo to yield intermediate 125 (105 mg, 30%) as a pale-yellow solid.

### Preparation of Intermediate 126

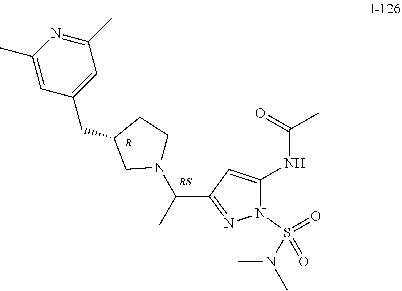

Intermediate 126 was prepared following an analogous procedure to the one described for the synthesis of intermediate 65 using intermediate 31 as starting material.

### Preparation of Intermediate 127

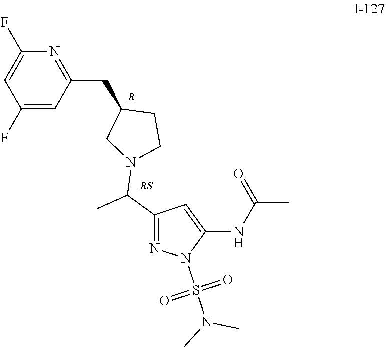

Intermediate 127 was prepared following an analogous procedure to the one described for the synthesis of intermediate 65 using intermediate 35 as starting material.

### Preparation of Intermediate 128

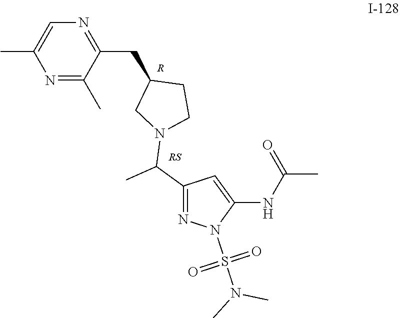

Intermediate 128 was prepared following an analogous procedure to the one described for the synthesis of intermediate 65 using intermediate 37 as starting material.

### Preparation of Intermediate 129

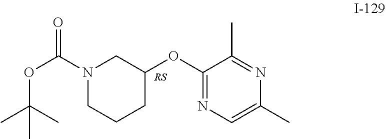 [SMILES: Cc1cnc(OC2CCCN(C(=O)OC(C)(C)C)C2)c(C)n1]

Intermediate 129 was prepared following an analogous procedure to the one described for the synthesis of intermediate 22 using 2-chloro-3,5-dimethylpyrazine (CAS 38557-72-1) as starting material and THF as solvent.

### Preparation of Intermediate 130

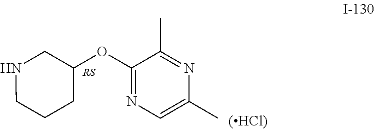

A 4 M HCl solution in 1,4-dioxane (5 mL, 20 mmol) was added to a solution of intermediate 129 (0.95 g, 3.09 mmol) in 1,4-dioxane and the mixture was stirred at rt for 16 h. Then the solvent was evaporated in vacuo. The solid formed was treated with DCM and sat NaHCO₃ solution and the product was extracted with a mixture of DCM/EtOH (9:1). The organic layer was separated, dried (MgSO₄), filtered and the solvents evaporated in vacuo to yield intermediate 130 as an oil.

### Preparation of Intermediate 131

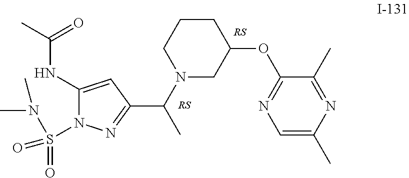

To a solution of intermediate 130 (0.10 g, 0.41 mmol) and DIPEA (CAS 7087-68-5, 0.14 mL, 0.82 mmol) in DCE (3 mL), titanium(IV)isopropoxide (CAS 546-68-9, 0.18 mL, 0.61 mmol), intermediate 54 (0.11 g, 0.41 g) were added and the reaction mixture was stirred at 80° C. for 5 h. Then the mixture was cooled to rt and sodium cyanoborohydride (CAS 25895-60-7, 0.031 g, 0.49 mmol) was added and the mixture was stirred for 16 h more. Then a saturated solution of NaHCO₃ was added and the mixture was diluted with DCM and filtered through a Celite® pad. The filtrates were extracted with DCM and the organic layer was separated, dried (MgSO₄), filtered and the solvents evaporated in vacuo. The mixture was concentrated in vacuo. The crude product was purified by flash column chromatography (silica; 7M ammonia solution in EtOH in DCM 0/100 to 05/95). The desired fractions were collected and concentrated in vacuo to yield intermediate 131 (0.14 g, 73%) as an oil.

### Preparation of Intermediate 132

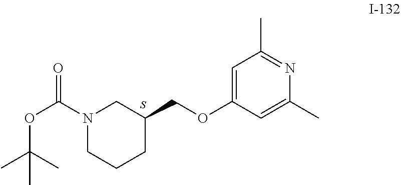

Intermediate 132 was prepared following an analogous procedure to the one described for the synthesis of intermediate 22 using 4-chloro-2,6-dimethylpyridine (CAS 3512-75-2) as starting material.

### Preparation of Intermediate 133

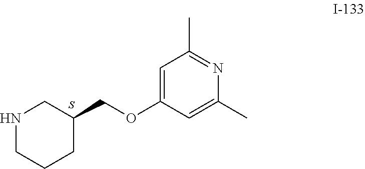

Intermediate 133 was prepared following an analogous procedure to the one described for the synthesis of intermediate 3 using intermediate 132 as starting material.

### Preparation of Intermediate 134

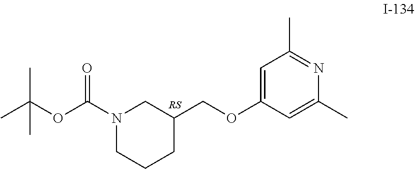

Diisopropyl azodicarboxylate (CAS 2446-83-5, 0.69 mL, 3.48 mmol) was added dropwise to a stirred solution of 1-Boc-3-(hydroxymethyl)piperidine (CAS 116574-71-1, 0.5 g, 2.32 mmol), 2,6-dimethyl-4-hydroxy pyridine (CAS 13603-44-6, 0.31 g, 2.55 mmol) and triphenylphosphine (CAS 603-35-0) in THF (50 mL) under nitrogen at rt. The reaction mixture was stirred at rt for 16 hour and then a sat NaHCO₃ solution and EtOAc were added. The organic layer was separated, dried (MgSO₄), filtered and the solvents evaporated in vacuo. The crude product was purified by flash column chromatography (silica; EtOAc in heptane, from 0/100 to 70/30). The desired fractions were collected and concentrated in vacuo to yield intermediate 134 (1.2 g, 78%, 70% purity) as a white solid.

### Preparation of Intermediate 135

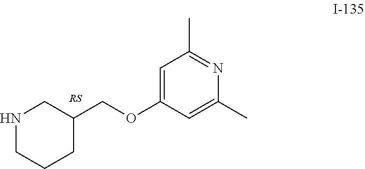 [SMILES: Cc1cc(OCC2CCCNC2)cc(C)n1]

TFA (2.72 mL, 36.7 mmol) was added to a stirred solution of intermediate 134 (0.84 g, 2.62 mmol) in DCM (15 mL) at 0° C. The mixture was stirred at rt for 1 h. The solvent was evaporated in vacuo and a sat K₂C03 solution was added and the mixture was extracted with DCM. The organic layer was separated, dried (MgSO₄), filtered and the solvent evaporated in vacuo. The crude product was purified by flash column chromatography (silica; NH₃ 7N in MeOH in DCM, from 0/100 to 100/0). The desired fractions were collected and concentrated in vacuo to yield intermediate 1345 (0.28 g, 49%) as a yellow oil.

### Preparation of Intermediate 136

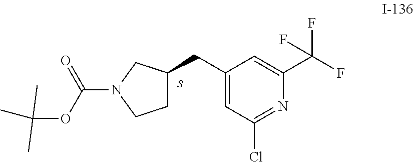

Intermediate 136 was prepared following an analogous procedure to the one described for the synthesis of intermediate 30 using 2-chloro-4-iodo-6-trifluoromethyl-pyridine (CAS 205444-22-0) as starting material.

### Preparation of Intermediate 137

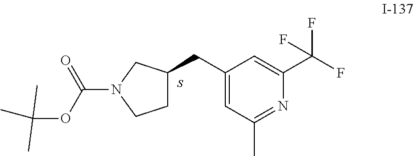

Intermediate 137 was prepared following an analogous procedure to the one described for the synthesis of intermediate 7 using intermediate 136 as starting material.

### Preparation of Intermediate 138

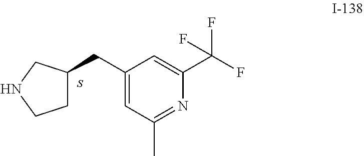

Intermediate 138 was prepared following an analogous procedure to the one described for the synthesis of intermediate 31 using intermediate 137 as starting material.

### Preparation of Intermediate 139

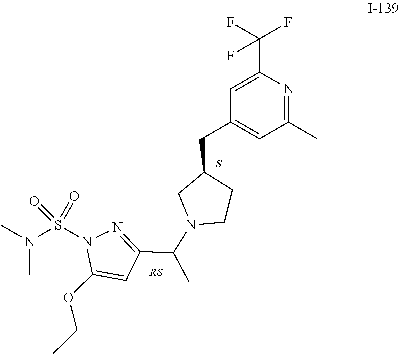

Intermediate 139 was prepared following an analogous procedure to the one described for the synthesis of intermediate 92 using intermediate 138 as starting material. Intermediate 139 was purified by flash column chromatography (silica; MeOH in EtOAc 0/100 to 10/90).

### Preparation of Intermediate 140

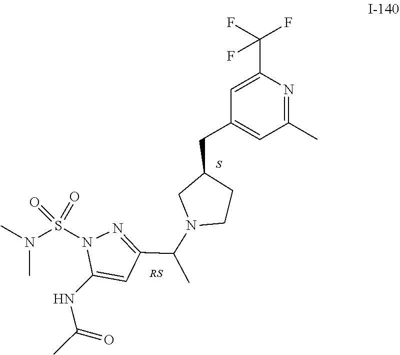

Intermediate 140 was prepared following an analogous procedure to the one described for the synthesis of intermediate 65 using intermediate 138 as starting material. Intermediate 140 was purified by flash column chromatography (silica; EtOAc in DCM 0/100 to 40/60).

### Preparation of Intermediate 141

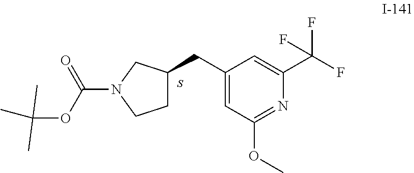

A 25% MeONa solution in MeOH (0.45 mL, 1.95 mmol) was added dropwise to a stirred solution of intermediate 136 in MeOH (0.7 mL) at rt. The mixture was stirred at rt for 16 h. Then more 25% MeONa solution in MeOH was added (0.90 mL, 3.91 mmol) and the mixture was stirred at rt for another 48 h. Then water was added and the desired product was extracted with DCM. The organic layer was separated, dried (Na₂SO₄), filtered and the solvent evaporated in vacuo to yield intermediate 141 (0.16 g, 91%) as a colorless oil.

### Preparation of Intermediate 142

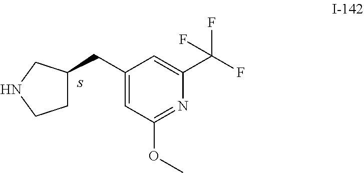

Intermediate 142 was prepared following an analogous procedure to the one described for the synthesis of intermediate 23 using intermediate 141 as starting material.

### Preparation of Intermediate 143

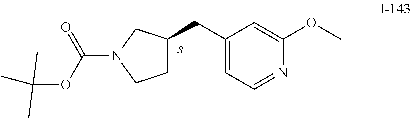

Intermediate 143 was prepared following an analogous procedure to the one described for the synthesis of intermediate 30 using 4-bromo-2-methoxypyridine (CAS 100367-39-3) as starting material.

### Preparation of Intermediate 144

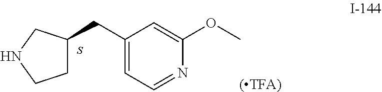

TFA (0.28 mL, 3.67 mmol) was added to a stirred solution of intermediate 134 (0.24 g, 0.73 mmol) in DCM. (20 mL) The mixture was stirred at rt for 16 h. The solvents were evaporated in vacuo to yield intermediate 144 as a brown oil.

### Preparation of Intermediate 145

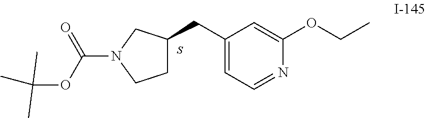

Intermediate 145 was prepared following an analogous procedure to the one described for the synthesis of intermediate 30 using 4-bromo-2-ethoxypyridine (CAS 57883-26-8) as starting material.

### Preparation of Intermediate 146

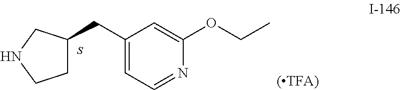

Intermediate 146 was prepared following an analogous procedure to the one described for the synthesis of intermediate 144 using intermediate 145 as starting material.

### Preparation of Intermediate 147

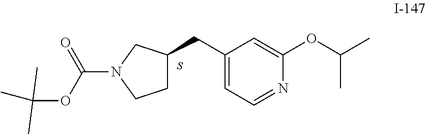

Intermediate 147 was prepared following an analogous procedure to the one described for the synthesis of intermediate 30 using 4-bromo-2-isopropoxypyridine (CAS 1142194-24-8) as starting material.

### Preparation of Intermediate 148

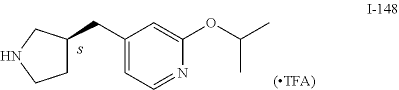

Intermediate 148 was prepared following an analogous procedure to the one described for the synthesis of intermediate 144 intermediate using 147 as starting material.

### Preparation of Intermediate 149

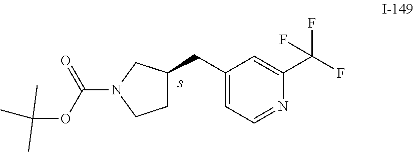

Intermediate 148 was prepared following an analogous procedure to the one described for the synthesis of intermediate 30 using 4-bromo-2-(trifluoromethyl)pyridine (CAS 887583-90-6) as starting material.

### Preparation of Intermediate 150

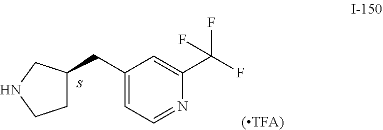

Intermediate 150 was prepared following an analogous procedure to the one described for the synthesis of intermediate 144 using intermediate 149 as starting material.

### Preparation of Intermediate 151

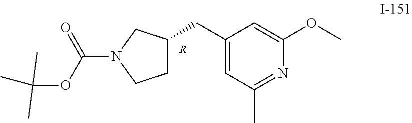

Intermediate 151 was prepared following an analogous procedure to the one described for the synthesis of intermediate 30 using 4-bromo-2-methoxy-6-methylpyridine (CAS 1083169-00-9) as starting material.

### Preparation of Intermediate 152

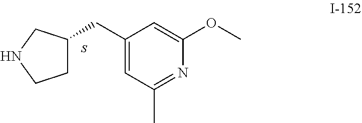

Intermediate 152 was prepared following an analogous procedure to the one described for the synthesis of intermediate 31 using intermediate 151 as starting material.

### Preparation of Intermediate 153

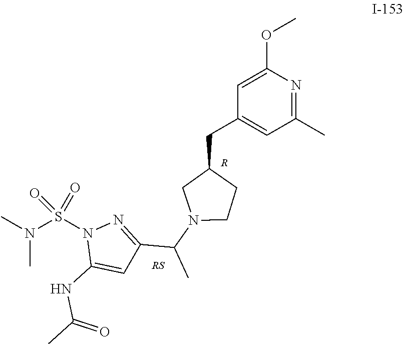

Intermediate 153 was prepared following an analogous procedure to the one described for the synthesis of intermediate 65 using intermediate 152 as starting material. Intermediate 153 was purified by flash column chromatography (silica; MeOH in DCM 0/100 to 5/95).

### Preparation of Intermediate 154

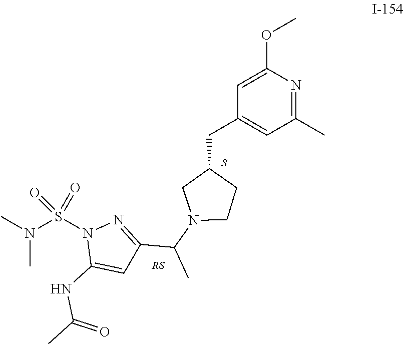

Intermediate 154 was prepared following an analogous procedure to the one described for the synthesis of intermediate 65 using intermediate 33 as starting material. Intermediate 1454 was purified by flash column chromatography (silica; MeOH in DCM 0/100 to 4/96) and by RP HPLC (Stationary phase: C18 XBridge 30×100 mm 5 μm), Mobile phase: Gradient from 67% NH₄HCO₃ 0.25% solution in Water, 33% CH₃CN to 50% NH₄HCO₃ 0.25% solution in Water, 50% CH₃CN).

### Preparation of Intermediate 155

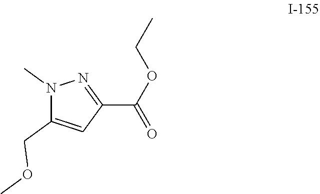

Sodium hydride (CAS 7646-69-7, 0.23 g, 10.1 mmol) was added to a stirred solution of intermediate 93 (1.7 g, 9.23 mmol) in anhydrous THF (50 mL) at 0° C. under N₂. After 30 min methyliodide (CAS 74-88-4, 0.632 mL, 10.1 mmol) was added and the reaction mixture was allowed to reach rt and was then stirred for 20 h. Then, the reaction was cooled to 0° C. and additional sodium hydride (0.106 g, 4.6 mmol) was added. After 30 min additional methyliodide (0.287 mL, 4.6 mmol) was added and the reaction mixture was stirred at rt for 5 days. Then a sat solution of NH₄Cl was added and the mixture was extracted with EtOAc. The organic layer was separated, dried (MgSO₄), filtered and the solvents evaporated in vacuo. The crude product was purified by flash column chromatography (silica; EtOAc in heptane 0/100 to 50/50). The desired fractions were collected and the solvents evaporated in vacuo to yield intermediate 155 (1.8 g, 50%) as a yellow oil.

### Preparation of Intermediate 156

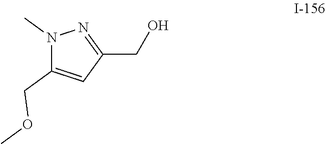

A 1 M Lithium aluminium hydride solution in THF (CAS 16853-85-3, 5.7 mL, 5.7 mmol) was added to a stirred solution of intermediate 155 (0.92 g, 4.61 mmol) in anhydrous THF (25 mL) at 0° C. and under N₂. The mixture was left warming slowly to rt and stirred for 2 h. The mixture was diluted with EtOAc and Na₂SO₄.10H₂O was added at 0° C. The mixture was stirred for 15 min at 0° C., filtered through Celite® and washed with additional EtOAc. The solvents were evaporated in vacuo to yield intermediate 155 (0.78 g, >100%) as a yellow oil which was used in next reaction without further purification.

### Preparation of Intermediate 157

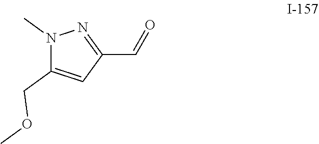

Manganese dioxide (CAS 1313-13-9, 2.1 g, 25.1 mmol) was added to a solution of intermediate 156 (0.78 g, 5.0 mmol) in DCM (20 mL) and the reaction mixture was stirred at 80° C. for 2 h. The solid was filtered off and washed with DCM and MeOH. The filtrate was evaporated in vacuo to yield intermediate 156 (0.44 g, 57%) as a brown wax which was used in next reaction without further purification.

### Preparation of Intermediate 158

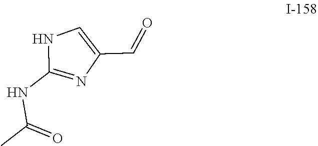

Pyridinium p-toluenesulfonate (CAS 24057-28-1, 0.053 g, 0.21 mmol) was added to a stirred solution of tert-butyl 2-acetamido-4-(dimethoxymethyl)imidazole-1-carboxylate (CAS 1000701-70-1, 0.40 g, 1.36 mmol) prepared according to a procedure described in Tetrahedron 66 (2010), 6224 in acetone (7.5 mL) and water (5 mL) in a sealed tube under N₂. The mixture was stirred at rt for 16 h. The crude was treated with brine and extracted with AcOEt. The organic layer was separated, dried (MgSO₄), filtered and the solvents evaporated in vacuo to intermediate (0.12 g, 57%) as a white solid.

### Preparation of Intermediate 159

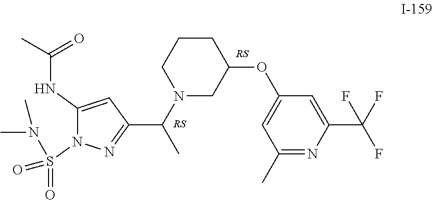 [SMILES: CC(=O)Nc1cc(C(C)N2CCCC(Oc3cc(C)nc(C(F)(F)F)c3)C2)nn1S(=O)(=O)N(C)C]

Intermediate 159 was prepared following an analogous procedure to the one described for the synthesis of intermediate 65 using intermediate 28 as starting material.

### Preparation of Intermediate 160

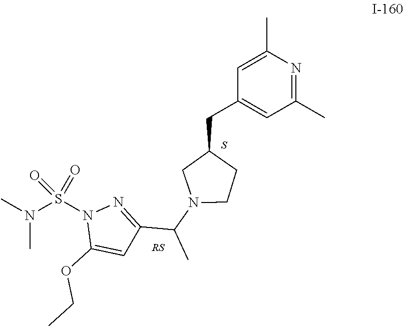

Intermediate 160 was prepared following an analogous procedure to the one described for the synthesis of intermediate 92 using intermediate 31 as starting material. Intermediate 160 was purified by flash column chromatography (silica; MeOH in DCM 0/100 to 10/90).

### Preparation of Intermediate 161

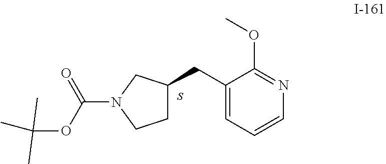

Intermediate 161 was prepared following an analogous procedure to the one described for the synthesis of intermediate 30 using 3-iodo-2-methoxypyridine (CAS 112197-15-6) as starting material.

### Preparation of Intermediate 162

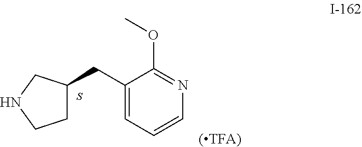

Intermediate 162 was prepared following an analogous procedure to the one described for the synthesis of intermediate 144 intermediate using 161 as starting material.

### Preparation of Intermediate 163

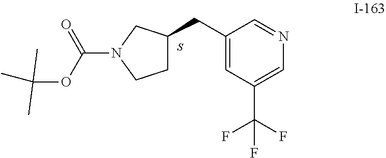

Intermediate 163 was prepared following an analogous procedure to the one described for the synthesis of intermediate 30 using 3-bromo-5-(trifluoromethyl)pyridine (CAS 436799-33-6) as starting material.

### Preparation of Intermediate 164

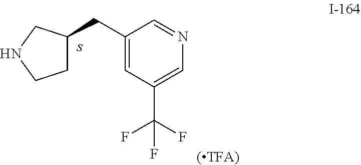

Intermediate 164 was prepared following an analogous procedure to the one described for the synthesis of intermediate 144 using intermediate 163 as starting material.

### Preparation of Intermediate 165

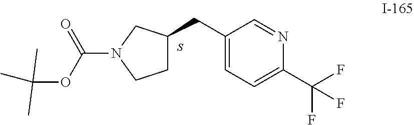

Intermediate 165 was prepared following an analogous procedure to the one described for the synthesis of intermediate 30 using 5-bromo-2-(trifluoromethyl)pyridine (CAS 436799-32-5) as starting material.

### Preparation of Intermediate 166

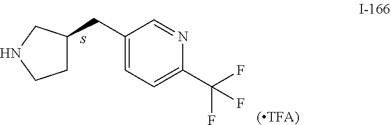

Intermediate 166 was prepared following an analogous procedure to the one described for the synthesis of intermediate 144 using intermediate 165 as starting material.

### Preparation of Intermediate 167

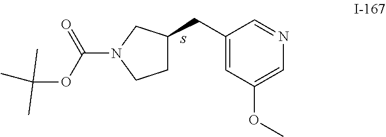

Intermediate 167 was prepared following an analogous procedure to the one described for the synthesis of intermediate 30 using 3-bromo-5-methoxypyridine (CAS 50720-12-2) as starting material.

### Preparation of Intermediate 168

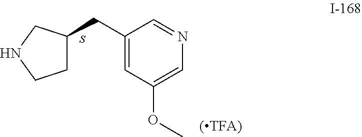

Intermediate 168 was prepared following an analogous procedure to the one described for the synthesis of intermediate 144 using intermediate 167 as starting material.

### Preparation of Intermediate 169

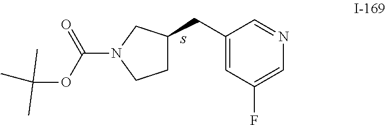

Intermediate 169 was prepared following an analogous procedure to the one described for the synthesis of intermediate 30 using 3-bromo-5-fluoropyridine (CAS 407-20-5) as starting material.

### Preparation of Intermediate 170

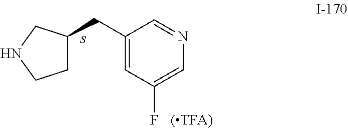

Intermediate 170 was prepared following an analogous procedure to the one described for the synthesis of intermediate 144 using intermediate 169 as starting material.

### Preparation of Intermediate 171

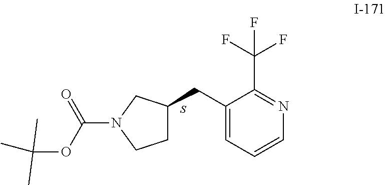

Intermediate 171 was prepared following an analogous procedure to the one described for the synthesis of intermediate 30 using 3-bromo-2-(trifluoromethyl)pyridine (CAS 590371-58-7) as starting material.

### Preparation of Intermediate 172

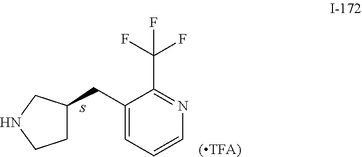

Intermediate 172 was prepared following an analogous procedure to the one described for the synthesis of intermediate 144 using intermediate 171 as starting material.

### Preparation of Intermediate 173

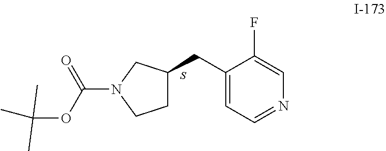

Intermediate 173 was prepared following an analogous procedure to the one described for the synthesis of intermediate 30 using 3-fluoro-4-iodo-pyridine (CAS 22282-75-3) as starting material.

### Preparation of Intermediate 174

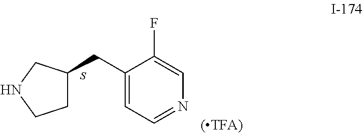

Intermediate 174 was prepared following an analogous procedure to the one described for the synthesis of intermediate 144 using intermediate 173 as starting material.

### Preparation of Intermediate 175

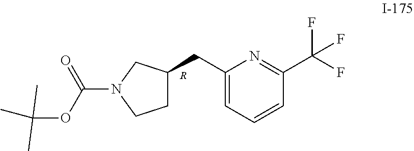

Intermediate 175 was prepared following an analogous procedure to the one described for the synthesis of intermediate 30 using 2-bromo-6-(trifluoromethyl)pyridine (CAS 189278-27-1) as starting material.

### Preparation of Intermediate 176

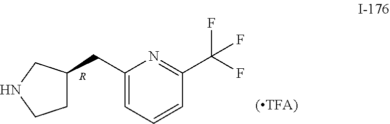

Intermediate 176 was prepared following an analogous procedure to the one described for the synthesis of intermediate 144 using intermediate 175 as starting material.

### Preparation of Intermediate 177

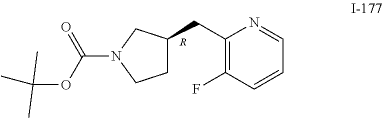

Intermediate 177 was prepared following an analogous procedure to the one described for the synthesis of intermediate 30 using 2-bromo-3-fluoropyridine (CAS 40273-45-8) as starting material.

### Preparation of Intermediate 178

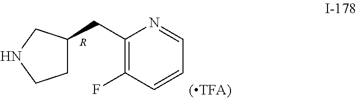

Intermediate 178 was prepared following an analogous procedure to the one described for the synthesis of intermediate 144 using intermediate 177 as starting material.

### Preparation of Intermediate 179

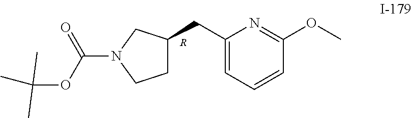

Intermediate 179 was prepared following an analogous procedure to the one described for the synthesis of intermediate 30 using 2-bromo-6-methoxypyridine (CAS 40473-07-2) as starting material.

### Preparation of Intermediate 180

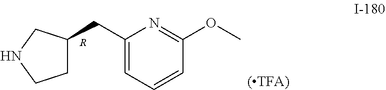

Intermediate 180 was prepared following an analogous procedure to the one described for the synthesis of intermediate 144 using intermediate 179 as starting material.

### Preparation of Intermediate 181

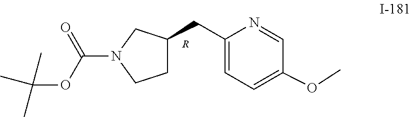

Intermediate 181 was prepared following an analogous procedure to the one described for the synthesis of intermediate 30 using 2-bromo-5-methoxypyridine (CAS 105170-27-2) as starting material.

### Preparation of Intermediate 182

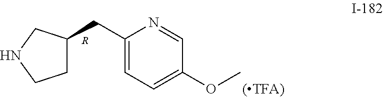

Intermediate 182 was prepared following an analogous procedure to the one described for the synthesis of intermediate 144 using intermediate 181 as starting material.

### Preparation of Intermediate 183

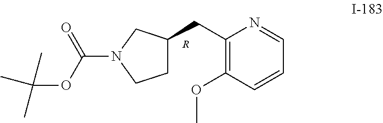

Intermediate 183 was prepared following an analogous procedure to the one described for the synthesis of intermediate 30 using 2-iodo-3-methoxypyridine (CAS 93560-55-5) as starting material.

### Preparation of Intermediate 184

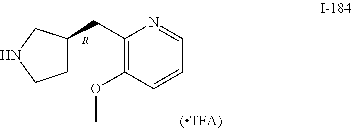

Intermediate 184 was prepared following an analogous procedure to the one described for the synthesis of intermediate 144 using intermediate 183 as starting material.

### Preparation of Intermediate 185

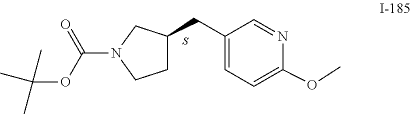

Intermediate 185 was prepared following an analogous procedure to the one described for the synthesis of intermediate 30 using 5-bromo-2-methoxypyridine (CAS 13472-85-0) as starting material.

### Preparation of Intermediate 186

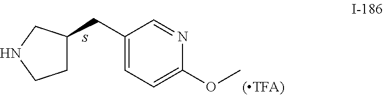

Intermediate 186 was prepared following an analogous procedure to the one described for the synthesis of intermediate 144 using intermediate 185 as starting material.

### Preparation of Intermediate 187

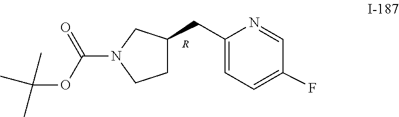

Intermediate 187 was prepared following an analogous procedure to the one described for the synthesis of intermediate 30 using 5-bromo-2-fluoropyridine (CAS 41404-58-4) as starting material.

### Preparation of Intermediate 188

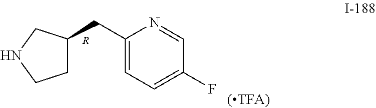

Intermediate 188 was prepared following an analogous procedure to the one described for the synthesis of intermediate 144 using intermediate 187 as starting material.

### Preparation of Intermediate 189

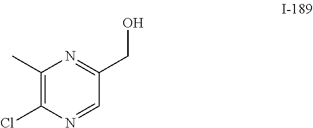

Sodium borohydride (CAS 16940-66-2, 202 mg, 5.36 mmol) was added portionwise to a suspension of calcium chloride (CAS 10043-52-4, 1.19 g mg, 10.7 mmol) in a mixture of anhydrous THF (15 mL) and EtOH (15 mL) at −10° C. under nitrogen. The mixture was stirred for 15 min. Then, a solution of methyl 5-chloro-6-methylpyrazine-2-carboxylate (CAS 77168-85-5, 500 mg, 2.68 mmol) in anhydrous THF (5 mL) was added dropwise to the mixture at −10° C. The reaction mixture was stirred at rt for 15 h. The mixture was cooled to 0° C. and carefully diluted with saturated Na₂CO₃ and saturated NaHCO₃ and EtOAc. The mixture was filtered over a Celite® pad. The organic layer was separated, dried (MgSO₄), filtered and the solvents evaporated in vacuo. The crude was purified by flash column chromatography (silica; EtOAc in heptane, from 0/100 to 80/20). The desired fractions were collected and concentrated in vacuo to yield intermediate 189 (398 mg, 93%) as a colourless oil.

### Preparation of Intermediate 190

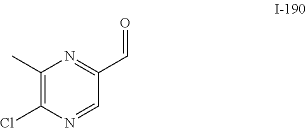

Dess-martin periodinane (CAS 87413-09-0, 624 mg, 1.47 mmol) was added portwise to a stirred solution of intermediate 189 (212 mg, 1.33 mmol) in DCM (39 mL) at 0° C. The mixture was stirred at rt for 1 h. The mixture was diluted with saturated NaHCO₃ and 10% Na₂S₂O₃ solutions and extracted with DCM. The organic layer was separated, dried (MgSO₄), filtered and the solvents evaporated in vacuo. The crude product was purified by flash column chromatography (silica; EtOAc in DCM 0/100 to 10/90). The desired fractions were collected and concentrated in vacuo to yield intermediate 190 (162 mg, 77%) as a colourless oil.

### Preparation of Intermediate 191

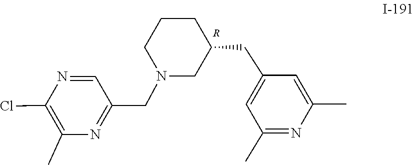

Acetic acid (0.047 mL, 0.81 mmol) and sodium cyanoborohydride (CAS 25895-60-7, 26 mg, 0.41 mmol) were added to a stirred solution of intermediate 3 (112 mg, 0.41 mmol), intermediate 190 (70 mg, 0.45 mmol) and anhydrous sodium acetate (CAS 127-09-3, 130 mg, 1.59 mmol) in MeOH (5 mL) at rt. The reaction mixture was stirred at rt for 16 hours and then more B intermediate 190 (87 mg, 0.56 mmol) and F sodium cyanoborohydride (CAS 25895-60-7, 25 mg, 0.40 mmol) were added and the mixture was stirred at rt for a further 16 hours more. The mixture was diluted with saturated NaHCO₃ solution and extracted with EtOAc. The organic layer was separated, dried (MgSO₄), filtered and the solvents evaporated in vacuo. The crude product was purificated by flash column chromatography (silica; MeOH in DCM (9:1) in DCM 0/100 to 50/50). The desired fractions were collected and concentrated in vacuo to yield intermediate 191 (118 mg, 80%) as a pale-yellow sticky solid.

### Preparation of Intermediate 192

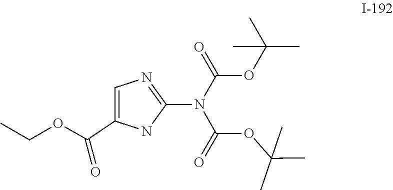 [SMILES: CCOC(=O)c1cnc(N(C(=O)OC(C)(C)C)C(=O)OC(C)(C)C)[nH]1]

A solution of Boc-anhydride (CAS 24424-99-5, 0.49 mL, 0.23 mmol) in THF (2 mL) followed by DMAP (CAS 1122-58-3, 26 mg, 0.21 mmol) were added drop and portionwise to a stirred suspension of ethyl 2-amino-1-methyl-1H-imidazole-5-carboxylate (CAS 177760-04-2, 329 mg, 1.95 mmol) in THF (8 mL) in a round-bottom flask and under N₂. The mixture was stirred at rt for 16 h. Then more BOC-anhydride (CAS 24424-99-5, 0.49 mL, 0.23 mmol) was added and the mixture was stirred at rt for 5 h. The solvent was evaporated in vacuo and the crude product was purified by flash column chromatography (silica; EtOAc in heptane 0/100 to 50/50). The desired fractions were collected and concentrated in vacuo to yield intermediate 192 (720 mg, 100%) as a white solid.

### Preparation of Intermediate 193

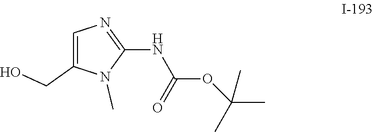 [SMILES: Cn1c(CO)cnc1NC(=O)OC(C)(C)C]

Lithium borohydride (CAS 16949-15-8, 95 mg, 4.4 mmol) was added to a stirred solution of intermediate 193 (483 mg, 1.31 mmol) in THF (10 mL) in a sealed tube and under N₂. The mixture was stirred at 120° C. for 10 min under microwave irradiation. Then more lithium borohydride (110 mg, 5.0 mmol) was added and the mixture was stirred at 120° C. for 15 min under microwave irradiation. The mixture was treated dropwise with MeOH and stirred at rt for 30 min. The solvents were evaporated in vacuo and the crude product was purified by flash column chromatography (silica; 7N NH₃ in MeOH solution in DCM 0/100 to 10/90). The desired fractions were collected and concentrated in vacuo to intermediate 193 (77 mg, 26%) as a white solid.

### Preparation of Intermediate 194

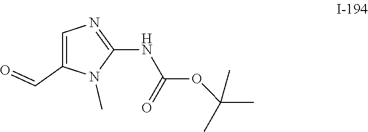 [SMILES: Cn1c(C=O)cnc1NC(=O)OC(C)(C)C]

Manganese (IV) oxide (CAS 1313-13-9, 295 mg, 2.88 mmol) was added to a stirred suspension of intermediate 193 in 1,4-dioxane (3. mL) in a sealed tube and under N₂. The mixture was stirred at rt for 5 days. The mixture was filtered through a Celite® pad and washed with DCM. The filtrate was concentrated in vacuo. The crude product was purified by flash column chromatography (silica; EtOAc in DCM 0/100 to 100/0). The desired fractions were collected and concentrated in vacuo to yield intermediate 194 (34 mg, 76%) a yellow solid.

### Preparation of Intermediate 195

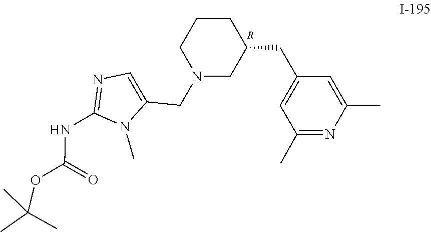

Sodium triacetoxyborohydride (CAS 56553-60-7, 80 mg, 0.38 mmol) was added to a stirred solution of intermediate 3 (46 mg, 0.23 mmol) and intermediate 194 (51 mg, 0.23 mmol) in DCM (1.1 mL) in a sealed tube and under N₂. The mixture was stirred at rt for 16 h. Then the mixture was treated with a saturated NaHCO₃ solution and extracted with DCM. The organic layer was separated, dried (MgSO₄), filtered and the solvents evaporated in vacuo. The crude product was purified by flash column chromatography (silica; 7N NH₃ in MeOH solution in DCM 0/100 to 5/95). The desired fractions were collected and concentrated in vacuo to yield intermediate 195 (65 mg, 94%) as a yellow oil.

### Preparation of Intermediates 196 and 197

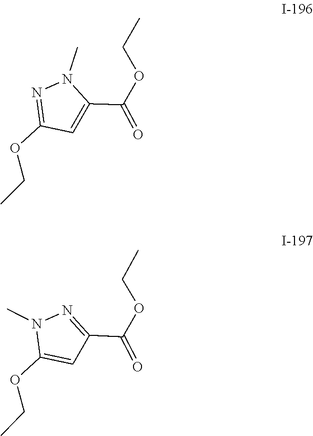

K₂CO₃ (3.15 g, 22.7 mmol) and methyl iodide (CAS 74-88-4, 0.93 mL, 14.8 mmol) were added to a stirred solution of ethyl 5-ethoxy-1H-pyrazole-3-carboxylate (CAS 1116656-05-3, 2.1 g, 11.4 mmol) in DMF (15 mL) under nitrogen at rt. The mixture was stirred at rt overnight. The mixture was diluted with water and extracted with DCM. The organic layer was separated, dried (Na₂SO₄), filtered and the solvents evaporated in vacuo. The crude product was purified by flash column chromatography (silica; EtOAc in heptane 0/100 to 20/80). The desired fractions were collected and concentrated in vacuo to intermediate 196 (0.51 g, 23%) as a white solid and intermediate 197 (2.3 g, 49%) as a colourless oil.

### Preparation of Intermediate 198

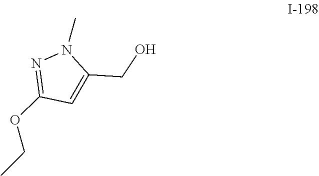

Lithium brorohydride (CAS 16949-15-8, 645 mg, 29.6 mmol) was added portionwise to a stirred solution of intermediate 196 (1.46 g, 7.41 mmol) in THF (15 mL) under nitrogen at 0° C. Then MeOH (0.3 mL) was added dropwise and the reaction mixture was stirred at rt for 4 h. Then, more lithium brorohydride (CAS 16949-15-8, 322 mg, 14.8 mmol) and MeOH (0.18 mL) were added, and the reaction mixture was stirred overnight. The mixture was diluted with water and extracted with EtOAc. The organic layer was separated, dried (MgSO₄), filtered and the solvents evaporated in vacuo. The crude product was purified by flash column chromatography (silica; MeOH in DCM 0/100 to 5/95). The desired fractions were collected and concentrated in vacuo to yield intermediate 198 (925 mg, 80%) as a colourless oil that precipitated upon standing.

### Preparation of Intermediate 199

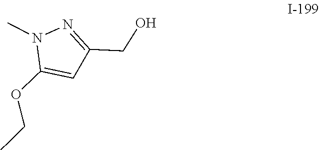

Intermediate 199 was prepared following an analogous procedure to the one described for the synthesis of intermediate 198 using intermediate 197 as starting material.

### Preparation of Intermediate 200

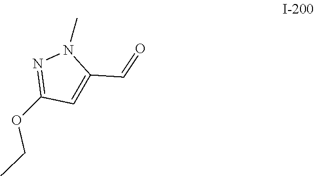

Intermediate 200 was prepared following an analogous procedure to the one described for the synthesis of intermediate 57 using intermediate 198 as starting material.

### Preparation of Intermediate 201

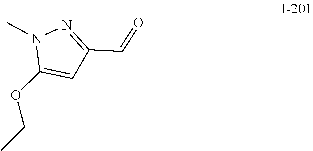

Intermediate 201 was prepared following an analogous procedure to the one described for the synthesis of intermediate 57 using intermediate 199 as starting material.

### Preparation of Intermediate 202

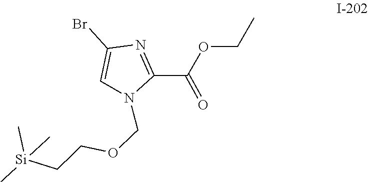 [SMILES: CCOC(=O)c1nc(Br)cn1COCC[Si](C)(C)C]

NaH (60% dispersion in mineral oil) (CAS 7646-69-7, 1.37 g, 34.2 mmol) was added to a stirred solution of ethyl imidazole-2-carboxylate (CAS 33543-78-1, 4 g, 28.5 mmol) in anhydrous THF (140 mL) portionwise at 0° C. and under N₂. The mixture was stirred at 0° C. for 20 min and then 2-(trimethylsilyl)ethoxymethyl chloride (CAS 76513-69-4, 5.56 mL, 31.4 mmol) was added dropwise. The resulting reaction mixture was stirred at rt for 2.5 h. The mixture was diluted with a saturated NH₄Cl solution and extracted with EtOAc. The organic layer was separated, dried (Na₂SO₄), filtered and the solvents evaporated in vacuo to yield a brown liquid oil. The crude product was purified by flash column chromatography (silica; EtOAc in Hept 0/100 to 50/50). The desired fractions were collected and the solvents evaporated in vacuo to yield intermediate 202 (7.34 g, 95%) as a yellow oil.

### Preparation of Intermediate 203

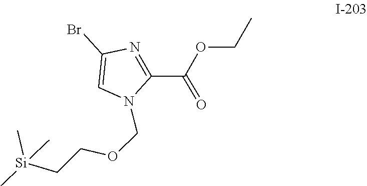 [SMILES: CCOC(=O)c1nc(Br)cn1COCC[Si](C)(C)C]

NBS (CAS 128-08-5, 4.83 g, 27.1 mmol) was added portionwise at 0° C. to a stirred solution of intermediate 202 in CAN (150 mL). The mixture was stirred at rt for 20 h. and then at 80° C. for a further 48 h. Then a saturated Na₂CO₃ solution was added and the product extracted with EtOAc. The organic layer was separated, dried (MgSO₄), filtered and the solvents evaporated in vacuo. The crude product was purified by flash column chromatography (silica; EtOAc in Heptane 0/100 to 30/70). The desired fractions were collected and the solvents evaporated in vacuo to intermediate 203 (4.46, 47%) as a yellow oil that crystallized upon standing.

### Preparation of Intermediate 205

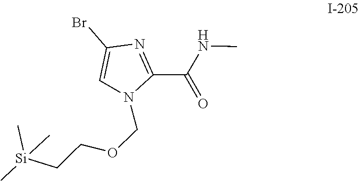

A 1M solution of lithium bis(trimethylsilyl)amide in THF (CAS 4039-32-1, 8.59 mL, 8.59 mmol) was added to a stirred solution of intermediate 203 (Ig, 2.86 mmol) and a 2M methylamine solution in THF (CAS 74-89-5, 2.14 mL, 4.29 mmol) in anhydrous THF (9.6 mL) at 0° C. and under nitrogen. The mixture was stirred at 0° C. for 30 min and then at rt for 1 h. The mixture was quenched with saturated NH₄Cl solution and extracted with EtOAc. The organic layer was separated, dried (Na₂SO₄), filtered and the solvent evaporated in vacuo. The crude product was purified by flash column chromatography (silica; EtOAc in DCM 0/100 to 20/80). The desired fractions were collected and the solvents evaporated in vacuo to yield intermediate 205 (704 mg, 74%) as a pale oil.

### Preparation of Intermediate 206

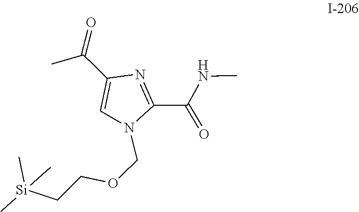

Bis(triphenylphosphine)palladium(ii) dichloride (CAS 13965-03-2, 112 mg, 0.157 mmol) was added to a stirred solution of intermediate 205 (552 mg, 1.65 mmol) and 1-ethoxy-1-(tributylstannyl)ethylene (CAS 97674-02-7, 0.725 mL, 2.14 mmol) in toluene (3.6 mL), in a sealed tube and under N₂. The mixture was stirred at 80° C. for 20 h. Additional bis(triphenylphosphine)palladium(ii) dichloride (45 mg, 0.063 mmol) and 1-ethoxy-1-(tributylstannyl)ethylene (0.29 mL, 0.85 mmol) were added and the mixture was stirred at 80° C. for a further 20 h. The mixture was added to a stirred mixture of saturated NaHCO₃ solution and ice and then extracted with EtOAc. The organic layer was separated and then a 1M HCl solution (1.6 mL) was added and the resulting mixture was stirred at 80° C. for 3 h. Additional 1M HCl solution (0.64 mL) was added and the resulting mixture was stirred at 80° C. for a further 20 h. The mixture was added to a stirred mixture of saturated NaHCO₃ solution and ice and then extracted with EtOAc. The organic layer was separated, dried (Na₂SO₄), filtered and the solvents evaporated in vacuo. The crude product was purified by flash column chromatography (silica; EtOAc in heptane 0/100 to 50/50). The desired fractions were collected and the solvents evaporated in vacuo to intermediate 206 (275, 56%) as yellow oil.

### Preparation of Intermediate 207

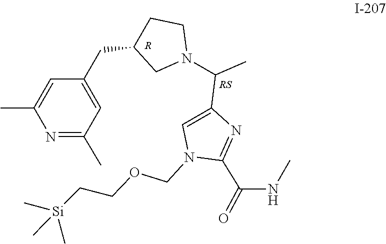

Sodium cyanoborohydride (CAS 25895-60-7, 32 mg, 0.51 mmol) was added to a stirred solution of intermediate 39 (81 mg, 0.43 mmol), intermediate 206 (130 mg, 0.44 mmol) and titanium(IV) isopropoxide (CAS 546-68-9, 0.25 mL, 0.85 mmol) in anhydrous THF (1.7 mL) at rt. The mixture was stirred at 70° C. for 20 h and then was treated with water and extracted with EtOAc. The mixture was filtered through Celite® and then the organic layer was separated, dried (Na₂SO₄), filtered and the solvents evaporated in vacuo. The crude product was purified by flash column chromatography (silica; EtOAc in Heptane 0/100 to 100/0) and (silica; methanol in DCM 0/100 to 10/90). The desired fractions were collected and the solvents evaporated in vacuo to yield intermediate 207 (73 mg, 36%) as a colourless oil.

### Preparation of Intermediate 208

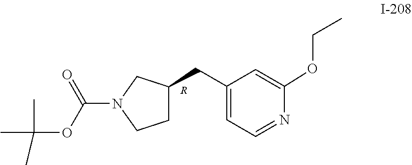

Intermediate 208 was prepared following an analogous procedure to the one described for the synthesis of intermediate 30 using 2-bromo-2-ethoxypyridine (CAS 57883-26-8) as starting material.

### Preparation of Intermediate 209

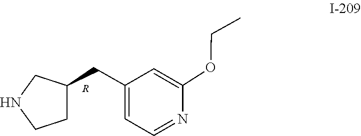

TFA (CAS 76-05-1, 0.61 mL, 8.0 mmol) was added to a stirred solution of intermediate 208 (604 mg, 1.60 mmol) in DCM. The mixture was stirred at rt for 72 h and then at 50° C. for a further 24 h. The solvent was evaporated in vacuo and the crude product was triturated with DCM. The solid was filtered off and the filtrate was concentrated in vacuo. The crude product was purified by ion exchange chromatography using an ISOLUTE SCX2 cartridge eluting first with methanol and then with a 7M solution of ammonia in methanol to yield intermediate 209 as a colourless oil.

### Preparation of Intermediate 210

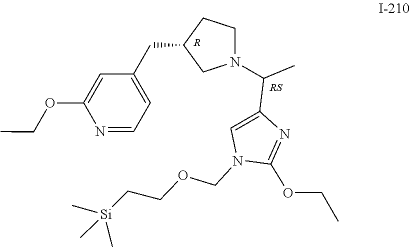

Pd/C (10%) (14 mg) was added to a stirred solution of intermediate 209 (54 mg, 0.26 mmol), 4-bromo-2-ethoxy-1-[[2-(trimethylsilyl)ethoxy]methyl]-1H-imidazole-5-carboxaldehyde (CAS 1073543-59-5, 148 mg, 0.39 mmol) and TEA (CAS 121-44-8, 0.109 mL, 0.785 mmol) in MeOH (0.5 mL) at 0° C. The mixture was hydrogenated at atmospheric pressure at 0° C. The mixture was allowed to warm to rt for 16 h and then was filtered through a Celite® pad. The pad was washed with EtOAc and the filtrate was concentrated in vacuo. The crude product was purified by flash column chromatography (silica; EtOAc in heptane 50/50 to 100/0). the desired fractions were collected and evaporated in vacuo to intermediate 210 as an orange oil.

### Preparation of Intermediate 211

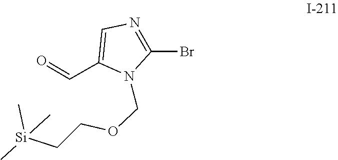 [SMILES: C[Si](C)(C)CCOCn1c(C=O)cnc1Br]

A freshly prepared 0.48M lithium tetramethylpiperidide solution in THF (CAS 38227-87-1, 5.37 mL, 2.58 mml) was added dropwise to a solution of 2-bromo-1-[[2-(trimethylsilyl)ethoxy]methyl]-1H-imidazole (CAS 134183-57-6, 0.65 g, 2.3 mmol) in THF (11.4 mL) at −78° C. and under N₂. The mixture was stirred at −78° C. for 1 hour and then a solution of DMF in THF (1.09 mL) was added at −78° C. The reaction was stirred at −78° C. for 10 min and allowed to warm to rt. Then a 10% Na₂SO₃ solution was added and the mixture was extracted with EtOAc. The crude product was purified by flash column chromatography (silica; EtOAc in heptane 10/90 to 40/60). The desired fractions were collected and the solvent evaporated in vacuo to yield intermediate 211 as a yellow oil.

### Preparation of Intermediate 212

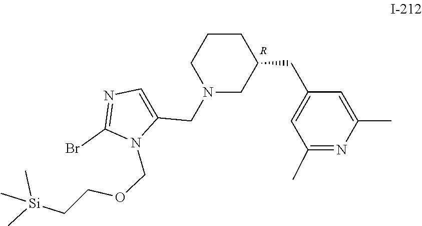

TEA (CAS 121-44-8, 0.49 mL, 3.55 mmol) was added to a stirred solution of intermediate 3 (354 mg, 1.28 mmol) in ACN (2.45 mL) at 10° C. under nitrogen. The mixture was allowed to warm to rt and then intermediate 211 (325 mg, 1.06 mmol) was added. The mixture was stirred at rt for 30 min and then sodium triacetoxyborohydride (CAS 56553-60-7, 564 mg, 2.66 mmol) was added portionwise. The mixture was stirred at rt for 2 h, warmed to 50° C. stirred at this temperature for 15 min. Then, the mixture was cooled down to rt and quenched with water and ammonium chloride. EtOAc was added and the pH of the aqueous layer was adjusted to >7 by addition of a 3N NaOH solution. The aqueous phase was extracted with EtOAc and the organic layer was separated, dried (MgSO₄), filtered and the solvents evaporated in vacuo. The crude product was purified by flash column chromatography (silica; MeOH in DCM 0/100 to 7/93). The desired fractions were collected and evaporated in vacuo to yield intermediate 212 (465 mg, 93%) as a colorless oil.

### Preparation of Intermediate 213

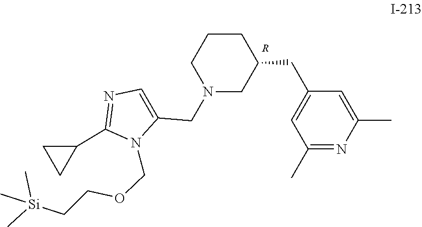

A 0.3M cyclopropylzinc bromide solution in THF (CAS 126403-68-7, 2.79 mL, 0.84 mmol) was added to mixture of intermediate 212 (165 mg, 0.33 mmol), tris(dibenzylideneacetone)dipalladium(0) (CAS 51364-51-3, 31 mg, 0.033 mmol) and SPhos (CAS657408-07-6, 27 mg, 0.067 mmol) under N₂ atmosphere. The mixture was stirred at rt for 3 h and at 50° C. for a further 4 hours. Then a saturated NH₄Cl solution was added and the mixture was extracted with EtOAc. The organic layer was separated, dried (Na₂SO₄), filtered and the solvents evaporated in vacuo. The crude product was purified by flash column chromatography (silica; MeOH in DCM 2/98 to 10/90) and by RP HPLC (Stationary phase: C18 XBridge 30×100 mm 5 um), Mobile phase: Gradient from 81% 0.1% NH₄CO₃H/NH₄OH pH 9 solution in Water, 19% CH₃CN to 64% 0.1% NH₄CO₃H/NH₄OH pH 9 solution in Water, 36% CH₃CN) to yield intermediate 213 (13 mg, 9%) as a colorless oil.

### Preparation of the Final Compounds

### E1. Preparation of Product 1

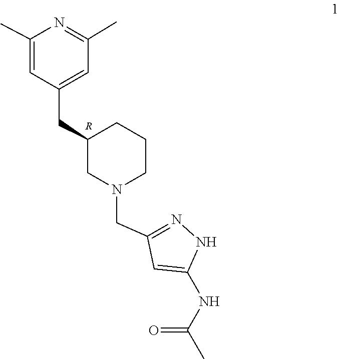

To a solution of intermediate 3 (0.08 g, 0.39 mmol) in DCE (1.6 mL) and DMF (0.30 mL), intermediate 51 (0.06 g, 0.39 mmol) was added and the reaction mixture was stirred at rt for 30 min. Then sodium triacetoxyborohydride (CAS 56553-60-7, 0.17 g, 0.78 mmol) was added and the reaction mixture was stirred at rt for 18 h. Then saturated solution of NaHCO₃ was added and the product extracted with DCM. The organic layer was separated, dried (MgSO₄), filtered and the solvents evaporated in vacuo. The product was purified by RP HPLC (Stationary phase: C18 XBridge 30×100 mm 5 um, mobile phase: Gradient from 80% 10 mM NH₄CO₃H pH 9 solution in H₂O, 20% CH₃CN to 63% 10 mM NH₄CO₃H pH 9 solution in H₂O, 37% CH₃CN) and then by flash column chromatography (silica; NH₃ in EtOH in DCM: 0/100 to 10/90). The desired fractions were collected and concentrated in vacuo to yield a compound that was further dried under vacuo at 50° C. for 24 h to yield product 1 (63 mg, 47%) as a white solid.

### E2. Preparation of Product 2

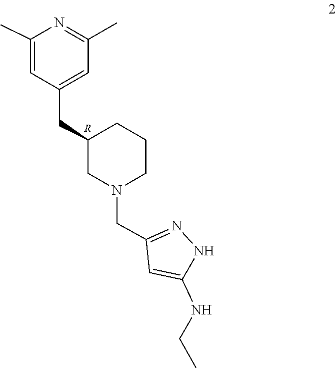

To a stirred solution of product 1 (0.05 g, 0.15 mmol) in dry THF (1 mL) under a N₂ atmosphere was added a 1M solution of LAH in THF (0.22 mL, 0.22 mmol) at 0° C., and the reaction mixture was stirred for 2 h. Then the reaction mixture was allowed to warm to rt and stirred for 18 h. The reaction mixture was quenched with a 1N solution of HCl in 1,4-dioxane. The mixture was filtered and the filtrate concentrated in vacuo. The residue was purified by flash column chromatography (silica; EtOH in DCM: 0/100 to 5/95). The desired fractions were collected and concentrated in vacuo to yield a product that was further purified by RP-HPLC (Stationary phase: C18 XBridge 30×100 mm 5 um, mobile phase: Gradient from 80% 10 mM NH₄CO₃H pH 9 solution in H₂O, 20% ACN to 63% 10 mM NH₄CO₃H pH 9 solution in H₂O, 37% ACN) to yield product 2 (15 mg, 31%) as a white solid.

### E3. Preparation of Product 3, 4 and 5

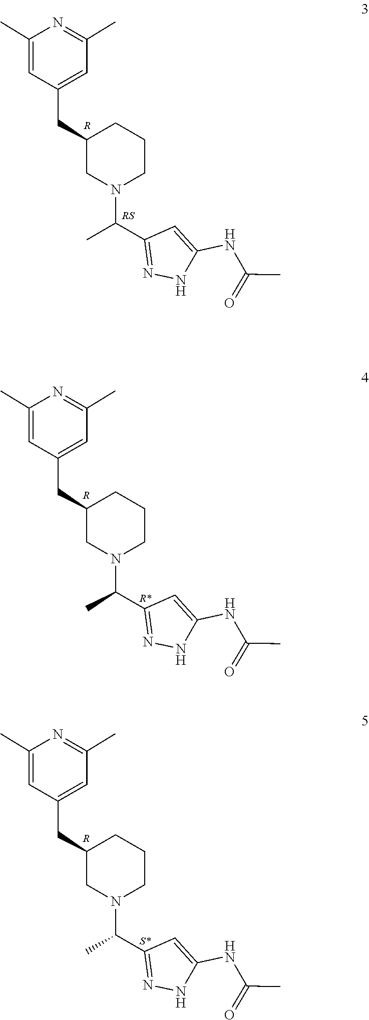

A 4M HCl solution in 1,4-dioxane (3.4 mL, 4.3 mmol) was added to intermediate 58 (0.2 g, 0.43 mmol) and the reaction mixture stirred at rt for 18 h. The reaction was concentrated to dryness and the residue was purified by ion exchange chromatography using an ISOLUTE SCX2 cartridge eluting first with EtOH and then with 7M solution of ammonia in EtOH. The desired fractions were collected and concentrated in vacuo. The residue was suspended in 1,4-dioxane and Ac₂O (2 eq, 0.86 mmol, 0.08 mL) was added. The reaction mixture was stirred at rt for 3 h. The reaction was concentrated to dryness and the residue was purified by flash column chromatography (silica; EtOH in DCM: 0/100 to 10/90). The desired fractions were collected and concentrated in vacuo to yield product 3 (90 mg, 58%) as a white solid.

Product 3 was purified by RP-HPLC (Stationary phase: C18 XBridge 30×100 mm 5 μm, mobile phase: Gradient from 90% NH₄HCO₃ 0.25% solution in H₂O, 10% ACN to 65% NH₄HCO₃ 0.25% solution in H₂O, 35% ACN), yielding flash column chromatography (silica; EtOH in DCM: 0/100 to 10/90). The desired fractions were collected and concentrated in vacuo and the product thus obtained was purified via chiral SFC (Stationary phase: Chiralcel OD-H 5 μm 250×21.2 mm, mobile phase: 85% C02, 15% EtOH (0.3% iPrNH₂)) to yield product 4 (21 mg, 26%) and product 5 (16 mg, 27%).

### E4. Preparation of Product 6

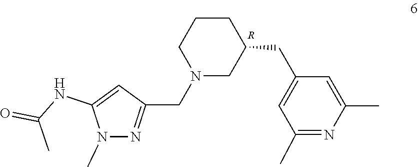

To a solution of intermediate 86 (15 mg, 0.048 mmol) in 1,4-dioxane (0.08 mL) was added acetic anhydride (0.01 mL, 0.11 mmol) dropwise and the reaction mixture was stirred at rt for 3 h and at 70° C. for 1 h. The reaction was concentrated in vacuo and the residue was purified by flash chromatography (silica; EtOH in DCM, 0/100 to 5/95). The desired fractions were collected and the solvents removed in vacuo to yield product 6 (15 mg, 88%) as a white solid.

### E5. Preparation of Product 7, 8 and 9

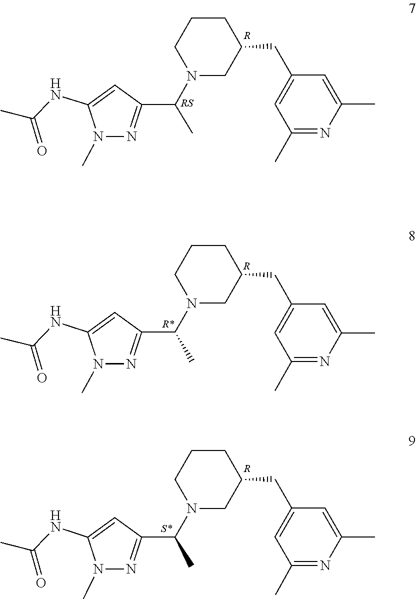

To a solution of intermediate 3 (0.42 g, 2.05 mmol) in DCM (7.9 mL), intermediate 57 (0.38 g, 2.26 mmol) and titanium(IV)isopropoxide (0.90 mL, 3.08 mmol) were added and the reaction mixture was stirred at rt overnight. Then the reaction was cooled to 0° C. and a 3M solution of methylmagnesium bromide in diethyl ether (3.43 mL, 10.28 mmol) was added dropwise and the reaction mixture was stirred at 0° C. for 5 min and at rt for 1 h. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 10/90). The desired fractions were collected and the solvents evaporated in vacuo to yield product 7 (0.39 g, 52%) as a yellow solid. Purification of product 7 was performed via chiral SFC (Stationary phase: CHIRALPAK AD-H 5 μm 250*30 mm, mobile phase: 70% C02, 30% iPrOH (0.3% iPrNH₂)) to yield product 8 (0.15 g, 39%) and product 9 (0.17 g, 44%).

### E6. Preparation of Product 10

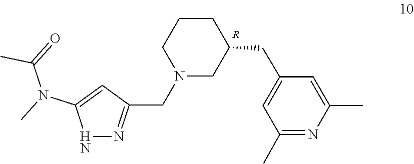

To a solution of intermediate 79 (40 mg, 0.08 mmol) in DCM (0.3 mL); TFA (0.066 mL, 0.86 mmol) was added and the reaction mixture was stirred at rt for 18 h. The reaction was concentrated to dryness and the residue was purified first by ion exchange chromatography using an ISOLUTE SCX2 cartridge eluting first with EtOH and then with 7M solution of ammonia in EtOH and then by RP HPLC (Stationary phase: C18 XBridge 30×100 mm 5 um, mobile phase: Gradient from 80% 10 mM NH₄CO₃H pH 9 solution in H₂O, 20% CH₃CN to 63% 10 mM NH₄CO₃H pH 9 solution in H₂O, 37% CH₃CN) to yield product 10 (28 mg, 91%) as a white solid.

### E4. Preparation of Product 11

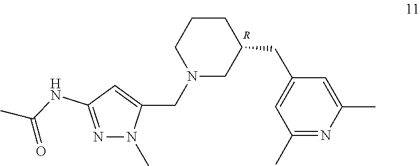

To a solution of intermediate 85 (0.11 g, 0.37 mmol) in 1,4-dioxane (0.62 mL) was added acetic anhydride (0.07 mL, 0.80 mmol) dropwise and the reaction mixture was stirred at rt for 3 h. The reaction was concentrated in vacuo and the residue was purified by flash chromatography (silica; EtOH in DCM, 0/100 to 5/95). The desired fractions were collected and the solvents removed in vacuo to yield product 11 (0.12 g, 96%) as a white solid.

### E6. Preparation of Products 12 and 13

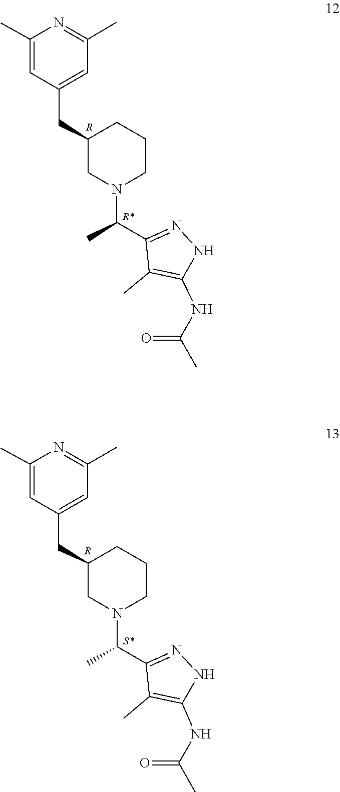

To a solution of intermediate 64 (0.06 g, 0.13 mmol) in DCM (0.6 mL); TFA (0.10 mL, 1.26 mmol) was added and the reaction mixture was stirred at rt for 18 h. The reaction was concentrated to dryness and the residue was purified first by ion exchange chromatography using an ISOLUTE SCX2 cartridge eluting first with EtOH and then with 7M solution of ammonia in EtOH and then by RP-HPLC (Stationary phase: C18 XBridge 30×100 mm 5 um, mobile phase: Gradient from 80% 10 mM NH₄CO₃H pH 9 solution in H₂O, 20% ACN to 63% 10 mM NH₄CO₃H pH 9 solution in H₂O, 37% ACN) to yield product 12 (15 mg. 32%) and product 13 (20 mg, 43%) as white solids.

### E3. Preparation of Product 14

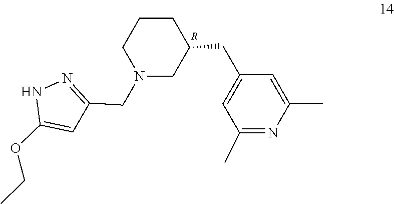

A 1.25M solution of hydrogen chloride in EtOH (2.1 mL, 2.63 mmol) was added to intermediate 91 (0.23 g, 0.53 mmol) and the reaction mixture was stirred at rt for 3 days. The solvent was evaporated in vacuo and the crude product was purified by ion exchange chromatography (ISOLUTE SCX2 cartridge, MeOH and then 7N solution of NH₃ in MeOH). The solvent was evaporated in vacuo. The residue was purified by flash column chromatography (silica; MeOH in DCM 0/100 to 6/94). The desired fractions were collected and the solvents evaporated in vacuo. The product was triturated with a mixture DIPE/Et₂O to yield product 14 (88 mg, 51%) as a white solid.

### E3. Preparation of Product 15 and 16

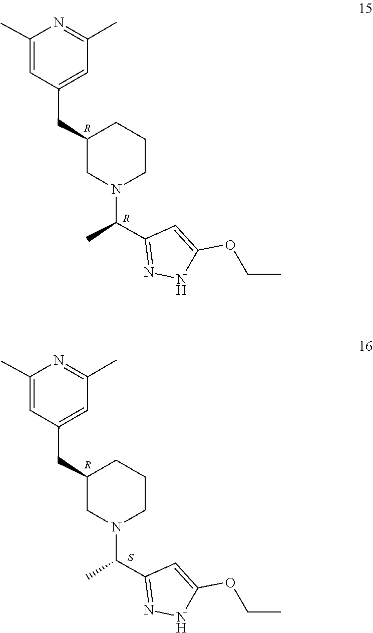

A 1.25M solution of hydrogen chloride in EtOH (1.8 mL, 2.3 mmol) was added to intermediate 92 (0.20 g, 0.47 mmol) and the reaction mixture was stirred at rt for 3 days. The solvent was evaporated in vacuo and the crude product was purified by ion exchange chromatography (ISOLUTE SCX2 cartridge, MeOH and then 7N solution of NH₃ in MeOH). The solvent was evaporated in vacuo. The residue was purified by RP-HPLC (Stationary phase: C18 XBridge 30×100 mm 5 μm, mobile phase: Gradient from 75% NH₄HCO₃ 0.25% solution in H₂O, 25% ACN to 57% NH₄HCO₃ 0.25% solution in H₂O, 43% ACN). The desired fractions were collected and the solvents evaporated in vacuo to yield product 15 (55 mg, 36%) and product 16 (35 mg, 22%) as white solids.

### E5. Preparation of Product 17

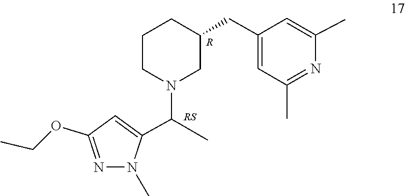

To a solution of intermediate 3 (0.22 g, 1.08 mmol) in DCM (4.15 mL), 3-ethoxy-1-methyl-1H-pyrazole-5-carboxaldehyde (CAS 1823354-98-8, 0.2 g, 1.29 mmol) and titanium(IV) isopropoxide (0.47 mL, 1.62 mmol) were added and the reaction mixture was stirred at rt overnight. Then the reaction was cooled to 0° C. and a 1.4M solution of methylmagnesium bromide in THF:toluene (1.80 mL, 5.40 mmol) was added dropwise and the reaction mixture was stirred at 0° C. for 5 min and at rt for 1 h. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 15/85). The desired fractions were collected and the solvents evaporated in vacuo to yield a yellow oil that was further purified by RP HPLC (Stationary phase: C18 XBridge 30×100 mm 5 μm, mobile phase: Gradient from 60% NH₄HCO₃ 0.25% solution in H₂O, 40% CH₃CN to 43% NH₄HCO₃ 0.25% solution in H₂O, 57% CH₃CN), yielding product 17 (40 mg, 13%) as a colorless oil.

### E5. Preparation of Product 18

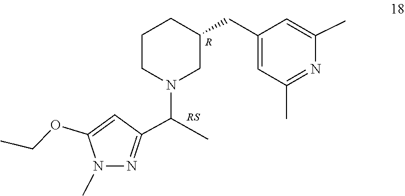

To a solution of intermediate 3 (0.24 g, 1.18 mmol) (0.24 g, 1.18 mmol) in DCM (4.5 mL), intermediate 87 (0.24 g, 1.18 mmol) and titanium(IV)isopropoxide (0.52 mL, 1.77 mmol) were added and the reaction mixture was stirred at rt overnight. Then the reaction was cooled to 0° C. and 1.4M solution of methylmagnesium bromide in THF:toluene (1.96 mL, 5.89 mmol) was added dropwise and the reaction mixture was stirred at 0° C. for 5 min and at rt for 1 h. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 10/90). The desired fractions were collected and the solvents evaporated in vacuo to yield product 18 (0.24 g, 65%) as a yellow oil.

### E3. Preparation of Product 19

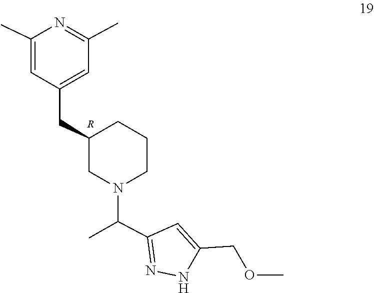

A 1.25M solution of hydrogen chloride in EtOH (1.65 mL, 2.1 mmol) was added to intermediate 98 (0.077 g, 0.17 mmol) and the reaction mixture was stirred at 50° C. for 5 h. The solvent was evaporated in vacuo and then, saturated solution of NaHCO₃ was added and the product extracted with EtOAc. The organic layer was separated, dried (MgSO₄), filtered and the solvents evaporated in vacuo. This crude was purified by RP-HPLC (Stationary phase: C18 XBridge 30×100 mm 5 μm, mobile phase: Gradient from 90% NH₄HCO₃ 0.25% solution in H₂O, 10% ACN to 65% NH₄HCO₃ 0.25% solution in H₂O, 35% ACN). The desired fractions were collected and the solvents evaporated in vacuo to yield product 19 (41 mg, 70%) as a yellow oil.

### E1. Preparation of Product 20

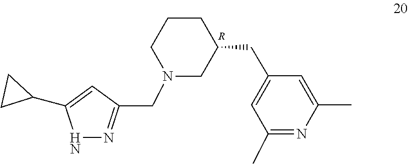

To a solution of intermediate 3 (80 mg, 0.39 mmol) in DCE (2 mL) and DMF (0.2 mL), 5-cyclopropyl-1H-pyrazole-3-carboxaldehyde (CAS 1284220-47-8, 0.054 g, 0.39 mmol) was added and the reaction mixture was stirred at rt for 30 min. Then sodium triacetoxyborohydride (0.16 g, 0.78 mmol) was added and the reaction mixture was stirred at rt for 1 h. Then saturated solution of NaHCO₃ was added and the product extracted with DCM. The organic layer was separated, dried (MgSO₄), filtered and the solvents evaporated in vacuo. The crude product was purified by RP HPLC (Stationary phase: C18 XBridge 30×100 mm 5 um, mobile phase: Gradient from 80% NH₄HCO₃ 0.25% solution in H₂O, 20% CH₃CN to 60% NH₄HCO₃ 0.25% solution in H₂O, 40% CH₃CN). The desired fractions were collected and concentrated in vacuo to yield a compound that was diluted with H₂O and extracted with DCM. The organic layer was separated, dried (MgSO₄), filtered and the solvents evaporated in vacuo to yield product 20 (17 mg, 13%) as a sticky white solid.

### E5. Preparation of Product 21

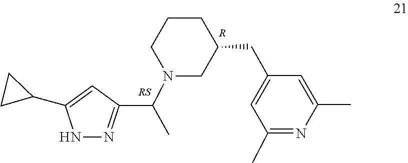

Titanium(IV)isopropoxide (1.5 equiv., 0.35 mL, 1.19 mmol) was added to a solution of intermediate 3 (0.081 g, 0.39 mmol) and 5-cyclopropyl-1H-pyrazole-3-carboxaldehyde (CAS 1284220-47-8, 0.081 g, 0.59 mmol) in DCM (1.5 mL). The mixture was stirred at rt for 2 h. Then, the mixture was cooled to 0° C., a 1.4M solution of methylmagnesium bromide in THF:toluene (5.0 eq, 2.73 mL, 1.95 mmol) was added dropwise and the reaction mixture was stirred at 0° C. for 5 min. and at rt for 2 h. Then, the mixture was cooled to 0° C., more 1.4M solution of methylmagnesium bromide in THF:toluene (5.0 eq, 2.73 mL 1.95 mmol) was added dropwise and the reaction mixture was stirred at 0° C. for 5 min. and at rt for 16 h. Then saturated solution of NH₄Cl was added and the product extracted with DCM. The organic layer was separated, dried (MgSO₄), filtered and the solvents evaporated in vacuo. The residue was dissolved in DCM (1.5 mL) and titanium(IV)isopropoxide (1.5 equiv., 0.35 mL, 1.19 mmol) was added. The mixture was stirred at rt for 18 h. Then the mixture was cooled to 0° C., a 1.4M solution of methylmagnesium bromide in THF:toluene (5.0 eq, 2.73 mL, 1.95 mmol) was added dropwise and the reaction mixture was stirred at 0° C. for 5 min and at rt for 2 h. Then, the mixture was cooled to 0° C., a 1.4M solution of methylmagnesium bromide in THF:toluene (5.0 eq, 2.73 mL, 1.95 mmol) was added dropwise and the reaction mixture was stirred at 0° C. for 5 min. and at rt for 3 days. Then saturated solution of NH₄Cl was added and the product extracted with DCM. The organic layer was separated, dried (MgSO₄), filtered and the solvents evaporated in vacuo. The crude product was purified by flash column chromatography (silica; MeOH in DCM 0/100 to 10/90). The desired fractions were collected and the solvents evaporated in vacuo to yield a colorless oil that was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and the solvents evaporated in vacuo. The product was triturated with a mixture DIPE to yield a white solid that was further dried under vacuo at 50° C. for 24 h to yield product 21 (21 mg, 16%) as a white solid.

### E6. Preparation of Product 22

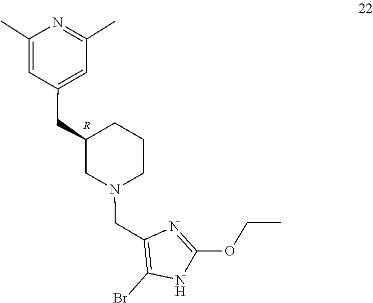

To a solution of intermediate 99 (0.20 g, 0.37 mmol) in DCM (3 mL), TFA (4 mL) was added, the reaction mixture was stirred at rt for 2 h. The crude was evaporated in vacuo. The residue was purified by flash column chromatography (silica; 7M solution of ammonia in MeOH in DCM 0/100 to 6/94). The desired fractions were collected and concentrated in vacuo to yield 140 mg of an impure compound as a transparent oil; 50 mg of this compound was purified by RP-HPLC (Stationary phase: C18 XBridge 30×100 mm 5 um, mobile phase: Gradient from 67% 0.1% NH₄CO₃H/NH₄OH pH 9 solution in H₂O, 33% ACN to 50% 0.1% NH₄CO₃H/NH₄OH pH 9 solution in H₂O, 50% CH₃CN), yielding product 22 as solid.

### E7. Preparation of Product 23

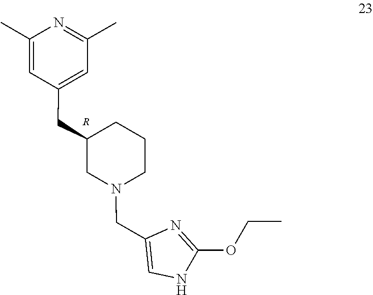

To a solution of product 22 (0.13 g, 0.32 mmol) in MeOH (5 mL), Pd/C (10%) (7.8 mg, 0.007 mmol) was added and the reaction was hydrogenated (atmospheric pressure) at rt for 24 h. The reaction mixture was filtered through a pad of diatomaceous earth and the filter cake was washed thoroughly with EtOAc. The crude was evaporated in vacuo and purified by RP-HPLC (Stationary phase: C18 XBridge 30×100 mm 5 um, mobile phase: Gradient from 81% 0.1% NH₄CO₃H/NH₄OH pH 9 solution in H₂O, 19% ACN to 64% 0.1% NH₄CO₃H/NH₄OH pH 9 solution in H₂O, 36% ACN), the corresponding fractions were evaporated in vacuo. The resulting oil was washed with a saturated NaHCO₃ solution and DCM, the crude was stirred during 30 min, the organic phase was separated dried and concentrated in vacuo to yield an oil which was purified again by RP-HPLC (Stationary phase: C18 XBridge 30×100 mm 5 um, mobile phase: Gradient from 81% 0.1% NH₄CO₃H/NH₄OH pH 9 solution in H₂O, 19% ACN to 64% 0.1% NH₄CO₃H/NH₄OH pH 9 solution in H₂O, 36% ACN), the corresponding fractions were evaporated in vacuo yielding product 23 (9 mg, 8%) as transparent oil.

### E5. Preparation of Product 24

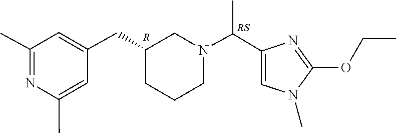

Titanium(IV)isopropoxide (0.13 mL, 0.45 mmol) was added to a stirred solution of intermediate 100 (0.046 g, 0.298 mmol) and intermediate 3 (0.060 g, 0.298 mmol) in DCM anhydrous (1.21 mL) at rt and under N₂. The mixture was stirred at rt for 16 h. Then the mixture was cooled at 0° C. and a 1.4M methylmagnesium bromide in THF:toluene (1.06 mL, 1.49 mmol) was added dropwise. The resulting mixture was stirred at this temperature for 15 min and then at rt for 2.5 h. The mixture was treated with sat. NH₄Cl and extracted with DCM. The phases were filtered through celite and then, the organic layer was separated, dried (Na₂SO₄), filtered and the solvents evaporated in vacuo. The crude product was purified by flash column chromatography (silica; 7N solution of ammonia in EtOH in DCM 0/100 to 4/96). The desired fractions were collected and the solvents evaporated in vacuo to yield product 24 (69 mg, 65%) as yellowish oil.

### E1. Preparation of Product 25

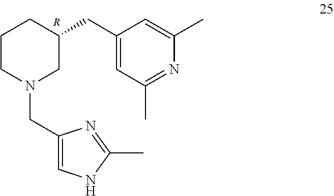

To a solution of intermediate 3 (0.10 g, 0.49 mmol) in DCE (15 mL) and AcOH (0.2 mL), 2-methyl 1H-imidazole-5-carboxaldehyde (CAS 35034-22-1 (54 mg, 0.49 mmol) and sodium triacetoxyborohydride (0.156 g, 0.736 mmol) were added at rt. The reaction mixture was stirred at rt during 24 h. Then more amount of 2-methyl-1H-imidazole-5-carboxaldehyde (CAS 35034-22-1, 27 mg, 0.24 mmol) and sodium triacetoxyborohydride (0.104 g, 0.49 mmol) were added, the mixture was stirred during 24 h at rt. The crude was treated with H₂O and stirred for 10 min. The two phases were separated, the compound was present in the aqueous phase which was evaporated. The crude was passed through a SCX-1 cartridge eluting first with EtOH and then with MeOH/NH₃ 7N. The desired fractions were concentrated to yield an oil which was purified by RP HPLC (Stationary phase: C18 XBridge 30×100 mm 5 um, mobile phase: Gradient from 80% 0.1% NH₄CO₃H/NH₄OH pH 9 solution in H₂O, 20% CH₃CN to 0% 0.1% NH₄CO₃H/NH₄OH pH 9 solution in H₂O, 100% CH₃CN). The desired fractions were concentrated to yield product 25 (101 mg, 69%) as yellow solid.

### E1. Preparation of Product 26

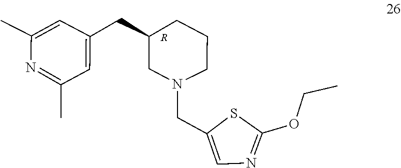

2-Ethoxy-1,3-thiazole-5-carbaldehyde (CAS 220389-76-4, 54 mg, 0.49 mmol) was added to a stirred solution of intermediate 3 (0.10 g, 0.49 mmol) in DCE (3 mL) and AcOH (0.2 mL). The mixture was stirred at rt for 1 h and then sodium triacetoxyborohydride (0.156 g, 0.736 mmol) was added. The resulting reaction mixture was stirred at rt for 3 days. The mixture was diluted with sat. NaHCO₃ and extracted with DCM. The organic layer was separated, dried (Na₂SO₄), filtered and the solvents evaporated in vacuo. The crude product was purified by flash column chromatography (silica; 7N solution of ammonia in EtOH in DCM 0/100 to 4/96). The desired fractions were collected and the solvents evaporated in vacuo to yield a pale yellow oil that was purified by RP HPLC (Stationary phase: C18 XBridge 30×100 mm 5 μm, mobile phase: Gradient from 60% NH₄HCO₃ 0.25% solution in H₂O, 40% CH₃CN to 43% NH₄HCO₃ 0.25% solution in H₂O, 57% CH₃CN). Desired fractions were evaporated in vacuo to yield product 26 as yellow oil.

### E8. Preparation of Product 27

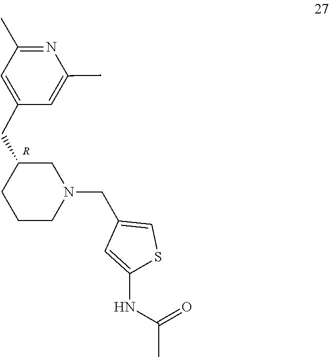

Iron (85 mg, 1.52 mmol) followed by a solution of ammonium chloride (22 mg, 0.41 mmol) in H₂O (0.7 mL) were added to a stirred solution of intermediate cmmartin_5525 (0.054 g, 0.15 mmol) in THF (1.5 mL) and EtOH (1.5 mL) in a sealed tube and under N₂. The mixture was stirred at 90° C. for 1 h. The mixture was treated with H₂O and extracted with EtOAc. The organic layer was separated, dried (MgSO₄), filtered and the solvents evaporated in vacuo. The crude was dissolved with DCM (1.5 mL) and TEA (0.050 mL) followed by acetyl chloride (0.020 mL, 0.28 mmol) were added. The mixture was stirred at rt for 1 h. The mixture was treated with sat NaHCO₃ and extracted with DCM. The organic layer was separated, dried (MgSO₄), filtered and the solvents evaporated in vacuo. The crude product was purified by flash column chromatography (silica; 7N solution of NH₃ in MeOH in DCM 0/100 to 10/90). The desired fractions were collected and concentrated in vacuo to yield product 27 (13 mg, 23%) as a dark oil.

### E1. Preparation of Product 28

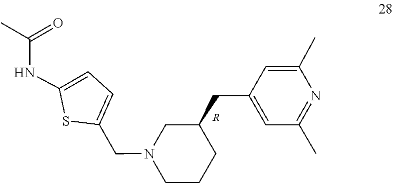

N-(5-Formyl-2-thienyl)acetamide (CAS 31167-35-8, 86 mg, 0.51 mmol) was added to a stirred mixture of intermediate 3 (0.112 g, 0.40 mmol), TEA (0.22 mL, 1.61 mmol) in DCM (2 mL) in a sealed tube and under N₂. The mixture was stirred at rt for 30 min and then triacetoxyborohydride (0.19 g, 0.91 mmol) was added. The mixture was stirred at rt for 18 h. The mixture was treated with sat NaHCO₃ and extracted with DCM. The organic layer was separated, dried (MgSO₄), filtered and the solvents evaporated in vacuo. The crude product was purified by flash column chromatography (silica; 7N solution of NH₃ in MeOH in DCM 0/100 to 7/93), the desired fractions were collected and concentrated in vacuo to yield product 28 (125 mg, 86%) as a yellow foam.

### E1. Preparation of Product 29

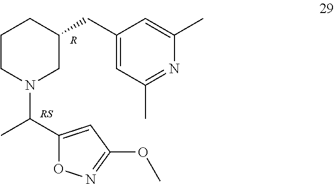

A solution of intermediate 3 (0.10 g, 0.49 mmol) in MeOH (2.8 mL) followed by titanium(IV) isopropoxide (0.29 mL, 0.8 mmol) and sodium cyanoborohydride (95 mg, 1.50 mmol) were added to 1-(3-methoxy-1,2-oxazol-5-yl)ethan-1-one (CAS 54258-26-3, 0.095 g, 0.67 mmol) in a sealed tube and under N₂. The mixture was stirred at 80° C. for 16 h. The solvent was evaporated in vacuo and the crude product was purified by flash column chromatography (silica; 7N solution of NH₃ in MeOH in DCM 0/100 to 10/90) and by RP HPLCHPLC (Stationary phase: C18 XBridge 30×100 mm 5 μm, mobile phase: Gradient from 67% NH₄HCO₃ 0.25% solution in H₂O, 33% CH₃CN to 50% NH₄HCO₃ 0.25% solution in H₂O, 50% CH₃CN). The desired fractions were collected and concentrated in vacuo to yield product 29 (27 mg, 17%) as a yellow oil.

### E5. Preparation of Product 30

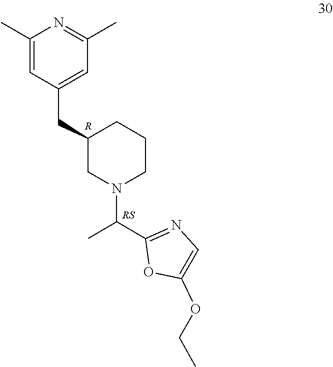

Intermediate 3 (0.15 g, 1.0 mmol) and titanium(IV)isopropoxide (0.69 mL, 2.35 mmol) were added to a solution of 5-ethoxy-2-oxazolecarboxaldehyde (CAS 956118-42-6, 0.16 g, 0.78 mmol) in anhydrous THF (3 mL) at rt. The reaction mixture was stirred at rt for 18 h. Then the mixture was cooled to 0° C. and a 1.4M solution of methylmagnesium bromide in THF:toluene (2.8 mL, 3.9 mmol) was added. The reaction mixture was stirred at 0° C. for 15 min and at rt for 1.5 h. NH₄Cl sat. was added and the mixture was extracted with DCM. The organic layers were dried over MgSO₄ and concentrated in vacuo. The crude product was purified by flash column chromatography (silica; MeOH in DCM 0/100 to 5/95). The desired fractions were collected and concentrated in vacuo to give product 30 (0.17 g, 63%) as a yellow oil.

### E9. Preparation of Product 31

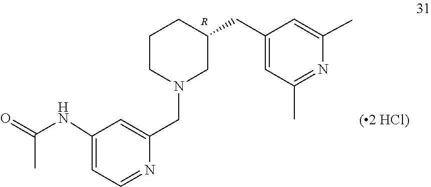

Palladium(II)acetate (3.8 mg, 0.017 mmol), 9,9-dimethyl-4,5-bis(diphenylphosphino)xanthene (22 mg, 0.038 mmol) and Cs₂CO₃ (0.28 g, 0.85 mmol) were added to a stirred solution of acetamide (37 mg, 0.64 mmol) and intermediate X (0.2 g, 0.42 mmol) in 1.4-dioxane (5 mL) under N₂. The reaction mixture was degassed with N₂ and stirred at 94° C. overnight. Pd₂(dba)₃ (15 mg, 0.02 mmol) and 9,9-dimethyl-4,5-bis(diphenylphosphino)xanthene (22 mg, 0.04 mmol) were added to 1.4-dioxane (5 mL) under N₂ and this mixture stirred at 40° C. for 20 min. This solution was added to the reaction mixture and heated at 95° C. overnight. The mixture was diluted with H₂O and extracted with EtOAc. The organic layer was separated, dried (MgSO₄), filtered and the solvents evaporated in vacuo. The crude product was purified by flash column chromatography (silica; MeOH-DCM (10:1) in DCM, from 0/100 to 100/0). The desired fractions were collected and the solvents evaporated in vacuo. This material was purified for reverse phase (from 72% (H₂O 25 mM NH₄HCO₃)-28% ACN-MeOH to 36% H₂O (25 mM NH₄HCO₃)-64% ACN-MeOH). The desired fractions were collected concentrated in vacuo to yield expected compound as a colorless sticky solid. The material was taken into DCM and treated with 2 eq of a 4N solution of HCl in 1,4-dioxane (0.04 mL). The solvents evaporated in vacuo and the product was triturated with diethyl ether to yield product 31 (33 mg, 18%) as a white solid.

### E5. Preparation of Product 32

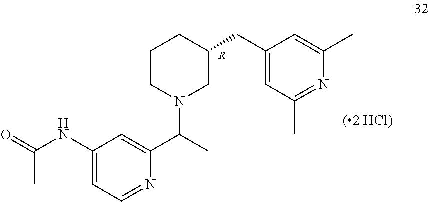

N-(2-Formyl-4-pyridinyl)-acetamide (CAS 120356-46-9, 0.10 g, 0.59 mmol) and titanium (IV)isopropoxide (0.35 mL, 1.18 mmol) were added to a solution of intermediate 3 (0.08 g, 0.39 mmol) in anhydrous THF (1 mL) at rt and the reaction mixture was stirred at rt for 18 h. The mixture was distillated and dried in vacuo. Then, THF (1 mL) was added and the reaction was cooled to 0° C. and 1.4M solution of methylmagnesium bromide in THF:toluene (1.40 mL, 1.97 mmol) was added dropwise and the reaction mixture was stirred at 0° C. for 15 min and at rt for 15 h. NH₄Cl sat was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo. The product was purified by phase reverse 72% [25 mM NH₄HCO₃]-28% [ACN: MeOH 1:1] to 36% [25 mM NH₄HCO₃]-64% [ACN:MeOH 1:1]. The solvents were concentrated in vacuo, ACN (10 mL×3 times) was added and was concentrated in vacuo at 60° C. The solvents evaporated in vacuo to yield a compound that was diluted in DCM and 4N solution in 1,4-dioxane was added. The solvents evaporated in vacuo and the product was triturated with diethyl ether to yield product 32 as a white solid.

### E9. Preparation of Product 33

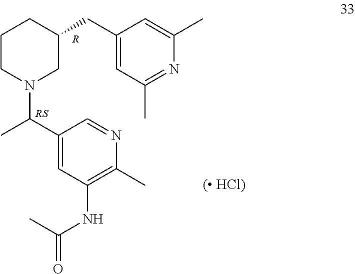

Palladium(II)acetate (1.86 mg, 0.008 mmol), 9,9-dimethyl-4,5-bis(diphenylphosphino)xanthene (11 mg, 0.02 mmol) and Cs₂CO₃ (0.13 g, 0.41 mmol) in anhydrous 1,4-dioxane (8 mL) were heated at 40° C. for 15 min while N₂ was bubbling. Then, acetamide (13 mg, 0.22 mmol) and intermediate X (0.10 g, 0.20 mmol) were added while N₂ was bubbling. The reaction mixture was stirred at 90° C. for 5 h. The reaction mixture was cooled to rt and filtered over a pad of diatomaceous earth. The solvents were evaporated in vacuo. The crude product was purified by flash column chromatography (silica; MeOH in DCM 0/100 to 10/90). The desired fractions were collected and concentrated in vacuo to yield expected product that was purified by RP-HPLC 90% [65 mM NH4OAc+ACN (90:10)]-10% [ACN: MeOH 1:1] to 54% [65 mM NH₄OAc+ACN (90:10)]-46% [ACN: MeOH 1:1]. The desired fractions were collected and the solvents were concentrated in vacuo to yield desired product that was purified by RP-HPLC 72% [25 mM NH₄HCO₃]-28% [ACN: MeOH 1:1] to 36% [25 mM NH₄HCO₃]-64% [ACN: MeOH 1:1]. The desired fractions were collected and the solvents were concentrated in vacuo at 60° C. ACN (5 mL×3 times) were added and concentrated in vacuo at 60° C. The product was dissolved in DCM (2 mL) and 4N solution of HCl in 1,4-dioxane was added. Finally, the product was obtained pure recrystallized from diisopropyl ether to yield to product 33 (27 mg, 32%) as pale yellow oil.

### E11. Preparation of Product 34

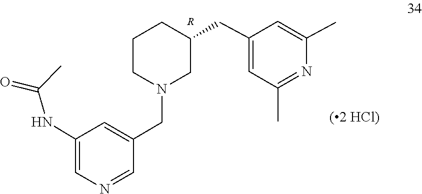

Acetic acid (0.0.32 mL, 0.56 mmol) and sodium cyanoborohydride (26 mg, 0.42 mmol) was added to a stirred solution of intermediate 3 (0.077 g, 0.28 mmol), N-(5-formyl-3-pyridinyl)-acetamide (CAS 1378821-86-3, 0.053 g, 0.31 mmol) and anhydrous sodium acetate (0.089 g, 1.09 mmol) in MeOH (8 mL) at rt. The reaction mixture was stirred at rt for 5 h. 4-(5-formyl-3-pyridinyl)-acetamide (CAS 1378821-86-3, 0.4 eq, 0.017 g, 0.11 mmol) and anhydrous sodium acetate (1 eq, 0.023 g, 0.28 mmol) were added at rt and the mixture was stirred for 5 min, after acetic acid (1 eq, 0.017 mL, 0.28 mmol) and sodium cyanoborohydride (0.6 eq, 0.009 g, 0.17 mmol) were added and the mixture was stirred at rt for 16 h more. H₂O was added and the mixture was extracted with EtOAc (20 ml×3 times). The two layers were concentrated in vacuo. The crude was purified by flash column chromatography (silica; MeOH/DCM (9:1) in DCM 0/100 to 60/40). The desired fractions were collected and concentrated in vacuo a as a white foam. Diethyl ether was added and the solvent was concentrate in vacuo. The product was purified by phase reverse 72% [25 mM NH₄HCO₃]-28% [ACN: MeOH 1:1] to 36% [25 mM NH₄HCO₃]-64% [ACN: MeOH 1:1]. The desired fractions were collected and concentrated in vacuo. The solvents were concentrated in vacuo and acetonitrile (10 ml×3 times) was added and concentrated at 60° C. in vacuo to yield as a colorless oil that was dissolved in DCM (3 mL) and 4N solution of HCl in 1,4-dioxane was added, the solvents were concentrated in vacuo to yield product 34 (47 mg, 38%) as a white solid.

### E5. Preparation of Product 35, 36 and 37

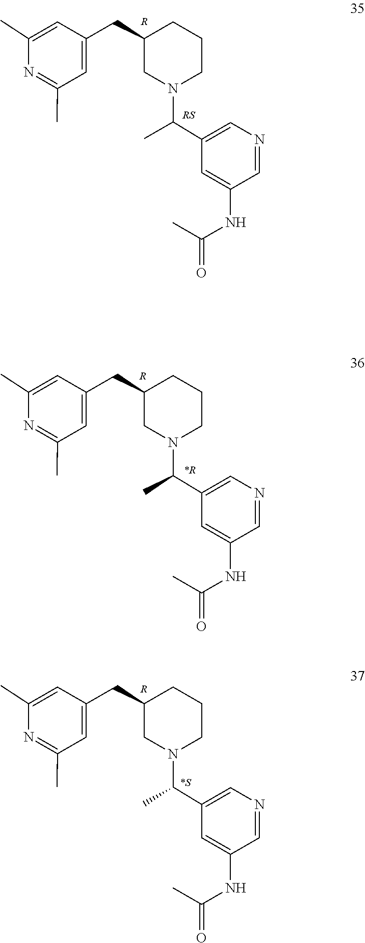

N-(5-Formyl-3-pyridinyl)-acetamide (CAS 1378821-86-3, 0.089 g, 0.54 mmol) and titanium(IV)isopropoxide (0.32 mL, 1.08 mmol) were added to a solution of intermediate 3 (0.073 g, 0.36 mmol) in anhydrous THF (1 mL) at rt and the reaction mixture was stirred at rt for 18 h. Then, the solvent was concentrated in vacuo and the mixture was added anhydrous THF (1 mL) under N₂. The mixture was cooled to 0° C. and a 1.4M solution of methyl magnesium bromide (1.29 mL, 1.80 mmol) was added. The reaction mixture was stirred at 0° C. for 15 min and at rt for 1.5 h. NH₄Cl sat was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo to yield a yellow foam that was purified by phase reverse 72% [25 mM NH₄HCO₃]-28% [ACN: MeOH 1:1] to 36% [25 mM NH₄HCO₃]-64% [ACN: MeOH 1:1] The solvents were concentrated in vacuo, ACN (10 mL×3 times) was added and was concentrated in vacuo at 60° C. The solvents evaporated in vacuo to yield product 35 (17 mg, 12%) as a white foam.

A purification was performed via chiral SFC (Stationary phase: CHIRALPAK AS-H 5 μm 250*20 mm, Mobile phase: 86% C02, 14% MeOH (0.3% iPrNH₂)). The two products obtained were made solid by adding heptane and DIPE to yield product 36 (25 mg, 38%) and product 37 (28 mg, 43%)

### E4. Preparation of Product 38

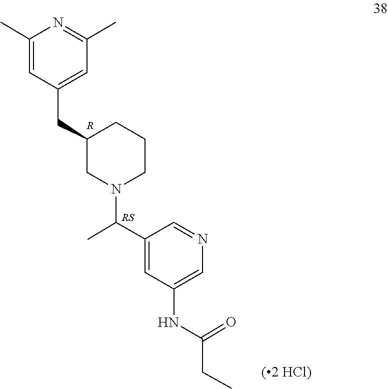

Propionic anhydride (0.11 mL, 0.89 mmol) was added to intermediate 110 (0.09 g, 0.29 mmol) in toluene (3 mL) at rt. The mixture was heated to 100° C. for 1 h. The mixture was concentrated in vacuo. The mixture was diluted with sat NaHCO₃ and extracted with EtOAc. The organics layers were dried (MgSO₄) and concentrated in vacuo. The crude product was purified by flash column chromatography (silica; MeOH (10:1) in DCM 0/100 to 90/10). The desired fractions were collected and concentrated in vacuo. The compound was diluted in DCM and HCl 4N in 1.4-dioxane was added. The solvents evaporated in vacuo and the product was triturated with diethyl ether to yield product 38 (0.08 g, 56%) as a white solid.

### E12. Preparation of Product 39

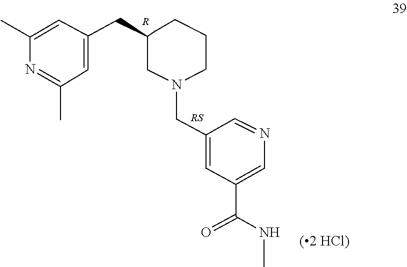

Palladium(II)acetate (1.98 mg, 0.0088 mmol) and bis[(2-diphenylphosphino)phenyl]ether, DPEPhos (15.8 mg, 0.03 mmol) were dissolved in toluene (5 mL) and stirred at rt for 5 min while N₂ was bubbling. A 12M solution of methylamine in H₂O (0.07 mL, 0.35 mmol) was added to the mixture at 35° C. while N₂ was bubbling. Chloroform (0.07 mL, 0.88 mmol) and, intermediate 102 (0.11 g, 0.29 mmol) in cesium hydroxide hydrate (0.49 g, 2.94 mmol), were added to the mixture at 35° C. while N₂ was bubbling. The mixture was stirred in a sealed tube at 110° C. for 18 h. The reaction mixture was diluted with EtOAc and a saturated NaHCO₃ solution. The organic layer was separated, dried (MgSO₄), filtered and the solvents evaporated in vacuo. The product was purified by RP-HPLC 72% [25 mM NH₄HCO₃]-28% [ACN: MeOH 1:1] to 36% [25 mM NH₄HCO₃]-64% [ACN: MeOH 1:1]. The solvents were concentrated in vacuo, ACN (10 mL×3 times) was added and evaporated in vacuo and the product was triturated with diethyl ether to yield product 39 (29 mg, 23%) as a white solid.

### E12. Preparation of Product 40

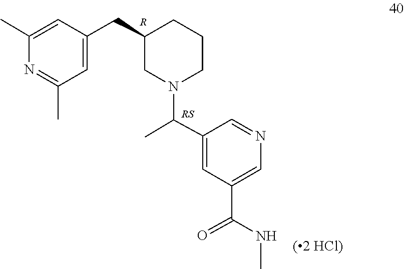

Palladium(II)acetate (1 mg, 0.005 mmol) and bis[(2-diphenylphosphino)phenyl]ether, DPEPhos (9 mg, 0.016 mmol) were dissolved in toluene (5 mL) and stirred at rt for 5 min while N₂ was bubbling. Cesium hydroxide hydrate (0.28 g, 1.65 mmol) was added to the mixture while N₂ was bubbling. Chloroform (0.04 mL, 0.49 mmol), intermediate 103 (0.064 g, 0.16 mmol) and a 12 M solution of methylamine in H₂O (0.04 mL, 0.19 mmol) were added under N₂. The mixture was stirred in a sealed tube at 110° C. for 18 h. The reaction mixture was diluted with EtOAc and a saturated solution of NaHCO₃. The organic layer was separated, dried (MgSO₄), filtered and the solvents evaporated in vacuo. The product was purified by RP-HPLC 90% [25 mM NH₄HCO₃]-10% [ACN: MeOH 1:1] to 54% [25 mM NH₄HCO₃]-46% [ACN: MeOH 1:1]. The solvents were concentrated in vacuo, ACN (10 mL×3 times) was added and was concentrated in vacuo at 60° C. The solvents evaporated in vacuo to yield a compound that was diluted in DCM and a 4N solution of HCl in 1.4-dioxane was added. The solvents evaporated in vacuo and the product was triturated with diethyl ether to yield product 40 (11 mg, 15%) as a white solid.

### E9. Preparation of Product 41 and 42

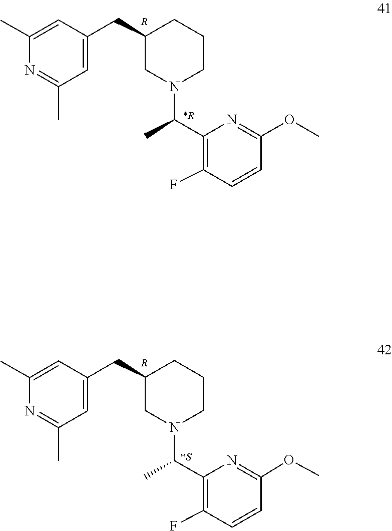

Sodium cyanoborohydride (0.13 g, 2.15 mmol) was added to a stirred solution of intermediate 3 (0.22 g, 1.08 mmol) and 1-(3-fluoro-6-methoxy-2-pyridinyl)-ethanone (CAS 1785479-37-9, 0.27 g, 1.61 mmol), titanium(IV) isopropoxide (0.6 mL, 2.26 mmol) in anhydrous THF (2 mL) under N₂. The mixture was stirred at 70° C. for 24 h in a sealed tube. Then acetic acid (0.1 mL) and MeOH (0.5 mL) were added and the mixture was stirred at 70° C. for 16 h. The solvent was evaporated in vacuo and the crude was purified by flash column chromatography (silica; MeOH in DCM 0/100 to 5/95) the corresponding fractions were collected and concentrated in vacuo, the crude was purified again by RP HPLC (Stationary phase: C18 XBridge 30×100 mm 5 um, mobile phase: Gradient from 54% 0.1% NH₄CO₃H/NH₄OH pH 9 solution in H₂O, 46% CH₃CN to 64% 0.1% NH₄CO₃H/NH₄OH pH 9 solution in H₂O, 36% CH₃CN), the desired fractions were collected and concentrated in vacuo to get yielding product 41 (60 mg, 16%) and impure product 42 as transparent oil that was treated with H₂O due the unknown impurities observed in the NMR spectrum and extracted with DCM, the organic phase was separated, dried (MgSO₄) and evaporated in vacuo to get product 42 (17 mg, 4%) as a transparent oil.

### E5. Preparation of Product 43

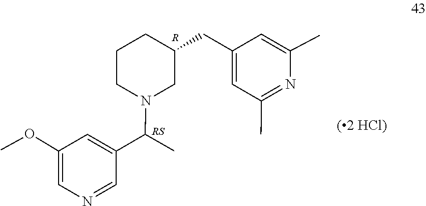

5-Methoxy-3-pyridinecarboxaldehyde (CAS 113118-83-5, 0.07 g, 0.49 mmol) and titanium(IV)isopropoxide (0.36 mL, 1.24 mmol) were added to a solution of intermediate 3 (0.084 g, 0.41 mmol) in anhydrous THF (1 mL) at rt and the reaction mixture was stirred at rt for 18 h. The mixture was distillated and dried in vacuo. Then, anhydrous THF (1 mL) was added and the reaction was cooled to 0° C. and a 1.4M solution of methylmagnesium bromide in THF:toluene (1.47 mL, 2.06) was added dropwise and the reaction mixture was stirred at 0° C. for 15 min and at rt for 15 h. NH₄Cl sat was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo to yield a compound that was diluted in DCM and 4N HCl in 1,4-dioxane was added. The solvents evaporated in vacuo and the product was triturated with diethyl ether to yield product 43 as a white solid.

### E5. Preparation of Product 44

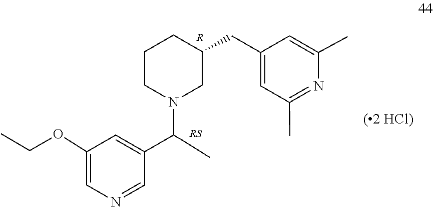

5-Ethoxy-3-pyridinecarboxaldehyde (CAS 227939-23-3, 0.10 g, 0.51 mmol) and titanium (IV)isopropoxide (0.45 mL, 1.54 mmol) were added to a solution of intermediate 3 (0.10 g, 0.51 mmol) in anhydrous THF (1 mL) at rt and the reaction mixture was stirred at rt for 18 h. The mixture was distilled and dried in vacuo. Then, anhydrous THF (1 mL) was added and the reaction was cooled to 0° C. and a 1.4M solution of methyl magnesium bromide in THF:toluene (1.83 mL, 2.57 mmol) was added dropwise and the reaction mixture was stirred at 0° C. for 15 min and at rt for 15 h. NH₄Cl sat was added and the mixture was extracted with DCM (10 mL×3 times). The organics layers were dried over MgSO₄ and concentrated in vacuo. The crude product was purified by flash column chromatography (silica; MeOH (10:1) in DCM 0/100 to 50/50). The desired fractions were collected and concentrated in vacuo to yield a compound that was diluted in DCM and a 4N solution of HCl in 1,4-dioxane. The solvents were evaporated in vacuo and the product was triturated with diethyl ether to yield product 44 as a white solid.

### E5. Preparation of Product 45

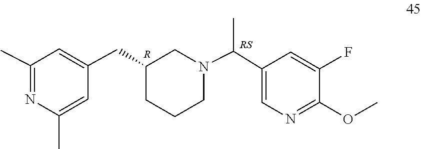

To a solution of intermediate 3 (0.10 g, 0.49 mmol) in anhydrous DCM (2 mL), 5-fluoro-6-methoxynicotinaldehyde (CAS 884494-73-9, 0.83 g, 0.54 mmol) and titanium(IV)isopropoxide (0.21 mL, 0.73 mmol) were added and the reaction mixture was stirred at rt for 24 h. Then the reaction was cooled to 0° C. and 1.4M solution of methyl magnesium bromide in THF:toluene (1.75 mL, 2.44 mmol) was added dropwise and the reaction mixture was stirred at 0° C. for 5 min and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 2/98). The desired fractions were collected and the solvents evaporated in vacuo to yield product 45 as a yellow oil.

### E9. Preparation of Product 46 and 47

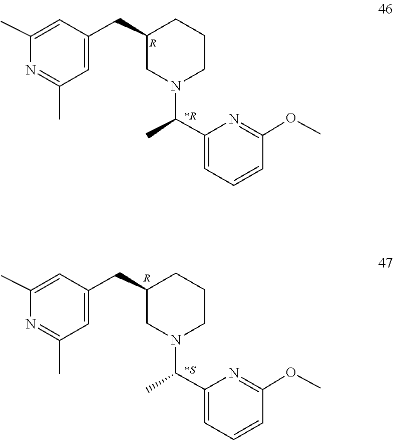

Titanium(IV)isopropoxide (2 eq, 0.29 mL, 0.90 mmol) was added to a stirred solution of intermediate 3 (0.10 g, 0.45 mmol) and 1-(6-methoxy-2-pyridinyl)-ethanone (CAS 21190-93-2, 0.11 g, 0.73 mmol) in anhydrous THF (2 mL) at rt. The mixture was stirred at 70° C. for 16 h. Then, additional titanium(IV)isopropoxide (2 eq, 0.29 mL, 0.90 mmol) and sodium cyanoborohydride (37 mg, 0.58 mmol) were added and the resulting mixture was stirred at 70° C. for 20 h more. The mixture was quenched with H₂O, diluted with EtOAc and filtered through a pad of diatomaceous earth. The organic layer was separated, dried (Na₂SO₄), filtered and the solvents evaporated in vacuo. The crude product was purified by flash column chromatography (silica; 7N solution of ammonia in methanol in DCM 0/100 to 5/95). The desired fractions were collected and the solvents evaporated in vacuo to yield the product was purified by RP HPLC (Stationary phase: C18 XBridge 30×100 mm 5 um, mobile phase: Gradient from 60% NH₄HCO₃ 0.25% solution in H₂O, 40% CH₃CN to 43% NH₄HCO₃ 0.25% solution in H₂O, 57% CH₃CN). The desired fractions were evaporated in vacuo to yield product 46 (26 mg, 15%) as a yellow oil, product 47 (22 mg, 13%) as a colorless oils.

### E5. Preparation of Product 48

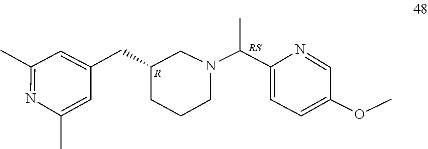

To a solution of intermediate 3 (0.10 g, 0.49 mmol) in anhydrous DCM (2 mL), 5-methoxypicolinaldehyde (CAS 22187-96-8, 0.074 g, 0.54 mmol) and titanium(IV)isopropoxide (0.21 mL, 0.73 mmol) were added and the reaction mixture was stirred at rt for 24 h. Then the reaction was cooled to 0° C. and a 1.4M solution of methylmagnesium bromide in THF:toluene (1.75 mL, 2.45 mmol) was added dropwise and the reaction mixture was stirred at 0° C. for 5 min. and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 4/96). The desired fractions were collected and the solvents evaporated in vacuo. The product was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and the solvents evaporated in vacuo to yield product 48 as a yellow oil.

### E9. Preparation of Product 49, 50 and 51

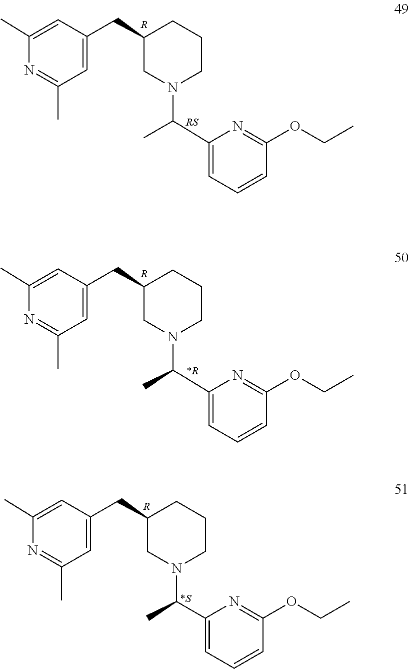

Sodium cyanoborohydride (0.05 g, 0.88 mmol) was added to a stirred solution of intermediate 3 (0.15 g, 0.73 mmol), 1-(6-ethoxy-2-pyridinyl)-ethanone (CAS 21190-90-9, 0.18 g, 1.1 mmol) and titanium(IV)isopropoxide (0.43 mL, 1.47 mmol) in anhydrous THF (3 mL) and the reaction mixture was stirred at 70° C. for 20 h. The mixture was quenched with sat. solution of NaHCO₃, diluted with EtOAc and filtered through a pad of diatomaceous earth. The organic layer was separated, dried (MgSO₄), filtered and the solvents evaporated in vacuo. The crude product was purified by flash column chromatography (silica; 7N solution of ammonia in methanol in DCM 0/100 to 5/95). The desired fractions were collected and the solvents evaporated in vacuo to yield a yellow oil that was purified by RP HPLC (Stationary phase: C18 XBridge 30×100 mm 5 μm, mobile phase: Gradient from 54% NH₄HCO₃ 0.25% solution in H₂O, 46% CH₃CN to 36% NH₄HCO₃ 0.25% solution in H₂O, 64% CH₃CN). The desired fractions were collected and the solvents evaporated in vacuo to yield product 49 (124 mg, 48%) as a yellow oil. Product 49 was purified via chiral SFC (Stationary phase: Chiralpak IC 5 μm 250*21.2 mm, mobile phase: 82% C02, 18% MeOH (0.3% iPrNH₂)) to yield product 50 (48 mg, 18%) and product 51 (43 mg, 16%).

### E5. Preparation of Product 52

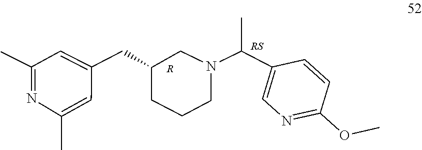

To a solution of intermediate 3 (0.10 g, 0.49 mmol) in anhydrous DCM (2 mL), 6-methoxypicolinaldehyde (CAS 65873-72-5, 0.074 g, 0.54 mmol) and titanium(IV)isopropoxide (0.21 mL, 0.73 mmol) were added and the reaction mixture was stirred at rt for 24 h. Then the reaction was cooled to 0° C. and a 1.4M solution of methylmagnesium bromide in THF:toluene (1.75 mL, 2.45 mmol) was added dropwise and the reaction mixture was stirred at 0° C. for 5 min. and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 4/96). The desired fractions were collected and the solvents evaporated in vacuo. The product was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and the solvents evaporated in vacuo to yield product 52 (107 mg, 64%) as a yellow oil.

### E5. Preparation of Product 53

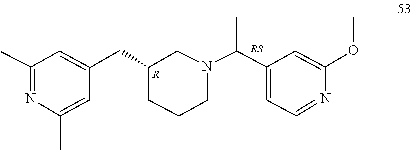

To a solution of intermediate 3 (0.05 g, 0.24 mmol) in anhydrous DCM (1 mL), 2-methoxy-4-pyridinecarboxaldehyde (CAS 72716-87-1, 0.050 g, 0.36 mmol) and titanium(IV)isopropoxide (0.10 mL, 0.36 mmol) were added and the reaction mixture was stirred at rt for 24 h. Then the reaction was cooled to 0° C. and a 1.4M solution of methylmagnesium bromide in THF:toluene (0.87 mL, 1.22 mmol) was added dropwise and the reaction mixture was stirred at 0° C. for 5 min and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 4/96). The desired fractions were collected and the solvents evaporated in vacuo. The product was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and the solvents evaporated in vacuo to yield product 53 (16 mg, 20%) as a yellow oil.

### E9. Preparation of Product 54 and 55

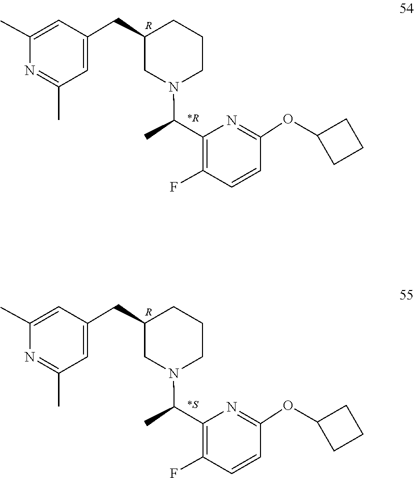

Titanium(IV)isopropoxide (0.36 mL, 1.23 mmol) was added to a stirred solution of intermediate 3 (0.12 g, 0.58 mmol) and intermediate 108 (0.16 g, 0.76 mmol) in anhydrous THF (2 mL) in a sealed tube and under N₂. The mixture was stirred for 15 min at rt and then sodium cyanoborohydride (0.82 g, 1.29 mmol) was added. The mixture was stirred at 90° C. for 24 h. The solvent was evaporated in vacuo and the crude product was purified by flash column chromatography (silica; MeOH in DCM 0/100 to 5/95) the corresponding fractions were collected and concentrated in vacuo, the crude was purified again by RP HPLC (Stationary phase: C18 XBridge 30×100 mm 5 um, mobile phase: Gradient from 54% 0.10% NH₄CO₃H/NH₄OH pH 9 solution in H₂O, 46% CH₃CN to 64% 0.1% NH₄CO₃H/NH₄OH pH 9 solution in H₂O, 36% CH₃CN), the desired fractions were collected and concentrated in vacuo to get impure product 54 and product 55 (34 mg, 14%) as a transparent oil. Product 54 was purified again by RP HPLC (Stationary phase: C18 XBridge 30×100 mm 5 um, mobile phase: Gradient from 54% 0.1% NH₄CO₃H/NH₄OH pH 9 solution in H₂O, 46% CH₃CN to 64% 0.1% NH₄CO₃H/NH₄OH pH 9 solution in H₂O, 36% CH₃CN), the desired fractions were collected and concentrated in vacuo to get yielding product 54 (40 mg, 17%) as and oil.

### E5. Preparation of Product 56

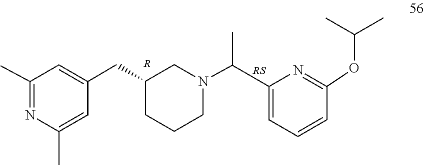

To a solution of intermediate 3 (0.05 g, 0.24 mmol) in anhydrous DCM (1 mL), 6-isopropoxypyridine-2-carbaldehyde (CAS 350697-31-31, 0.060 g, 0.36 mmol) and titanium(IV)isopropoxide (0.10 mL, 0.36 mmol) were added and the reaction mixture was stirred at rt for 24 h. Then the reaction was cooled to 0° C. and a 1.4M solution of methylmagnesium bromide in THF:toluene (0.87 mL, 1.22 mmol) was added dropwise and the reaction mixture was stirred at 0° C. for 5 min. and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 4/96). The desired fractions were collected and the solvents evaporated in vacuo. The product was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and the solvents evaporated in vacuo to yield product 56 (49 mg, 54%) as a yellow oil.

### E5. Preparation of Product 57 and 58

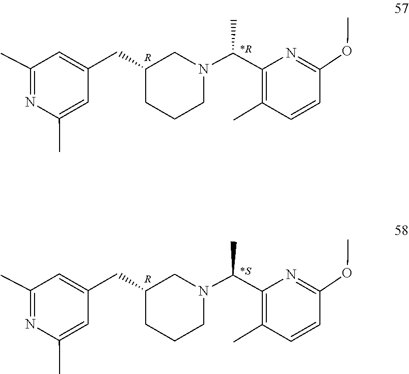

To a solution of intermediate 3 (0.10 g, 0.49 mmol) in anhydrous DCM (2 mL), 6-methoxy-3-methyl-2-pyridinecarboxaldehyde (CAS 123506-64-9, 0.110 g, 0.73 mmol) and titanium(IV)isopropoxide (0.21 mL, 0.73 mmol) were added and the reaction mixture was stirred at rt for 24 h. Then the reaction was cooled to 0° C. and a 1.4M solution of methylmagnesium bromide in THF:toluene (1.75 mL, 2.45 mmol) was added dropwise and the reaction mixture was stirred at 0° C. for 5 min and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 4/96). The desired fractions were collected and the solvents evaporated in vacuo. The product was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected, the solvents evaporated in vacuo and the residues were diluted with sat NaHCO₃ and extracted with DCM to yield product 57 (17 mg, 10%) and product 58 (17 mg, 10%) as yellow oils.

### E5. Preparation of Product 59 and 60

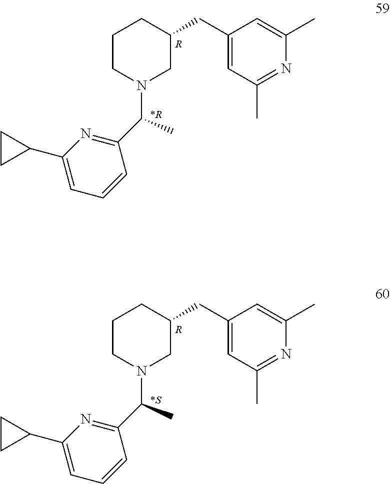

6-Cyclopropyl-2-pyridinecarboxaldehyde (CAS 208111-24-4, 0.12 g, 0.84 mmol) and titanium(IV)isopropoxide (0.61 mL, 2.09 mmol) were added to a stirred solution of intermediate 3 (0.14 g, 0.69 mmol) in DCM (3.25 mL) at rt and under N₂. The mixture was stirred at rt for 16 h. Then it was cooled at 0° C. and a 1.4M solution of methylmagnesium bromide in THF:toluene (2.49 mL, 3.48 mmol) was added dropwise followed by THF (0.7 mL). The mixture was stirred at this temperature for 25 min and at rt for 2 h. The mixture was treated with sat NH₄Cl and extracted with DCM. The organic layer was separated, dried (Na₂SO₄), filtered and the solvent was evaporated in vacuo. The crude product was purified by flash column chromatography (silica amino functionalized, MeOH in DCM 0/100 to 4/96). The desired fractions were collected and concentrated in vacuo to yield a sticky oil, which was purified by RP HPLC (Stationary phase: C18 XBridge 30×100 mm 5 μm, mobile phase: Gradient from 67% NH₄HCO₃ 0.25% solution in H₂O, 33% CH₃CN to 50% NH₄HCO₃ 0.25% solution in H₂O, 50% CH₃CN), the fractions were combined and partially concentrated, then washed with H₂O and extracted with DCM. The organic layer was separated, dried (Na₂SO₄), filtered and the solvent was evaporated in vacuo to yield a mixture of products that was purified again by RP HPLC (Stationary phase: C18 XBridge 30×100 mm 5 μm, mobile phase: Gradient from 67% NH₄HCO₃ 0.25% solution in H₂O, 33% CH₃CN to 50% NH₄HCO₃ 0.25% solution in H₂O, 50% CH₃CN), the fractions were combined and partially concentrated, then washed with H₂O and extracted with DCM. The organic layer was separated, dried (Na₂SO₄), filtered and the solvent was evaporated in vacuo to yield product 59 (4 mg, 2%), and product 60 (3 mg, 1%).

### E5. Preparation of Product 61

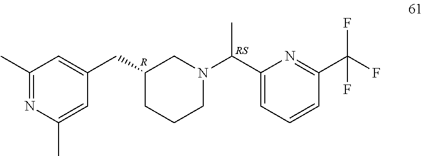

To a solution of intermediate 3 (0.050 g, 0.24 mmol) in anhydrous DCM (1 mL), 6-(trifluoromethyl)picolinaldehyde (CAS 131747-65-4) and titanium(IV)isopropoxide (0.10 mL, 0.37 mmol) were added and the reaction mixture was stirred at rt for 48 h. Then the reaction was cooled to 0° C. and a 1.4M solution of methylmagnesium bromide in THF:toluene (0.87 mL, 1.22 mmol) was added dropwise and the reaction mixture was stirred at 0° C. for 5 min. and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 2/98). The desired fractions were collected and the solvents evaporated in vacuo to yield product 61 (53 mg, 57%) as a yellow oil.

### E4. Preparation of Product 62

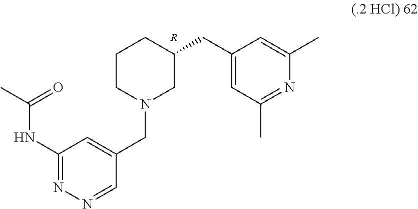

Acetic anhydride (0.03 mL, 0.29 mmol) was added to intermediate 116 (0.031 g, 0.099 mmol) in toluene (3 mL) at rt. The mixture was heated to 100° C. for 1 h. The mixture was concentrated in vacuo. The desired fractions were collected and concentrated in vacuo. The product was purified by phase reverse 81% [25 mM NH₄HCO₃]-19% [ACN: MeOH 1:1] to 45% [25 mM NH₄HCO₃]-55% [ACN: MeOH 1:1]. The solvents were concentrated in vacuo, ACN (10 mL×3 times) was added and was concentrated in vacuo at 60° C. The solvents evaporated in vacuo to yield a compound that was diluted in DCM and 4N solution of HCl in 1,4-dioxane was added. The solvents evaporated in vacuo and the product was triturated with diethyl ether to yield product 62 (14 mg, 33%) as a white solid.

### E1. Preparation of Product 63

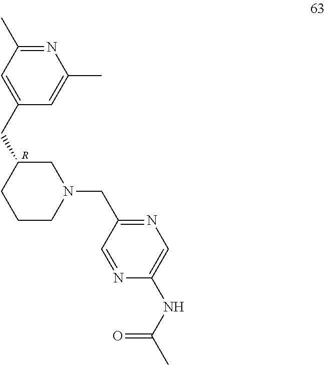

Sodium triacetoxyborohydride (0.30 g, 1.45 mmol) was added to the mixture intermediate 3 (0.27 g, 0.96 mmol), intermediate 118 (0.15 g, 0.96 mmol) and TEA (0.40 mL, 2.89 mmol) in DCM (5 mL). The reaction mixture was stirred at rt for 16 h. Then H₂O was added and extracted with DCM. The organic layer was separated, dried (Na₂SO₄), filtered and concentrated in vacuo. The resultant oil was purified by flash column chromatography (silica; 7M solution of ammonia in methanol in DCM 0/100 to 05/95). The desired fractions were collected and concentrated in vacuo to yield product 63 (0.23 g, 67%) as a colorless oil that solidified upon standing.

### E5. Preparation of Product 64

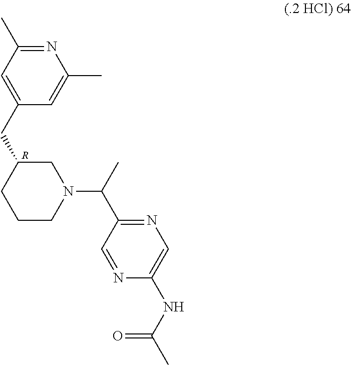

Intermediate 118 (0.12 g, 0.73 mmol) and titanium(IV)isopropoxide (0.43 mL, 1.45 mmol) were added to a solution of intermediate 3 (99 mg, 0.48 mmol) in anhydrous THF (1 mL) at rt and the reaction mixture was stirred at rt for 18 h. Then, the reaction was cooled to 0° C. and a 1.4M solution of methylmagnesium bromide in THF:toluene (1.73 mL, 2.42 mmol) was added dropwise followed by anhydrous THF (1 mL) and the reaction mixture was stirred at 0° C. for 15 min and at rt for 1.5 h. The mixture was stirred at rt for 16 h more. NH₄Cl sat. was added and the mixture was extracted with DCM (10 mL×3 times). The organic layers were separated, dried (MgSO₄) and concentrated in vacuo. The crude product was purified by flash column chromatography (silica; MeOH (10:1) in DCM 0/100 to 50/50). The desired fractions were collected and concentrated in vacuo to yield an orange oil that was purified by phase reverse 70% [25 mM NH₄HCO₃]-30% [ACN: MeOH 1:1] to 27% [25 mM NH₄HCO₃]-73% [ACN: MeOH 1:1]. The solvents were concentrated in vacuo and ACN (10 mL×3 times) was added and the mixture was concentrated in vacuo at 60° C. to yield product 64 (25 mg, 11%) as a yellow oil.

### E5. Preparation of Product 65

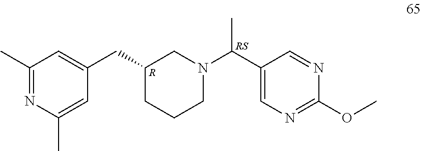

To a solution of intermediate 3 (0.50 g, 0.24 mmol) in anhydrous DCM (1 mL), 2-methoxypyrimidine-5-carbaldehyde (CAS 90905-32-1, 0.05 g, 0.36 mmol) and titanium(IV)isopropoxide (0.10 mL, 0.36 mmol) were added and the reaction mixture was stirred at rt for 24 h. Then the reaction was cooled to 0° C. and a 1.4M solution of methylmagnesium bromide in THF:toluene (0.87 mL, 1.22 mmol) was added dropwise and the reaction mixture was stirred at 0° C. for 5 min and at rt for 3.5 h. Then saturated solution of NH₄Cl was added and the product extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 2/98). The desired fractions were collected and the solvents evaporated in vacuo. The desired fractions were collected and the solvents evaporated in vacuo. The resulting mixture was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and the solvents evaporated in vacuo to yield product 65 (37 mg, 44%) as a yellow oil.

### E5. Preparation of Product 66, 67 and 68

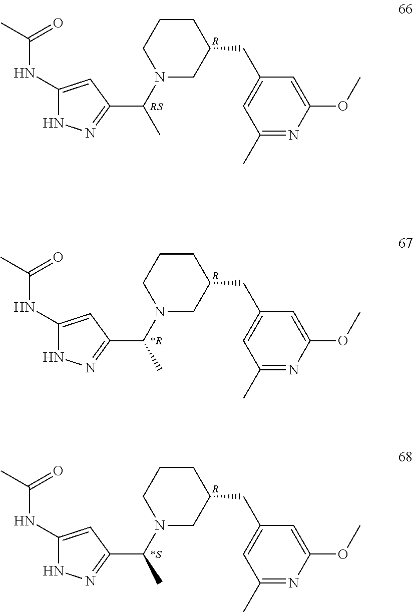

Product 66 was prepared following an analogous procedure to the one described for the synthesis of product 7 using intermediate 65 as starting material. A purification performed via chiral SFC (Stationary phase: CHIRALPAK AD-H 5 μm 250*30 mm, mobile phase: 50% C02, 50% mixture of EtOH/iPrOH 50/50 v/v (+0.3% iPrNH₂)) yielded impure product 67 and product 68 that were dissolved in DCM and washed with a sat sol of NaHCO₃. The organic layer was separated, dried (Na₂SO₄), filtered and concentrated in vacuo to yield product 67 (35 mg, 35%) and impure product 68 that was purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected and concentrated in vacuo. The residue was dissolved in EtOAc and washed with a sat sol of NaHCO₃. The organic layer was separated, dried (Na₂SO₄), filtered and concentrated in vacuo to yield product 68 (9 mg, 9%) as a white wax.

### E5. Preparation of Product 69

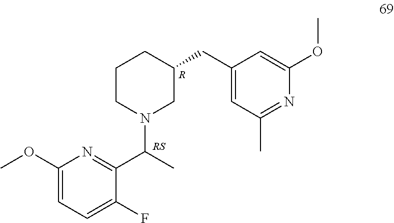

Product 69 was prepared following an analogous procedure to the one described for the synthesis of product 7 using intermediate 5 as starting material. The crude product was purified by flash column chromatography (silica; 7M solution of ammonia in MeOH in DCM 0/100 to 3/97). The desired fractions were collected and the solvents evaporated in vacuo to yield product 69 (114 mg, 67%) as a colorless oil.

### E6. Preparation of Product 70

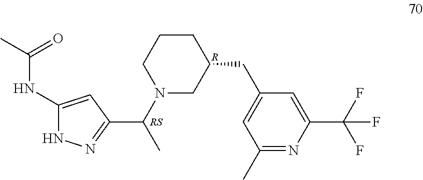

Product 70 was prepared following an analogous procedure to the one described for the synthesis of products 12 and 13 using intermediate 8 as starting material. The residue was purified by flash column chromatography (silica; 7N solution of ammonia in MeOH in DCM (0/100 to 10/90). The desired fractions were collected and concentrated in vacuo to yield product 70 (113 mg, 84%) as a white solid.

### E5. Preparation of Product 71

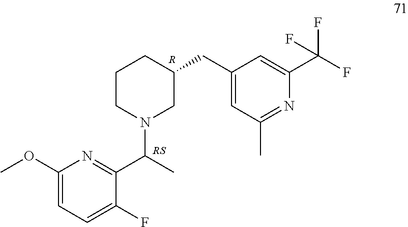

Product 71 was prepared following an analogous procedure to the one described for the synthesis of product 7 using intermediate 8 as starting material. The crude product was purified by flash column chromatography (silica; 7M solution of ammonia in MeOH in DCM 0/100 to 3/97). The desired fractions were collected and the solvents evaporated in vacuo. The residue was purified by RP HPLC (Stationary phase: C18 XBridge 30×100 mm 5 μm, mobile phase: Gradient from 40% NH₄HCO₃ 0.25% solution in H₂O, 60% CH₃CN to 23% NH₄HCO₃ 0.25% solution in H₂O, 77% CH₃CN), yielding product 71 (103 mg, 65%) as a colorless oil.

### E6. Preparation of Product 72

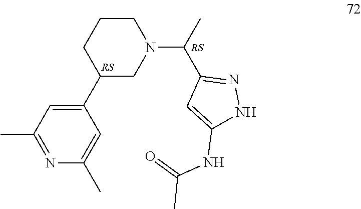 [SMILES: CC(=O)Nc1cc(C(C)N2CCCC(c3cc(C)nc(C)c3)C2)n[nH]1]

Product 72 was prepared following an analogous procedure to the one described for the synthesis of products 12 and 13 using intermediate 11 as starting material. The residue was purified first by ion exchange chromatography using an ISOLUTE SCX2 cartridge eluting first with methanol and then with 7M solution of ammonia in methanol to yield a yellow film that was triturated with diethylether to yield product 72 (33 mg, 62%) as a white solid.

### E5. Preparation of Product 73

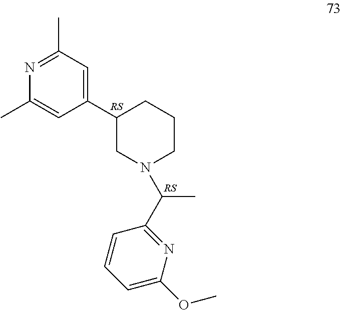

Product 73 was prepared following an analogous procedure to the one described for the synthesis of product 7 using intermediate 11 and 6-methoxy-2-pyridinecarboxaldehyde (CAS 54221-96-4) as starting materials. The crude product was purified by flash column chromatography (silica; MeOH in EtOAc in 0/100 to 10/90). The desired fractions were collected and concentrated in vacuo to yield that was re-purified by RP HPLC (Stationary phase: C18 XBridge 30×100 mm 5 μm, mobile phase: Gradient from 60% NH₄HCO₃ 0.25% solution in H₂O, 40% CH₃CN to 43% NH₄HCO₃ 0.25% solution in H₂O, 57% CH₃CN), product 73 (69 mg, 40%) as a colorless oil.

### E5. Preparation of Product 74

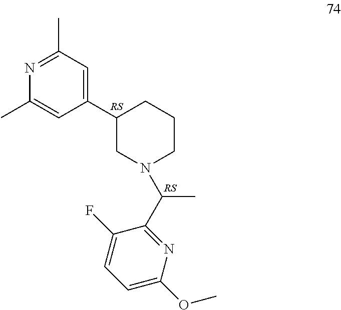

Product 74 was prepared following an analogous procedure to the one described for the synthesis of product 7 using intermediate 11 as starting material. The crude product was purified by flash column chromatography (silica; 7N solution of ammonia in methanol in DCM 0/100 to 2/98). The desired fractions were collected and the solvents evaporated in vacuo to yield the impure product that was purified by RP HPLC (Stationary phase: C18 XBridge 30×100 mm 5 μm, mobile phase: Gradient from 54% NH₄HCO₃ 0.25% solution in H₂O, 46% CH₃CN to 36% NH₄HCO₃ 0.25% solution in H₂O, 64% CH₃CN). The desired fractions were collected and the solvents partially concentrated in vacuo. The aqueous phase was extracted with EtOAc, separated, dried (Na₂SO₄), filtered and the solvent evaporated in vacuo to yield product 74 (92 mg, 72%) as colorless oil.

### E6. Preparation of Product 75

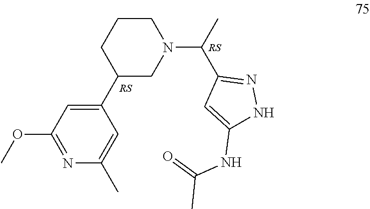 [SMILES: COc1cc(C2CCCN(C(C)c3cc(NC(C)=O)[nH]n3)C2)cc(C)n1]

Product 75 was prepared following an analogous procedure to the one described for the synthesis of products 12 and 13 using intermediate 14 as starting material. The reaction was concentrated to dryness and the residue was purified first by ion exchange chromatography using an ISOLUTE SCX2 cartridge eluting first with methanol and then with 7M solution of ammonia in methanol, the desired fractions were collected and evaporated to give a residue that was purified by RP HPLC (Stationary phase: C18 XBridge 30×100 mm 5 μm, mobile phase: Gradient from 90% NH₄HCO₃ 0.25% solution in H₂O, 10% CH₃CN to 65% NH₄HCO₃ 0.25% solution in H₂O, 35% CH₃CN), yielding impure product as a white solid that was washed with NaHCO₃ saturated solution and EtOAc. The organic layer was separated, dried (Na₂SO₄), filtered and the solvent evaporated in vacuo to yield product 75 (45 mg, 45%) as a white solid.

### E5. Preparation of Product 76

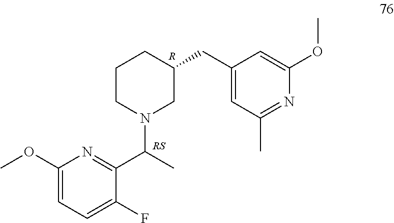

Product 76 was prepared following an analogous procedure to the one described for the synthesis of product 7 using intermediate 14 as starting material. The crude product was purified by flash column chromatography (silica; MeOH in EtOAc in 0/100 to 10/90). The desired fractions were collected and concentrated in vacuo to yield a residue that was purified by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN), yielding product 76 (85 mg, 49%) as a colorless oil.

### E5. Preparation of Product 77

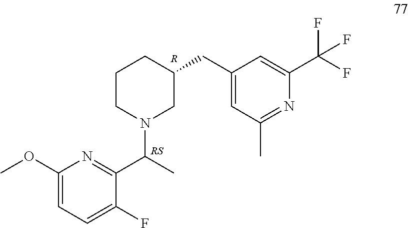

Product 78 was prepared following an analogous procedure to the one described for the synthesis of product 7 using intermediate 18 as starting material. The crude product was purified by flash column chromatography (silica; MeOH in EtOAc in 0/100 to 10/90). The desired fractions were collected and concentrated in vacuo to yield a residue that was purified by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN), yielding product 77 (55 mg, 28%) as a colorless oil.

### E5. Preparation of Product 78

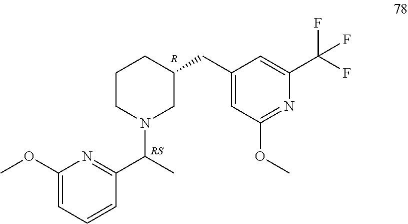

Product 78 was prepared following an analogous procedure to the one described for the synthesis of product 7 using intermediate 20 and 6-methoxy-2-pyridinecarboxaldehyde (CAS 54221-96-4) as starting materials. The crude product was purified by flash column chromatography (silica; MeOH in EtOAc in 0/100 to 10/90). The desired fractions were collected and concentrated in vacuo to yield a residue that was purified by RP HPLC (Stationary phase: C18 XBridge 30×100 mm 5 μm, mobile phase: Gradient from 35% NH₄HCO₃ 0.25% solution in H₂O, 65% CH₃CN to 5% NH₄HCO₃ 0.25% solution in H₂O, 95% CH₃CN), yielding product 78 (15 mg, 10%) as a colorless oil.

### E5. Preparation of Product 79

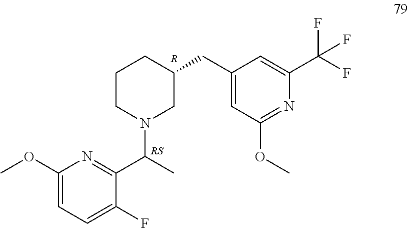

Product 79 was prepared following an analogous procedure to the one described for the synthesis of product 7 using intermediate 20 as starting material. The crude product was purified by flash column chromatography (silica; MeOH in EtOAc in 0/100 to 10/90). The desired fractions were collected and concentrated in vacuo to yield a residue that was purified by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN), yielding product 79 (75 mg, 47%) as a colorless oil.

### E6. Preparation of Product 80

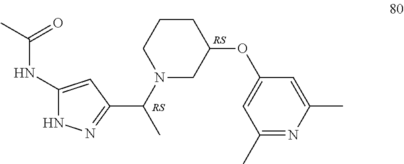 [SMILES: CC(=O)Nc1cc(C(C)N2CCCC(Oc3cc(C)nc(C)c3)C2)n[nH]1]

Product 80 was prepared following an analogous procedure to the one described for the synthesis of products 12 and 13 using intermediate 70 as starting material. The residue was purified by flash column chromatography (silica; 7N solution of ammonia in MeOH in DCM (0/100 to 10/90). The desired fractions were collected and concentrated in vacuo to yield a residue that was purified by RP HPLC (Stationary phase: C18 XBridge 30×100 mm 5 μm, mobile phase: Gradient from 80% NH₄HCO₃ 0.25% solution in H₂O, 20% CH₃CN to 0% NH₄HCO₃ 0.25% solution in H₂O, 100% CH₃CN). The desired fractions were collected and concentrated in vacuo. The residue was dissolved in EtOAc and washed with a sat sol of NaHCO₃. The organic layer was separated, dried (Na₂SO₄), filtered and concentrated in vacuo to yield a product that was triturated with DIPE, filtered and concentrated in vacuo to yield product 80 (18 mg. 21%) as a white solid.

### E5. Preparation of Product 81

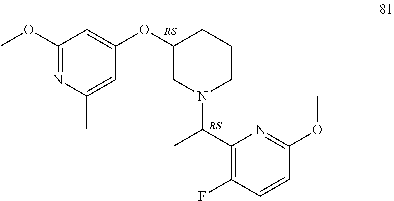

Product 81 was prepared following an analogous procedure to the one described for the synthesis of product 7 using intermediate 25 as starting material. The crude product was purified by flash column chromatography (silica; 7M solution of ammonia in MeOH in DCM 0/100 to 30/70). The desired fractions were collected and the solvents evaporated in vacuo to yield product 81 (105 mg, 62%) as a yellow oil.

### E5. Preparation of Product 82

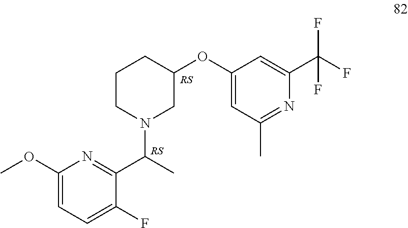

Product 82 was prepared following an analogous procedure to the one described for the synthesis of product 7 using intermediate 28 as starting material. The residue was purified by flash column chromatography (silica; 7N solution of ammonia in MeOH in DCM 0/100 to 5/95). the desired fractions were collected and concentrated in vacuo to yield mllinare_7143_1 as a colorless oil.

### E6. Preparation of Product 83

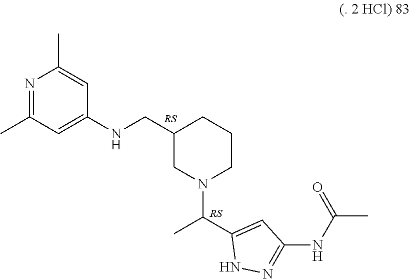 [SMILES: CC(=O)Nc1cc(C(C)N2CCCC(CNc3cc(C)nc(C)c3)C2)[nH]n1]

Product 83 was prepared following an analogous procedure to the one described for the synthesis of products 12 and 13 using intermediate 71 as starting material. The residue was purified by phase reverse 95% [65 mM NH₄OAc+ACN (90:10)]-5% [MeCN:MeOH (1:1)] to 63% [65 mM NH₄OAc+ACN (90:10)]-37% [MeCN: MeOH (1:1)]. The desired fractions were collected and the solvents were concentrated in vacuo. ACN and MeOH were concentrated in vacuo at 60° C., the crude was extracted with DCM (10 mL×3 times) and the organic layer was separated, dried (MgSO₄), filtered and the solvents evaporated in vacuo to yield a residue that was purified by phase reverse 95% [25 mM NH₄HCO₃]-5% [MeCN: MeOH (1:1)] to 63% [25 mM NH₄HCO₃]-37% [MeCN: MeOH (1:1)]. The desired fractions were collected and the solvents were concentrated in vacuo. ACN and MeOH were concentrated in vacuo at 60° C., ACN (10 mL×3 times) was added and concentrated in vacuo to yield a white foam that was taken into DCM and treated with a solution of 4N HCl in 1,4-dioxane. The solvents evaporated in vacuo and the product was triturated with diethyl ether, filtered and dried to yield product 83 (46 mg, 78%) as a white solid.

### E13. Preparation of Products 84 and 85

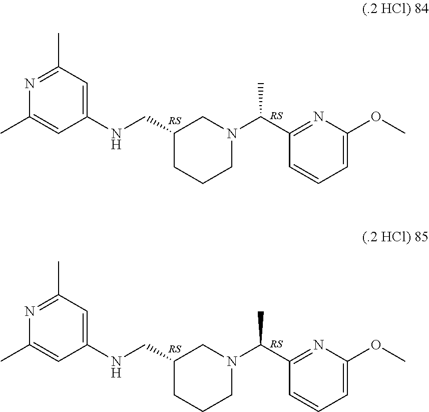

N₂ was bubbled through a solution of 4-bromo-2,6-dimethylpyridine (0.10 g, 0.56 mmol) in 1,4-dioxane (6 mL). Then sodium tert-butoxide (0.15 g, 0.59 mmol), Dave-Phos (22 mg, 0.056 mmol) and Pd₂dba₃ (26 mg, 0.028 mmol) were added to the stirred solution of 4-bromo-2,6-dimethylpyridine (CAS 5093-O-9, 0.10 g, 0.56 mmol) in 1,4-dioxane (6 mL) at rt while N₂ was bubbled in a closed tube. intermediate 122 (0.026 g, 0.028 mmol) was added and the mixture was stirred at 100° C. overnight in a heated bath. The mixture was diluted with EtOAc and 0.5 ml of NH₄Cl sat., filtered over a pad of diatomaceous earth and the solvents evaporated in vacuo. The crude product was purified by reverse phase from 95% H₂O [0.1% TFA]-5% [ACN] to 63% H₂O [0.1% TFA]-37% [ACN]. The desired fractions were collected, neutralized with NaHCO₃ sat. and concentrated in vacuo. To remove salts it was purified again by reverse phase from 95% [H₂O (25 mM NH₄HCO₃)-5%[ACN] to 0% [H₂O (25 mM NH₄HCO₃)]-100% [ACN)]. The desired fractions were collected organic solvent were concentrated to give impure products 84 and 85 as colorless sticky solids. These materials were taken into DCM and treated with 2 eq of HCl 4N in 1.4-dioxane. The solvents evaporated in vacuo and the product was tritured with diethyl ether to yield product 84 (45 mg, 18%) as a white solid and product 85 (70 mg, 29%) as a white solid.

### E5. Preparation of Product 86

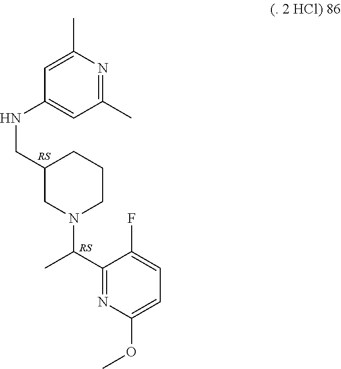 [SMILES: COc1ccc(F)c(C(C)N2CCCC(CNc3cc(C)nc(C)c3)C2)n1]

Product 86 was prepared following an analogous procedure to the one described for the synthesis of product 7 using intermediate 43 as starting material. The crude was purified by reverse phase (from 72% (H₂O 25 mM NH₄HCO₃)-28% MeCN-MeOH to 36% H₂O (25 mM NH₄HCO₃)-64% MeCN-MeOH). The desired fractions were collected concentrated in vacuo to yield a colorless sticky solid that was taken into DCM and treated with 2 eq of HCl 4N in 1.4-dioxane. The solvents evaporated in vacuo and the product was triturated with diethyl ether to yield product 86 as a white solid.

### E6. Preparation of Product 87

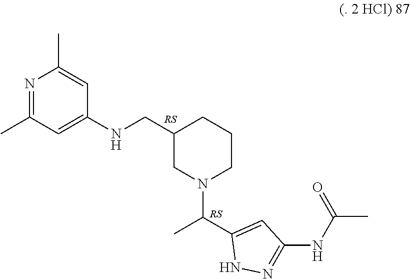 [SMILES: CC(=O)Nc1cc(C(C)N2CCCC(CNc3cc(C)nc(C)c3)C2)[nH]n1]

Product 87 was prepared following an analogous procedure to the one described for the synthesis of products 12 and 13 using intermediate 72 as starting material. The crude product was purified by phase reverse 90% [25 mM NH₄HCO₃]-10% [MeCN: MeOH (1:1)] to 54% [25 mM NH₄HCO₃]-46% [MeCN: MeOH (1:1)]. The desired fractions were collected and the solvents were concentrated in vacuo. ACN and MeOH were concentrated in vacuo at 60° C., ACN (10 mL×3 times) was added and concentrated in vacuo to yield a white foam that was taken into DCM and treated with 2 eq of HCl 4N in 1.4-dioxane. The solvents evaporated in vacuo and the product was triturated with diethyl ether to yield product 87 (60 mg, 43%) as a white solid.

### E13. PREPARATION OF PRODUCTS 88 AND 89

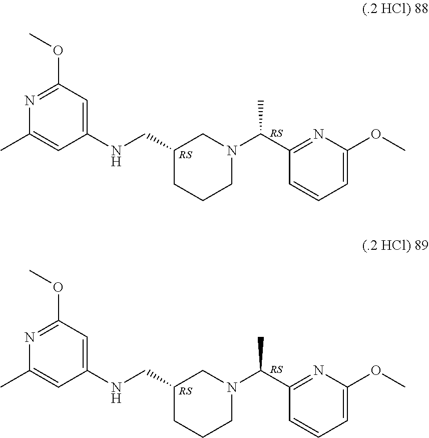

Products 88 and 89 were prepared following an analogous procedure to the one described for the synthesis of products 84 and 85 using intermediate 45 as starting material. The crude product was purified by reverse phase from 95% H₂O [0.1% TFA]-5% [MeOH] to 63% H₂O [0.1% TFA]-37% [MeOH]. The desired fractions were collected, neutralized with NaHCO₃ sat. and concentrated in vacuo. To remove salts we purified again by reverse phase from 95% [H₂O (25 mM NH₄HCO₃)-5%[ACN] to 0% [H₂O (25 mM NH₄HCO₃)]-100% [ACN)]. The desired fractions were collected organic solvent were concentrated to give impure products 88 and 89 as colorless sticky solids. These materials were taken into DCM and treated with 2 eq of HCl 4N in 1.4-dioxane. The solvents evaporated in vacuo and the product was triturated with diethyl ether to yield product 88 (44 mg, 18%) as a white solid and product 89 (66 mg, 27%) as a white solid.

### E13. Preparation of Product 90

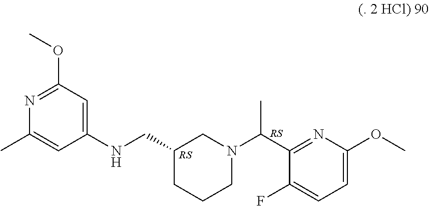

Product 90 was prepared following an analogous procedure to the one described for the synthesis of products 84 and 85 using intermediate 45 as starting material. The crude product was purified by reverse phase from 95% H₂O [0.1% TFA]-5% [MeOH] to 63% H₂O [0.1% TFA]-37% [MeOH]. The desired fractions were collected, neutralized with NaHCO₃ sat. and concentrated in vacuo to yield a residue that was purified again by reverse phase from 95% [H₂O (25 mM NH₄HCO₃)-5%[ACN] to 0% [H₂O (25 mM NH₄HCO₃)]-100% [ACN)]. The desired fractions were collected organic solvent were concentrated to give a colorless sticky solid. This material was taken into DCM and treated with 1 eq of HCl 4N in 1.4-dioxane. The solvents evaporated in vacuo and the product was triturated with diethyl ether to yield product 90 (41 mg, 13%) as a white solid.

### E6. Preparation of Product 91

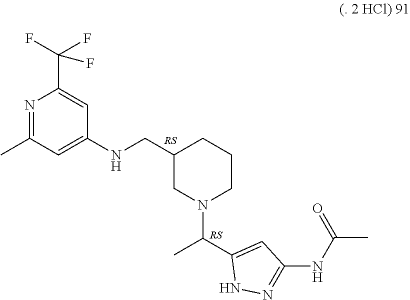 [SMILES: CC(=O)Nc1cc(C(C)N2CCCC(CNc3cc(C)nc(C(F)(F)F)c3)C2)[nH]n1]

Product 91 was prepared following an analogous procedure to the one described for the synthesis of products 12 and 13 using intermediate 73 as starting material. The crude material was purified by chromatography (silica; DCM/MeOH (9:1) in DCM 0/100 to 10/90). The desired fractions were collected and concentrated in vacuo to yield as a colorless foam. The crude product was triturated with diethylether, the solid was filtered and dried to yield a white solid that was purified by phase reverse 72% [25 mM NH₄HCO₃]-28% [MeCN: MeOH (1:1)] to 36% [25 mM NH₄HCO₃]-64% [MeCN: MeOH (1:1)]. The solvents were concentrated in vacuo at 60° C. and ACN (10 mL×3 times) were concentrated in vacuo to yield product 91 (62 mg, 66%) as a white foam.

### E14. Preparation of Product 92, 93, 94 AND 95

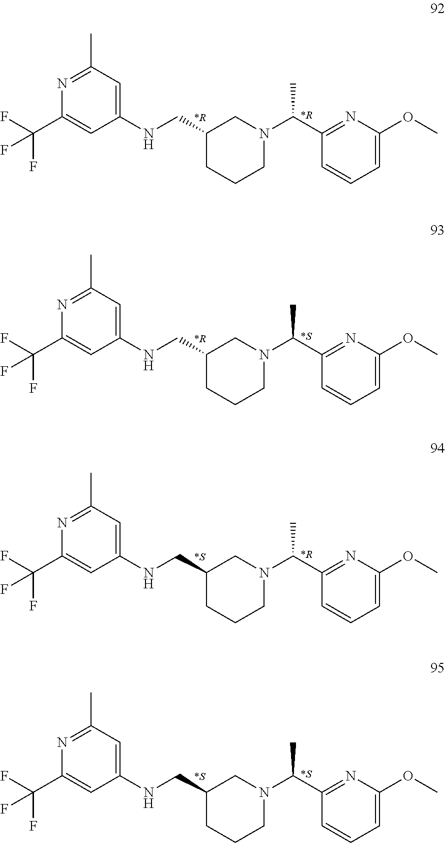 [SMILES: COc1cccc([C@@H](C)N2CCC[C@H](CNc3cc(C)nc(C(F)(F)F)c3)C2)n1 ; COc1cccc([C@H](C)N2CCC[C@H](CNc3cc(C)nc(C(F)(F)F)c3)C2)n1 ; COc1cccc([C@@H](C)N2CCC[C@@H](CNc3cc(C)nc(C(F)(F)F)c3)C2)n1 ; COc1cccc([C@H](C)N2CCC[C@@H](CNc3cc(C)nc(C(F)(F)F)c3)C2)n1]

Intermediate 123 (0.14 g, 0.33 mmol) and trimethylboroxine (CAS 823-96-1, 0.055 mL, 0.39 mmol) were added to the mixture of K₃PO₄ (0.104 g, 0.49 mmol), X-Phos (16 mg, 0.033 mmol), Pd₂(dba)₃ (15 mg, 0.033 mmol) in 1,4-dioxane (5 mL) at 100° C. under N₂ and in a sealed tube. The mixture was cooled to rt and K₃PO₄ (0.026 g, 0.12 mmol), Pd₂(dba)₃ (4 mg, 0.004 mmol), X-Phos (4 mg, 0.008 mmol) and trimethylboroxine (12 mg, 0.098 mmol) were added under N₂ and the mixture was stirred in a sealed tube at 100° C. for 4 h more. The mixture was cooled to rt and water and AcOEt were added. The organic layer was separated, dried (MgSO₄) and filtered and the solvents evaporated in vacuo. The crude was purified by flash column chromatography (silica; EtOAc in Heptane 0/100 to 50/50). The desired fractions were collected and concentrated in vacuo to yield a yellow solid. The mixture was purified by phase reverse 95% [0.1% HCOOH]-5% [ACN: MeOH 1:1] to 63% [0.1% HCOOH]-37% [ACN: MeOH 1:1]. The desired fractions were collected and the mixture was added NaHCO₃ until pH basic. ACN and MeOH were concentrated and the mixture was extracted with DCM (12 mL×3 times). The organic layer was separated, dried over MgSO₄, filtered and the solvents evaporated in vacuo. Diethyl ether (3 mL) was added and the solvent was concentrated in vacuo to yield a mixture of products that was purified via chiral SFC (Stationary phase: Lux Cellulose-2 5 μm 250*21.2 mm, mobile phase: 85% C02, 15% iPrOH (0.3% iPrNH₂)) yielding product 92 (11 mg, 14%) and product 93 (14 mg, 18%) as pale yellow oils. Also purification via chiral SFC (Stationary phase: CHIRACEL OJ-H 5 μm 250*20 mm, mobile phase: 88% C02, 12% iPrOH (0.3% iPrNH₂)) yielded product 94 (11 mg, 14%) and product 95 (10 mg, 13%).

### E9. Preparation of Product 96

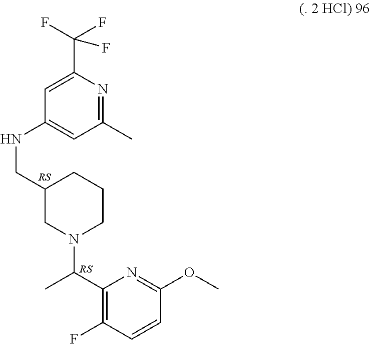 [SMILES: COc1ccc(F)c(C(C)N2CCCC(CNc3cc(C)nc(C(F)(F)F)c3)C2)n1]

Product 96 was prepared following an analogous procedure to the one described for the synthesis of products 41 and 42 using intermediate 48 as starting material. The crude product was purified by flash column chromatography (silica; DCM/MeOH (9:1) in DCM 0/100 to 60/40). The desired fractions were collected and concentrated to yield a yellow oil that was purified by phase reverse 95% [0.1% HCOOH]-5% [MeCN: MeOH (1:1)] to 63% [0.1% HCOOH]-37% [MeCN: MeOH (1:1)]. The desired fractions were collected and NaHCO₃ sat. was added until pH basic and concentrated to yield a product that was purified by phase reverse 38% [25 mM NH₄HCO₃]-62% [MeCN:MeOH (1:1)] to 0% [25 mM NH₄HCO₃]-100%[MeCN: MeOH (1:1)]. The desired fractions were collected and the solvents was concentrated in vacuo. ACN (15 mL×3 times) was added and the solvent was evaporated in vacuo to yield as a yellow oil that was taken into DCM and treated with 2 eq of HCl 4N in 1.4-dioxane. The solvents evaporated in vacuo and the product was triturated with diethyl ether, filtered and dried to yield product 96 (107 mg, 43%) as a white solid.

### E6. Preparation of Product 97

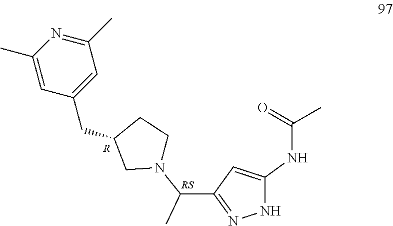

Product 97 was prepared following an analogous procedure to the one described for the synthesis of products 12 and 13 using intermediate 126 as starting material. The crude product was purified by RP HPLC (Stationary phase: C18 XBridge 30×100 mm 5 μm, mobile phase: Gradient from 90% NH₄HCO₃ 0.25% solution in H₂O, 10% CH₃CN to 65% NH₄HCO₃ 0.25% solution in H₂O, 35% CH₃CN). The desired fractions were collected and concentrated in vacuo to yield product 96 (114 mg, 96%) as a foam.

### E9. Preparation of Product 98

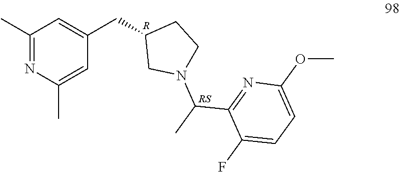

Product 98 was prepared following an analogous procedure to the one described for the synthesis of products 41 and 42 using intermediate 48 as starting material. The crude product was purified by flash column chromatography (silica; 7N solution of ammonia in methanol in DCM 0/100 to 4/96). The desired fractions were collected and the solvents evaporated in vacuo to yield impure product that was purified by RP HPLC (Stationary phase: C18 XBridge 30×100 mm 5 μm, mobile phase: Gradient from 60% NH₄HCO₃ 0.25% solution in H₂O, 40% CH₃CN to 43% NH₄HCO₃ 0.25% solution in H₂O, 57% CH₃CN). The desired fractions were collected and the solvents partially concentrated in vacuo. The aqueous phase was extracted with EtOAc, separated, dried (Na₂SO₄), filtered and the solvent evaporated in vacuo to yield product 98 (38 mg, 35%) as colorless oil.

E5. Preparation of Product 99

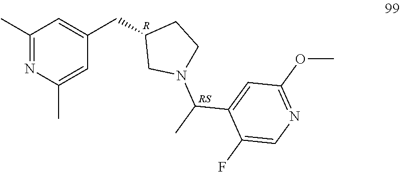

Titanium(IV) isopropoxide (0.23 mL, 0.79 mmol) was added to a stirred solution of intermediate 39 (0.10 g, 0.53 mmol) and 5-fluoro-2-methoxyisonicotinaldehyde (CAS 884495-00-5, 0.098 g, 0.63 mmol) in anhydrous DCM (2.3 ml) at rt and under N₂. The mixture was stirred at rt for 16 h. Then the mixture was cooled at 0° C. and a 1.4M solution of methyl magnesium bromide in THF:toluene (1.9 mL, 2.66 mmol) was added dropwise. The mixture was stirred at this temperature for 15 min and then at rt for 2 h. The mixture was treated with sat. NH₄Cl and extracted with DCM. The phases were filtered through a pad of diatomaceous earth and then the organic layer was separated, dried (Na₂SO₄), filtered and the solvents evaporated in vacuo. The crude product was purified by flash column chromatography (silica; EtOAc in Heptane 0/100 to 60/40). The desired fractions were collected and the solvents evaporated in vacuo to yield a colorless oil that was purified by RP HPLC (Stationary phase: C18 XBridge 30×100 mm 5 μm, mobile phase: Gradient from 60% NH₄HCO₃ 0.25% solution in H₂O, 40% CH₃CN to 43% NH₄HCO₃ 0.25% solution in H₂O, 57% CH₃CN). The desired fractions were collected and the solvents partially concentrated in vacuo. The aqueous phase was extracted with EtOAc, separated, dried (Na₂SO₄), filtered and the solvent evaporated in vacuo to yield a yellow oil that was purified by RP HPLC (Stationary phase: C18 XBridge 30×100 mm 5 μm, mobile phase: Gradient from 60% NH₄HCO₃ 0.25% solution in H₂O, 40% CH₃CN to 43% NH₄HCO₃ 0.25% solution in H₂O, 57% CH₃CN). The desired fractions were collected and the solvents partially concentrated in vacuo. The aqueous phase was extracted with EtOAc, separated, dried (Na₂SO₄), filtered and the solvent evaporated in vacuo to yield product 99 (48 mg, 26%) as yellow oil.

### E9. Preparation of Product 100

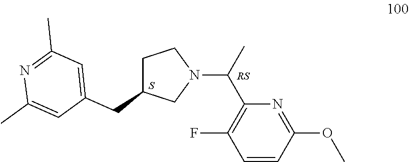

Product 100 was prepared following an analogous procedure to the one described for the synthesis of products 41 and 42 using intermediate 31 as starting material. The crude product was purified by flash column chromatography (silica; 7N solution of NH₃ in MeOH in DCM 0/100 to 3/97). The desired fractions were collected and concentrated in vacuo to yield product 100 (110 mg, 61%) as a yellowish oil.

### E11. Preparation of Product 101

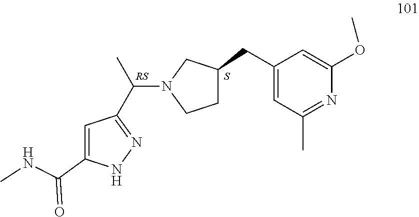

Intermediate 124 (0.042 g, 0.25 mmol) and titanium(IV)isopropoxide (0.10 mL, 0.36 mmol) were added to a solution of intermediate 33 (0.05 g, 0.24 mmol) in DCE (0.96 mL) and the reaction mixture was stirred at 80° C. for 4 h. Then the reaction was cooled to rt and sodium cyanoborohydride (18 mg, 0.29 mmol) was added and the mixture was stirred overnight. Then saturated solution of NaHCO₃ was added and the mixture was extracted with DCM. The organic layer was separated, dried (MgSO₄), filtered and the solvents evaporated in vacuo. The crude product was purified by RP HPLC (Stationary phase: C18 XBridge 30×100 mm 5 μm, mobile phase: Gradient from 80% NH₄HCO₃ 0.25% solution in H₂O, 20% CH₃CN to 60% NH₄HCO₃ 0.25% solution in H₂O, 40% CH₃CN), yielding product 101 (12 mg, 14%) as a white foam.

### E5. Preparation of Product 102

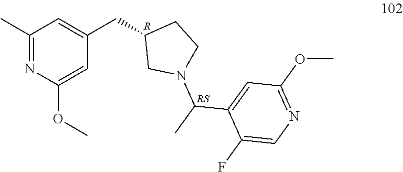

Product 102 was prepared following an analogous procedure to the one described for the synthesis of product 7 using intermediate 33 as starting material. The crude product was purified by flash column chromatography (silica; EtOAc in Heptane 0/100 to 30/70). The desired fractions were collected and the solvents evaporated in vacuo to yield a yellow oil that was purified by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN). The desired fractions were collected and the solvents partially concentrated in vacuo. The aqueous phase was extracted with EtOAc, separated, dried (Na₂SO₄), filtered and the solvent evaporated in vacuo to yield a yellow oil that was purified by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN). The desired fractions were collected and the solvents partially concentrated in vacuo. The aqueous phase was extracted with EtOAc, separated, dried (Na₂SO₄), filtered and the solvent evaporated in vacuo to yield product 102 (16 mg, 9%) as yellow oil.

### E11. Preparation of Product 103

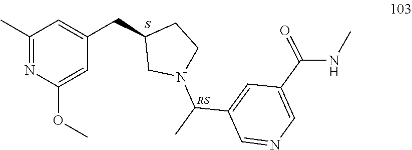

Sodium cyanoborohydride (36 mg, 0.58 mmol) was added to a stirred solution of intermediate 33 (0.10 g, 0.48 mmol), intermediate 125 (0.103 g, 0.58 mmol) and titanium(IV)isopropoxide (0.58 mL, 0.97 mmol) in anhydrous THF (2 mL) at rt. The mixture was stirred at 70° C. for 16 h. The mixture was treated with H₂O and extracted with EtOAc. The phases were filtered through a pad of diatomaceous earth and then the organic layer was separated, dried (Na₂SO₄), filtered and the solvents evaporated in vacuo. The residue was purified by RP HPLC (Stationary phase: C18 XBridge 50×150 mm 5 μm, mobile phase: Gradient from 84% NH₄HCO₃ 0.25% solution in H₂O, 16% CH₃CN to 60% NH₄HCO₃ 0.25% solution in H₂O, 40% CH₃CN). The desired fractions were collected and the solvents evaporated in vacuo. The residue was purified by flash column chromatography (silica; MeOH in DCM 0/100 to 7/93). The desired fractions were collected and the solvents evaporated in vacuo to yield product 103 (67 mg, 37%) as an oil.

### E6. Preparation of Products 104, 105 and 106

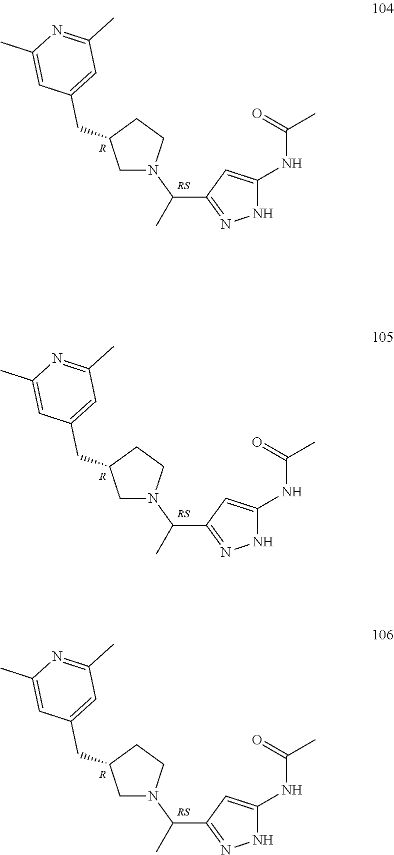

Product 104 was prepared following an analogous procedure to the one described for the synthesis of products 12 and 13 using intermediate 127 as starting material. The residue was purified by ion exchange chromatography using an ISOLUTE SCX2 cartridge eluting first with methanol which was discarded and then with 7M solution of ammonia in methanol. The filtrate was concentrated in vacuo to yield product 104 (85 mg, 77%) as a white foam. A purification was performed via chiral SFC (Stationary phase: CHIRALPAK yielded product 105 (33 mg, 30%) and product 106 (36 mg, 33%) as beige foams.

### E5. Preparation of Products 107, 108 and 109

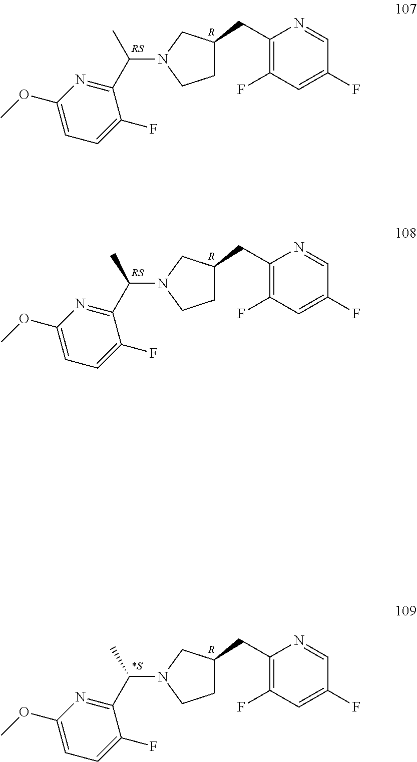

Product 109 was prepared following an analogous procedure to the one described for the synthesis of product 7 using intermediate 35 as starting material. The crude product was purified by flash column chromatography (silica; ethyl acetate in DCM 50/50 to 100/0 and methanol in ethyl acetate 0/100 to 5/95). The desired fractions were collected and concentrated in vacuo to yield product 107 (126 mg, 71%) as a pale brown oil. A purification was performed via achiral SFC (Stationary phase: CHIRALPAK IC 5 μm 250*30 mm, Mobile phase: 93% CO₂, 7% iPrOH (0.3% iPrNH₂)) yielded product 108 (47 mg, 26%) and impure product 109 that was purified via preparative LC (Stationary phase: irregular bare silica 40 g, mobile phase: 0.3% NH₄OH, 95% DCM, yielding product 109 (28 mg, 16%) as a pale brown oil.

### E6. Preparation of Products 110, 111 and 112

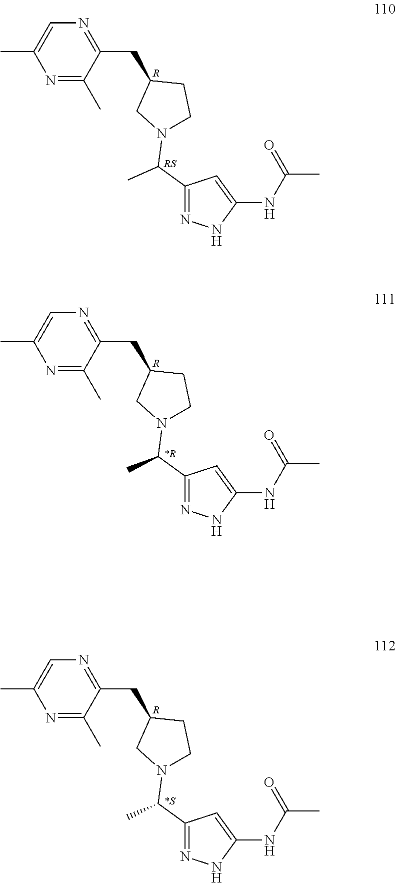

Product 110 was prepared following an analogous procedure to the one described for the synthesis of products 12 and 13 using intermediate 128 as starting material. The residue was purified by ion exchange chromatography using an ISOLUTE SCX2 cartridge eluting first with methanol which was discarded and then with 7M solution of ammonia in methanol. The filtrate was concentrated in vacuo to yield product 110 (70 mg, 74%) as a white foam. A purification was performed via chiral SFC (Stationary phase: CHIRALPAK AD-H 5 μm 250*30 mm, mobile phase: 50% CO₂, 50% EtOH (0.3% iPrNH₂)) yielding product 111 (33 mg, 35%) and product 112 (34 mg, 36%) as beige foams.

### E5. Preparation of Products 113, 114 and 115

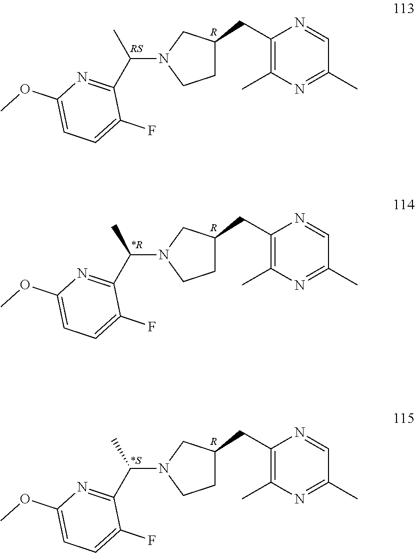

Product 113 was prepared following an analogous procedure to the one described for the synthesis of product 7 using intermediate 37 as starting material. The crude product was purified by flash column chromatography (silica; ethyl acetate in DCM 50/50 to 100/0 and methanol in ethyl acetate 0/100 to 10/90). The desired fractions were collected and concentrated in vacuo to yield product 113 (124 mg, 69%) as a brown oil. A purification was performed via chiral SFC (Stationary phase: Lux-Cellulose-4 5 μm 250*21.2 mm, mobile phase: 80% CO₂, 20% MeOH (0.3% iPrNH₂)) yielding product 114 (54 mg, 30%) and product 115 (43 mg, 24%) as a pale-brown oils.

### E6. Preparation of Product 116

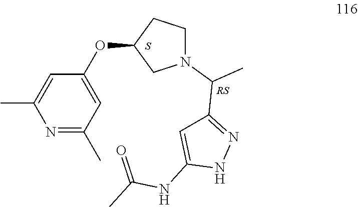

Product 116 was prepared following an analogous procedure to the one described for the synthesis of products 12 and 13 using intermediate 74 as starting material. The residue was purified by flash column chromatography (silica; 7N solution of ammonia in MeOH in DCM (0/100 to 10/90). The desired fractions were collected and concentrated in vacuo to yield product 116 (48 mg, 43%) as a cream solid.

### E6. Preparation of Product 117

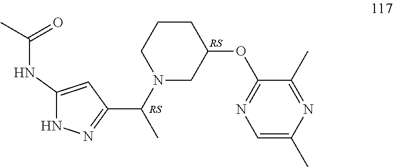 [SMILES: CC(=O)Nc1cc(C(C)N2CCCC(Oc3ncc(C)nc3C)C2)n[nH]1]

Product 117 was prepared following an analogous procedure to the one described for the synthesis of products 12 and 13 using intermediate 131 as starting material. The crude product was purified by RP HPLC (Stationary phase: C18 XBridge 30×100 mm 5 μm, mobile phase: Gradient from 80% NH₄HCO₃ 0.25% solution in H₂O, 20% CH₃CN to 60% NH₄HCO₃ 0.25% solution in H₂O, 40% CH₃CN). The desired fractions were collected and concentrated in vacuo to yield product 117 (55 mg, 51%) as a foam.

### E5. Preparation of Product 118

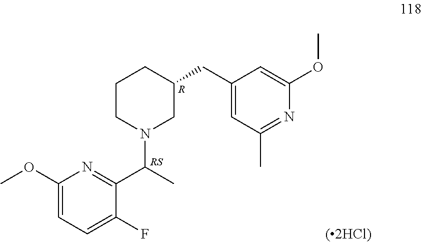

Product 118 was prepared following an analogous procedure to the one described for the synthesis of product 7 using intermediate 133 as starting material. The crude product was purified by flash column chromatography (silica; 7M solution of ammonia in MeOH in DCM 0/100 to 2/98). The desired fractions were collected, and the solvents evaporated in vacuo and the residue was purified by RP HPLC (Stationary phase: C18 XBridge 30×100 mm 5 μm, mobile phase: Gradient from 54% NH₄HCO₃ 0.25% solution in H₂O, 46% CH₃CN to 36% NH₄HCO₃ 0.25% solution in H₂O, 64% CH₃CN). The organic solvents were evaporated in vacuo and the aqueous layer was extracted with EtOAc. The organic layer was separated, dried (Na₂SO₄), filtered and the solvent evaporated in vacuo. The residue was dissolved in diethyl ether and a 2 N HCl solution in diethyl ether was added. The mixture was stirred at rt for 2 h and then the solvent was evaporated in vacuo. The residue was triturated with diethyl ether to yield product 118 (36 mg, 18%) as a light brown solid.

### E9. Preparation of Product 119

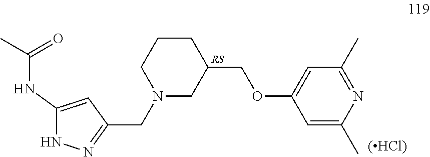

Sodium cyanoborohydride (CAS 25895-60-7, 0.028 g, 0.36 mmol) was added to a stirred solution of intermediate 51 (0.066 g, 0.26 mmol), intermediate 135 (0.068 g, 0.31 mmol) and acetic acid (0.029 mL, 0.52 mmol) in MeOH. The mixture was stirred at rt for 18 h and the solvent evaporated in vacuo. The crude product was purificated by RP-HPLC from 81% [25 mM NH₄HCO₃]-19% [MeCN:MeOH (1:1)] to 45% [25 mM NH₄HCO₃]-55%[MeCN: MeOH (1:1)]. The desired fractions were collected and concentrated in vacuo. The residue was dissolved in DCM and treated with a 4N HCl solution in 1,4-dioxane and converted into its hydrochloric acid salt. The solid was filtered off and tritured with DIPE to yield product 119 (37 mg, 36%) as a white solid.

### E6. Preparation of Product 120

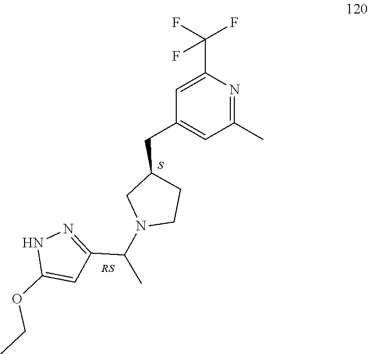

TFA (0.76 mL, 9.96 mmol) was added to intermediate 139 (0.14 g, 0.28 mmol). The mixture was stirred at rt for 16 h. The solvent was evaporated in vacuo and the residue was dissolved in DCM and washed with sat Na₂CO₃. The organic layer was separated, dried (Na₂SO₄), filtered and the solvents evaporated in vacuo. The crude product was purified by RP HPLC (Stationary phase: C18 XBridge 30×100 mm 5 μm), Mobile phase: Gradient from 67% NH₄HCO₃ 0.25% solution in Water, 33% CH₃CN to 50% NH₄HCO₃ 0.25% solution in Water, 50% CH₃CN) to yield product 120 (45 mg, 28%) as a colorless oil.

### E5. Preparation of Product 121

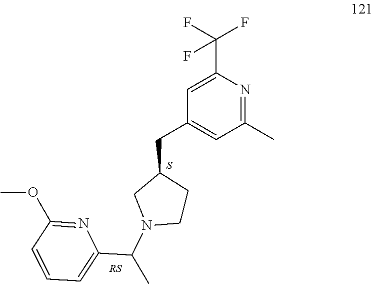

Product 121 was prepared following an analogous procedure to the one described for the synthesis of product 7 using intermediate 139 and 6-methoxy-2-pyridinecarboxaldehyde (CAS 54221-96-4) as starting materials. The crude product was purified by flash column chromatography (silica; MeOH in EtOAc in 0/100 to 10/90). The desired fractions were collected and concentrated in vacuo and the residue was re-purified by RP HPLC (Stationary phase: C18 XBridge 30×100 mm 5 μm, mobile phase: Gradient from 54% NH₄HCO₃ 0.25% solution in H₂O, 46% CH₃CN to 36% NH₄HCO₃ 0.25% solution in H₂O, 64% CH₃CN), to yield product 121 (120 mg, 77%) as a colorless oil.

### E6. Preparation of Product 122

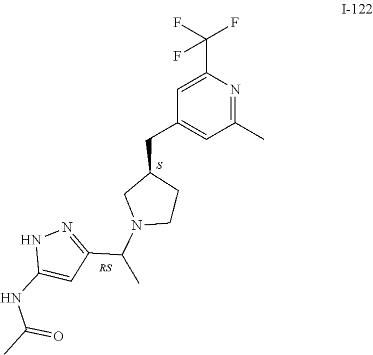

Product 122 was prepared following an analogous procedure to the one described for the synthesis of products 12 and 13 using intermediate 140 as starting material. The crude product was purified by ISOLUTE SCX2 cartridge eluting first with EtOH and then with 7M solution of ammonia in EtOH. The residue was triturated with diethylether to yield product 122 (33 mg. 66%) as an off-white solid.

### E9. Preparation of Product 123

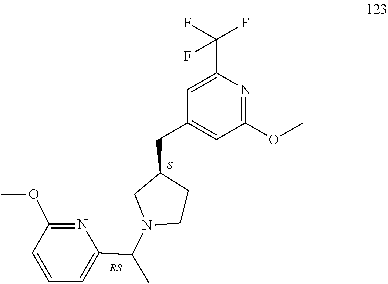

Sodium cyanoborohydride (0.024 g, 0.39 mmol) was added to a stirred solution of intermediate 142 (0.098 g, 0.32 mmol) and 1-(3-fluoro-6-methoxy-2-pyridinyl)-ethanone (CAS 1785479-37-9, 0.063 g, 0.42 mmol), titanium(IV) isopropoxide (0.064 mL, 0.42 mmol) in anhydrous DCM (1 mL) under N₂. The mixture was stirred at 80° C. for 16 h. Then water was added and the mixture was extracted with EtOAc. The organic layer was separated, dried (Na₂SO₄), filtered and the solvents evaporated in vacuo. The crude product was purified by flash column chromatography (silica; EtOAc in heptane 0/100 to 20/80). The desired fractions were collected and concentrated in vacuo, and the residue was purified again by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN), to yield product 123 (31 mg, 24%) as a yellow oil.

### E9. Preparation of Product 124

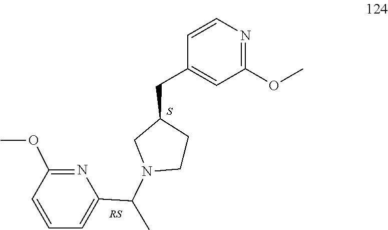

Product 124 was prepared following an analogous procedure to the one described for the synthesis of product 123 using intermediate 144 as starting material. The crude product was purified by flash column chromatography (silica; 7M solution of ammonia in MeOH in DCM 0/100 to 5/95). The desired fractions were collected and concentrated in vacuo, and the residue was purified again by RP HPLC (Stationary phase: C18 XBridge 30×100 mm 5 μm, mobile phase: Gradient from 67% NH₄HCO₃ 0.25% solution in H₂O, 33% CH₃CN to 50% NH₄HCO₃ 0.25% solution in H₂O, 50% CH₃CN), to yield product 124 (36 mg, 36%) as a colorless oil.

### E9. Preparation of Product 125

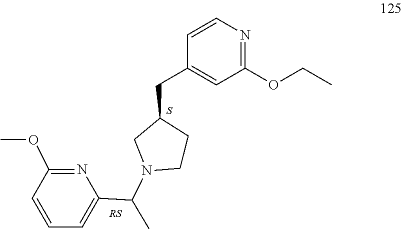

Product 125 was prepared following an analogous procedure to the one described for the synthesis of product 123 using intermediate 146 as starting material. The crude product was purified by flash column chromatography (silica; 7M solution of ammonia in MeOH in DCM 0/100 to 5/95). The desired fractions were collected and concentrated in vacuo, and the residue was purified again by RP HPLC (Stationary phase: C18 XBridge 30×100 mm 5 um, mobile phase: Gradient from 60% NH₄HCO₃ 0.25% solution in H₂O, 40% CH₃CN to 43% NH₄HCO₃ 0.25% solution in H₂O, 57% CH₃CN), to yield product 125 (40 mg, 36%).

### E9. Preparation of Product 126

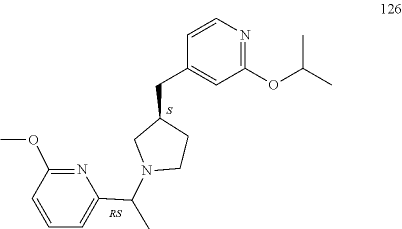

Product 126 was prepared following an analogous procedure to the one described for the synthesis of product 123 using intermediate 148 as starting material. The crude product was purified by flash column chromatography (silica; EtOAc in heptane 0/100 to 20/80). The desired fractions were collected and concentrated in vacuo, and the residue was purified again by RP HPLC (Stationary phase: C18 XBridge 30×100 mm 5 μm, mobile phase: Gradient from 54% NH₄HCO₃ 0.25% solution in H₂O, 46% CH₃CN to 36% NH₄HCO₃ 0.25% solution in H₂O, 64% CH₃CN), to yield product 126 (29 mg, 16%).

### E9. Preparation of Product 127

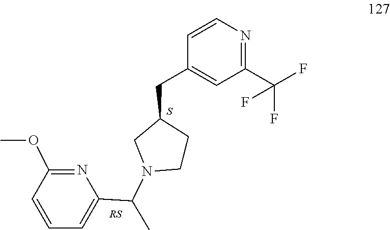

Product 127 was prepared following an analogous procedure to the one described for the synthesis of product 123 using intermediate 150 as starting material. The crude product was purified by flash column chromatography (silica; 7M solution of ammonia in MeOH in DCM 0/100 to 5/95). The desired fractions were collected and concentrated in vacuo to yield product 127 (132 mg, 55%) as a yellow oil.

### E6. Preparation of Product 128

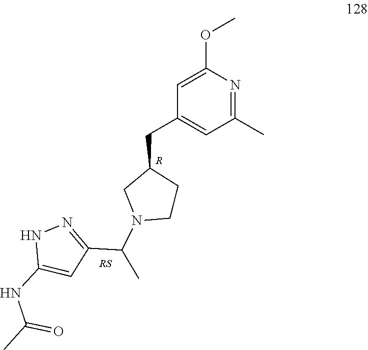

Product 128 was prepared following an analogous procedure to the one described for the synthesis of products 12 and 13 using intermediate 153 as starting material. The crude product was purified by RP HPLC (Stationary phase: C18 XBridge 30×100 mm 5 μm, mobile phase: Gradient from 80% NH₄HCO₃ 0.25% solution in H₂O, 20% CH₃CN to 60% NH₄HCO₃ 0.25% solution in H₂O, 40% CH₃CN). The desired fractions were collected and concentrated in vacuo to yield product 128 (80 mg, 63%) as a foam.

### E5. Preparation of Product 129

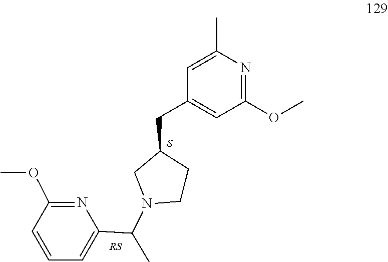

Product 129 was prepared following an analogous procedure to the one described for the synthesis of product 99 using 6-methoxy-2-pyridinecarboxaldehyde (CAS 54221-96-4) and intermediate 33 as starting materials. The crude product was purified by flash column chromatography (amino functionalized silica; MeOH in DCM 0/100 to 4/96). The desired fractions were collected and concentrated in vacuo to yield product 129 (108 mg, 82%) as a sticky oil.

### E5. Preparation of Product 130

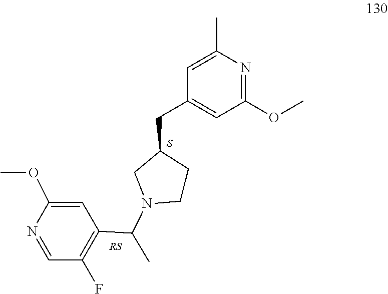

Product 130 was prepared following an analogous procedure to the one described for the synthesis of product 99 using intermediate 33 as starting material. The crude product was purified by flash column chromatography (silica; 7M solution of ammonia in MeOH in DCM 0/100 to 2/98). The desired fractions were collected and concentrated in vacuo and the residue was further purified by RP HPLC (Stationary phase: C18 XBridge 30×100 mm 5 μm, mobile phase: Gradient from 47% NH₄HCO₃ 0.25% solution in H₂O, 53% CH₃CN to 30% NH₄HCO₃ 0.25% solution in H₂O, 70% CH₃CN). The desired fractions were collected, and the organic solvent evaporated in vacuo. The aqueous layer was extracted with EtOAc. The organic layer was separated, dried (Na₂SO₄) filtered and the solvents evaporated in vacuo to yield product 130 (92 mg, 75%) as a colorless oil.

### E6. Preparation of Product 131

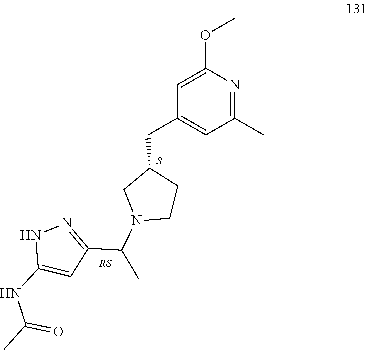

Product 131 was prepared following an analogous procedure to the one described for the synthesis of products 12 and 13 using intermediates 33 and 154 as starting material. The crude product was purified by ISOLUTE SCX2 cartridge eluting first with MeOH and then with 7M solution of ammonia in MeOH to yield product 131 (71 mg. 77%) as a white solid.

### E5. Preparation of Product 132

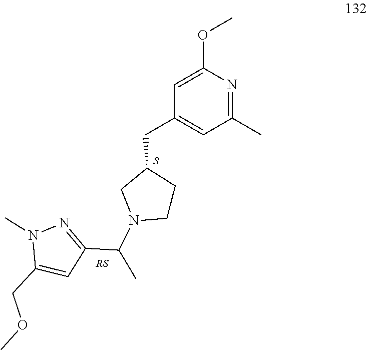

Product 132 was prepared following an analogous procedure to the one described for the synthesis of product 99 using intermediates 33 and 157 as starting materials. The crude product was purified by flash column chromatography (silica; 7M solution of ammonia in MeOH in DCM 0/100 to 2/98). The desired fractions were collected and concentrated in vacuo and the residue was further purified by RP HPLC (Stationary phase: C18 XBridge 30×100 mm 5 μm, mobile phase: Gradient from 75% NH₄HCO₃ 0.25% solution in H₂O, 25% CH₃CN to 57% NH₄HCO₃ 0.25% solution in H₂O, 43% CH₃CN). The desired fractions were collected, and the organic solvent evaporated in vacuo. The aqueous layer was extracted with DCM. The organic layer was separated, dried (Na₂SO₄) filtered and the solvents evaporated in vacuo to yield product 132 (41 mg, 24%) as a yellow oil.

### E5. Preparation of Product 133

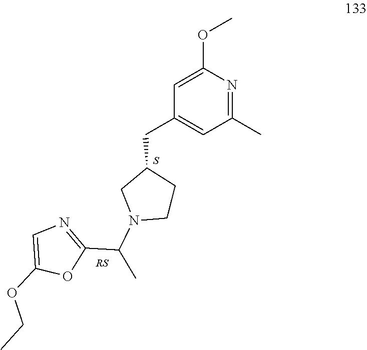

Product 133 was prepared following an analogous procedure to the one described for the synthesis of products 99 using intermediate 33 as starting material. The crude product was purified by flash column chromatography (silica; 7M solution of ammonia in MeOH in DCM 0/100 to 2/98). The desired fractions were collected and evaporated in vacuo to yield product 133 (16 mg, 19%) as a yellowish oil.

### E5. Preparation of Product 134

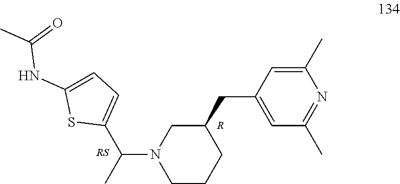

Product 134 was prepared following an analogous procedure to the one described for the synthesis of product 30 using intermediate 3 and N-(5-formyl-2-thienyl)acetamide (CAS 31167-35-8) as starting materials. The crude product was purified by flash column chromatography (silica; MeOH in DCM 0/100 to 5/95), the desired fractions were collected and concentrated in vacuo and the residue was purified by RP HPLC (Stationary phase: C18 XBridge 30×100 mm 5 μm), Mobile phase: Gradient from 90% NH₄HCO₃ 0.25% solution in Water, 10% CH₃CN to 65% NH₄HCO₃ 0.25% solution in Water, 35% CH₃CN) and then repurified by RP HPLC (Stationary phase: C18 XBridge 30×100 mm 5 μm), Mobile phase: Gradient from 75% NH₄HCO₃ 0.25% solution in Water, 25% CH₃CN to 57% NH₄HCO₃ 0.25% solution in Water, 43% CH₃CN), to yield compound 134 (30 mg, 16%) as a cream sticky solid.

### E1. Preparation of Product 135

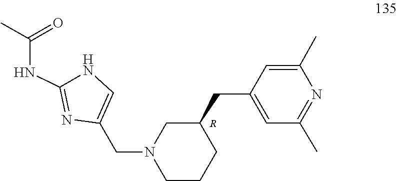

Product 135 was prepared following an analogous procedure to the one described for the synthesis of product 1 using intermediates 3 and 158 as starting materials. The product was purified by RP HPLC (Stationary phase: C18 XBridge 30×100 mm 5 um, mobile phase: 90% NH₄HCO₃ 0.25% solution in Water, 10% CH₃CN to 65% NH₄HCO₃ 0.25% solution in Water, 35% CH₃CN), to yield compound 135 (52 mg, 44%) as a colorless oil that crystallized upon standing.

### E5. Preparation of Product 136

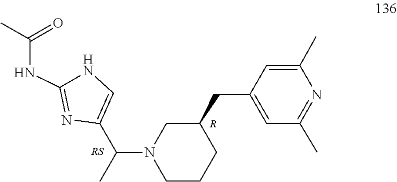

Product 136 was prepared following an analogous procedure to the one described for the synthesis of product 7 using intermediates 3 and 158 as starting materials. The product was purified by RP HPLC (Stationary phase: C18 XBridge 30×100 mm 5 um, mobile phase: 80% NH₄HCO₃ 0.25% solution in Water, 20% CH₃CN to 60% NH₄HCO₃ 0.25% solution in Water, 40% CH₃CN), to yield compound 136 (13 mg, 9%) as a pale-yellow oil.

### E5. Preparation of Product 137

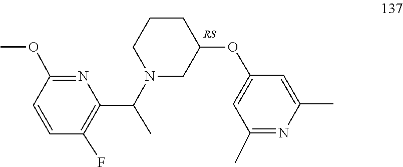

Product 137 was prepared following an analogous procedure to the one described for the synthesis of product 7 using intermediate 23 as starting material. The product was purified was purified by flash column chromatography (silica; 7M solution of ammonia in MeOH in DCM 0/100 to 30/70). The desired fractions were collected and concentrated in vacuo to yield compound 137 (75 mg, 43%) as a yellow oil.

### E6. Preparation of Product 138

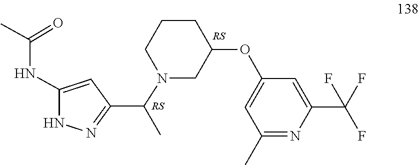 [SMILES: CC(=O)Nc1cc(C(C)N2CCCC(Oc3cc(C)nc(C(F)(F)F)c3)C2)n[nH]1]

Product 138 was prepared following an analogous procedure to the one described for the synthesis of products 12 and 13 using intermediate 159 as starting material. The product was purified was purified by flash column chromatography (silica; 7M solution of ammonia in MeOH in DCM 0/100 to 5/95). The desired fractions were collected and concentrated in vacuo to yield compound 138 (76 mg, 54%) as a white solid.

### E3. Preparation of Product 139

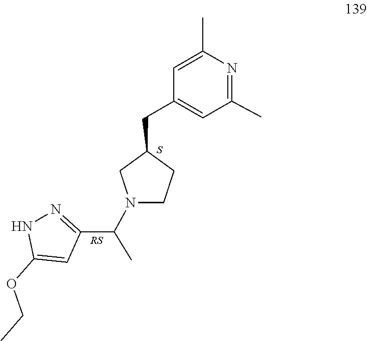

Product 139 was prepared following an analogous procedure to the one described for the synthesis of product 120 using intermediate 160 as starting material. The product was purified by ion exchange chromatography using ISOLUTE SCX2 cartridge eluting first with MeOH and then with 7M solution of ammonia in MeOH followed by RP HPLC (Stationary phase: C18 XBridge 30×100 mm 5 um, mobile phase: 80% NH₄HCO₃ 0.25% solution in Water, 20% CH₃CN to 60% NH₄HCO₃ 0.25% solution in Water, 40% CH₃CN), to yield compound 139 (69 mg, 67%) as a colorless oil

### E9. Preparation of Product 140

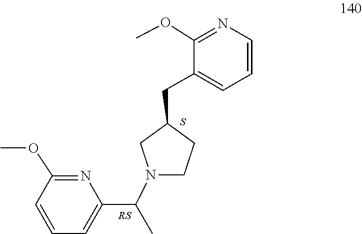

Product 140 was prepared following an analogous procedure to the one described for the synthesis of product 123 using intermediate 162 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 67% NH₄HCO₃ 0.25% solution in Water, 33% CH₃CN to 50% NH₄HCO₃ 0.25% solution in Water, 40% CH₃CN), to yield compound 140 (107 mg, 35%) as a colorless oil.

### E9. Preparation of Product 141

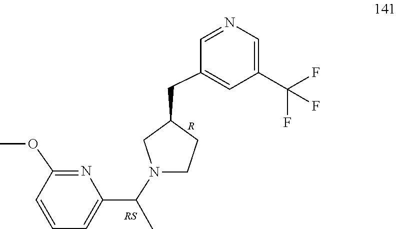

Product 141 was prepared following an analogous procedure to the one described for the synthesis of product 123 using intermediate 164 as starting material. The crude product was purified by flash column chromatography (silica; 7M solution of ammonia in MeOH in DCM 0/100 to 5/95) to yield compound 141 (177 mg, 51%) as a colorless oil.

### E9. Preparation of Product 142

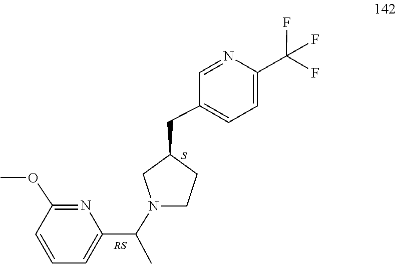

Product 142 was prepared following an analogous procedure to the one described for the synthesis of product 123 using intermediate 166 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 60% NH₄HCO₃ 0.25% solution in Water, 40% CH₃CN to 43% NH₄HCO₃ 0.25% solution in Water, 57% CH₃CN), to yield compound 142 (65 mg, 24%) as a yellow oil.

### E9. Preparation of Product 143

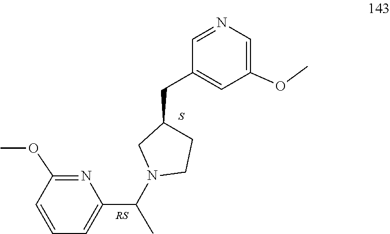

Product 143 was prepared following an analogous procedure to the one described for the synthesis of product 123 using intermediate 168 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 143 (70 mg, 26%) as a yellow oil.

### E9. Preparation of Product 144

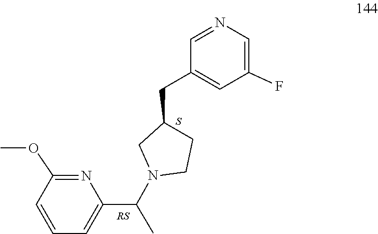

Product 144 was prepared following an analogous procedure to the one described for the synthesis of product 123 using intermediate 170 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 144 (80 mg, 34%) as a yellow oil.

### E9. Preparation of Product 145

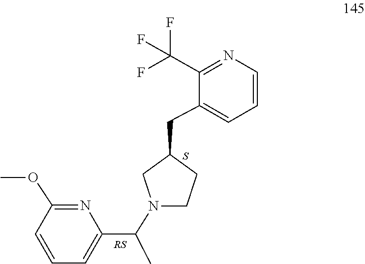

Product 145 was prepared following an analogous procedure to the one described for the synthesis of product 123 using intermediate 172 as starting material. The crude product was purified by flash column chromatography (silica; EtOAc in Heptane 0/100 to 20/80) followed by RP HPLC (Stationary phase: C18 XBridge 30×100 mm 5 um, mobile phase: 60% NH₄HCO₃ 0.25% solution in Water, 40% CH₃CN to 43% NH₄HCO₃ 0.25% solution in Water, 57% CH₃CN), to yield compound 145 (105 mg, 34%) as a yellow oil.

### E9. Preparation of Product 146

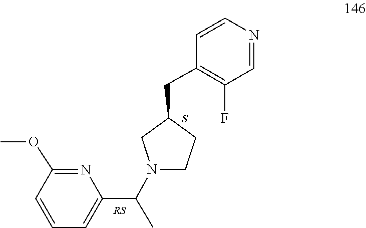

Product 146 was prepared following an analogous procedure to the one described for the synthesis of product 123 using intermediate 174 as starting material. The crude product was purified by flash column chromatography (silica; EtOAc in Heptane 0/100 to 20/80) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 146 (57 mg, 43%) as a colorless oil.

### E9. Preparation of Product 147

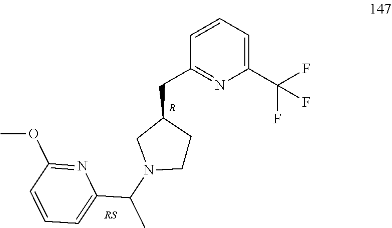

Product 147 was prepared following an analogous procedure to the one described for the synthesis of product 123 using intermediate 176 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 147 (140 mg, 28%).

### E9. Preparation of Product 148

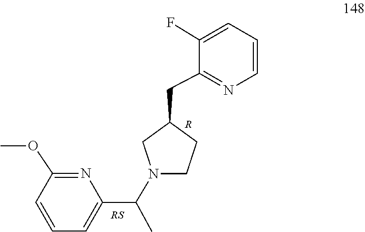

Product 148 was prepared following an analogous procedure to the one described for the synthesis of product 123 using intermediate 178 as starting material. The crude product was purified by flash column chromatography (silica; 7M solution of ammonia in MeOH in DCM 0/100 to 5/95) to yield compound 148 (30 mg, 27%) as a colorless oil.

### E9. Preparation of Product 149

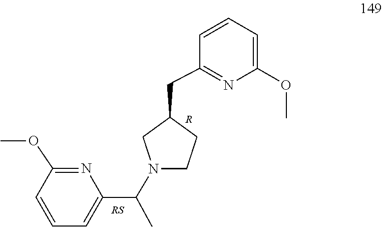

Product 149 was prepared following an analogous procedure to the one described for the synthesis of product 123 using intermediate 180 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 149 (35 mg, 7%) as a colorless oil.

### E9. Preparation of Product 150

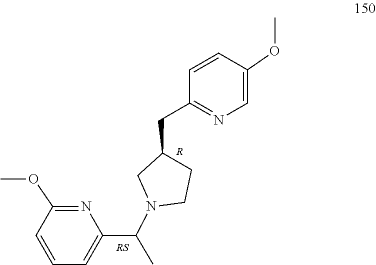

Product 150 was prepared following an analogous procedure to the one described for the synthesis of product 123 using intermediate 182 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 150 (82 mg, 34%) as a colorless oil.

### E9. Preparation of Product 151

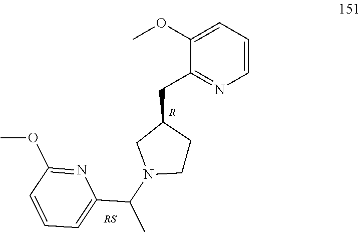

Product 151 was prepared following an analogous procedure to the one described for the synthesis of product 123 using intermediate 184 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 151 (26 mg, 20%) as a yellow oil.

### E9. Preparation of Products 152, 153 and 154

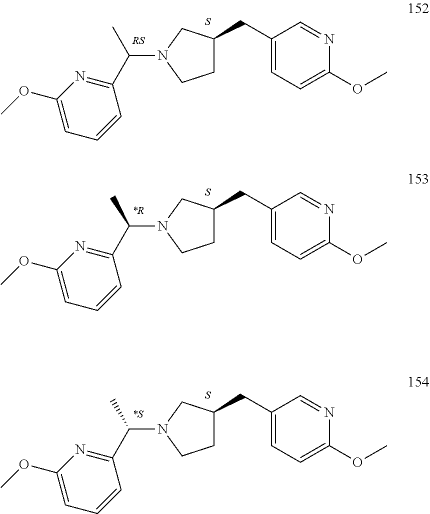

Product 152 was prepared following an analogous procedure to the one described for the synthesis of product 123 using intermediate 186 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 67% NH₄HCO₃ 0.25% solution in Water, 33% CH₃CN to 50% NH₄HCO₃ 0.25% solution in Water, 50% CH₃CN), to yield compound 152 (90 mg, 29%) as a colorless oil. A purification was performed via achiral SFC (Stationary phase: CHIRALPAK AD-H 5 μm 250×30 mm, Mobile phase: 85% C02, 15% EtOH (0.3% iPrNH₂)) followed by achiral SFC (Stationary phase: CYANO 6 μm 150×21.2 mm, Mobile phase: 85% C02, 15% MeOH (0.3% iPrNH₂)) yielding product 153 (31 mg, 30%) and product 154 (15 mg, 24%).

### E9. Preparation of Products 155, 156 and 157

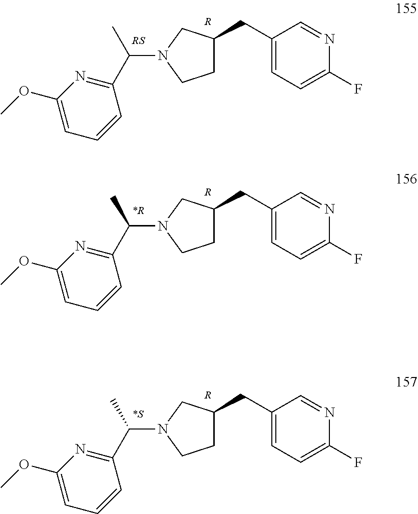

Product 155 was prepared following an analogous procedure to the one described for the synthesis of product 123 using intermediate 188 as starting material. The crude product was purified by flash column chromatography (silica; 7M solution of ammonia in MeOH in DCM 0/100 to 5/95) followed by RP HPLC (Stationary phase: C18 XBridge 30×100 mm 5 um, mobile phase: 75% NH₄HCO₃ 0.25% solution in Water, 25% CH₃CN to 57% NH₄HCO₃ 0.25% solution in Water, 43% CH₃CN), to yield compound 155 (30 mg, 25%) as a colorless oil. A purification was performed via chiral SFC (Stationary phase: Lux-amylose-2 5 μm 250×21.2 mm, Mobile phase: 85% CO₂, 15% EtOH (0.3% iPrNH₂)) yielding product 156 (11 mg, 9%) and product 157 (10 mg, 24%).

### E9. Preparation of Product 158

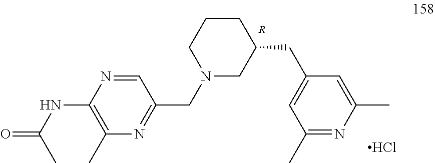

Palladium(II) acetate (CAS 3375-31-3, 2.1 mg, 0.009 mmol) 9,9-dimethyl-4,5-bis(diphenylphosphino)xanthene (CAS 161265-03-8, 12 mg, 0.02 mmol) and cesium carbonate (CAS 534-17-8, 151 mg, 0.46 mmol) were added to a stirred solution of acetamide (CAS 60-35-5, 27 mg, 0.46 mmol) and intermediate 191 (100 mg, 0.23 mmol) in 1,4-dioxane (5 ml) under nitrogen. The reaction mixture was degassed with N₂ and stirred at 94° C. overnight. Pd₂(dba)₃ (CAS 51364-51-3, 8.5 mg, 0.009 mmol) and 9,9-dimethyl-4,5-bis(diphenylphosphino)xanthene (CAS 161265-03-8, 12 mg, 0.021 mmol) were added to 1,4-dioxane (5 ml) under nitrogen and this mixture was stirred at 40° C. for 20 min. This solution was added to the reaction mixture and heated at 95° C. overnight. The mixture was diluted with water and extracted with EtOAc. The organic layer was separated, dried (MgSO₄), filtered and the solvents evaporated in vacuo. The crude product was purified by flash column chromatography (silica; MeOH in DCM (10:1) in DCM, from 0/100 to 100/0). The desired fractions were collected, and the solvents evaporated in vacuo. This material was purified for reverse phase (from 72% (H₂O 25 mM NH₄HCO₃)-28% MeCN-MeOH to 36% H₂O (25 mM NH₄HCO₃)-64% MeCN-MeOH). The desired fractions were collected concentrated in vacuo to yield a colorless sticky solid. The material was taken into DCM and treated with 2 eq of a 4n solution of HCl in 1,4-dioxane (0.030 ml). The solvents evaporated in vacuo and the product was tritured with diethyl ether to yield product 158 (25 mg, 24%) as a white solid.

### E4. Preparation of Product 159

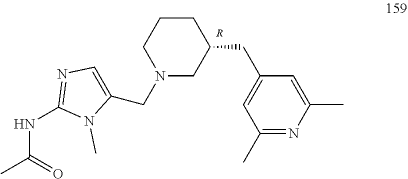

TFA (CAS 76-05-1, 0.06 mL, 0.78 mmol) was added to a stirred solution of intermediate 195 (65 mg, 0.16 mmol) in DCM (1.2 mL) in a sealed tube and under N₂. The mixture was stirred at rt for 17 h. Then more TFA (CAS 76-05-1, 0.12 mL, 1.57 mmol) was added and the mixture was stirred at rt for 24 h. The solvent was evaporated in vacuo and the crude was treated dissolved in DCM (1.6 mL), cooled at 0° C. and Et₃N (CAS 121-44-8, 0.12 mL, 0.73 mmol) and acetyl chloride (CAS 75-36-5, 0.015 mL, 0.21 mmol) were added. The mixture was stirred at 0° C. for 5 min and at rt for 2.5 h. The mixture was treated with a saturated NaHCO₃ solution and extracted with more DCM. The organic layer was separated, dried (MgSO₄), filtered and the solvent evaporated in vacuo. The crude was purified by RP HPLC (Stationary phase: C18 XBridge 30×100 mm 5 μm), Mobile phase: Gradient from 80% NH₄HCO₃ 0.25% solution in Water, 20% CH₃CN to 60% NH₄HCO₃ 0.25% solution in Water, 40% CH₃CN). The desired fractions were collected and extracted with EtOAc. The organic layer was separated, dried (MgSO₄), filtered and the solvent evaporated in vacuo to yield product 159 (8 mg, 14%) as a pale-purple oil.

### E9. Preparation of Product 160

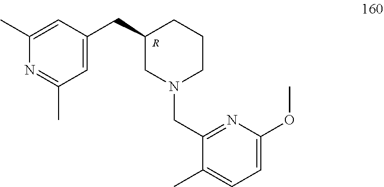

6-Methoxy-3-methyl-2-pyridinecarboxaldehyde (CAS 123506-64-9, 55 mg, 0.37 mmol) was added to a solution of intermediate 3 (0.05 g, 0.49 mmol) in DCE (2 mL) and the reaction mixture was stirred at rt for 1 h. Then sodium triacetoxyborohydride (CAS 56553-60-7, 114 mg, 0.54 mmol) was added and the reaction mixture was stirred at rt for 20 h. Then a saturated NaHCO₃ solution was added and the mixture was extracted with DCM. The organic layer was separated, dried (MgSO₄), filtered and the solvents evaporated in vacuo. The crude product was purified by flash column chromatography (silica; 7M solution of ammonia in MeOH in DCM 0/100 to 2/98). The desired fractions were collected and the solvents evaporated in vacuo to yield product 160 (83 mg, 66%) as a yellow oil.

### E9. Preparation of Product 161

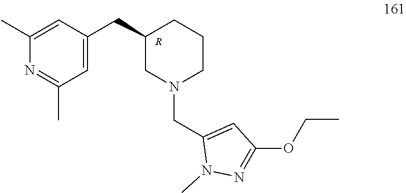

Product 161 was prepared following an analogous procedure to the one described for the synthesis of product 158 using intermediate 200 as starting material. The crude product was purified by flash column chromatography (silica; MeOH in DCM 0/100 to 15/85) to yield compound 161 (111 mg, 45%) as a yellow oil.

### E9. Preparation of Product 162

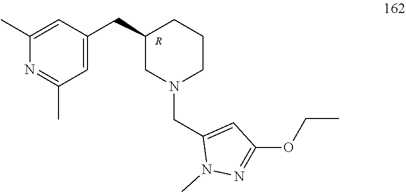

Product 162 was prepared following an analogous procedure to the one described for the synthesis of product 158 using intermediate 201 as starting material. The crude product was purified by flash column chromatography (silica; MeOH in DCM 0/100 to 5/95) to yield compound 162 (124 mg, 61%) as a colorless oil.

### E6 PREPARATION OF PRODUCT 163

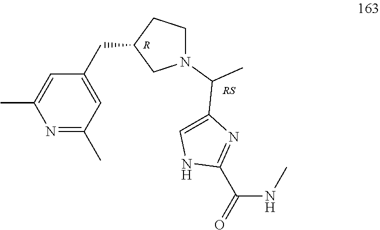

Product 163 was prepared following an analogous procedure to the one described for the synthesis of product 10 using intermediate 207 as starting material. The crude product was purified by flash column chromatography (silica; MeOH in DCM 0/100 to 3/98) to yield product 163 (17 mg, 34%) as a white foam.

### E6 PREPARATION OF PRODUCT 164

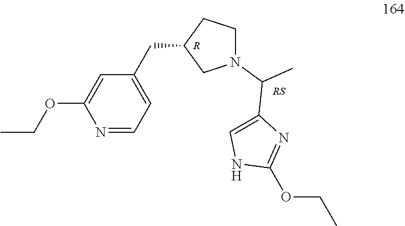

Product 164 was prepared following an analogous procedure to the one described for the synthesis of product 10 using intermediate 210 as starting material. The crude product was purified by ion exchange chromatography using an ISOLUTE SCX2 cartridge eluting first with methanol which was discarded and then with a 7M solution of ammonia in methanol. The filtrate was concentrated in vacuo to yield product 164 (5 mg, 11%) as colorless oil.

### E6 PREPARATION OF PRODUCT 165

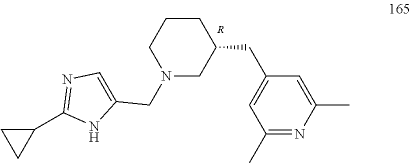

Product 165 was prepared following an analogous procedure to the one described for the synthesis of product 10 using intermediate 213 as starting material. The crude product was purified by RP HPLC (Stationary phase: C18 XBridge 30×100 mm 5 um), Mobile phase: Gradient from 81% 0.1% NH₄CO₃H/NH₄OH pH 9 solution in Water, 19% CH₃CN to 64% 0.1% NH₄CO₃H/NH₄OH pH 9 solution in Water, 36% CH₃CN) to yield product 165 (8 mg, 84%).

### Analytical Part

### Melting Points

Values are peak values, and are obtained with experimental uncertainties that are commonly associated with this analytical method. Mettler Toledo MP50: For a number of compounds, melting points were determined in open capillary tubes on a Mettler Toledo MP50. Melting points were measured with a temperature gradient of 10° C./minute. Maximum temperature was 300° C. The melting point data was read from a digital display and checked from a video recording system.

DSC823e: For a number of compounds, melting points were determined with a DSC823e (Mettler-Toledo) apparatus. Melting points were measured with a temperature gradient of 10° C./min. Maximum temperature was 300° C. Values are peak values.

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). For molecules with multiple isotopic patterns (Br, Cl.), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica; “CSH” charged surface hybrid, “UPLC” Ultra Performance Liquid Chromatography, “DAD” Diode Array Detector.

TABLE 1
LC-MS Methods (Flow expressed in mL/min; column temperature (T) in ° C.;
Run time in min).

| Method code | Instrument | Column | Mobile phase | Gradient | $\frac{Flow}{Col T}$ | Run time |
|---|---|---|---|---|---|---|
| 1 | Waters: Acquity ® | Waters: BEH C18 (1.7 μm, | A: 95% CH₃COONH₄ | From 95% A to | $\frac{1}{50}$ | 5 |
| | IClass | 2.1 × 50 mm) | 6.5 mM + 5% | 5% A n | | |
| | UPLC ®- | | CH₃CN, | 4.6 min, | | |
| | DAD and | | B: CH₃CN | held for | | |
| | Xevo G2-S | | | 0.4 min | | |
| | QTOF | | | | | |
| 2 | Waters: Acquity ® | Waters: BEH C18 (1.7 μm, | A: 95% CH₃COONH₄ | From 95% A to | $\frac{0.8}{- 50}$ | 5 |
| | UPLC ®- | 2.1 × 50 mm) | 6.5 mM + 5% | 5% A in | | |
| | DAD and | | CH₃CN, | 4.5 min, | | |
| | SQD | | B: CH₃CN | held for | | |
| | | | | 0.5 min | | |
| 3 | Waters: Acquity UPLC ®-DAD | Waters: BEH C18 (1.7 μm, | A: 95% CH₃COONH₄ | 84.2% A for | $\frac{0.343}{40}$ | 6.2 |
| | and Quattro | 2.1 × 100 mm) | 7 mM/5% | 0.49 min, | | |
| | Micro ™ | | CH₃CN, B: | to 10.5% | | |
| | | | CH₃CN | A in | | |
| | | | | 2.18 min, | | |
| | | | | held for | | |
| | | | | 1.94 min, | | |
| | | | | back to | | |
| | | | | 84.2% A | | |
| | | | | in | | |
| | | | | 0.73 min, | | |
| | | | | held for | | |
| | | | | 0.73 min. | | |
| 4 | Waters: Acquity ® | Waters: BEH C18 (1.7 μm, | A: 95% CH₃COONH₄ | From 95% A to | $\frac{1}{50}$ | 2 |
| | UPLC ®- | 2.1 × 50 mm) | 6.5 mM + 5% | 40% A in | | |
| | DAD and | | CH₃CN, | 1.2 min, to | | |
| | SQD | | B: CH₃CN | 5% A in | | |
| | | | | 0.6 min, | | |
| | | | | held for | | |
| | | | | 0.2 min | | |
| 5 | Agilent: 1100-DAD | YMC: Pack ODS-AQ | A: HCOOH 0.1% in water, | 95% A to 5% A in | $\frac{2.6}{35}$ | 6 |
| | and MSD | (3 μm, | B: CH₃CN | 4.8 min, | | |
| | | 4.6 × 50 mm) | | held for | | |
| | | | | 1 min, | | |
| | | | | back to | | |
| | | | | 95% A in | | |
| | | | | 0.2 min. | | |
| 6 | Agilent 1260 Infinity DAD | YMC-pack ODS-AQ | A: 0.1% HCOOH in | From 95% A to | $\frac{2.6}{35}$ | 6.8 |
| | TOF-LC/MS | C18 (50 × | H₂O | 5% A in | | |
| | G6224A | 4.6 mm, 3 | B: CH₃CN | 4.8 min, | | |
| | | μm) | | held for | | |
| | | | | 1.0 min, | | |
| | | | | to 95% A | | |
| | | | | in 0.2 | | |
| | | | | min. | | |
| 7 | Agilent 1100 HPLC DAD | YMC-pack ODS-AQ | A: 0.1% HCOOH in | 100% A held for | $\frac{2.6}{35}$ | 6.2 |
| | LC/MS | C18 (50 × | H₂O | 0.2. From | | |
| | G1956A | 4.6 mm, 3 | B: CH₃CN | 100% A | | |
| | | μm) | | to 50% A | | |
| | | | | in 4.5 | | |
| | | | | min, and | | |
| | | | | to 5% A | | |
| | | | | in 0.1 | | |
| | | | | min, held | | |
| | | | | for 1.0 | | |
| | | | | min, to | | |
| | | | | 95% A in | | |
| | | | | 0.2 min. | | |
| 8 | Waters: Acquity ® | Waters: BEH C18 (1.7 μm, | A: 95% CH₃COONH₄ | From 95% A to | $\frac{0.8}{- 50}$ | 2.5 |
| | UPLC ®- | 2.1 × 50 mm) | 6.5 mM + 5% | 5% A in | | |
| | DAD and | | CH₃CN, | 2.0 min, | | |
| | SQD | | B: CH₃CN | held for | | |
| | | | | 0.5 min | | |
| 9 | Waters: Acquity ® | Agilent: RRHD | A: 95% CH₃COONH₄ | From 95% A to | $\frac{1}{- 50}$ | 2 |
| | IClass | (1.8 μm, | 6.5 mM + 5% | 40 % A in | | |
| | UPLC ®- | 2.1 × 50 mm) | CH₃CN, | 1.2 min, to | | |
| | DAD and | | B: CH₃CN | 5% A in | | |
| | SQD | | | 0.6 min, | | |
| | | | | held for | | |
| | | | | 0.2 min | | |
| 10 | Waters: Acquity ® | Agilent: RRHD | A: 95% CH₃COONH₄ | From 95% A to | $\frac{0.8}{- 50}$ | 5 |
| | IClass | (1.8 μm, | 6.5 mM + 5% | 5% A in | | |
| | UPLC ®- | 2.1 × 50 mm) | CH₃CN, | 4.5 min, | | |
| | DAD and | | B: CH₃CN | held for | | |
| | SQD | | | 0.5 min | | |
| 11 | Waters: Acquity ® | Waters: BEH C18 (1.7 μm, | A: 95% CH₃COONH₄ | From 95% A to | $\frac{1}{- 50}$ | 2 |
| | IClass | 2.1 × 50 mm) | 6.5 mM + 5% | 40% A in | | |
| | UPLC ®- | | CH₃CN, | 1.2 min, to | | |
| | DAD and | | B: CH₃CN | 5% A in | | |
| | Xevo G2-S | | | 0.6 min, | | |
| | QTOF | | | held for | | |
| | | | | 0.2 min | | |

TABLE 2
Analytical data-melting point (M.p.) and LCMS: [M + H]⁺ means the
protonated mass of the free base of the compound, [M − H]⁻ means the deprotonated mass
of the free base of the compound or the type of adduct specified [M + CH₃COO]⁻). Rₜ
means retention time (in min). For some compounds, exact mass was determined.

| | | | | | LCMS |
|---|---|---|---|---|---|
| Co. No. | M.p. (° C.) | [M + H]⁺ | [M − H]⁻ | Rₜ | Method |
| 1 | n.m. | 342 | − | 0.96 | 1 |
| 2 | n.m. | 328 | − | 1.16 | 1 |
| 3 | done | 356 | − | 1.00/1.03^{&} | 1 |
| 4 | n.m. | 356 | 354 | 1.81 | 3 |
| 5 | n.m. | 356 | 354 | 1.8 | 3 |
| 6 | n.d. | 356 | − | 0.87 | 1 |
| 7 | n.m. | 370 | − | 1.48/1.50^{&} | 1 |
| 8 | n.m. | 370 | − | 2.27 | 3 |
| 9 | n.m. | 370 | − | 2.25 | 3 |
| 10 | n.m. | 356 | − | 1.24 | 1 |
| 11 | n.d. | 356 | − | 1.37 | 1 |
| 12 | n.m. | 370 | − | 1.06 | 1 |
| 13 | n.m. | 370 | − | 1.11 | 1 |
| 14 | 112.89 | 329 | − | 1.54 | 1 |
| 15 | 88.44 | 343 | − | 1.63 | 1 |
| 16 | 138.21 | 343 | − | 1.63 | 1 |
| 17 | n.m. | 357 | − | 2.27 | 1 |
| 18 | n.m. | 357 | − | 1.47/1.51^{&} | 1 |
| 19 | n.m. | 343 | − | 1.16/1.18^{&} | 1 |
| 20 | n.m. | 325 | − | 1.35 | 1 |
| 21 | n.m. | 339 | − | 1.34/1.39^{&} | 1 |
| 22 | n.m. | 407 | − | 1.72 | 1 |
| 23 | n.m. | 329 | − | 0.64 | 9 |
| 24 | n.m. | 357 | − | 0.82 | 11 |
| 25 | n.m. | 299 | − | 0.45 | 4 |
| 26 | n.m. | 346 | − | 2.22 | 1 |
| 27 | n.m. | 358 | − | 1.14 | 1 |
| 28 | n.d. | — | 356 | 1.31 | 1 |
| 29 | | No hay | − | | |
| | | lcms | | | |
| 30 | n.m. | 344 | − | 2.08/2.11^{&} | 1 |
| 31 | 133 | 353 | − | 0.86 | 5 |
| 32 | 138 | 367 | − | 1 | 6 |
| 33 | 147.5 | 381 | − | 0.29 | 7 |
| 34 | 186.5 | 353 | − | 1.54 | 7 |
| 35 | n.m. | 367 | − | 1.59 | 7 |
| 36 | n.d. | 367 | − | 1.28 | 1 |
| 37 | n.d. | 367 | − | 1.28 | 1 |
| 38 | 188.1 | 381 | − | 1.05 | 5 |
| 39 | 250.1 | 353 | − | 0.64 | 5 |
| 40 | 251.2 | 367 | − | 1.54 | 5 |
| 41 | n.m. | 358 | − | 1.29 | 8 |
| 42 | n.m. | 358 | − | 1.32 | 8 |
| 43 | 161.4 | 340 | − | 0.84 | 5 |
| 44 | 138 | 354 | − | 1.15 | 5 |
| 45 | n.m. | 358 | − | 2.38/2.40^{&} | 1 |
| 46 | n.m. | 340 | − | 1.59 | 1 |
| 47 | n.m. | 340 | − | 1.64 | 1 |
| 48 | n.m. | 340 | − | 1.38/1.40^{&} | 1 |
| 49 | n.m. | 354 | − | 1.87 | 1 |
| 50 | n.m. | 354 | − | 1.98 | 1 |
| 51 | n.m. | 354 | − | 2.03 | 1 |
| 52 | n.m. | 340 | − | 1.78 | 1 |
| 53 | n.m. | 340 | − | 2.25/2.27^{&} | 1 |
| 54 | n.m. | 398 | − | 1.69 | 8 |
| 55 | n.m. | 398 | − | 2.96 | 1 |
| 56 | n.m. | 368 | − | 2.24/2.26^{&} | 1 |
| 57 | n.m. | 354 | − | 1.53 | 1 |
| 58 | n.m. | 354 | − | 1.64 | 1 |
| 59 | n.m. | 350 | − | 1.9 | 1 |
| 60 | n.m. | 350 | − | 1.94 | 1 |
| 61 | n.m. | 378 | − | 2.54/2.58^{&} | 1 |
| 62 | 206.5 | 354 | − | 0.58 | 5 |
| 63 | n.d. | 354 | − | 1.25 | 1 |
| 64 | 211.6 | 368 | − | 1.67 | 7 |
| 65 | n.m. | 341 | − | 1.73 | 1 |
| 66 | n.m. | 372 | − | 1.34/1.36^{&} | 1 |
| 67 | n.m. | 372 | − | 1.34 | 1 |
| 68 | n.m. | 372 | − | 1.36 | 1 |
| 69 | n.m. | 374 | − | 2.63-2.70^{&} | 1 |
| 70 | n.m. | 410 | − | 1.66/1.68^{&} | 10 |
| 71 | n.m. | 412 | − | 2.90/2.96^{&} | 1 |
| 72 | 184.68 | 342 | − | 1.05/1.07^{&} | 1 |
| 73 | n.m. | 326 | − | 1.97/2.01^{&} | 1 |
| 74 | n.m. | 344 | − | 3.06/3.10 | 3 |
| 75 | n.m. | 358 | − | 1.34/1.36^{&} | 1 |
| 76 | n.m. | 360 | − | 3.19/3.23^{&} | 10 |
| 77 | n.m. | 398 | − | 3.09 | 2 |
| 78 | n.m. | 396 | − | 3.31/3.34^{&} | 1 |
| 79 | n.m. | 414 | − | 3.38/3.40^{&} | 1 |
| 80 | n.m. | 358 | − | 1.02 | 1 |
| 81 | n.m. | 376 | − | 2.82/2.87^{&} | 1 |
| 82 | n.m. | 414 | − | 3.06/3.10^{&} | 1 |
| 83 | 245 | 371 | − | 1.57 | 7 |
| 84 | 159.8 | 355 | − | 1.32 | 6 |
| 85 | 235 | 355 | − | 1.39 | 6 |
| 86 | 206.8 | 373 | − | 1.18 | 5 |
| 87 | 245 | 387 | − | 0.83 | 7 |
| 88 | 136.2 | 371 | − | 1.14 | 5 |
| 89 | 128 | 371 | − | 1.17 | 5 |
| 90 | 151.3 | 389 | | 1.19/1.25^{&} | 5 |
| 91 | 261.7 | 425 | − | 1.06 | 5 |
| 92 | n.m. | 409 | 407 | 2.76 | 3 |
| 93 | n.m. | 409 | 407 | 2.76 | 3 |
| 94 | n.m. | 409 | 407 | 2.77 | 3 |
| 95 | n.m. | 409 | 407 | 2.83 | 3 |
| 96 | 211.6 | 428 | − | 1.6 | 5 |
| 97 | n.m. | 342 | − | 0.76 | 1 |
| 98 | n.m. | 344 | − | 1.49/1.51^{&} | 1 |
| 99 | n.m. | 344 | − | 2.20/2.21^{&} | 1 |
| 100 | n.m. | 344 | − | 1.59/1.63^{&} | 1 |
| 101 | n.m. | 358 | − | 1.08 | 1 |
| 102 | n.m. | 360 | − | 2.69/2.71^{&} | 1 |
| 103 | n.m. | 369 | − | 1.36/1.39^{&} | 1 |
| 104 | n.m. | 350 | 348 | 1.71 | 3 |
| 105 | n.m. | 350 | 348 | 1.75 | 3 |
| 106 | n.m. | 350 | 348 | 1.73 | 3 |
| 107 | n.m. | 352 | − | 1.71/1.74^{&} | 1 |
| 108 | n.m. | 352 | − | 2.59 | 3 |
| 109 | n.m. | 352 | − | 2.56 | 3 |
| 110 | n.m. | 343 | − | 0.65 | 1 |
| 111 | n.m. | 343 | 341 | 1.19 | 3 |
| 112 | n.m. | 343 | 341 | 1.17 | 3 |
| 113 | n.m. | 345 | − | 2.18 | 1 |
| 114 | n.m. | 345 | − | 2.18 | 3 |
| 115 | n.m. | 345 | − | 2.19 | 3 |
| 116 | n.m. | 344 | − | 0.75/0.79^{&} | 1 |
| 117 | n.m. | 359 | − | 1.26/1.28^{&} | 1 |
| 118 | n.m. | 374 | − | 2.16 | 1 |
| 119 | 192.2 | 358 | − | 0.578 | 5 |
| 120 | n.m. | 383 | − | 1.92/1.94& | 1 |
| 121 | n.m. | 325 | − | 1.97/2.01& | 1 |
| 122 | n.d. | 396 | − | 1.30 | 1 |
| 123 | n.m. | 396 | − | 2.37 | 1 |
| 124 | n.m. | 328 | − | 1.39 | 1 |
| 125 | n.m. | 342 | − | 1.64 | 1 |
| 126 | n.m. | 356 | − | 1.92 | 1 |
| 127 | n.m. | 366 | − | 1.69 | 1 |
| 128 | n.m. | 358 | 356 | 1.16 | 10 |
| 129 | n.m. | 242 | − | 1.69 | 1 |
| 130 | n.m. | 360 | − | 2.70/2.74& | 1 |
| 131 | n.m. | 358 | − | 1.06 | 1 |
| 132 | n.m. | 359 | − | 1.33 | 10 |
| 133 | n.m. | 346 | − | 2.19 | 1 |
| 134 | n.m. | 372 | − | 1.40 | 1 |
| 135 | n.d. | 342 | − | 0.78 | 1 |
| 136 | n.m. | 356 | − | 0.86 | 1 |
| 137 | n.m. | 360 | − | 2.31/2.35& | 1 |
| 138 | n.m. | 412 | − | 1.65 | 1 |
| 139 | n.m. | 329 | − | 1.31/1.33 | 1 |
| 140 | n.m. | 328 | − | 1.48 | 1 |
| 141 | n.m. | 366 | − | 1.66 | 1 |
| 142 | n.m. | 366 | − | 1.70 | 1 |
| 143 | n.d. | 328 | − | 1.21 | 1 |
| 144 | n.m. | 316 | − | 1.27 | 1 |
| 145 | n.m. | 366 | − | 1.70 | 1 |
| 146 | n.m. | 316 | − | 1.28 | 1 |
| 147 | n.m. | 366 | − | 1.85 | 1 |
| 148 | n.m. | 316 | − | 1.30 | 1 |
| 149 | n.m. | 328 | − | 1.57 | 1 |
| 150 | n.m. | 328 | − | 1.41 | 1 |
| 151 | n.m. | 328 | − | 1.37 | 1 |
| 152 | n.m. | 328 | − | 1.39 | 1 |
| 153 | n.m. | 328 | − | 2.26 | 3 |
| 154 | n.m. | 328 | − | 2.28 | 3 |
| 155 | n.m. | 316 | − | 1.31 | 1 |
| 156 | n.m. | 316 | − | 2.12 | 3 |
| 157 | n.m. | 316 | − | 2.13 | 3 |
| 158 | 158.1 | 368 | − | 0.472 | 5 |
| 159 | n.m. | 356 | − | 0.15 | 1 |
| 160 | n.m. | 340 | − | 1.65 | 1 |
| 161 | n.m. | 343 | − | 2.07 | 1 |
| 162 | n.m. | 342 | − | 1.44 | 1 |
| 163 | n.m. | 342 | − | 0.68 | 1 |
| 164 | n.m. | 331 | − | 1.02 | 1 |
| 165 | n.m. | 325 | − | 0.87 | 11 |

n.d. means not determined,
n.m. means not measured.
^{&}Mixture of diastereomers

### Optical Rotations

Optical rotations were measured on a Perkin-Elmer 341 polarimeter with a sodium lamp and reported as follows: [α]^{o} (λ, c g/100 ml, solvent, T ° C.).

[α]_{λ}^{T}=(100α)/(l× c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation, the concentration and solvent are always provided in parentheses after the rotation. The rotation is reported using degrees and no units of concentration are given (it is assumed to be g/100 mL).

TABLE 3
Optical Rotation data.

| Co. | | Wavelength | Concentration | | Temp. |
|---|---|---|---|---|---|
| No. | α_{D} (°) | (nm) | w/v % | Solvent | (° C.) |
| 4 | +7.5 | 589 | 0.5 | DMF | 20 |
| 5 | −59.0 | 589 | 0.49 | DMF | 20 |
| 6 | −13.9 | 589 | 0.48 | DMF | 20 |
| 10 | +14.7 | 589 | 0.55 | DMF | 20 |
| 11 | −14.8 | 589 | 0.51 | DMF | 20 |
| 14 | −17.2 | 589 | 0.58 | DMF | 20 |
| 15 | −29.9 | 589 | 0.62 | DMF | 20 |
| 16 | −5.2 | 589 | 0.66 | DMF | 20 |
| 28 | −13.3 | 589 | 0.54 | DMF | 20 |
| 29 | −21.3 | 589 | 0.83 | DMF | 20 |
| 36 | +1.4 | 589 | 0.93 | DMF | 20 |
| 37 | −19.8 | 589 | 1.15 | DMF | 20 |
| 41 | +46.5 | 589 | 0.53 | DMF | 20 |
| 46 | −2.3 | 589 | 0.5 | DMF | 20 |
| 54 | −113.6 | 589 | 0.5 | DMF | 20 |
| 135 | −8.4 | 589 | 0.76 | DMF | 20 |
| 153 | −26.2 | 589 | 0.55 | DMF | 20 |
| 154 | +17.7 | 589 | 0.51 | DMF | 20 |
| 156 | −27.0 | 589 | 0.3 | DMF | 20 |
| 157 | +1.4 | 589 | 0.2 | DMF | 20 |
| 159 | −6.4 | 589 | 0.32 | DMF | 20 |
| 161 | −5.6 | 589 | 0.31 | DMF | 20 |
| 162 | −13.1 | 589 | 0.53 | DMF | 20 |

### SFCMS-Methods

### General Procedure for SFC-MS Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 4
Analytical SFC-MS Methods (Flow expressed in mL/min; column
temperature (T) in ° C.; Run time in minutes, Backpressure (BPR) in bars.

| Method code | Column | Mobile phase | Gradient | $\frac{Flow}{Col T}$ | $\frac{Run Time}{BPR}$ |
|---|---|---|---|---|---|
| 1 | Daicel Chiralcel ® OD- 3 column (3 μm, 100 × | A: CO₂ B: | 20% B hold 3 min, | $\frac{3.5}{35}$ | $\frac{3}{103}$ |
| | 4.6 mm) | EtOH(+0.3% | | | |
| | | iPrNH₂) | | | |
| 2 | Daicel Chiralpak ® AD-3 column (3 μm, | A: CO₂ B: | 30% B hold 3 min, | $\frac{3.5}{35}$ | $\frac{3}{103}$ |
| | 100 × 4.6 mm) | iPrOH(+0.3% | | | |
| | | iPrNH₂) | | | |
| 3 | Daicel Chiralpak ® AS- 3 column (3 μm, 100 × | A: CO₂ B: | 10% B hold 3 min, | $\frac{3.5}{35}$ | $\frac{3}{103}$ |
| | 4.6 mm) | MeOH(+0.3% | | | |
| | | iPrNH₂) | | | |
| 4 | Daicel Chiralpak ® IC- 3 column (3 μm, 100 × | A: CO₂ B: | 25% B hold 6 min, | $\frac{3.5}{35}$ | $\frac{6}{103}$ |
| | 4.6 mm) | MeOH(+0.3% | | | |
| | | iPrNH₂) | | | |
| 5 | Daicel Chiralpak ® AD- 3 column (3 μm, 100 × | A: CO₂ B: | 50% B hold 3 min, | $\frac{3.5}{35}$ | $\frac{3}{103}$ |
| | 4.6 mm) | EtOH(+0.3% | | | |
| | | iPrNH₂) | | | |
| 6 | Phenomenex Lux Cellulose 2 | A: CO₂ B: | 20% B hold 6 min, | $\frac{3.5}{35}$ | $\frac{6}{103}$ |
| | column (3 μm, 100 × | iPrOH(+0.3% | | | |
| | 4.6 mm) | iPrNH₂) | | | |
| 7 | Daicel Chiralpak ® AD- 3 column (3 μm, 100 × | A: CO₂ B: | 50% B hold 4 min, | $\frac{3.5}{35}$ | $\frac{4}{103}$ |
| | 4.6 mm) | EtOH(+0.3% | | | |
| | | iPrNH₂) | | | |
| 8 | Daicel Chiralpak ® IC- 3 column (3 μm, 100 × | A: CO₂ B: | 5% B hold 6 min, | $\frac{3.5}{35}$ | $\frac{6}{103}$ |
| | 4.6 mm) | iPrOH(+0.3% | | | |
| | | iPrNH₂) | | | |
| 9 | Daicel Chiralpak ® AD- 3 column (3 μm, 100 × | A: CO₂ B: | 45% B hold 3 min, | $\frac{3.5}{35}$ | $\frac{3}{103}$ |
| | 4.6 mm) | EtOH(+0.3% | | | |
| | | iPrNH₂) | | | |
| 10 | Phenomenex Lux Cellulose 4 | A: CO₂ B: | 30% B hold 3 min, | $\frac{3.5}{35}$ | $\frac{3}{103}$ |
| | column (3 μm, 100 × | MeOH(+0.3% | | | |
| | 4.6 mm) | iPrNH₂) | | | |
| 11 | Daicel Chiralpak ® AD- 3 column (3 μm, 100 × | A: CO₂ B: | 15% B hold 3 min, | $\frac{3.5}{35}$ | $\frac{3}{103}$ |
| | 4.6 mm) | EtOH(+0.3% | | | |
| | | iPrNH₂) | | | |
| | phenomenex Lux amylose2 3 μm, | A: CO2 B: | 15% B hold 3 min, | $\frac{3.5}{35}$ | $\frac{3}{103}$ |
| | 100 × 4.6 mm | EtOH(+0.3% | | | |
| | | iPrNH₂) | | | |

TABLE 5
Analytical SFC data-Rₜ means retention time (in min),
[M + H]⁺ means the protonated mass of the compound,
method refers to the method used for (SFC)MS
analysis of enantiomerically pure compounds.

| | | | | | Isomer |
|---|---|---|---|---|---|
| Co. | | Rt | UV | | Elution |
| No. | [M + H]⁺ | (min) | Area % | Method | Order |
| 3 | 356 | 1.05 | 100.00 | 1 | A |
| 4 | 356 | 1.57 | 98.24 | 1 | B |
| 8 | 370 | 0.82 | 99.25 | 2 | A |
| 9 | 370 | 1.30 | 100.00 | 2 | B |
| 36 | 367 | 0.86 | 100.00 | 3 | A |
| 37 | 367 | 1.07 | 98.04 | 3 | B |
| 50 | 354 | 2.68 | 100.00 | 4 | A |
| 51 | 354 | 3.15 | 100.00 | 4 | B |
| 67 | 372 | 0.77 | 98.80 | 5 | A |
| 68 | 372 | 2.56 | 100.00 | 5 | B |
| 92 | 409 | 1.76 | 100.00 | 5 | C |
| 93 | 409 | 3.01 | 100.00 | 5 | D |
| 94 | 409 | 1.10 | 100.00 | 6 | A |
| 95 | 409 | 1.43 | 98.58 | 6 | B |
| 105 | 350 | 0.84 | 100.00 | 7 | A |
| 106 | 350 | 2.65 | 99.87 | 7 | B |
| 108 | 352 | 2.17 | 96.09 | 8 | A |
| 109 | 352 | 2.44 | 100.00 | 8 | B |
| 111 | 343 | 1.03 | 100.00 | 9 | A |
| 112 | 343 | 2.04 | 99.79 | 9 | B |
| 114 | 345 | 1.36 | 98.33 | 10 | A |
| 115 | 345 | 1.59 | 99.51 | 10 | B |
| 153 | 328 | 0.89 | 100 | 11 | A |
| 154 | 328 | 1.21 | 100 | 11 | B |
| 156 | 316 | 0.96 | 100 | 12 | A |
| 157 | 316 | 1.28 | 100 | 12 | B |

### NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d6 sulfoxide) as solvent. Chemical shifts (6) are reported in parts per million (ppm) relative to tetramethylsilane (TMS), which was used as internal standard.

TABLE 6
¹H NMR results

| Co. | |
|---|---|
| No. | ¹H NMR result |
| 1 | ¹H NMR (500 MHz, CHLOROFORM-d) δ ppm 0.88-1.02 (m, 1 H) 1.43- |
| | 1.54 (m, 1 H) 1.62-1.70 (m, 2 H) 1.74-1.88 (m, 2 H) 1.95-2.03 (m, 1 H) |
| | 2.15 (s, 3 H) 2.33-2.39 (m, 1 H) 2.40-2.45 (m, 1 H) 2.47 (s, 6 H) 2.74 (br d, |
| | J = 10.11 Hz, 1 H) 2.77 (br d, J = 10.98 Hz, 1 H) 3.42-3.60 (m, 2 H) 6.51 |
| | (s, 1 H) 6.72 (s, 2 H) 8.17 (br s, 1 H) |
| 2 | ¹H NMR (500 MHz, CHLOROFORM-d) δ ppm 0.79-0.99 (m, 2 H) 1.22 (t, |
| | J = 7.08 Hz, 3 H) 1.55-1.62 (m, 1 H) 1.64-1.71 (m, 2 H) 1.80-1.88 (m, 1 H) |
| | 1.91-2.00 (m, 1 H) 2.05 (br t, J = 10.11 Hz, 1 H) 2.30-2.38 (m, 1 H) 2.42-2.51 |
| | (m, 7 H) 2.85 (br t, J = 11.41 Hz, 2 H) 3.17 (q, J = 7.22 Hz, 2 H) 3.44-3.59 |
| | (m, 2 H) 5.44-5.57 (m, 1 H) 6.67-6.79 (m, 2 H) |
| 14 | ¹H NMR (500 MHz, CHLOROFORM-d) δ ppm 0.87-0.99 (m, 1 H) 1.38 (t, |
| | J = 7.08 Hz, 3 H) 1.43-1.56 (m, 1 H) 1.60-1.69 (m, 2 H) 1.73-1.79 (m, 1 H) |
| | 1.84 (dddd, J = 13.80, 10.40, 6.86, 3.32 Hz, 1 H) 1.96 (br t, J = 10.55 Hz, |
| | 1 H) 2.32-2.47 (m, 2 H) 2.48 (s, 6 H) 2.70-2.79 (m, 2 H) 3.35-3.49 (m, 2 H) |
| | 4.18 (q, J = 7.13 Hz, 2 H) 5.55 (s, 1 H) 6.72 (s, 2 H) 9.36 (br s, 1 H) |
| 20 | ¹H NMR (400 MHz, CHLOROFORM-d) δ ppm 0.65-0.74 (m, 2 H) 0.83- |
| | 1.00 (m, 3 H) 1.40-1.57 (m, 1 H) 1.40-1.57 (m, 1 H) 1.59-1.69 (m, 2 H) |
| | 1.70-1.78 (m, 1 H) 1.80-2.00 (m, 3 H) 2.30-2.51 (m, 8 H) 2.70-2.81 (m, 2 |
| | H) 3.37-3.53 (m, 2 H) 5.80 (s, 1 H) 6.72 (s, 1 H) |
| 26 | ¹H NMR (400 MHz, CHLOROFORM-d) δ ppm 0.88-1.05 (m, 1 H) 1.42 (t, |
| | J = 7.17 Hz, 3 H) 1.45-1.59 (m, 1 H) 1.60-1.71 (m, 2 H) 1.72-1.95 (m, 2 H) |
| | 2.00 (br t, J = 10.17 Hz, 1 H) 2.33-2.42 (m, 1 H) 2.42-2.54 (m, 7 H) 2.69- |
| | 2.82 (m, 2 H) 3.46-3.60 (m, 2 H) 4.41 (q, J = 7.17 Hz, 2 H) 6.74 (s, 2 H) |
| | 6.87 (s, 1 H) |
| 27 | ¹H NMR (400 MHz, CHLOROFORM-d) δ ppm 1.41-1.95 (m, 6 H) 2.05 (br |
| | s, 2 H) 2.16 (s, 3 H) 2.31-2.45 (m, 2 H) 2.47 (s, 5 H) 2.68-2.80 (m, 2 H) 3.25- |
| | 3.44 (m, 2 H) 6.54-6.64 (m, 2 H) 6.73 (s, 2 H) 8.91 (s, 1 H) |
| 45^{&} | ¹H NMR (400 MHz, CHLOROFORM-d) δ ppm 0.84-0.97 (m, 1 H) 1.31 (dd, |
| | J = 6.70, 1.85 Hz, 3 H) 1.39-1.55 (m, 1 H) 1.55-2.02 (m, 6 H) 2.47 (d, J = |
| | 5.09 Hz, 7 H) 2.60-2.69 (m, 1 H) 2.75 (br d, J = 9.25 Hz, 1 H) 3.41-3.49 (m, |
| | 1 H) 4.02 (s, 3 H) 6.72 (d, J = 12.48 Hz, 2 H) 7.35 (ddd, J = 11.15, 8.26, 1.85 |
| | Hz, 1 H) 7.78 (dd, J = 5.32, 1.85 Hz, 1 H) |
| 52^{&} | ¹H NMR (400 MHz, CHLOROFORM-d) δ ppm 0.81-0.96 (m, 1 H) 1.33 (dd, |
| | J = 6.82, 3.35 Hz, 3 H) 1.38-1.56 (m, 1 H) 1.56-2.03 (m, 6 H) 2.47 (d, J = |
| | 6.70 Hz, 7 H) 2.60-2.85 (m, 2 H) 3.40-3.50 (m, 1 H) 3.93 (s, 3 H) 6.66- |
| | 6.76 (m, 3 H) 7.53 (ddd, J = 8.38, 5.61, 2.43 Hz, 1 H) 8.02 |
| | (dd, J = 6.94, 2.31 Hz, 1 H) |
| 65^{&} | ¹H NMR (400 MHz, CHLOROFORM-d) δ ppm 0.82-0.99 (m, 1 H) 1.35 (dd, |
| | J = 6.82, 4.97 Hz, 3 H) 1.41-1.55 (m, 1 H) 1.56-2.05 (m, 5 H) 2.48 (d, J = |
| | 2.08 Hz, 8 H) 2.62-2.78 (m, 2 H) 3.48-3.62 (m, 1 H) 3.97-4.06 (m, 3 H) 6.73 |
| | (d, J = 6.70 Hz, 2 H) 8.43 (d, J = 6.24 Hz, 2 H) |
| 69^{&} | ¹H NMR (400 MHz, CHLOROFORM-d) δ ppm 0.73-1.01 (m, 2 H) 1.41 (dd, |
| | J = 10.17, 6.94 Hz, 3 H) 1.70-1.92 (m, 1 H) 1.96-2.16 (m, 1 H) 2.24-2.56 |
| | (m, 5 H) 2.93 (br dd, J = 16.18, 8.32 Hz, 3 H) 3.85 (s, 2 H) 3.87-3.93 (m, 6 H) |
| | 4.05-4.18 (m, 1 H) 6.29 (d, J = 3.93 Hz, 1 H) 6.51 (d, J = 1.85 Hz, 1 H) 6.57 |
| | (dd, J = 8.79, 3.01 Hz, 1 H) 7.24-7.31 (m, 1 H) |
| 73^{&} | ¹H NMR (500 MHz, CHLOROFORM-d) δ ppm 1.29-1.46 (m, 4 H) 1.60- |
| | 1.79 (m, 2 H) 1.83-1.90 (m, 1 H) 2.08-2.28 (m, 2 H) 2.48 (d, J = 3.76 Hz, |
| | 6 H) 2.66-2.84 (m, 1 H) 2.96-3.10 (m, 2 H) 3.65-3.76 (m, 1 H) 3.90 (d, |
| | J = 8.67 Hz, 3 H) 6.59 (dd, J = 8.24, 2.46 Hz, 1 H) 6.79 (d, J = 8.38 Hz, 2 H) |
| | 686-6.93 (m, 1 H) 6.86-6.93 (m, 1 H) 7.46-7.60 (m, 1 H) |
| 76^{&} | ¹H NMR (500 MHz, CHLOROFORM-d) δ ppm 1.20-1.37 (m, 1 H) 1.44- |
| | 1.49 (m, 3 H) 1.53-1.89 (m, 3 H) 2.03-2.25 (m, 2 H) 2.33-2.46 (m, 3 H) |
| | 2.60-2.82 (m, 1 H) 2.95-3.22 (m, 2 H) 3.83-3.92 (m, 1 H) 3.85 (s, 1 H) 4.12- |
| | 4.28 (m, 1 H) 6.36 (s, 1 H) 6.53-6.60 (m, 2 H) 7.16-7.34 (m, 1 H) |
| 81^{&} | ¹H NMR (500 MHz, CHLOROFORM-d) δ ppm 1.19-1.49 (m, 4 H) 1.51- |
| | 1.85 (m, 3 H) 1.93-2.33 (m, 3 H) 2.36 (d, J = 4.05 Hz, 3 H) 2.87 (br d, J = |
| | 11.27 Hz, 1 H) 3.11-3.37 (m, 1 H) 3.88 (dd, J = 4.62, 2.31 Hz, 6 H) 4.17-4.44 |
| | (m, 1 H) 6.02 (t, J = 2.17 Hz, 1 H) 6.30 (d, J = 2.02 Hz, 1 H) 6.58 (ddd, J = |
| | 8.74, 4.26, 2.89 Hz, 1 H) 7.22-7.34 (m, 1 H) |
| 97^{&} | ¹H NMR (500 MHz, CHLOROFORM-d) δ ppm 1.37 (br d, J = 6.65 Hz, 1 H) |
| | 1.37 (br d, J = 6.65 Hz, 1 H) 1.40-1.51 (m, 1 H) 1.85-2.01 (m, 1 H) 2.13 (s, 2 |
| | H) 2.17 (td, J = 9.61, 7.37 Hz, 1 H) 2.37-2.51 (m, 1 H) 2.46 (s, 2 H) 2.46 (s, 2 |
| | H) 2.51-2.59 (m, 2 H) 2.62 (td, J = 8.74, 5.92 Hz, 1 H) 2.67 (dd, J = 9.25, |
| | 7.51 Hz, 1 H) 2.74-2.84 (m, 1 H) 3.48-3.59 (m, 1 H) 6.51 (br s, 1 H) 6.72 |
| | (s, 1 H) 6.72 (s, 1 H) 9.18 (br s, 1 H) |
| 101^{&} | ¹H NMR (500 MHz, CHLOROFORM-d) δ ppm 1.45 (br d, J = 6.65 Hz, 1 H) |
| | 1.46 (br d, J = 6.65 Hz, 1 H) 1.48-1.59 (m, 1 H) 1.90-2.07 (m, 1 H) 2.21- |
| | 2.37 (m, 1 H) 2.39 (s, 1 H) 2.40 (s, 1 H) 2.44-2.58 (m, 3 H) 2.59-2.68 (m, 1 |
| | H) 2.69-2.77 (m, 1 H) 2.79-2.94 (m, 1 H) 2.97 (d, J = 4.91 Hz, 3 H) 3.77 (q, |
| | J = 6.65 Hz, 1 H) 3.89 (s, 1 H) 3.89 (s, 1 H) 6.30 (br s, 1 H) 6.31 (s, 1 H) 6.50 |
| | (br s, 1 H) 6.51 (s, 1 H) 6.66 (s, 1 H) 6.90 (br d, J = 4.62 Hz, 1 H) |
| 117^{&} | ¹H NMR (500 MHz, CHLOROFORM-d) δ ppm 1.34 (br d, J = 6.65 Hz, 3 H) |
| | 1.34-1.37 (m, 1 H) 1.46-1.74 (m, 3 H) 1.78-1.89 (m, 1 H) 1.94 (dt, J = 7.66, |
| | 3.68 Hz, 1 H) 2.02-2.09 (m, 1 H) 2.11-2.21 (m, 3 H) 2.30-2.47 (m, 5 H) |
| | 2.49 (s, 1 H) 2.51-2.58 (m, 1 H) 2.58-2.68 (m, 1 H) 2.86-2.98 (m, 1 H) 3.81- |
| | 3.97 (m, 1 H) 5.04 (tt, J = 8.38, 3.90 Hz, 1 H) 5.13 (tt, J = 7.37, 3.76 Hz, 1 H) |
| | 6.53 (s, 1 H) 7.70 (s, 1 H) 7.72 (s, 1 H) 8.04 (s, 1 H) 8.08 (br d, J = 2.02 Hz |
| | 1 H) 9.53-10.52 (m, 1 H) |
| 118^{&} | ¹H NMR (400 MHz, CHLOROFORM-d) δ ppm 0.98-1.12 (m, 1 H) 1.45 (dd, |
| | J = 6.94, 3.47 Hz 3 H) 1.67 (br s, 1 H) 2.00-2.28 (m, 3 H) 2.46 (d, J = |
| | 1.39 Hz, 6 H) 2.90 (br t, J = 12.14 Hz, 1 H) 3.02-3.14 (m, 1 H) 3.77-3.94 (m, |
| | 5 H) 4.12-4.23 (m, 1 H) 6.47 (s, 2 H) 6.57 (ddd, J = 8.67, 5.55, 2.89 Hz 1 H) |
| | 7.21-7.30 (m, 1 H) |
| 122^{&} | ¹H NMR (400 MHz, CHLOROFORM-d) δ ppm 1.38 (d, J = 6.70 Hz, 3 H) |
| | 1.46 (dq, J = 6.65, 6.40 Hz, 1 H) 1.90-2.02 (m, 1 H) 2.13-2.26 (m, 4 H) 2.42- |
| | 2.61 (m, 5 H) 2.62-2.77 (m, 4 H) 3.58 (q, J = 6.70 Hz, 1 H ) 6.51 (br s, 1 H) |
| | 7.11 (s, 1 H) 7.28 (s, 1 H) 7.87 (br s, 1 H) 9.42-10.15 (m, 1 H) |
| 134^{&} | ¹H NMR (400 MHz, CHLOROFORM-d) δ ppm 0.80-0.98 (m, 1 H) 1.32- |
| | 1.43 (m, 3 H) 2.18 (s, 1 H) 2.19 (s, 1 H) 2.65-2.82 (m, 2 H) 3.65-3.82 (m, 2 |
| | H) 3.74-3.93 (m, 1 H) 6.46 (d, J = 3.70 Hz, 1 H) 6.47 (d, J = 3.90 Hz, 1 H) |
| | 6.54 (d, J = 3.50 Hz, 1 H) 6.74 (s, 2 H) 7.95 (br s, 1 H) |
| 136^{&} | ¹H NMR (400 MHz, CHLOROFORM-d) δ ppm 0.77-1.02 (m, 2 H) 1.17- |
| | 1.34 (m, 6 H) 1.39-1.73 (m, 4 H) 1.76-2.04 (m, 3 H) 2.27 (d, J = 7.17 Hz, 1 |
| | H) 2.48 (d, J = 12.25 Hz, 7 H) 2.55-2.76 (m, 2 H) 3.72 (q, J = 6.47 Hz, 1 H) |
| | 6.48 (d, J = 4.39 Hz, 1 H) 6.74 (d, J = 9.48 Hz, 2 H) 10.76 (br s, 1 H) |
| | 13.44 (br s, 1 H) |
| 137^{&} | ¹H NMR (500 MHz, CHLOROFORM-d) δ ppm 1.20-1.41 (m, 1 H) 1.46 (dd, |
| | J = 6.94, 4.62 Hz, 3 H) 1.52-1.86 (m, 2 H) 1.94-2.10 (m, 2 H) 2.11-2.36 (m, |
| | 2 H) 2.45 (d, J = 5.49 Hz, 6 H) 2.90 (br d, J = 11.56 Hz, 1 H) 3.13-3.35 |
| | (m, 1 H) 3.87 (d, J = 6.07 Hz, 3 H) 4.16-4.27 (m, 1 H) 4.29-4.45 (m, 1 H) 6.47 |
| | (d, J = 7.22 Hz, 2 H) 6.47-6.48 (m, 1 H) 6.59 (ddd, J = 8.89, 5.85, 2.89 Hz, |
| | 1 H) 7.19-7.33 (m, 1 H) |

^{&}Mixture of diastereomers

### Pharmacological Examples

### 1) OGA—Biochemical Assay

The assay is based on the inhibition of the hydrolysis of fluorescein mono-β-D-N-Acetyl-Glucosamine (FM-GlcNAc) (Mariappa et al. 2015, Biochem J 470:255) by the recombinant human Meningioma Expressed Antigen 5 (MGEA5), also referred to as O-GcNAcase (OGA). The hydrolysis FM-GlcNAc (Marker Gene technologies, cat #M1485) results in the formation of B-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OnGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GlcNAc at a 100 uM final concentration. Dilutions were prepared in assay buffer.

50 nl of a compound dissolved in DMSO was dispensed on Black Proxiplate TM 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N₄R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting O-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10 fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20×objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 7
Results in the biochemical and cellular assays.

| | | | Cellular | |
|---|---|---|---|---|
| Co. | Enzymatic | Enzymatic | hOGA; | Cellular Eₘₐₓ |
| No. | hOGA; pIC₅₀ | Eₘₐₓ (%) | pEC₅₀ | (%) |
| 1 | 7.67 | 101 | 6.16 | 56 |
| 2 | 5.98 | 93 | | |
| 3 | 8.59 | 102 | 7.32 | 84 |
| 4 | 6.61 | 100 | | |
| 5 | 8.91 | 104 | 7.44 | 80 |
| 6 | 6.76 | 100 | <6 | 11 |
| 7 | 7.58 | 102 | <6 | 13 |
| 8 | 5.16 | 58 | | |
| 9 | 7.91 | 101 | | |
| 10 | 5.88 | 90 | | |
| 11 | 5.87 | 90 | | |
| 12 | <5 | 36 | | |
| 13 | <5 | 7 | | |
| 14 | 5.56 | 81 | | |
| 15 | 5.27 | 65 | | |
| 16 | 7.45 | 101 | 6.05 | 31 |
| 17 | 6.61 | 96 | | |
| 18 | 6.24 | 95 | | |
| 19 | 7.36 | 101 | <6 | 42 |
| 20 | 6.07 | 96 | <6 | 11 |
| 21 | 7.61 | 101 | 6.33 | 59 |
| 22 | 5.18 | 63 | | |
| 23 | 7.22 | 102 | 6.37 | 70 |
| 24 | 5.7 | 84 | | |
| 25 | 5.52 | 82 | | |
| 26 | 5.34 | 71 | | |
| 27 | 5.98 | 89 | | |
| 28 | 7.72 | 100 | 6.51 | 75 |
| 29 | 5.9 | 86 | | |
| 30 | 7.99 | 101 | 6.31 | 60 |
| 31 | 5.19 | 63 | | |
| 32 | 6.31 | 95 | | |
| 33 | 6.63 | 99 | | |
| 34 | 6.31 | 91 | <6 | 4 |
| 35 | 7.87 | 102 | <6 | 23 |
| 36 | 5.3 | 67 | | |
| 37 | 8.08 | 101 | 6 | 40 |
| 38 | 7.15 | 99 | <6 | 3 |
| 39 | 6.58 | 98 | <6 | 11 |
| 40 | 7.94 | 101 | 6.39 | 72 |
| 41 | <5 | 44 | | |
| 42 | 7.88 | 103 | 7.03 | 100 |
| 43 | 7.42 | 101 | 6.03 | 53 |
| 44 | 7.57 | 100 | <6 | 41 |
| 45 | 6.02 | 92 | | |
| 46 | <5 | 12 | | |
| 47 | 7.28 | 102 | 6.29 | 67 |
| 48 | 6.79 | 101 | | |
| 49 | 6.66 | 98 | | |
| 50 | <5 | 16 | | |
| 51 | 7.01 | 102 | | |
| 52 | 6.64 | 98 | | |
| 53 | 6.55 | 99 | | |
| 54 | <5 | 10 | | |
| 55 | 6.52 | 97 | | |
| 56 | 5.02 | 51 | | |
| 57 | <5 | 6 | | |
| 58 | 6.1 | 96 | | |
| 59 | | | | |
| 60 | 5.81 | 88 | | |
| 61 | 5.64 | 84 | | |
| 62 | 6.29 | 94 | | |
| 63 | 5.92 | 93 | <6 | 25 |
| 64 | 6.16 | 95 | <6 | 26 |
| 65 | 6.03 | 93 | | |
| 66 | 7.73 | 102 | 6.7 | 83 |
| 67 | 5.71 | 85 | | |
| 68 | 8.12 | 104 | 7.09 | 86 |
| 69 | 7.12 | 103 | | |
| 70 | 7.28 | 101 | 6.28 | 57 |
| 71 | 7.05 | 104 | | |
| 72 | 6.34 | 97 | <6 | 31 |
| 73 | 6.13 | 96 | | |
| 74 | 7.45 | 102 | 6.17 | 48 |
| 75 | 6.25 | 95 | | |
| 76 | 7.28 | 101 | <6 | 30 |
| 77 | 7.83 | 101 | 6.02 | 49 |
| 78 | 6.45 | 101 | | |
| 79 | 7.36 | 104 | <6 | 8 |
| 80 | 6.99 | 102 | <6 | 7 |
| 81 | 6.25 | 99 | | |
| 82 | 6.05 | 97 | <6 | 6 |
| 83 | 7.22 | 103 | | |
| 84 | <5 | 35 | | |
| 85 | 7.39 | 102 | 7.1 | 74 |
| 86 | 7.77 | 102 | 7.25 | 94 |
| 87 | 7.54 | 103 | | |
| 88 | <5 | 32 | | |
| 89 | 7.6 | 102 | 6.85 | 73 |
| 90 | 7.91 | 102 | 6.93 | 79 |
| 91 | 6.89 | 104 | | |
| 92 | 5.31 | 74 | | |
| 93 | <5 | 29 | | |
| 94 | 5.24 | 72 | | |
| 95 | 7.8 | 103 | <6 | 44 |
| 96 | 7.87 | 104 | <6 | 23 |
| 97 | 6.24 | 94 | | |
| 98 | 5.95 | 92 | | |
| 99 | 6.19 | 91 | | |
| 100 | 6.04 | 97 | <6 | 10 |
| 101 | 5.5 | 75 | | |
| 102 | 5.77 | 85 | | |
| 103 | 5.78 | 85 | | |
| 104 | 5.55 | 82 | | |
| 105 | 5.94 | 89 | | |
| 106 | <5 | 29 | | |
| 107 | 5.03 | 48 | | |
| 108 | <5 | 6 | | |
| 109 | 5.12 | 60 | | |
| 110 | 6.09 | 92 | | |
| 111 | 6.54 | 97 | | |
| 112 | <5 | 20 | | |
| 113 | 5.41 | 74 | | |
| 114 | <5 | 9 | | |
| 115 | 5.72 | 88 | | |
| 116 | 5.08 | 57 | | |
| 117 | 7.19 | 102 | <6 | 30 |
| 118 | 7.06 | 99 | 6.66 | 82 |
| 119 | 5.91 | 92 | | |
| 120 | 5.38 | 75 | | |
| 121 | 6.13 | 96 | | |
| 122 | 6 | 92 | <6 | 17 |
| 123 | 5.55 | 86 | | |
| 124 | 5.06 | 51 | | |
| 125 | 5.38 | 76 | | |
| 126 | 5.02 | 56 | | |
| 127 | 5.14 | 60 | | |
| 128 | 6.24 | 94 | | |
| 129 | 5.36 | 75 | | |
| 130 | 6.06 | 95 | <6 | 4 |
| 131 | 6.42 | 97 | | |
| 132 | 5.11 | 59 | | |
| 133 | 6.12 | 98 | | |
| 134 | 8.33 | 101 | 6.62 | 59 |
| 135 | 6.31 | 98 | | |
| 136 | 6.96 | 102 | <6 | 12 |
| 137 | 6.97 | 102 | <6 | 4 |
| 138 | 5.61 | 81 | | |
| 139 | 5.4 | 73.285 | | |
| 140 | <5 | 29.315 | | |
| 141 | <5 | 35.575 | | |
| 142 | <5 | 30.465 | | |
| 143 | <5 | 38.045 | | |
| 144 | <5 | 43.41 | | |
| 145 | <5 | 44.255 | | |
| 146 | <5 | 33.06 | | |
| 147 | <5 | 14.445 | | |
| 148 | <5 | 11.305 | | |
| 149 | <5 | 30.265 | | |
| 150 | <5 | 45.985 | | |
| 151 | <5 | 11.835 | | |
| 152 | | | | |
| 153 | <5 | 43 | | |
| 154 | <5 | 39 | | |
| 155 | | | | |
| 156 | <5 | 31 | | |
| 157 | <5 | 16 | | |
| 158 | <5 | 13 | | |
| 159 | <5 | 41 | | |
| 160 | <5 | 24 | | |
| 161 | <5 | 52 | | |
| 162 | <5 | 36 | <6 | −6 |
| 163 | <5 | 29 | | |
| 164 | <5 | 26 | | |
| 165 | <5 | 24 | | |

1. A compound of Formula (I) 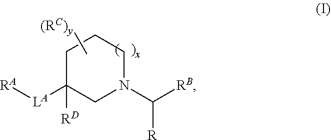
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C₁₋₄alkyl optionally substituted with 1, 2, or 3 independently selected halo substituents; —C(O)NR^{a}R^{aa}; NR^{a}R^{aa}; and
C₁₋₄alkyloxy optionally substituted with 1, 2, or 3 independently selected halo substituents;
wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —O—, —CH₂—, —OCH₂—, —CH₂O—, —NH—, —N(CH₃)—, —NH—CH₂—, and —CH₂—NH—;
x represents 0 or 1;
R is H or CH₃; and
R^{B} is a radical selected from the group consisting of (b-1) to (b-4) 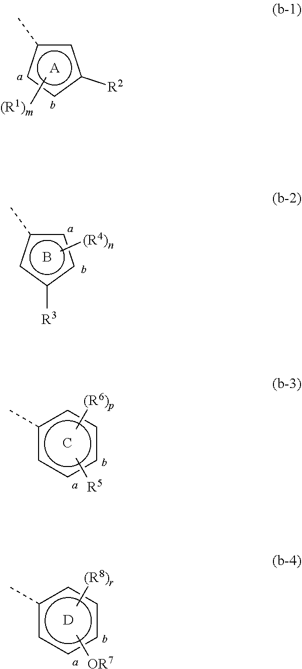
wherein
m, n, p and r each represent 0 or 1;
ring A represents a 5-membered heteroaromatic selected from the group consisting of 1H-pyrazolyl, imidazolyl, isoxazolyl and thienyl;
R¹ when present, is C₁₋₄alkyl, bound at position a or b of the A ring;
R² is selected from the group consisting of C₁₋₄alkyl, C₃₋₆cycloalkyl, —NR^{a}R^{aa}, —NR^{a}COC₁₋₄alkyl, and —CONR^{a}R^{aa}; wherein R^{a} represents hydrogen or C₁₋₄alkyl; and R^{aa} is C₁₋₄alkyl;
ring B represents a 5-membered heteroaromatic selected from the group consisting of oxazolyl, thiazolyl, imidazolyl, 1H-pyrazolyl, isoxazolyl and thienyl; wherein
R³ is —OC₁₋₄alkyl or —C₁₋₄alkyloxyC₁₋₄alkyl;
R⁴ when present, is a halo substituent bound to a carbon atom at position a or b of the B ring, or is a C₁₋₄alkyl substituent bound to a Nitrogen atom at position a or b of the B ring;
rings C and D each represent a 6-membered heteroaromatic selected from the group consisting of pyridinyl, pyridazinyl, pyrazinyl, and pyrimidinyl; wherein
R⁵ is bound at position a or b and is selected from the group consisting of C₁₋₄alkyl optionally substituted with 1, 2, or 3 independently selected halo substituents;
C₃₋₆cycloalkyl; —NR^{b}COC₁₋₄alkyl; and —CONR^{b}R^{bb}; wherein R^{b} represents hydrogen or C₁₋₄alkyl; and R^{bb} is C₁₋₄alkyl;
R⁶, when present, is C₁₋₄alkyl;
OR⁷ is bound at position a or b, wherein R⁷ is C₁₋₄alkyl or C₃₋₆cycloalkyl;
R⁸ when present, is halo or C₁₋₄alkyl, bound to a carbon atom;
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D}; and
c) R^{D} is not hydroxy or methoxy when L^{A} is —O—, —OCH₂—, —CH₂O—, —NH—, —N(CH₃)—, —NHCH₂— or —CH₂NH—;
or a pharmaceutically acceptable addition salt or a solvate thereof.

2. The compound according to claim 1, wherein R^{A} is a heteroaryl radical selected from the group consisting of pyridin-2-yl, pyridin-4-yl, and pyrazin-2-yl, each of which may be optionally substituted with 1, 2 or 3 substituents each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents.

3. The compound according to claim 1, wherein L^{A} is selected from the group consisting of a covalent bond, —O—, —CH₂—, —NH—CH₂—.

4. The compound of claim 1, wherein R^{B} is (b-1), (b-2), (b-3) or (b-4), wherein
m, n, and r each represent 0 or 1;
ring A represents a 5-membered heteroaromatic selected from the group consisting of 1H-pyrazolyl, imidazolyl, and thienyl; wherein
R¹ when present, is C₁₋₄alkyl bound to a Nitrogen atom at position a or b;
R² is selected from the group consisting of C₁₋₄alkyl, C₃₋₆cycloalkyl, —NR^{a}R^{aa}, and —NR^{a}COC₁₋₄alkyl; wherein R^{a} represents hydrogen or C₁₋₄alkyl; and R^{aa} is C₁₋₄alkyl;
ring B represents a 5-membered heteroaromatic selected from the group consisting of oxazolyl, thiazolyl, imidazolyl, 1H-pyrazolyl, and isoxazolyl; wherein
R³ is —OC₁₋₄alkyl or —C₁₋₄alkyloxyC₁₋₄alkyl;
R⁴ when present, is a halo substituent bound to a carbon atom at position a or b or is a C₁₋₄alkyl substituent bound to a Nitrogen atom at position a or b;
rings C and D each represent a 6-membered heteroaromatic selected from the group consisting of pyridinyl, pyridazinyl, pyrazinyl, and pyrimidinyl;
R⁵ is bound at position a or b and is selected from the group consisting of —NR^{b}COC₁₋₄alkyl and —CONR^{b}R^{bb}; wherein R^{b} represents hydrogen or C₁₋₄alkyl; and R^{bb} is C₁₋₄alkyl;
OR⁷ is bound at position a or b, wherein R⁷ is C₁₋₄alkyl; and
R⁸ when present, is a halo or C₁₋₄alkyl substituent, bound to a carbon atom.

5. (canceled)

6. The compound of claim 1, wherein R^{B} is (b-1), (b-2), (b-3a) or (b-4a) 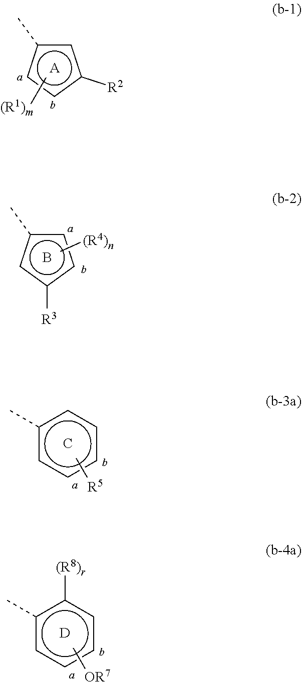
wherein
m, n, and r each represent 0 or 1;
ring A represents a 5-membered heteroaromatic selected from the group consisting of 1H-pyrazolyl, imidazolyl, and thienyl;
R¹ when present, is C₁₋₄alkyl, bound to a Nitrogen atom at position a or b;
R² is selected from the group consisting of C₃₋₆cycloalkyl, and —NR^{a}COC₁₋₄alkyl; wherein R^{a} represents hydrogen or C₁₋₄alkyl; and R^{aa} is C₁₋₄alkyl;
ring B represents a 5-membered heteroaromatic selected from the group consisting of oxazolyl, thiazolyl, and imidazolyl; wherein
R³ is —OC₁₋₄alkyl;
R⁴ when present, is a halo substituent bound to a carbon atom at position a or b, or is a C₁₋₄alkyl substituent bound to a Nitrogen atom at position a or b;
rings C and D each represent pyridinyl; wherein
R⁵ is bound at position a or b and is selected from the group consisting of —NR^{b}COC₁₋₄alkyl and —CONR^{b}R^{bb}; wherein R^{b} represents hydrogen or C₁₋₄alkyl; and R^{bb} is C₁₋₄alkyl;
OR⁷ is bound at position a or b, wherein R⁷ is C₁₋₄alkyl; and
R⁸ when present, is halo, bound to a carbon atom.

7. The compound of claim 1, wherein R^{D} is hydrogen.

8. The compound of claim 1, wherein y is 0.

9. A pharmaceutical composition comprising a prophylactically or a therapeutically effective amount of a compound of claim 1 and a pharmaceutically acceptable carrier.

10. (canceled)

11. (canceled)

12. (canceled)

13. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound of claim 1.

14. (canceled)
